(19) United States
(12) Patent Application Publication
(10) Pub. No.: US 2024/0398920 A1
(43) Pub. Date: Dec. 5, 2024

(54) TRIMERIC ACTIVATABLE CYTOKINE CONSTRUCTS AND RELATED COMPOSITIONS AND METHODS

(71) Applicant: CYTOMX THERAPEUTICS, INC., South San Francisco, CA (US)

(72) Inventors: Erwan LE SCOLAN, South San Francisco, CA (US); Amanda C. KOHLER, South San Francisco, CA (US); Madan M. PAIDHUNGAT, South San Francisco, CA (US); Dylan L. DANIEL, South San Francisco, CA (US); Lauren KADAVY, South San Francisco, CA (US)

(73) Assignee: CYTOMX THERAPEUTICS, INC., South San Francisco, CA (US)

(21) Appl. No.: 18/698,166

(22) PCT Filed: Oct. 12, 2022
(86) PCT No.: PCT/US2022/077943

§ 371 Date: Apr. 3, 2024

Related U.S. Application Data
(60) Provisional application No. 63/255,340, filed on Oct. 13, 2021.

(51) Int. Cl. A61K 39/00; A61P 35/00; C07K 14/525; C07K 14/765

(52) U.S. Cl. CPC A61K 39/001138; A61P 35/00; C07K 14/525; C07K 14/765; C07K 2319/30; C07K 2319/50

(57) ABSTRACT

Provided herein are activatable cytokine constructs that include: a first monomer construct comprising a first cytokine protein (CP1), a first cleavable moiety (CM1), and a first steric masking moiety (SMM1), wherein the CM1 is positioned between the CP1 and the SMM1; a second monomer construct comprising a second cytokine protein (CP2), a second cleavable moiety (CM2), and a second steric masking moiety (SMM2), wherein the CM2 is positioned between the CP2 and the SMM2; and a third monomer construct comprising a third cytokine protein (CP3), a third cleavable moiety (CM3), and a third steric masking moiety (SMM3), wherein the CM3 is positioned between the CP3 and the SMM3, wherein: the CP1, the CP2, and the CP3 bind to one another thereby forming a trimer of the first, the second, and the third monomer constructs; and the SMM1, the SMM2, and the SMM3 are globular molecules.

### CROSS-REFERENCE TO RELATED APPLICATIONS

This application claims the benefit of U.S. Provisional Application No. 63/255,340, filed Oct. 13, 2021. The entire contents of the above-identified application are hereby fully incorporated herein by reference.

### REFERENCE TO AN ELECTRONIC SEQUENCE LISTING

The contents of the electronic sequence listing (CYTX088.xml; Size: 741,530 bytes; and Date of Creation: Oct. 3, 2022) is herein incorporated by reference in its entirety.

### TECHNICAL FIELD

The present disclosure relates to the field of biotechnology, and more specifically, to activatable cytokine constructs.

### BACKGROUND

Cytokines are a family of naturally-occurring small proteins and glycoproteins produced and secreted by most nucleated cells in response to viral infection and/or other antigenic stimuli. LIGHT (lymphotoxin-like inducible protein that competes with glycoprotein D for herpes virus entry on T cells) is a cytokine in the tumor necrosis factor (TNF) ligand superfamily that is expressed on activated T cells, monocytes, granulocytes and immature dendritic cells. LIGHT is also known as tumor necrosis factor superfamily member 14 (TNFSF14). LIGHT is a homotrimer and binds as a trimer to two known cellular receptors: the herpesvirus entry mediator (HVEM) and lymphotoxin-beta receptor (LTbetaR). Through the activation of the HVEM pathway and LT-beta-Receptor, LIGHT can activate T cells and stimulate the production of chemokines, to ultimately recruit T cells. It has been shown to trigger apoptosis of various tumor cells. (Rooney, I A, et. Al., J. Biol. Chem. 275(19):14307-15 (2000)). If expressed in the tumor micro-environment, LIGHT can recruit and activate T-cells in the tumor micro-environment. LIGHT thus has been considered as a therapeutic agent for cancer. However, constitutive LIGHT activity in the peripherial system can lead to lymphocyte activation, inflammation, and tissue destruction. Significant side effects and off-target toxicity have hampered the development of LIGHT as a therapeutic agent.

The need and desire for improved specificity and selectivity of cytokine therapy to the desired target is of great interest. Increased targeting of cytokine therapeutics to the disease site could reduce systemic mechanism-based toxicities and lead to broader therapeutic utility.

### SUMMARY

The present disclosure provides trimeric activatable cytokine constructs and methods of using and making thereof. In one aspect, the present disclosure provides an activatable cytokine construct (ACC) comprising: a first monomer construct comprising a first cytokine protein (CP1), a first cleavable moiety (CM1), and a first steric masking moiety (SMM1), wherein the CM1 is positioned between the CP1 and the SMM1; a second monomer construct comprising a second cytokine protein (CP2), a second cleavable moiety (CM2), and a second steric masking moiety (SMM2), wherein the CM2 is positioned between the CP2 and the SMM2; and a third monomer construct comprising a third cytokine protein (CP3), a third cleavable moiety (CM3), and a third steric masking moiety (SMM3), wherein the CM3 is positioned between the CP3 and the SMM3, wherein: the CP1, the CP2, and the CP3 bind to one another thereby forming a trimer of the first, the second, and the third monomer constructs; and the SMM1, the SMM2, and the SMM3 are globular molecules.

In some embodiments, the CP1, the CP2, and the CP3 are the same cytokine. In some embodiments, the cytokine is a member of tumor necrosis factor or tumor necrosis factor super family. In some embodiments, the CP1, the CP2 and the CP3 are tumor necrosis factor superfamily member 14 (TNFSF14 also known as LIGHT). In some embodiments, each of the CP1, the CP2, and the CP3 comprises a sequence that is at least 80%, 90%, 95%, or 99% identical to SEQ ID NO: 54.

In some embodiments, the SMM1, the SMM2, and the SMM3 are the same globular molecule. In some embodiments, the globular molecule is an albumin. In some embodiments, the albumin is a human serum albumin. In some embodiments, the albumin comprises a sequence that is at least 80%, 90%, 95%, or 99% identical to a human serum albumin.

In some embodiments, the first monomer construct comprises at least one linker. In some embodiments, the at least one linker comprises a linker L1 disposed between the CP1 and the CM1, and/or a linker L2 between the CM1 and the SMM1. In some embodiments, the second monomer construct comprises at least one linker. In some embodiments, the at least one linker comprises a linker L3 disposed between the CP2 and the CM2, and/or a linker L4 between the CM2 and the SMM2. In some embodiments, the third monomer construct comprises at least one linker. In some embodiments, the at least one linker comprises a linker L5 disposed between the CP3 and the CM3, and/or a linker L6 between the CM3 and the SMM3.

In some embodiments, the first monomer construct further comprises a first affinity masking moiety (AMM1) and optionally a fourth cleavable moiety (CM4) positioned between the AMM1 and the CP1, the second monomer construct further comprises a second affinity masking moiety (AMM2) and optionally a fifth cleavable moiety (CM5) positioned between the AMM2 and the CP2, and the third monomer construct further comprises a third affinity masking moiety (AMM3) and optionally a sixth cleavable moiety (CM6) positioned between the AMM3 and the CP3. In some embodiments, the AMM1, the AMM2, and the AMM3 are the same. In some embodiments, each of the AMM1, the AMM2, and the AMM3 comprises a sequence of SEQ ID NO: 61. In some embodiments, each of the AMM1, the AMM2, and the AMM3 comprises a sequence that at least 80%, 90%, 95%, or 99% identical to SEQ ID NO: 61. In some embodiments, the CM1, the CM2, and the CM3 comprise a substrate of the same protease. In some embodiments, the CM1, the CM2, and the CM3 comprise substrates of different proteases. In some embodiments, each of the CM1, the CM2, and the CM3 comprises a sequence that is at least 95% identical to SEQ ID NO: 62 or 63. In some embodiments, the CM4, the CM5, and the CM6 comprise a substrate of the same protease. In some embodiments, the CM4, the CM5, and the CM6 comprise substrates of different proteases. In some embodiments, each of the CM4, the CM5, and the CM6 comprises a sequence that is at least 95% identical to SEQ ID NO: 62 or 63.

In some embodiments, the protease(s) is/are produced by a tumor in a subject. In some embodiments, the protease(s) is/are selected from the group consisting of: ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27, activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, and TMPRSS4.

In some embodiments, the first monomer construct further comprises a linker L7 between the AMM1 and the CM4 and/or a linker L8 between the CM4 and the CP1. In some embodiments, the second monomer construct further comprises a linker L9 between the AMM2 and the CM5 and/or a linker L10 between the CM5 and the CP2. In some embodiments, the third monomer construct further comprises a linker L11 between the AMM3 and the CM6 and/or a linker L12 between the CM6 and the CP3. In some embodiments, each of the linkers L1-L12 has a total length of 2 to 30 amino acids. In some embodiments, each of the linkers L1-L12 independently comprises a sequence of any one of SEQ ID NO: 64-69, 75-77, GGS, SGG, GSG, GS, or G.

In some embodiments, in a N- to C-terminal direction: the first monomer construct comprises the CP1, the CM1, and the SMM1, the second monomer construct comprises the CP2, the CM2, and the SMM2, and the third monomer construct comprises the CP3, the CM3, and the SMM3. In some embodiments, in a N- to C-terminal direction: the first monomer construct comprises the SMM1, the CM1, and the CP1, the second monomer construct comprises the SMM2, the CM2, and the CP2, and the third monomer construct comprises the SMM3, the CM3, and the CP3. In some embodiments, in a N- to C-terminal direction: the first monomer construct comprises the AMM1, the CM4, the CP1, the CM1, and the SMM1, the second monomer construct comprises the AMM2, the CM5, the CP2, the CM2, and the SMM2, and the third monomer construct comprises the AMM3, the CM6, the CP3, the CM3, and the SMM3. In some embodiments, in a N- to C-terminal direction: the first monomer construct comprises the SMM1, the AMM1, the CM1, and the CP1; the second monomer construct comprises the SMM2, the AMM2, the CM2, and the CP2, and the third monomer construct comprises the SMM3, the AMM3, the CM3, and the CP3. In some embodiments, in a N- to C-terminal direction: the first monomer construct comprises, the AMM1, the SMM1, the CM1, and the CP1; the second monomer construct comprises the AMM2, the SMM2, the CM2, and the CP2, and the third monomer construct comprises the AMM3, the SMM3, the CM3, and the CP3. In some embodiments, in a N- to C-terminal direction: the first monomer construct comprises the SMM1, the CM1, the CP1, the CM4, and AMM1, the second monomer construct comprises the SMM2, the CM2, the CP2, the CM5, and AMM2, and the third monomer construct comprises the SMM3, the CM3, the CP3, the CM6, and the AMM3. In some embodiments, in a N- to C-terminal direction: the first monomer construct comprises the CP1, the CM1, the AMM1, and the SMM1; the second monomer construct comprises the CP2, the CM2, the AMM2, and the SMM2; and the third monomer construct comprises the CP3, the CM3, the AMM3, and the SMM3. In some embodiments, in a N- to C-terminal direction: the first monomer construct comprises the CP1, the CM1, the SMM1, and the AMM1; the second monomer construct comprises the CP2, the CM2, the SMM2, and the AMM2; and the third monomer construct comprises the CP3, the CM3, the SMM3, and the AMM3.

In some embodiments, in an inactive state, the ACC is characterized by having a reduced level of an activity of at least one of the CP1, the CP2, the CP3, or the trimer thereof as compared to a control level of the activity of at least one of the CP1, the CP2, the CP3, or the trimer thereof. In some embodiments, the ACC is characterized by at least a 2-fold, 5-fold, 10-fold, 500-fold, 10³-fold, 10⁴-fold, 10⁵-fold, or 10⁶-fold reduction in the activity of the trimer of CP1, CP2, and CP3 as compared to the activity of a control trimer of CP1, CP2, and CP3 that does not comprise a steric masking moiety or an affinity masking moiety. In some embodiments, the activity is activation of herpes virus entry mediator (HVEM). In some embodiments, the activity is activation of lymphotoxin beta receptor. In some embodiments, the activity is activation of herpes virus entry mediator (HVEM) and activation of lymphotoxin beta receptor. In some embodiments, the control trimer of CP1, CP2, and CP3 results from activation of the ACC.

In some embodiments, the first monomer construct, the second monomer construct, and the third monomer construct are identical. In some embodiments, the ACC does not comprise any domain that facilitates formation of a trimer other than CP1, CP2, and CP3. In some embodiments, the ACC does not comprise any domain other than the CP1, the CP2, and the CP3 that covalently links the first, the second, and the third monomer constructs. In some embodiments, the ACC does not comprise a coil-coiled domain, an Fc domain, or a domain other than the CP1, the CP2, or the CP3 that is capable forming a disulfide bond. In some embodiments, the ACC does not comprise a tumor-directing molecule (e.g., the ACC does not comprise an Fab or scFv fragment that recognizes an antigen found on cancer cells). In some embodiments, the CP1, CP2, and CP3 are identical and each comprises the amino acid sequence of SEQ ID NO: 54. In some embodiments, the first monomer construct, the second monomer construct, and the third monomer construct are identical and each monomer comprises: the amino acid sequence of SEQ ID NO: 54; and an AMM comprising an amino acid sequence that is at least 95% identical to that of SEQ ID NO: 61; and an SMM comprising an albumin.

In another aspect, the present disclosure provides an activatable cytokine construct (ACC) comprising: a first monomer construct comprising a first cytokine protein (CP1), a first cleavable moiety (CM1), and a first affinity masking moiety (AMM1), wherein the CM1 is positioned between the CP1 and the AMM1; a second monomer construct comprising a second cytokine protein (CP2), a second cleavable moiety (CM2), and a second affinity masking moiety (AMM2), wherein the CM2 is positioned between the CP2 and the AMM2; and a third monomer construct comprising a third cytokine protein (CP3), a third cleavable moiety (CM3), and a third affinity steric masking moiety (AMM3), wherein the CM3 is positioned between the CP3 and the AMM3, wherein the CP1, the CP2, and the CP3 bind to one another thereby forming a trimer of the first, the second, and the third monomer constructs.

In another aspect, the present disclosure provides a composition comprising the ACC herein. In some embodiments, the composition is a pharmaceutical composition.

In another aspect, the present disclosure provides a container, vial, syringe, injector pen, or kit comprising at least one dose of the composition herein.

In another aspect, the present disclosure provides a nucleic acid encoding a polypeptide that comprises at least one of the first monomer construct, the second monomer construct, or the third monomer construct of the ACC herein. In some embodiments, the nucleic acid comprises a sequence of any one of SEQ ID NOs: 9, 11, 13, 25, 31, 41, 43, 45, or 47.

In another aspect, the present disclosure provides a set of nucleic acids that together encode polypeptides that comprise the first monomer construct, the second monomer construct, and the third monomer construct in the ACC herein.

In another aspect, the present disclosure provides a vector comprising the nucleic acid or a set of nucleic acids herein.

In another aspect, the present disclosure provides a cell comprising the nucleic acid or the vector herein. In some aspects, the cell is a mammalian cell.

In another aspect, the present disclosure provides a method of treating a subject in need thereof comprising administering to the subject a therapeutically effective amount of the ACC or the composition herein. In some embodiments, the subject has been identified or diagnosed as having a cancer. In some embodiments, the method further comprises administering an immune checkpoint inhibitor. In some embodiments, the immune checkpoint inhibitor is an anti-PD-1 antibody or anti-PD-L1 antibody.

In another aspect, the present disclosure provides a method of producing an ACC comprising: culturing a cell of claim 61 in a liquid culture medium under conditions sufficient to produce the ACC; and recovering the ACC from the cell or the liquid culture medium. In some embodiments, the method further comprises purifying the recovered ACC using affinity chromatography. In some embodiments, the method further comprises formulating the recovered ACC into a pharmaceutical composition.

### BRIEF DESCRIPTION OF DRAWINGS

FIGS. 1A-1H show exemplary activatable cytokine constructs according to some embodiments of the present disclosure. FIGS. 1A-1B illustrate exemplary cytokine constructs that utilize human serum albumin (HSA) as a steric masking moiety. FIGS. 1C-1D illustrate exemplary cytokine constructs that utilize a peptide masking moiety. FIGS. 1E-1H illustrate exemplary cytokine constructs that utilize both a steric masking moiety and a peptide masking moiety. FIGS. 1E-1F illustrate exemplary cytokine constructs that have a steric masking moiety and an affinity masking moiety coupled to different sides of the cytokine component. FIGS. 1G-1H illustrate exemplary cytokine constructs that have a steric masking moiety and an affinity masking moiety coupled to the same side of the cytokine component.

FIGS. 2A-2B show that the addition of HSA steric masking moiety to LIGHT reduces LIGHT signaling activity in both the herpes virus entry mediator (HVEM) reporter assay and Lymphotoxin beta receptor cell-based assay (A375 IL-8 ELISA).

FIGS. 3A-3B show that the addition of cleavable peptide mask to LIGHTreduces LIGHT signaling activity in both the HVEM reporter assay and A375 IL-8 ELISA.

FIGS. 4A-4C show that the addition of a cleavable peptide mask to the N-terminus of LIGHT-HSA further reduces LIGHT signaling activity in both the HVEM reporter assay and A375 IL-8 ELISA.

FIGS. 5A-5B show in vitro activity of single and dual masked LIGHT activatable cytokine constructs in the HVEM reporter assay and A375 IL-8 ELISA.

FIGS. 6A and 6B show in vitro activity of additional ACCs.

FIGS. 7A-7D show in vitro activity of mouse-human cross-reactive LIGHT activatable cytokine constructs.

FIGS. 8A-8B show the activity of ProC1486, ProC1487 and ProC2076 determined by HVEM assays.

FIG. 9 shows tumor growth inhibition profiles of human LIGHT ACC ProC1491 engineered with a cleavable HSA domain at the C-terminal extremity of LIGHT and human LIGHT ProC1 189 engineered with a His tag at the C-terminal extremity of LIGHT.

FIGS. 10A-10B show antitumor activity of ProC1486 and ProC1487 in the MC38 syngeneic mouse model.

FIGS. 11A-11B show that at day 6 post treatment initiation in the MC38 syngeneic mice model, ProC1487, dosed as a single agent or in combination with an anti-PD-1 antibody, was able to increase the level of CD8+ T cells in the tumor micro-environment (FIG. 11A) as well as to promote the production of Th1 cytokines (IFNγ, TNFα) by CD8+ T cells (FIG. 11B).

### DETAILED DESCRIPTION

Provided herein are trimeric activatable cytokine constructs (ACCs) that include trimers of three monomer constructs. Each of the monomer constructs includes a cytokine, which can bind to one another and form a trimer (e.g., homotrimer or heterotrimer). Upon activation, the active cytokine products remain in a trimeric form.

Each of the monomer constructs may further comprise one or more masking moieties, coupled with the cytokine via one or more cleavable moieties. In some embodiments, the masking moieties may be steric masking moieties that do not bind to the cytokine, but, in an inactive state, reduce, inhibit, or interfere with binding between the cytokine and its binding partner (e.g., a ligand or receptor) via steric hindrance. In some embodiments, the masking moieties may be affinity masking moieties that specifically bind to the cytokine and reduce, inhibit, or interfere with binding between the cytokine and its binding partner in an inactivate state. In some embodiments, each monomer construct of the trimeric ACC comprises a steric masking moiety. In some examples, each monomer construct may be dual masked and include both a steric masking moiety and an affinity masking moiety. In such monomer constructs, the steric masking moiety and the affinity masking moiety may be coupled to different sides of the cytokine, with each masking moiety coupled to the cytokine with its own cleavable moiety. Alternatively, the steric masking moiety and the affinity masking moiety may be on the same side of the cytokine in the monomer construct. In such cases, the masking moieties may be coupled with the cytokine via one cleavable moiety (e.g., positioned between the cytokine and the masking moiety closer to the cytokine).

In an active state (e.g., when the ACC is exposed to a protease that cleaves the cleavable moieties), the one or more masking moieties may be released from the cytokines, yielding a cytokine product with substantially restored activity. In the active state, the cytokines may be in a trimeric form. The ACC may be designed to selectively activate upon exposure to diseased tissue, and not in normal tissue. For example, the ACC may be designed with one or more cleavable moieties (CMs) that are cleaved by a protease. The protease(s) that cleave the one or more CMs may be over-expressed in diseased tissue (e.g., tumor tissue) relative to healthy tissue. The ACC may be activated upon cleavage of the CM(s) so that the cytokine may exert its activity in the diseased tissue (e.g., in a tumor microenvironment) while the cytokine activity is attenuated in the context of healthy tissue. Thus, the ACCs provided herein may provide reduced toxicity relative to traditional cytokine therapeutics, enable higher effective dosages of cytokine, and/or increase the therapeutic window for the cytokine. As such, these compounds have the potential for conferring the benefit of a cytokine-based therapy, with potentially less of the toxicity associated with certain cytokine-based therapies.

Also provided herein are related intermediates, compositions, kits, nucleic acids, vectors, and recombinant cells, as well as related methods, including methods of using and methods of producing any of the activatable cytokine constructs described herein. Provided herein are ACCs produced by any one of the methods described herein. Also provided herein are compositions comprising any one the ACCs described herein. Also provided herein are compositions of any one of the compositions described herein, wherein the composition is a pharmaceutical composition. Also provided herein are kits comprising at least one dose of any one of the compositions described herein.

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

The term “a” and “an” refers to one or more (i.e., at least one) of the grammatical object of the article. By way of example, “a cell” encompasses one or more cells.

As used herein, the terms “about” and “approximately,” when used to modify an amount specified in a numeric value or range, indicate that the numeric value as well as reasonable deviations from the value known to the skilled person in the art. For example ±20%, ±10%, or ±5%, are within the intended meaning of the recited value where appropriate.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of” would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain molecules, constructs, compositions, elements, moieties, excipients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of constructs, molecules, method steps, kits, or compositions described with respect to a construct, composition, or method is intended to and does find direct support for embodiments related to constructs, compositions, formulations, and methods described in any other part of this disclosure, even if those method steps, active agents, kits, or compositions are not re-listed in the context or section of that embodiment or aspect.

Unless otherwise specified, a “nucleic acid sequence encoding a protein” includes all nucleotide sequences that are degenerate versions of each other and thus encode the same amino acid sequence.

The term “N-terminally positioned” when referring to a position of a first domain or sequence relative to a second domain or sequence in a polypeptide primary amino acid sequence means that the first domain or sequence is located closer to the N-terminus of the polypeptide primary amino acid sequence than the second domain or sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain or sequence is located closer to the C-terminus of the polypeptide primary amino acid sequence than the second domain or sequence. In some embodiments, there may be additional sequences and/or domains between the first domain or sequence and the second domain or sequence.

The term “exogenous” refers to any material introduced from or originating from outside a cell, a tissue, or an organism that is not produced by or does not originate from the same cell, tissue, or organism in which it is being introduced.

The term “transduced,” “transfected,” or “transformed” refers to a process by which an exogenous nucleic acid is introduced or transferred into a cell. A “transduced,” “transfected,” or “transformed” cell (e.g., mammalian cell) is one that has been transduced, transfected, or transformed with exogenous nucleic acid (e.g., a vector) that includes an exogenous nucleic acid encoding any of the activatable cytokine constructs described herein.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), alphatic family (e.g., alanine, valine, leucine and isoleucine), aromatic family (e.g., phenylalanine, tryptophan, and tyrosine).

As used herein the phrase “specifically binds,” or “immunoreacts with” means that a protein or protein complex reacts with one or more binding partners and does not react with other polypeptides, or binds at much lower affinity, e.g., about or greater than 10⁻⁶ M.

The term “treatment” refers to ameliorating at least one symptom of a disorder. In some embodiments, the disorder being treated is a cancer and to ameliorate at least one symptom of a cancer.

### Activatable Cytokine Constructs

In one aspect, the present disclosure provides activatable cytokine constructs (ACCs) that include three monomer constructs forming a trimer through their cytokine components. Each of the monomer construct may comprise a cytokine protein (CP), one or more masking moieties (MMs), and one or more cleavable moieties (CMs) positioned between the MMs and the CP. In some embodiments, the MMs may be steric masking moieties (SMMs). In some embodiments, the MMs may be affinity masking moieties (AMMs). In some embodiments, the MMs may include both SMMs and AMMs. In some embodiments, the ACC does not include any covalent bonds between the monomeric units that form the trimer. In some embodiments, the ACC does not include any domain, other than the cytokine itself, that promotes formation of trimers. In some embodiments, the ACC does not include any domain, other than the cytokine itself, that reinforces the formation of trimers. For example, in some embodiments, the ACC may not comprise any domain other than the CP1, the CP2, and the CP3 that covalently links the first, the second, and the third monomer constructs. In some examples, the ACC does not comprise a coil-coiled domain, an Fc domain, or a domain other than the CP1, the CP2, or the CP3 that is capable forming a disulfide bond across separate monomers.

Upon activation, the CMs may be cleaved and the MMs may be released from the ACC, resulting in an active cytokine product. The active cytokine product may remain in a trimeric form, e.g., comprising a trimer formed by the three CPs.

In a specific embodiment, provided herein is an activatable cytokine construct (ACC) that includes a first monomer construct, a second monomer construct, and a third monomer construct, wherein:
the first monomer construct comprises a first cytokine protein (CP1), a first cleavable moiety (CM1), and a first steric masking moiety (SMM1), wherein the CM1 is positioned between the CP1 and the SMM1;
the second monomer construct comprising a second cytokine protein (CP2), a second cleavable moiety (CM2), and a second steric masking moiety (SMM2), wherein the CM2 is positioned between the CP2 and the SMM2; and
the a third monomer construct comprising a third cytokine protein (CP3), a third cleavable moiety (CM3), and a third steric masking moiety (SMM3), wherein the CM3 is positioned between the CP3 and the SMM3.

The CP1, the CP2, and CP3 may bind to one another (e.g., by covalent or non-covalent bonding) thereby forming a trimer of the first, the second, and the third monomer constructs. In some embodiments, each of the SMM1, the SMM2, and the SMM3 is a globular molecule. In one example, the SMM1, the SMM2 and the SMM3 are the same globular molecule (e.g., human serum albumin). In some examples the CP1, the CP2, and the CP3 are tumor necrosis factor superfamily member 14 (TNFSF14, also known as LIGHT).

The ACC may comprise a linker between two of the components described herein. In some examples, the first monomer construct comprises at least one linker, e.g., a linker L1 disposed between the CP1 and the CM1, and/or a linker L2 between the CM1 and the SMM1. In some examples, the second monomer construct comprises at least one linker, e.g., a linker L3 disposed between the CP2 and the CM2, and/or a linker L4 between the CM2 and the SMM2. In some examples, the third monomer construct comprises at least one linker, e.g., a linker L5 disposed between the CP3 and the CM3, and/or a linker L6 between the CM3 and the SMM3.

In some embodiments, the first monomer construct may further comprise a first affinity masking moiety (AMM1) and a fourth cleavable moiety (CM4) positioned between the AMM1 and the CP1, the second monomer construct may further comprise a second affinity masking moiety (AMM2) and a fifth cleavable moiety (CM5) positioned between the AMM2 and the CP2, and the third monomer construct may further comprise a third affinity masking moiety (AMM3) and a sixth cleavable moiety (CM6) positioned between the AMM3 and the CP3.

In some examples, the first monomer construct may further comprise a linker L7 between the AMM1 and the CM4 and/or a linker L8 between the CM4 and the CP1. In some examples, the second monomer construct further comprise a linker L9 between the AMM2 and the CM5 and/or a linker L10 between the CM5 and the CP2. In some examples, the third monomer construct may further comprise a linker L11 between the AMM3 and the CM6 and/or a linker L12 between the CM6 and the CP3.

In another specific embodiment, provided herein is an activatable cytokine construct (ACC) that includes a first monomer construct, a second monomer construct, and a third monomer construct, wherein
a first monomer construct comprising a first cytokine protein (CP1), a first cleavable moiety (CM1), and a first affinity masking moiety (AMM1), wherein the CM1 is positioned between the CP1 and the AMM1;
a second monomer construct comprising a second cytokine protein (CP2), a second cleavable moiety (CM2), and a second affinity masking moiety (AMM2), wherein the CM2 is positioned between the CP2 and the AMM2; and
a third monomer construct comprising a third cytokine protein (CP3), a third cleavable moiety (CM3), and a third affinity steric masking moiety (AMM3), wherein the CM3 is positioned between the CP3 and the AMM3.

The CP1, the CP2, and CP3 may bind to one another thereby forming a trimer of the first, the second, and the third monomer constructs.

The ACC may further comprise one or more spacers, which are an amino acid residue or a peptide incorporated at a free terminus of the mature ACC, for example between the signal peptide and the N-terminus of the mature ACC. In some aspects, a spacer (or “header”) may contain glutamine (Q) residues. In some aspects, residues in the spacer minimize aminopeptidase and/or exopeptidase action to prevent cleavage of N-terminal amino acids. Illustrative and non-limiting spacer amino acid sequences may comprise or consist of any of the following exemplary amino acid sequences: QGQSGS (SEQ ID NO: 76); GQSGS (SEQ ID NO: 1); QSGS (SEQ ID NO: 70); SGS; GS; S; QGQSGQG (SEQ ID NO: 71); GQSGQG (SEQ ID NO: 72); QSGQG (SEQ ID NO: 73); SGQG (SEQ ID NO: 74); GQG; QG; G; QGQSGQ (SEQ ID NO: 80); GQSGQ (SEQ ID NO: 136); QSGQ (SEQ ID NO: 137); QGQSG (SEQ ID NO: 138); QGQS (SEQ ID NO: 139); SGQ; GQ; and Q. In some embodiments, spacer sequences may be omitted.

The term “activatable” when used in reference to a cytokine construct, refers to a cytokine construct that exhibits a first level of one or more activities, whereupon exposure to a condition that causes cleavage of at least one cleavable moiety results in the generation of a cytokine construct that exhibits a second level of the one or more activities, where the second level of activity is greater than the first level of activity. Non-limiting examples of an activities include any of the exemplary activities of a cytokine (e.g., a TNF or TNF sumper family member) described herein or known in the art.

The terms “masking moiety” and “MM” are used interchangeably herein to refer to an amino acid sequence that reduces or inhibits one or more activities of a cytokine protein. In some embodiments, the MM may be a steric masking moiety (SMM), which does not specifically bind to the CP, but rather interferes with CP's binding to its binding partner through steric hindrance. For example, the SMM may be positioned in the uncleaved ACC such that the tertiary or quaternary structure of the ACC allows the SMM to mask the CP through positioning between the SMM and CP and/or charge-based interaction, thereby holding the SMM in place to interfere with binding partner access to the CP. In some embodiments, the MM may be an affinity masking moiety (AMM), which interacts with the CP, thus reducing, inhibiting, or interfering the interaction between the CP and its binding partner. In some embodiments, the AMM may be a peptide mask (“PM”).

The terms “peptide mask” and “PM” are used interchangeably herein to refer to an amino acid sequence of less than 50 amino acids that reduces or inhibits one or more activities of a cytokine protein. The PM may bind to the cytokine and limit the interaction of the cytokine with its receptor. In some embodiments, the PM is no more than 40 amino acids in length. In preferred embodiments, the PM is no more than 20 amino acids in length. In some embodiments, the PM is no more than 19, 18, 17, 16, or 15 amino acids in length.

As used herein, the term “masking efficiency” refers to the activity (e.g., EC50) of the uncleaved ACC divided by the activity of a control cytokine, wherein the control cytokine may be either cleavage product of the ACC or the cytokine used as the CP of the ACC. An ACC having a reduced level of at least one of the CP activity has a masking efficiency that is greater than 10. In some embodiments, the ACCs described herein have a masking efficiency that is greater than 10, greater than 100, greater than 1000, or greater than 5000. Illustrative assays for determining masking efficiency include those described in Example 1.

The terms “cleavable moiety” and “CM” are used interchangeably herein to refer to a peptide, the amino acid sequence of which comprises a substrate for a sequence-specific protease. Cleavable moieties that are suitable for use in the ACC herein include any of the protease substrates that are known the art. Exemplary cleavable moieties are described in more detail below.

As used herein, a polypeptide, such as a cytokine or the steric masking moiety (e.g., albumin such as human serum albumin), may be a wild-type polypeptide (e.g., a naturally-existing polypeptide) or a variant of the wild-type polypeptide. A variant may be a polypeptide modified by substitution, insertion, deletion and/or addition of one or more amino acids of the wild-type polypeptide, provided that the variant retains the basic function or activity of the wild-type polypeptide. In some examples, a variant may have altered (e.g., increased or decreased) function or activity comparing with the wild-type polypeptide. In some aspects, the variant may be a functional fragment of the wild-type polypeptide. The term “functional fragment” means that the sequence of the polypeptide (e.g., cytokine) may include fewer amino acids than the full-length polypeptide sequence, but sufficient polypeptide chain length to confer activity (e.g., cytokine activity).

As used herein, the term “linker” refers to a peptide, the amino acid sequence of which is not a substrate for a protease. A linker may comprise a stretch of amino acid sequence that links two components in the ACC. Exemplary linkers are described in more detail below.

The organization of the components in each of the first, the second, and the third monomer constructs may be arranged in the same order in each monomer construct. In some embodiments, the organization of the components in each of the first, the second, and the third monomer constructs may be arranged in different orders in each monomer construct. In some examples, the corresponding components in each monomer construct (e.g., CP1, CP2, and CP3; or SMM1, SMM2, and SMM3; CM1, CM2, and CM3; AMM1, AMM2, and AMM3; or CM4, CM5, CM6) may be the same in terms of, for example, molecular weights, sizes, amino acid sequences, and the like. In some examples, the corresponding components in each monomer construct (e.g., CP1, CP2, and CP3; or SMM1, SMM2, and SMM3; CM1, CM2, and CM3; AMM1, AMM2, and AMM3; or CM4, CM5, CM6) may be different in terms of, for example, molecular weights, sizes, amino acid sequences, and the like. Thus, the trimeric ACC may have symmetrical or asymmetrical monomer construct components.

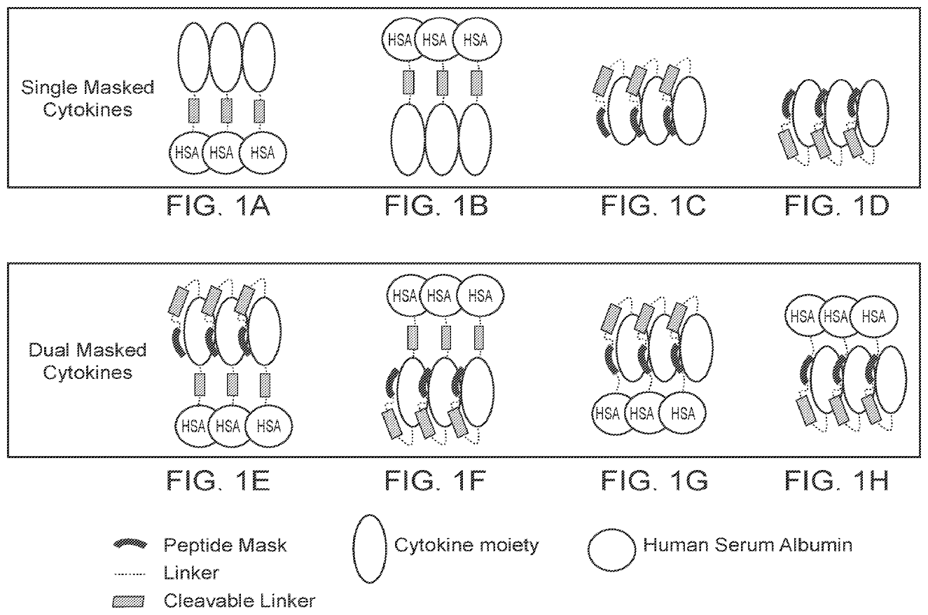

In some embodiments, in an N- to C-terminal direction, the first monomer construct comprises the CP1, the CM1, and the SMM1, the second monomer construct comprises the CP2, the CM2, and the SMM2, and the third monomer construct comprises the CP3, the CM3, and the SMM3. An example of such ACCs is shown in FIG. 1A.

In some embodiments, in an N- to C-terminal direction, the first monomer construct comprises the SMM1, the CM1, and the CP1, the second monomer construct comprises the SMM2, the CM2, and the CP2, and the third monomer construct comprises the SMM3, the CM3, and the CP3. An example of such ACCs is shown in FIG. 1B.

In some embodiments, in an N- to C-terminal direction, the first monomer construct comprises the AMM1, the CM1, and the CP1, the second monomer construct comprises the AMM2, the CM2, and the CP2, and the third monomer construct comprises the AMM3, the CM3, and the CP3. An example of such ACCs is shown in FIG. 1C.

In some embodiments, in an N- to C-terminal direction, the first monomer construct comprises the CP1, the CM1, and the AMM1, the second monomer construct comprises the CP2, the CM2, and the AMM2, and the third monomer construct comprises the CP3, the CM3, and the AMM3. An example of such ACCs is shown in FIG. 1D.

In some embodiments, in an N- to C-terminal direction, the first monomer construct comprises the AMM1, the CM4, the CP1, the CM1, and the SMM1, the second monomer construct comprises the AMM2, the CM5, the CP2, the CM2, and the SMM2, and the third monomer construct comprises the AMM3, the CM6, the CP3, the CM3, and the SMM3. An example of such ACCs is shown in FIG. 1E.

In some embodiments, in an N- to C-terminal direction, the first monomer construct comprises the SMM1, the CM1, the CP1, the CM4, and AMM1, the second monomer construct comprises the SMM2, the CM2, the CP2, the CM5, and AMM2, and the third monomer construct comprises the SMM3, the CM3, the CP3, the CM6, and the AMM3. An example of such ACCs is shown in FIG. 1F.

In some embodiments, the AMM and SMM may be on the same side relative to the CP in the monomer construct. The AMM and the SMM may be coupled with the CP with a CM between the CP and the MM closer to the CP. The cleavage of the CM may release both the AMM and SMM from the CP, resulting in the active cytokine product.

In some embodiments, in an N- to C-terminal direction, the first monomer construct comprises the CP1, the CM1, the AMM1, and the SMM1; the second monomer construct comprises the CP2, the CM2, the AMM2, and the SMM2; and the third monomer construct comprises the CP3, the CM3, the AMM3, and the SMM3. An example of such ACCs is shown in FIG. 1H.

In some embodiments, in an N- to C-terminal direction, the first monomer construct comprises the CP1, the CM1, the SMM1, and the AMM1; the first monomer construct comprises the CP2, the CM2, the SMM2, and the AMM2; and the first monomer construct comprises the CP3, the CM3, the SMM3, and the AMM3.

In some embodiments, in an N- to C-terminal direction, the first monomer construct comprises the SMM1, the AMM1, the CM1, and the CP1; the second monomer construct comprises the SMM2, the AMM2, the CM2, and the CP2, and the third monomer construct comprises the SMM3, the AMM3, the CM3, and the CP3. An example of such ACCs is shown in FIG. 1G.

In some embodiments, in an N- to C-terminal direction, the first monomer construct comprises, the AMM1, the SMM1, the CM1, and the CP1; the second monomer construct comprises the AMM2, the SMM2, the CM2, and the CP2, and the third monomer construct comprises the AMM3, the SMM3, the CM3, and the CP3.

In some embodiments, the ACC may be characterized by a reduction in an activity of at least one of the CP1, the CP2, the CP3, or the trimer thereof, as compared to a control level of an activity of at least one of the CP1, the CP2, the CP3, or the trimer thereof. In some embodiments, a control level can be the level of the activity for a recombinant CP1, CP2, CP3, or the trimer thereof (e.g., a commercially available recombinant CP1, CP2, CP3, or the trimer thereof, a recombinant wild type CP1, CP2, CP3, or the trimer thereof, and the like). In some embodiments, a control level can be the level of the activity of a cleaved (activated) form of the ACC.

In some embodiments, the binding affinity (K_{D}) of the CP1, CP2, CP3, or the trimer thereof for its binding partner (e.g., a cognate receptor) may be determined using surface plasmon resonance (e.g., performed in phosphate buffered saline at 25° C.). In certain embodiments, the activity may be the level of herpes virus entry mediator (HVEM) activation (e.g., as evaluated using HVEM cell-based assay described in the Example section below). In some embodiments, the activity may be the capability of stimulating the production of IL-5 when engaging the lymphotoxin beta receptor on the surface of the A375 human melanoma cell line (e.g., as evaluated using Lymphotoxin beta receptor cell-based assay as described in the Example section below). In some examples, the ACC shows a reduced activity in the activation of herpes virus entry mediator (HVEM) compared to a control trimer of the CP1, CP2, and CP3. In some examples, the ACC shows a reduced activity in the activation of lymphotoxin beta receptor compared to a control trimer of the CP1, CP2, and CP3. In some examples, the ACC shows a reduced activity in the activation of herpes virus entry mediator (HVEM) and the activation of lymphotoxin beta receptor compared to a control trimer of the CP1, CP2, and CP3. In some examples, the control trimer of CP1, CP2, and CP3 results from activation of the ACC.

In some embodiments, the ACC is characterized by at least a 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 10⁴-fold, 10⁵-fold, 10⁶-fold, 10⁷-fold, or 10⁸-fold reduction in an activity of at least one of the CP1, the CP2, the CP3, or the trimer thereof as compared to the control level.

In some embodiments, the ACC is characterized by a 1- to 20-fold reduction, a 200- to 500-fold reduction, a 300- to 500-fold reduction, a 400- to 500-fold reduction, a 500- to 600-fold reduction, a 600- to 700-fold reduction, a 150- to 1000-fold reduction, a 100- to 1500-fold reduction, a 200- to 1500-fold reduction, a 300- to 1500-fold reduction, a 400- to 1500-fold reduction, a 500- to 1500-fold reduction, a 1000- to 1500-fold reduction, a 100- to 1000-fold reduction, a 200- to 1000-fold reduction, a 300- to 1000-fold reduction, a 400- to 1000-fold reduction, a 500- to 1000-fold reduction, a 100- to 500-fold reduction, a 20- to 50-fold reduction, a 30- to 50-fold reduction, a 40- to 50-fold reduction, a 100- to 400-fold reduction, a 200- to 400-fold reduction, or a 300- to 400-fold reduction, a 100- to 300-fold reduction, a 200- to 300-fold reduction, or a 100- to 200-fold reduction in in an activity of at least one of the CP1, the CP2, the CP3, or the trimer thereof as compared to the control level.

In some embodiments, the control level of the activity of the CP1, the CP2, the CP3, or the trimer thereof is the activity of the CP1, the CP2, the CP3, or the trimer thereof released from the ACC following cleavage of CMs by the protease(s) (the “cleavage product”). In some embodiments, the control level of the at least one activity of the CP1, the CP2, the CP3, or the trimer thereof is the activity of a corresponding wild type mature cytokine (e.g., recombinant wild type mature cytokine) or the trimer thereof.

In some embodiments, incubation of the ACC with the protease yields an activated cytokine product(s), where the activity of the CP1, the CP2, the CP3, or the trimer thereof is greater than the one or more activities of the CP1, the CP2, the CP3, or the trimer thereof of the intact ACC (uncleaved). In some embodiments, the activity of the CP1, the CP2, the CP3, or the trimer thereof is at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, or 1000-fold, 10⁴-fold, 10⁵-fold, 10⁶-fold, 10⁷-fold, or 10⁸-fold greater than the activity of the CP1, the CP2, the CP3, or the trimer thereof of the ACC. In some embodiments, the activity of the CP1, the CP2, the CP3, or the trimer thereof is 1- to 20-fold greater, a 200- to 500-fold greater, a 300- to 500-fold greater, a 400- to 500-fold greater, a 500- to 600-fold greater, a 600- to 700-fold greater, a 150- to 1000-fold greater, a 100- to 1500-fold greater, a 200- to 1500-fold greater, a 300- to 1500-fold greater, a 400- to 1500-fold greater, a 500- to 1500-fold greater, a 1000- to 1500-fold greater, a 100- to 1000-fold greater, a 200- to 1000-fold greater, a 300- to 1000-fold greater, a 400- to 1000-fold greater, a 500- to 1000-fold greater, a 100- to 500-fold greater, a 20- to 50-fold greater, a 30- to 50-fold greater, a 40- to 50-fold greater, a 100- to 400-fold greater, a 200- to 400-fold greater, or a 300- to 400-fold greater, a 100- to 300-fold greater, a 200- to 300-fold greater, or a 100- to 200-fold greater than the activity of CP1, the CP2, the CP3, or the trimer thereof of the ACC.

In some embodiments, each of the first, the second, and/or the third monomer construct may independently comprise a total of about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 800 amino acids, about 250 amino acids to about 750 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 850 amino acids, about 650 amino acids to about 800 amino acids, about 650 amino acids to about 750 amino acids, about 650 amino acids to about 700 amino acids, about 700 amino acids to about 850 amino acids, about 700 amino acids to about 800 amino acids, about 700 amino acids to about 750 amino acids, about 750 amino acids to about 800 amino acids, or about 800 amino acids to about 850 amino acids.

In some embodiments, one or more monomer constructs in an ACC may comprise a sequence of any one of SEQ ID NOs: 8, 10, 12, 24, 30, 40, 42, 44, or 46. In some embodiments, one or more monomer constructs in an ACC may comprise a sequence that is at least 80% (e.g., at least 82%, at least 84%, at least 86%, at least 88%, at least 90%, at least 92%, at least 94%, at least 96%, at least 98%, at least 99%, or 100% identical to any one of SEQ ID NOs: 8, 10, 12, 24, 30, 40, 42, 44, or 46. In some embodiments, each of the three monomer constructs in the ACC is identical, and comprises a sequence that is at least 80% (e.g., at least 82%, at least 84%, at least 86%, at least 88%, at least 90%, at least 92%, at least 94%, at least 96%, at least 98%, at least 99%, or 100% identical to any one of SEQ ID NOs. 8, 10, 12, 24, 30, 40, 42, 44, or 46.

In some embodiments, one or more monomer constructs in an ACC may be encoded by a nucleic acid comprising a sequence of any one of SEQ ID NO: 9, 11, 13, 25, 31, 41, 43, 45, or 47.

In some embodiments, one or more monomer constructs in an ACC may be encoded by a nucleic acid comprising a sequence that is at least 80% (e.g., at least 82%, at least 84%, at least 86%, at least 88%, at least 90%, at least 92%, at least 94%, at least 96%, at least 98%, at least 99%, or 100%) identical to any one of SEQ ID NO: 9, 11, 13, 25, 31, 41, 43, 45, or 47.

In some aspects, the present disclosure provides nucleic acids comprising a sequence of any one of SEQ ID NO; 9, 11, 13, 25, 31, 41, 43, 45, or 47. In some aspects, the present disclosure provides nucleic acids comprising a sequence that is at least 80% (e.g., at least 82%, at least 84%, at least 86%, at least 88%, at least 90%, at least 92%, at least 94%, at least 96%, at least 98%, at least 99%, or 100%) identical to any one of SEQ ID NO: 9, 11, 13, 25, 31, 41, 43, 45, or 47. In some aspects, the present disclosure provides one or more vector comprising any of the nucleic acids described herein.

In some aspects, one or more monomer constructs in an ACC may include such sequences but either without the signal sequences of those sequences. Signal sequences are not particularly limited. Some examples of signal sequences include SEQ ID NO: 78.

### Masking Moieties (MMs)

The ACCs herein may comprise one or more masking moieties (MMs) capable of interfering with the binding of the CP to its binding partner (e.g., ligand or receptor).

A MM may be a steric masking moiety (SMM) or an affinity masking moiety (AMM) as described herein. A MM may be coupled to a CP by a CM and optionally one or more linkers described herein. In some embodiments, when an ACC is not activated, the MM prevents the CP from target binding; but when the ACC is activated (when the CM is cleaved by a protease), the MMs does not substantially or significantly interfere with the CP's binding to the target.

In the ACC, a MM interfering with the target binding of a CP may be coupled to the CP. Alternatively, a MM interfering with the target binding of a CP may be coupled to a component of the ACC that is not the CP. For example, the MM may be coupled to a different CP. In either case, in the tertiary or quaternary structure of the activatable structure, the MM may be in a position (e.g., proximal to the CP to be masked) that allows the MM to mask the CP.

In some embodiments, a MM may interact with the CP, thus reducing or inhibiting the interaction between the CP and its binding partner. In some embodiments, the MM may comprise at least a partial or complete amino acid sequence of a naturally occurring binding partner of the CP. For example, the MM may be a fragment of a naturally occurring binding partner. The fragment may retain no more than 95%, 90%, 80%, 75%, 70%, 60%, 50%, 40%0, 30%, 25%, or 20% nucleic acid or amino acid sequence homology to the naturally occurring binding partner.

In some embodiments, the MM may not specifically bind to the CP, but still interfere with CP's binding to its binding partner through non-specific interactions such as steric hindrance. For example, the MM may be positioned in the ACC such that the tertiary or quaternary structure of the ACC allows the MM to mask the CP through charge-based interaction, thereby holding the MM in place to interfere with binding partner access to the CP.

In some embodiments, the masking moiety (e.g., the steric masking moiety such as albumin (e.g., HSA)) may stabilize the ACC in the inactivated state.

In some examples, a SMM may be a peptide whose size, structure, conformation, and/or position in the ACC prevents, inhibits, or interfere the binding of the CP to its binding partner. In some examples, the SMM may be a globular protein, e.g., an albumin such as ovalbumin, human serum albumin (HSA), and bovine serum albumin (BSA). In a particular example, the SMM may be a human serum albumin, e.g., SEQ ID NO: 56. In some examples, the SMM may comprise a sequence that is at least at least 80% (e.g., at least 82%, at least 84%, at least 86%, at least 88%, at least 90%, at least 92%, at least 94%, at least 96%, at least 98%, at least 99%, or 100%) identical to SEQ ID NO: 56.

In some embodiments, an AMM may be a cognate peptide of the CP. For example, the MM may comprise a sequence of the CP's epitope, ligand, or receptor, or a fragment thereof. In cases where the CP is a TNFSF14, an AMM may be a receptor or a portion thereof of the TNFSF14, e.g., SEQ ID NO: 61.

The term “naturally occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally occurring.

In some embodiments, the MM may comprise an amino acid sequence that is not naturally occurring or does not contain the amino acid sequence of a naturally occurring binding partner or target protein. In certain embodiments, the MM is not a natural binding partner of the CP. The MM may be a modified binding partner for the CP which contains amino acid changes that decrease affinity and/or avidity of binding to the CP. In some embodiments the MM may contain no or substantially no nucleic acid or amino acid homology to the CP's natural binding partner. In other embodiments the MM is no more than 5%, 10%, 15%, 20%, 25%, 30%, 35%, 40%, 45%, 50%, 55%, 60%, 65%, 70%, 75%, or 80% similar to the natural binding partner of the CP.

In some embodiments, the MM may have a dissociation constant for binding to the CP that is no more than the dissociation constant of the CP to the target. In some embodiments, the MM may not interfere or compete with the CP for binding to the target in a cleaved state.

The structural properties of the MMs may be selected according to factors such as the minimum amino acid sequence required for interference with protein binding to target, the target protein-protein binding pair of interest, the size of the CP, the presence or absence of linkers, and the like.

In some embodiments, the MM may be unique for the coupled CP. Examples of MMs include MMs that were specifically screened to bind a binding domain of the CP or fragment thereof (e.g., affinity masks). Methods for screening MMs to obtain MMs unique for the CP and those that specifically and/or selectively bind a binding domain of a binding partner/target are provided herein and can include protein display methods.

As used herein, the term “masking efficiency” refers to the activity (e.g., EC₅₀) of the ACC in the inactivated state divided by the activity of a control antibody, wherein the control antibody may be either cleavage product of the ACC or the antibody or fragment thereof used as the CP of the activatable target-binding protein. An ACC having a reduced level of a CP activity may have a masking efficiency that is greater than 10. In some embodiments, the activatable target-binding proteins described herein may have a masking efficiency that is greater than 10, 100, 1000, or 5000.

In some embodiments, the MM may be a polypeptide of about 2 to 50 amino acids in length. For example, the MM may be a polypeptide of from 2 to 40, from 2 to 30, from 2 to 20, from 2 to 10, from 5 to 15, from 10 to 20, from 15 to 25, from 20 to 30, from 25 to 35, from 30 to 40, from 35 to 45, from 40 to 50 amino acids in length. For example, the MM may be a polypeptide with 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the MM may be a polypeptide of more than 50 amino acids in length, e.g., 100, 200, 300, 400, 500, 600, 700, 800, or more amino acids.

In some embodiments, in an inactive state of the ACC with an CP and an interfering MM, in the presence of the target of an CP, there is no binding or substantially no binding of the CP to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the CP to its target, as compared to the binding of an counterpart antibody without the interfering MM, for at least 0.1, 0.5, 1, 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, or 12 months when measured in vitro immunoabsorbant assay, e.g., as described in US20200308243A1.

The binding affinity of the CP towards the target or binding partner with an interfering MM may be at least 5, 10, 25, 50, 100, 250, 500, 1,000, 2,500, 5,000, 10,000, 50,000, 100,000, 500,000, 1,000,000, 5,000,000, 10,000,000, 50,000,000 times lower than the binding affinity of the CP towards its binding partner without an interfering MM, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1000-10,000,000, 10,000-100,000, 10,000-1,000,000, 10,000-10,000,000, 100,000-1,000,000, or 100,000-10,000,000 times lower than the binding affinity of the CP towards its binding partner when there is no interfering MM.

The dissociation constant of the MM towards the CP it masks, may be greater than the dissociation constant of the CP towards the target. The dissociation constant of the MM towards the masked CP may be at least 5, 10, 25, 50, 100, 250, 500, 1,000, 2,500, 5,000, 10,000, 100,000, 1,000,000 or even 10,000,000 times greater than the dissociation constant of the CP towards the target. Conversely, the binding affinity of the MM towards the masked CP may be lower than the binding affinity of the CP towards the target. The binding affinity of MM towards the CP may be at least 5, 10, 25, 50, 100, 250, 500, 1,000, 2,500, 5,000, 10,000, 100,000, 1,000,000 or even 10,000,000 times lower than the binding affinity of the CP towards the target.

In some embodiments, the MMs may contain genetically encoded or genetically non-encoded amino acids. Examples of genetically non-encoded amino acids are but not limited to D-amino acids, β-amino acids, and γ-amino acids. In specific embodiments, the MMs contain no more than 50%, 40%, 30%, 20%, 15%, 10%, 5% or 1% of genetically non-encoded amino acids.

In some embodiments, once released from the ACC and in a free state, the MM may have a biological activity or a therapeutic effect, such as binding capability. For example, the free peptide may bind with the same or a different binding partner. In certain embodiments, the free MM may exert a therapeutic effect, providing a secondary function to the compositions disclosed herein. In some embodiments, once uncoupled from the ACC and in a free state, the MM may advantageously not exhibit biological activity. For example, in some embodiments the MM in a free state does not elicit an immune response in the subject.

Suitable MMs may be identified and/or further optimized through a screening procedure from a library of candidate activatable target-binding proteins having variable MMs. For example, a CP and a CM may be selected to provide for a desired enzyme/target combination, and the amino acid sequence of the MM can be identified by the screening procedure described below to identify a MM that provides for an activatable phenotype. For example, a random peptide library (e.g., of peptides comprising 2 to 40 amino acids or more) may be used in the screening methods disclosed herein to identify a suitable MM.

In some embodiments, MMs with specific binding affinity for a CP may be identified through a screening procedure that includes providing a library of peptide scaffolds comprising candidate MMs wherein each scaffold is made up of a transmembrane protein and the candidate MM. The library may then be contacted with an entire or portion of a protein such as a full length protein, a naturally occurring protein fragment, or a non-naturally occurring fragment containing a protein (also capable of binding the binding partner of interest), and identifying one or more candidate MMs having detectably bound protein. The screening may be performed by one more rounds of magnetic-activated sorting (MACS) or fluorescence-activated sorting (FACS), as well as determination of the binding affinity of MM towards the CP and subsequent determination of the masking efficiency, e.g., as described in WO2009025846 and US20200308243A1, which are incorporated herein by reference in their entireties.

### Cytokine Proteins

The ACC may employ any of a variety of cytokine proteins that can form a trimer. Examples of such cytokine proteins include members of the tumor necrosis factor (TNF) ligand superfamily, such as a member of TNF or TNF superfamily member. Examples of the cytokine proteins include tumor necrosis factor superfamily member 14 (TNFSF14, also known as LIGHT), tumor necrosis factor TNF (e.g., TNF-alpha, -beta, or -C), TNFSF4, TNFSF5, TNFSF6, TNFSF7, TNFSF8, TNFSF9, TNFSF10, TNFSF1, TNFSF12, TNFSF13, TNFSF13B, TNFSF15, and TNFSF18. In one example, the cytokine proteins are LIGHT (also known as TNFSF14). In some examples, the ACC comprises a cytokine that is not TNF (e.g., a member of the TNF superfamily other than TNF).

In some embodiments, the cytokine proteins may be mature cytokine proteins. The term “mature cytokine protein” refers herein to a cytokine protein that lacks a signal sequence. A signal sequence is also referred to herein as a “signal peptide.” A mature cytokine protein may also lack intracellular and/or transmembrane domain(s). A cytokine protein (CP) may be a mature cytokine protein or a cytokine protein with a signal peptide, intracellular domain, transmembrane domain, or a portion thereof. In some embodiments, the cytokine proteins may comprise a signal peptide. In some examples, the ACCs of the present disclosure may include sequences disclosed herein, including or lacking the signal sequences recited herein.

For example, sequences of such proteins include those exemplified herein and additional sequences can be obtained from ncbi.nlm.nih.gov/protein. Truncation variants that are suitable for use in the ACCs of the present invention include any N- or C-terminally truncated cytokine that retains a cytokine activity. In some examples, the truncation variants may be cytokine polypeptides that are N- and/or C-terminally truncated by 1 to about 200 amino acids, 1 to about 150 amino acids, 1 to about 100 amino acids, 1 to about 95 amino acids, 1 to about 90 amino acids, 1 to about 85 amino acids, 1 to about 80 amino acids, 1 to about 75 amino acids, 1 to about 70 amino acids, 1 to about 65 amino acids, 1 to about 60 amino acids, 1 to about 55 amino acids, 1 to about 50 amino acids, 1 to about 45 amino acids, 1 to about 40 amino acids, 1 to about 35 amino acids, 1 to about 30 amino acids, 1 to about 25 amino acids, 1 to about 20 amino acids, 1 to about 15 amino acids, 1 to about 10 amino acids, 1 to about 8 amino acids, 1 to about 6 amino acids, 1 to about 4 amino acids, that retain a cytokine activity. In some of the foregoing embodiments, the truncated CP is an N-terminally truncated CP. In other embodiments, the truncated CP is a C-terminally truncated CP. In certain embodiments, the truncated CP is a C- and an N-terminally truncated CP. In some embodiments, the CP is truncated to remove a naturally-occurring protease recognition sequence (i.e., to remove a site that may be susceptible to cleavage by a protease).

In some examples, each of the CP1, the CP2, and the CP3 may independently comprise a cytokine (e.g., mutant of a wild type cytokine) that is cross-reactive among multiple species. The cross-reactive cytokine may bind to receptors in different species and activate the corresponding signaling pathways. In some examples, the cross-reactive cytokine is mouse-human cross-reactive, i.e., can bind to receptors in both human and mouse and activate the corresponding signaling pathway(s). In some examples, the cross-reactive cytokine is a mouse-human cross-reactive TNFSF14. The mouse-human cross-reactive TNFSF14 may comprise one or more mutations on human TNFSF14 protein. In one example, the mouse-human cross-reactive TNFSF14 comprises a sequence of SEQ ID NO: 55. Additional cross-reactive cytokines may be identified by screening a random error mutagenesis library of a cytokine (e.g., a wild type cytokine) using yeast surface display, e.g., as described in Tang et al. Cancer Cell. 2016 Mar. 14; 29(3):285-296, which is incorporated by reference in its entirety.

In some embodiments, each of the CP1, the CP2, and the CP3 may independently comprise an amino acid sequence that is at least 80% identical (e.g., at least 82%, at least 84%, at least 86%, at least 88%, at least 90%, at least 92%, at least 94%, at least 95%, at least 96%, at least 97%, at least 98%, at least 99%, or 100% identical) to a cytokine reference sequence of SEQ ID NO: 54 or 55.

The percentage of sequence identity refers to the level of amino acid sequence identity between two or more peptide sequences when aligned using a sequence alignment program, e.g., the suite of BLAST programs, publicly available on the Internet at the NCBI website. See also Altschul et al., J. Mol. Biol. 215:403-10, 1990.

In some embodiments, the CP1, CP2, and/or CP3 exhibit(s) an activity of a member of tumor necrosis factor or tumor necrosis factor super family (e.g., TNFSF14) and include(s) an amino acid sequence that is at least 80% identical, at least 82% identical, at least 84% identical, at least 86% identical, at least 88% identical, at least 90% identical, at least 92% identical, at least 94% identical, at least 96% identical, at least 98% identical, or at least 99% identical, or 100% identical to a sequence of SEQ ID NO: 54 or 55.

The number of amino acids in the sequence of the cytokine proteins employed may vary, depending on the specific cytokine protein employed. In some embodiments, the CP1, the CP2, and/or the CP3 includes a total of about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 250 amino acids, about 10 amino acids to about 200 amino acids, about 10 amino acids to about 150 amino acids, about 10 amino acids to about 100 amino acids, about 10 amino acids to about 80 amino acids, about 10 amino acids to about 60 amino acids, about 10 amino acids to about 40 amino acids, about 10 amino acids to about 20 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 250 amino acids, about 20 amino acids to about 200 amino acids, about 20 amino acids to about 150 amino acids, about 20 amino acids to about 100 amino acids, about 20 amino acids to about 80 amino acids, about 20 amino acids to about 60 amino acids, about 20 amino acids to about 40 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 250 amino acids, about 40 amino acids to about 200 amino acids, about 40 amino acids to about 150 amino acids, about 40 amino acids to about 100 amino acids, about 40 amino acids to about 80 amino acids, about 40 amino acids to about 60 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 250 amino acids, about 60 amino acids to about 200 amino acids, about 60 amino acids to about 150 amino acids, about 60 amino acids to about 100 amino acids, about 60 amino acids to about 80 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 250 amino acids, about 80 amino acids to about 200 amino acids, about 80 amino acids to about 150 amino acids, about 80 amino acids to about 100 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 250 amino acids, about 100 amino acids to about 200 amino acids, about 100 amino acids to about 150 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 250 amino acids, about 150 amino acids to about 200 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 250 amino acids, about 250 amino acids to about 700 amino acids, about 250 amino acids to about 650 amino acids, about 250 amino acids to about 600 amino acids, about 250 amino acids to about 550 amino acids, about 250 amino acids to about 500 amino acids, about 250 amino acids to about 450 amino acids, about 250 amino acids to about 400 amino acids, about 250 amino acids to about 350 amino acids, about 250 amino acids to about 300 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 700 amino acids, about 500 amino acids to about 650 amino acids, about 500 amino acids to about 600 amino acids, about 500 amino acids to about 550 amino acids, about 550 amino acids to about 700 amino acids, about 550 amino acids to about 650 amino acids, about 550 amino acids to about 600 amino acids, about 600 amino acids to about 700 amino acids, about 600 amino acids to about 650 amino acids, or about 650 amino acids to about 700 amino acids. In some embodiments, CP1, the CP2, and/or the CP3 is a mature wild type human cytokine protein.

### Cleavable Moieties

In some aspects, positioned between a CP and an MM (e.g., SMM and/or AMM) components in an ACC, either directly or indirectly (e.g., via a linker), is a cleavable moiety (CM) that comprises a substrate for a protease. In some embodiments, each of the CMs in the ACC may independently comprise a substrate for a protease selected from the group consisting of ADAM8, ADAM9, ADAM10, ADAM12, ADAM15, ADAM17/TACE, ADEMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin A, Cathepsin B, Cathepsin C, Cathepsin G, Cathepsin K, Cathepsin L, Cathepsin S, Cathepsin V/L2, Cathepsin X/Z/P, Chymase, Cruzipain, DESC1, DPP-4, FAP, Legumain, Otubain-2, Elastase, FVIIa, FiXA, FXa, FXIa, FXIIa, Granzyme B, Guanidinobenzoatase, Hepsin, HtrA1, Human Neutrophil Elastase, KLK4, KLK5, KLK6, KLK7, KLK8, KLK10, KLK11, KLK13, KLK14, Lactoferrin, Marapsin, Matriptase-2, Meprin, MT-SP1/Matriptase, Neprilysin, NS3/4A, PACE4, Plasmin, PSMA, PSA, BMP-1, MMP1, MMP2, MMP3, MMP7, MMP8, MMP9, MMP10, MMP11, MMP12, MMPP13, MMP14, MMP15, MMP16, MMP17, MMPP19, MMP20, MMP23, MMP24, MMP26, MMP27, TMPRSS2, TMPRSS3, TMPRSS4, tPA, Thrombin, Tryptase, and uPA.

In some embodiments, the protease that cleaves any of the CMs described herein can be ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27, activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, or TMPRSS4.

In some embodiments, the protease is selected from the group of: uPA, legumain, MT-SP1, ADAM17, BMP-1, TMPRSS3, TMPRSS4, MMP-2, MMP-9, MMP-12, MMP-13, and MMP-14.

In some embodiments, the CM is selected for use with a specific protease. The protease may be one produced by a tumor cell (e.g., the tumor cell may express greater amounts of the protease than healthy tissues). In some embodiments, the CM is a substrate for at least one protease selected from the group of an ADAM 17, a BMP-1, a cysteine protease such as a cathepsin, a HtrA1, a legumain, a matriptase (MT-SP1), a matrix metalloprotease (MMP), a neutrophil elastase, a TMPRSS, such as TMPRSS3 or TMPRSS4, a thrombin, and a u-type plasminogen activator (uPA, also referred to as urokinase).

In some embodiments, a CM is a substrate for at least one matrix metalloprotease (MMP). Examples of MMPs include MMP1, MMP2, MMP3, MMP7, MMP8, MMP9, MMP10, MMP11, MMPP12, MMP13, MMP14, MMP15, MMP16, MMPP17, MMP19, MMP20, MMP23, MMP24, MMP26, and MMP27. In some embodiments, the CM is a substrate for MMP9, MMP14, MMP1, MMP3, MMP13, MMP17, MMP11, and MMP19. In some embodiments, the CM is a substrate for MMP7. In some embodiments, the CM is a substrate for MMP9. In some embodiments, the CM is a substrate for MMP14. In some embodiments, the CM is a substrate for two or more MMPs. In some embodiments, the CM is a substrate for at least MMP9 and MMP14. In some embodiments, the CM includes two or more substrates for the same MMP. In some embodiments, the CM includes at least two or more MMP9 substrates. In some embodiments, the CM includes at least two or more MMP14 substrates.

Increased levels of proteases having known substrates have been reported in a number of cancers. See, e.g., La Roca et al., British J. Cancer 90(7):1414-1421, 2004. Substrates suitable for use in the CM components employed herein include those which are more prevalently found in cancerous cells and tissue. Thus, in certain embodiments, each of the CMs in the ACC may independently comprise a substrate for a protease that is more prevalently found in diseased tissue associated with a cancer. In some embodiments, the cancer is selected from the group of: gastric cancer, breast cancer, osteosarcoma, and esophageal cancer. In some embodiments, the cancer is breast cancer. In some embodiments, the cancer is a HER2-positive cancer. In some embodiments, the cancer is Kaposi sarcoma, hairy cell leukemia, chronic myeloid leukemia (CML), follicular lymphoma, renal cell cancer (RCC), melanoma, neuroblastoma, basal cell carcinoma, cutaneous T-cell lymphoma, nasopharyngeal adenocarcimoa, breast cancer, ovarian cancer, bladder cancer, BCG-resistant non-muscle invasive bladder cancer (NMIBC), endometrial cancer, pancreatic cancer, non-small cell lung cancer (NSCLC), colorectal cancer, esophageal cancer, gallbladder cancer, glioma, head and neck carcinoma, uterine cancer, cervical cancer, or testicular cancer, and the like. In some of the above-described embodiments, the CM components comprise substrates for protease(s) that is/are more prevalent in tumor tissue.

In some embodiments, the CM may be or comprise a sequence of encompassed by the consensus of sequence of any one of the sequences in Table 1 below and SEQ ID NOs: 62, 63, and 81. In some embodiments, the CM is at least 95%, 98% or 99% identical to a sequence selected from the group consisting of SEQ ID Nos: 62, 63, and 81.

TABLE 1
Exemplary CM sequences

| | SEQ |
|---|---|
| Sequences | ID NO |
| AANALAHGLF | 140 |
| AANL | 141 |
| AANLGSGGSS | 142 |
| AAPRS | 143 |
| AAPRSF | 144 |
| AARGPAIH | 145 |
| AAYHLVSQ | 146 |
| AFPDMRSVRS | 147 |
| AFQALRM | 148 |
| AFRHLR | 149 |
| AGLGISST | 150 |
| AGLGVVER | 151 |
| AGPR | 152 |
| AHGL | 153 |
| AHGLF | 154 |
| AHQALRM | 155 |
| AIPRVRLFDV | 156 |
| ALAHG | 157 |
| ALAHGL | 158 |
| ALAHGLF | 159 |
| ALAHGLFAPRSF | 160 |
| ALAHGLFSGRSA | 161 |
| N | |
| ALAHGLPTFVHL | 162 |
| ALGLLRLP | 163 |
| ALPSVKMVSE | 164 |
| ALRAP | 165 |
| ANQALRM | 166 |
| ANQALRMA | 167 |
| APPLVKSMVV | 168 |
| APPSFKLVNA | 169 |
| APRS | 170 |
| APRSALAHGLF | 171 |
| APRSF | 172 |
| AQFVLTEG | 173 |
| AQNLLGMV | 174 |
| ARGP | 175 |
| ARGPS | 176 |
| ARGPSF | 177 |
| ARGPSFK | 178 |
| ASGLLRFP | 179 |
| ASPTMKTVGL | 180 |
| AVGLLAPP | 181 |
| AVGLLAPPGGLS | 182 |
| GRSANI | |
| AVGLLAPPGGLS | 183 |
| GRSANP | |
| AVGLLAPPGGLS | 184 |
| GRSDDH | |
| AVGLLAPPGGLS | 185 |
| GRSDIH | |
| AVGLLAPPGGLS | 186 |
| GRSDNH | |
| AVGLLAPPGGLS | 187 |
| GRSDNI | |
| AVGLLAPPGGLS | 188 |
| GRSDNP | |
| AVGLLAPPGGLS | 189 |
| GRSDQH | |
| AVGLLAPPGGLS | 190 |
| GRSDTH | |
| AVGLLAPPGGLS | 191 |
| GRSDYH | |
| AVGLLAPPGGLS | 192 |
| GRSNI | |
| AVGLLAPPGGLS | 193 |
| GRSNIG | |
| AVGLLAPPGGLS | 194 |
| GRSNIGS | |
| AVGLLAPPGGTS | 195 |
| TSGRSANPRG | |
| AVGLLAPPSGRS | 196 |
| ANPRG | |
| AVGLLAPPTSGR | 197 |
| SANPRG | |
| AVPKVRVVPE | 198 |
| CGPPLGR | 199 |
| CSPPLGR | 200 |
| DEVDGSGGSS | 201 |
| DEXXXC(A/S) | 202 |
| DISHWRRS | 203 |
| DLAHPLL | 204 |
| DLPLVKSLPS | 205 |
| DLXXT(A/S) | 206 |
| DRLSGRSANHK | 207 |
| K | |
| DRLSGRSDNHK | 208 |
| K | |
| DRPEMKSLSG | 209 |
| DRPKVKTMDF | 210 |
| DVAQFVLT | 211 |
| DVPPMKTLRP | 212 |
| DWLYWMGI | 213 |
| DWLYWMSI | 214 |
| DWLYWPGI | 215 |
| DWLYWPSI | 216 |
| EAPKVKALPK | 217 |
| EHPRVKVVSE | 218 |
| EKPRMKLFQG | 219 |
| EPQALAMS | 220 |
| EQPEVKMVKG | 221 |
| ERPGVKSLVL | 222 |
| ESLPVVAV | 223 |
| ESPVMKSMAL | 224 |
| ESRRW | 225 |
| ESRRWM | 226 |
| ESRRWMP | 227 |
| ETPSVKTMGR | 228 |
| ETPSVKTMGRSS | 229 |
| FPRPLGITGL | 230 |
| FRLLDWQW | 231 |
| GCGPPLGR | 232 |
| GCSPPLGR | 233 |
| GFPHMKTFQH | 234 |
| GFPHMKTFQHSS | 235 |
| GGGPPLGR | 236 |
| GGPPLGR | 237 |
| GGQPSGMWGW | 238 |
| GGSIDGR | 239 |
| GGSPPLGR | 240 |
| GGWHTGRN | 241 |
| GIAGQ | 242 |
| GLGTPRGLFA | 243 |
| GLPTFV | 244 |
| GLPTFVH | 245 |
| GLPTFVHL | 246 |
| GLPTFVHLPRQV | 247 |
| GLSGRSDNHGSS | 248 |
| GPEGLRVG | 249 |
| GPLGIAGI | 250 |
| GPLNGRSDNHK | 251 |
| A | |
| GPLNGRSDNHK | 252 |
| K | |
| GPLNGRSDNHK | 253 |
| R | |
| GPLNGRSDNHQ | 254 |
| A | |
| GPLNGRSDNHQ | 255 |
| K | |
| GPLNGRSDNHQ | 256 |
| R | |
| GPLNGRSDNHR | 257 |
| A | |
| GPLNGRSDNHR | 258 |
| K | |
| GPLNGRSDNHR | 259 |
| R | |
| GPLSGRSDNHKA | 260 |
| GPLSGRSDNHKK | 261 |
| GPLSGRSDNHKR | 262 |
| GPLSGRSDNHQA | 263 |
| GPLSGRSDNHQK | 264 |
| GPLSGRSDNHQR | 265 |
| GPLSGRSDNHRA | 266 |
| GPLSGRSDNHRK | 267 |
| GPLSGRSDNHRR | 268 |
| GPPLGR | 269 |
| GPQGIAGQ | 270 |
| GPQGLLGA | 271 |
| GPRSFG | 272 |
| GPRSFGL | 273 |
| GPSHLVLT | 274 |
| GPTN | 275 |
| GPTNALAHGLF | 276 |
| GRSML | 277 |
| GRSMLL | 278 |
| GRSMLLG | 279 |
| GRSMLLGG | 280 |
| GRSMLLGP | 281 |
| GRSMLLGS | 282 |
| GRSMLLP | 283 |
| GRSMLLPG | 284 |
| GRSMLLPP | 285 |
| GRSMLLPS | 286 |
| GRSMLLS | 287 |
| GRSMLLSG | 288 |
| GRSMLLSP | 289 |
| GRSMLLSS | 290 |
| GRSMLM | 291 |
| GRSMLMG | 292 |
| GRSMLMGG | 293 |
| GRSMLMGP | 294 |
| GRSMLMGS | 295 |
| GRSMLMP | 296 |
| GRSMLMPG | 297 |
| GRSMLMPP | 298 |
| GRSMLMPS | 299 |
| GRSMLMS | 300 |
| GRSMLMSG | 301 |
| GRSMLMSP | 302 |
| GRSMLMSS | 303 |
| GSGPPLGR | 304 |
| GSPPLGR | 305 |
| GSSPPLGR | 306 |
| GTGRGPSWVGS | 307 |
| S | |
| HMMQYARH | 308 |
| HTGRSGAL | 309 |
| HVPRQ | 310 |
| HVPRQV | 311 |
| HVPRQVAPRSF | 312 |
| HVPRQVLSGRS | 313 |
| HVPRQVLSGRSA | 314 |
| N | |
| HWHLGPPT | 315 |
| IANLLSMV | 316 |
| IDGR | 317 |
| IEGR | 318 |
| ILNLLSMV | 319 |
| ILPRSPAF | 320 |
| IPFSWSRF | 321 |
| IQNLLSMV | 322 |
| ISSGL | 323 |
| ISSGLL | 324 |
| ISSGLLS | 325 |
| ISSGLLSGRSANI | 326 |
| ISSGLLSGRSANP | 327 |
| ISSGLLSGRSANP | 328 |
| RG | |
| ISSGLLSGRSDDH | 329 |
| ISSGLLSGRSDIH | 330 |
| ISSGLLSGRSDNH | 331 |
| ISSGLLSGRSDNI | 332 |
| ISSGLLSGRSDNP | 333 |
| ISSGLLSGRSDQH | 334 |
| ISSGLLSGRSDTH | 335 |
| ISSGLLSGRSDYH | 336 |
| ISSGLLSGRSGNH | 337 |
| ISSGLLSGRSNI | 338 |
| ISSGLLSGRSNIG | 339 |
| ISSGLLSGRSNIGS | 340 |
| ISSGLLSS | 341 |
| ISSGLLSSGGSGG | 342 |
| SLSGRSDNH | |
| ISSGLLSSGGSGG | 343 |
| SLSGRSGNH | |
| ISSGLSS | 344 |
| IVSRSA | 345 |
| KGLTGRSDRHQA | 346 |
| KGPKVKVVTL | 347 |
| KNLYGRSENNGN | 348 |
| KRMPVQFL | 349 |
| LAAPLGLL | 350 |
| LAHG | 351 |
| LAHGL | 352 |
| LAHGLF | 353 |
| LAPLGLQRR | 354 |
| LARAG | 355 |
| LARAGI | 356 |
| LARAGL | 357 |
| LKAAPRWA | 358 |
| LKAAPRWF | 359 |
| LKAAPVWA | 360 |
| LKAAPVWF | 361 |
| LKGRSYYY | 362 |
| LLAPSHRA | 363 |
| LLEALRAL | 364 |
| LLESLRAL | 365 |
| LLLPAHGG | 366 |
| LLLPLLGS | 367 |
| LLNALRAL | 368 |
| LLNSLRAL | 369 |
| LLQALRAL | 370 |
| LLQSLRAL | 371 |
| LLSALRAL | 372 |
| LLSSLRAL | 373 |
| LNGRSDNH | 374 |
| LPAGLLL | 375 |
| LPAGLLLR | 376 |
| LPAHLVLL | 377 |
| LPAHLVLV | 378 |
| LPGGLSPW | 379 |
| LPSHLVLL | 380 |
| LPSHLVLV | 381 |
| LPTFV | 382 |
| LPTFVH | 383 |
| LPTFVHL | 384 |
| LRSGW | 385 |
| LSGR | 386 |
| LSGRS | 387 |
| LSGRSA | 388 |
| LSGRSALAHGLF | 389 |
| LSGRSAN | 390 |
| LSGRSANI | 391 |
| LSGRSANP | 392 |
| LSGRSD | 393 |
| LSGRSDD | 394 |
| LSGRSDDH | 395 |
| LSGRSDI | 396 |
| LSGRSDIH | 397 |
| LSGRSDN | 398 |
| LSGRSDNH | 399 |
| LSGRSDNH | 400 |
| LSGRSDNHGGAV | 401 |
| GLLAPP | |
| LSGRSDNHGGSG | 402 |
| GSISSGLLSS | |
| LSGRSDNHGGSG | 403 |
| GSQNQALRMA | |
| LSGRSDNHGGVH | 404 |
| MPLGFLGP | |
| LSGRSDNI | 405 |
| LSGRSDNP | 406 |
| LSGRSDQ | 407 |
| LSGRSDQH | 408 |
| LSGRSDT | 409 |
| LSGRSDTH | 410 |
| LSGRSDY | 411 |
| LSGRSDYH | 412 |
| LSGRSENH | 413 |
| LSGRSG | 414 |
| LSGRSGN | 415 |
| LSGRSGNH | 416 |
| LSGRSGNHGGSG | 417 |
| GSISSGLLSS | |
| LSGRSGNHGGSG | 418 |
| GSQNQALRMA | |
| LSGRSGNP | 419 |
| LSGRSVTQ | 420 |
| LSQARWRK | 421 |
| LTFPTYIF | 422 |
| LTFPTYWF | 423 |
| LTGRSDRH | 424 |
| LTGRSGA | 425 |
| LTGRSGA | 426 |
| LYAAPRWA | 427 |
| LYAAPRWF | 428 |
| LYAAPVWA | 429 |
| LYAAPVWF | 430 |
| LYGRSENN | 431 |
| MDAFLESS | 432 |
| MGLFSEAG | 433 |
| MGPWFM | 434 |
| MIAPVAYR | 435 |
| MLRSGW | 436 |
| MLRSGWR | 437 |
| MLRSGWRG | 438 |
| MLRSGWRL | 439 |
| MLRSGWRS | 440 |
| MTFPTYIF | 441 |
| MTFPTYWF | 442 |
| NHRIGRSDNHRR | 443 |
| NMPSFKLVTG | 444 |
| NTLSGRSENHSG | 445 |
| NTLSGRSGNHGS | 446 |
| NZPRVRLVLP | 447 |
| PAGLWLDP | 448 |
| PAGRR | 449 |
| PAGRRS | 450 |
| PAGRRSL | 451 |
| PASLWYTQ | 452 |
| PESRRWMP | 453 |
| PFHLSR | 454 |
| PHGFFQ | 455 |
| PLARAGI | 456 |
| PLARAGL | 457 |
| PLGL | 458 |
| PLGLAG | 459 |
| PLGLWA | 460 |
| PLGVRGK | 461 |
| PLTGRSGG | 462 |
| PLTGRSGGGGSS | 463 |
| PPLGR | 464 |
| PPPDMKLFPG | 465 |
| PPPEVRSFSV | 466 |
| PPPVLKLLEW | 467 |
| PPSIARSDNLAN | 468 |
| PQHRIVSF | 469 |
| PRFKIIGG | 470 |
| PRFRIIGG | 471 |
| PRPFVKSVDQ | 472 |
| PRQV | 473 |
| PRSF | 474 |
| PSPPVKMMPE | 475 |
| PTNGGSGGSS | 476 |
| PTNL | 477 |
| PTNLGSGGSS | 478 |
| PVGYTSSL | 479 |
| PVPRLKLIKD | 480 |
| PVQPIGPQ | 481 |
| QALAMSAI | 482 |
| QFQALRM | 483 |
| QGPMFKSLWD | 484 |
| QGRAITFI | 485 |
| QHQALRM | 486 |
| QNQALRIA | 487 |
| QNQALRM | 488 |
| QNQALRMA | 489 |
| QNQALRMAGGSGG | 490 |
| SLSGRSDNH | |
| QNQALRMAGGSGG | 491 |
| SLSGRSGNH | |
| QSRRVP | 492 |
| QSRRVPL | 493 |
| QSRRVPV | 494 |
| QTRRVP | 495 |
| QTRRVPL | 496 |
| QTRRVPV | 497 |
| QYIVSRSA | 498 |
| RALRAP | 499 |
| REPFMKSLPW | 500 |
| RFPLKV | 501 |
| RFPSLKSFPL | 502 |
| RFPYGVW | 503 |
| RFYRNQFF | 504 |
| RGPA | 505 |
| RGPAFNPM | 506 |
| RGPATPIM | 507 |
| RGPKLYW | 508 |
| RHLAKL | 509 |
| RIGRSDNH | 510 |
| RKMPNITV | 511 |
| RKSSIIIRMRDVVL | 512 |
| RKTVQHWW | 513 |
| RLGRSDNN | 514 |
| RMHLRSLG | 515 |
| RPLARAGI | 516 |
| RPLARAGL | 517 |
| RPLNGRSDNHKA | 518 |
| RPLNGRSDNHKK | 519 |
| RPLNGRSDNHKR | 520 |
| RPLNGRSDNHQA | 521 |
| RPLNGRSDNHQK | 522 |
| RPLNGRSDNHQR | 523 |
| RPLNGRSDNHRA | 524 |
| RPLNGRSDNHRK | 525 |
| RPLNGRSDNHRR | 526 |
| RPLSGRSDNHKA | 527 |
| RPLSGRSDNHKK | 528 |
| RPLSGRSDNHKR | 529 |
| RPLSGRSDNHQA | 530 |
| RPLSGRSDNHQK | 531 |
| RPLSGRSDNHQR | 532 |
| RPLSGRSDNHRA | 533 |
| RPLSGRSDNHRK | 534 |
| RPLSGRSDNHRR | 535 |
| RPSPMWAY | 536 |
| RRHDGLRA | 537 |
| RRHDGLRS | 538 |
| RSLVFAPI | 539 |
| RSPSRLKC | 540 |
| RVPKVKVMLD | 541 |
| SAGFSLPA | 542 |
| SAPAVESE | 543 |
| SAPYFRMMDM | 544 |
| SARGPSRW | 545 |
| SCGPPLGR | 546 |
| SCSPPLGR | 547 |
| SGGPLGVR | 548 |
| SGGPPLGR | 549 |
| SGPPLGR | 550 |
| SGRS | 551 |
| SGRSA | 552 |
| SGRSAN | 553 |
| SGRSANI | 554 |
| SGRSANP | 555 |
| SGRSANPRG | 556 |
| SGRSD | 557 |
| SGRSDD | 558 |
| SGRSDDH | 559 |
| SGRSDI | 560 |
| SGRSDIH | 561 |
| SGRSDN | 562 |
| SGRSDNI | 563 |
| SGRSDNP | 564 |
| SGRSDQ | 565 |
| SGRSDQH | 566 |
| SGRSDT | 567 |
| SGRSDTH | 568 |
| SGRSDY | 569 |
| SGRSDYH | 570 |
| SGRSG | 571 |
| SGRSGN | 572 |
| SGRSGNH | 573 |
| SGSPPLGR | 574 |
| SIARSDNL | 575 |
| SISSGLLSGRSDNI | 576 |
| SNPFKY | 577 |
| SPLPLRVP | 578 |
| SPLPLRVP | 579 |
| SPLTGRSG | 580 |
| SPPLGR | 581 |
| SRRVP | 582 |
| SRRVPL | 583 |
| SRRVPV | 584 |
| SSGPPLGR | 585 |
| SSPPLGR | 586 |
| SSRGPAYL | 587 |
| SSRHRRALD | 588 |
| SSSFDKGKYKKGD | 589 |
| DA | |
| SSSFDKGKYKRGD | 590 |
| DA | |
| SSSPPLGR | 591 |
| STFPFGMF | 592 |
| STVFHM | 593 |
| SVHHLI | 594 |
| SVSGLLSH | 595 |
| SVSGLLSS | 596 |
| SVSGLRSH | 597 |
| SVSGLRSS | 598 |
| TARG | 599 |
| TARGP | 600 |
| TARGPALAHGLF | 601 |
| TARGPS | 602 |
| TARGPSF | 603 |
| TARGPSFK | 604 |
| TARGPSW | 605 |
| TARGPSW | 606 |
| TARGPVPRQV | 607 |
| TFVH | 608 |
| TGLSGRSVTQTS | 609 |
| TGRGPSWV | 610 |
| TLRLGRSDNNKN | 611 |
| TLSGLRSP | 612 |
| TSGRSANP | 613 |
| TSGRSGNP | 614 |
| TSLSGRSANPRG | 615 |
| TSLSGRSGNPRG | 616 |
| TSSGLRSP | 617 |
| TSTSGRSANPRG | 618 |
| TSTSGRSANPRGGG | 619 |
| AVGLLAPP | |
| TSTSGRSANPRGGG | 620 |
| VHMPLGFLGP | |
| TSTSGRSGNPRG | 621 |
| TVSGLRSP | 622 |
| VAGRSMRP | 623 |
| VAPQLKSLVP | 624 |
| VAQFVLTE | 625 |
| VHMPLGFLGP | 626 |
| VHMPLGFLGPGGL | 627 |
| SGRSDNH | |
| VHMPLGFLGPGGT | 628 |
| STSGRSANPRG | |
| VLPELRSVFS | 629 |
| VLSKQMSF | 630 |
| VPAGRRS | 631 |
| VPAGRRSL | 632 |
| VPRQ | 633 |
| VPRQV | 634 |
| VSRSA | 635 |
| VVPEGRRS | 636 |
| WATPRPMR | 637 |
| WDHPISLL | 638 |
| XXQAR(A/V)X | 639 |
| YDPZVKVVLA | 640 |
| YGAGLGVV | 641 |
| YIVSRSA | 642 |
| YKKFVGSL | 643 |
| YVPRVKALEM | 644 |

Examples of the CMs further include truncation variants of the aforementioned amino acid sequences that retain the recognition site for the corresponding protease. These include C-terminal and/or N-terminal truncation variants comprising at least 3 contiguous amino acids of the above-described amino acid sequences, or at least 4, or at least 5, or at least 6, or at least 7 amino acids of the foregoing amino acid sequences that retain a recognition site for a protease. In certain embodiments, the truncation variant of the above-described amino acid sequences is an amino acid sequence corresponding to any of the above, but that is C- and/or N-terminally truncated by 1 to about 10 amino acids, 1 to about 9 amino acids, 1 to about 8 amino acids, 1 to about 7 amino acids, 1 to about 6 amino acids, 1 to about 5 amino acids, 1 to about 4 amino acids, or 1 to about 3 amino acids, and which: (1) has at least three amino acid residues; and (2) retains a recognition site for a protease. In some of the foregoing embodiments, the truncated CM is an N-terminally truncated CM. In some embodiments, the truncated CM is a C-terminally truncated CM. In some embodiments, the truncated C is a C- and an N-terminally truncated CM.

In some embodiments, each of the CMs in the ACC may independently comprise a total of about 3 amino acids to about 25 amino acids. In some embodiments, each of the CMs in the ACC may independently comprise a total of about 3 amino acids to about 25 amino acids, about 3 amino acids to about 20 amino acids, about 3 amino acids to about 15 amino acids, about 3 amino acids to about 10 amino acids, about 3 amino acids to about 5 amino acids, about 5 amino acids to about 25 amino acids, about 5 amino acids to about 20 amino acids, about 5 amino acids to about 15 amino acids, about 5 amino acids to about 10 amino acids, about 10 amino acids to about 25 amino acids, about 10 amino acids to about 20 amino acids, about 10 amino acids to about 15 amino acids, about 15 amino acids to about 25 amino acids, about 15 amino acids to about 20 amino acids, or about 20 amino acids to about 25 amino acids.

In some embodiments, the ACC may comprise multiple CMs that comprise substrates for different proteases. In some embodiments, some or all of the CMs in the ACC comprise substrates for different proteases. In some embodiments, the CMs comprise substrates for the same protease.

### Linkers

The monomer constructs may comprise one or more linkers between two components. In some embodiments, the first monomer can include a linker disposed between the CP1 and the CM1. In some embodiments, the CP1 and the CM1 directly abut each other in the first monomer. In some embodiments, the first monomer comprises a linker disposed between the CM1 and the SMM1. In some embodiments, the CM1 and the SMM1 directly abut each other in the first monomer. In some embodiments, the first monomer comprises a linker disposed between the CP1 and the CM4 (the CM coupling the CP1 and the AMM1). In some embodiments, the CP1 and the CM4 directly abut each other in the first monomer. In some embodiments, the first monomer comprises a linker disposed between the CM4 and the AMM1. In some embodiments, the CM4 and the AMM1 directly abut each other in the first monomer.

In some embodiments, the second monomer can include a linker disposed between the CP2 and the CM2. In some embodiments, the CP2 and the CM2 directly abut each other in the second monomer. In some embodiments, the second monomer comprises a linker disposed between the CM2 and the SMM2. In some embodiments the CM2 and the SMM2 directly abut each other in the second monomer. In some embodiments, the second monomer comprises a linker disposed between the CP2 and the CM5 (the CM coupling the CP2 and the AMM2). In some embodiments, the CP2 and the CM5 directly abut each other in the second monomer. In some embodiments, the second monomer comprises a linker disposed between the CM5 and the AMM2. In some embodiments, the CM5 and the AMM2 directly abut each other in the second monomer.

In some embodiments, the third monomer can include a linker disposed between the CP3 and the CM3. In some embodiments, the CP3 and the CM3 directly abut each other in the third monomer. In some embodiments, the third monomer comprises a linker disposed between the CM3 and the SMM3. In some embodiments, the CM3 and the SMM3 directly abut each other in the third monomer. In some embodiments, the third monomer comprises a linker disposed between the CP3 and the CM6 (the CM coupling the CP3 and the AMM3). In some embodiments, the CP3 and the CM6 directly abut each other in the third monomer. In some embodiments, the third monomer comprises a linker disposed between the CM6 and the AMM3. In some embodiments, the CM6 and the AMM3 directly abut each other in the third monomer.

In some embodiments, one or more linkers (e.g., flexible linkers) can be introduced into the activatable cytokine construct to provide flexibility at one or more of the junctions between domains, between moieties, between moieties and domains, or at any other junctions where a linker would be beneficial. In some embodiments, where the ACC is provided as a conformationally constrained construct, a flexible linker can be inserted to facilitate formation and maintenance of a structure in the uncleaved activatable cytokine construct. Any of the linkers described herein can provide the desired flexibility to facilitate the inhibition of the binding of a target (e.g., a receptor of a cytokine), or to facilitate cleavage of a CM by a protease. In some embodiments, linkers are included in the ACC that are all or partially flexible, such that the linker can include a flexible linker as well as one or more portions that confer less flexible structure to provide for a desired ACC. Some linkers may include cysteine residues, which may form disulfide bonds and reduce flexibility of the construct. A linker length may be determined by counting, in a N- to C-direction, the number of amino acids from the N-terminus of the linker adjacent to the C-terminal amino acid of the preceding component, to the C-terminus of the linker adjacent to the N-terminal amino acid of the following component (i.e., where the linker length does not include either the C-terminal amino acid of the preceding component or the N-terminal amino acid of the following component).

In some embodiments, a linker can include a total of about 1 amino acid to about 25 amino acids (e.g., about 1 amino acid to about 24 amino acids, about 1 amino acid to about 22 amino acids, about 1 amino acid to about 20 amino acids, about 1 amino acid to about 18 amino acids, about 1 amino acid to about 16 amino acids, about 1 amino acid to about 15 amino acids, about 1 amino acid to about 14 amino acids, about 1 amino acid to about 12 amino acids, about 1 amino acid to about 10 amino acids, about 1 amino acid to about 8 amino acids, about 1 amino acid to about 6 amino acids, about 1 amino acid to about 5 amino acids, about 1 amino acid to about 4 amino acids, about 1 amino acid to about 3 amino acids, about 1 amino acid to about 2 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 5 amino acids, about 2 amino acids to about 4 amino acids, about 2 amino acids to about 3 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 4 amino acids to about 5 amino acids, about 5 amino acids to about 25 amino acids, about 5 amino acids to about 24 amino acids, about 5 amino acids to about 22 amino acids, about 5 amino acids to about 20 amino acids, about 5 amino acids to about 18 amino acids, about 5 amino acids to about 16 amino acids, about 5 amino acids to about 15 amino acids, about 5 amino acids to about 14 amino acids, about 5 amino acids to about 12 amino acids, about 5 amino acids to about 10 amino acids, about 5 amino acids to about 8 amino acids, about 5 amino acids to about 6 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 15 amino acids, about 8 amino acids to about 14 amino acids, about 8 amino acids to about 12 amino acids, about 8 amino acids to about 10 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 15 amino acids, about 10 amino acids to about 14 amino acids, about 10 amino acids to about 12 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 15 amino acids, about 12 amino acids to about 14 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 14 amino acids to about 15 amino acids, about 15 amino acids to about 25 amino acids, about 15 amino acids to about 24 amino acids, about 15 amino acids to about 22 amino acids, about 15 amino acids to about 20 amino acids, about 15 amino acids to about 18 amino acids, about 15 amino acids to about 16 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 25 amino acids, about 18 amino acids to about 24 amino acids, about 18 amino acids to about 22 amino acids, about 18 amino acids to about 20 amino acids, about 20 amino acids to about 25 amino acids, about 20 amino acids to about 24 amino acids, about 20 amino acids to about 22 amino acids, about 22 amino acid to about 25 amino acids, about 22 amino acid to about 24 amino acids, or about 24 amino acid to about 25 amino acids).

In some embodiments of any of the ACCs described herein, the linker includes a total of about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, or about 25 amino acids.

In some embodiments, a linker can be rich in glycine (Gly or G) residues. In some embodiments, the linker can be rich in serine (Ser or S) residues. In some embodiments, the linker can be rich in glycine and serine residues. In some embodiments, the linker has one or more glycine-serine residue pairs (GS) (e.g., 1, 2, 3, 4, 5, 6, 7, 8, 9, or 10 or more GS pairs). In some embodiments, the linker has one or more Gly-Gly-Gly-Ser (GGGS) sequences (e.g., 1, 2, 3, 4, 5, 6, 7, 8, 9, or 10 or more GGGS sequences). In some embodiments, the linker has one or more Gly-Gly-Gly-Gly-Ser (GGGGS) sequences (e.g., 1, 2, 3, 4, 5, 6, 7, 8, 9, or 10 or more GGGGS sequences). In some embodiments, the linker has one or more Gly-Gly-Ser-Gly (GGSG) sequences (e.g., 1, 2, 3, 4, 5, 6, 7, 8, 9, or 10 or more GGSG sequences).

In some embodiments of any of the ACCs described herein, a linker includes any one of or a combination of one or more of: (GS)n, (GGS)n, (GSGGS)n (SEQ ID NO: 645), (GGGGS)n (SEQ ID NO: 646), (GGGS)n (SEQ ID NO: 647), GGSG (SEQ ID NO: 648), GGSGG (SEQ ID NO: 649), GSGSG (SEQ ID NO: 650), GSGGG (SEQ ID NO: 651), GGGSG (SEQ ID NO: 652), GSSSG (SEQ ID NO: 653), GSSGGSGGSGG (SEQ ID NO: 654), GGGS (SEQ ID NO: 655), GGGSGGGS (SEQ ID NO: 656), GGGSGGGSGGGS (SEQ ID NO: 657), GGGGSGGGGSGGGGS (SEQ ID NO: 658), GGGGSGGGGSGGGGSGGGGS (SEQ ID NO: 659), GGGGSGGGGSGGGGSGGGGSGGGGS (SEQ ID NO: 660), GGGGSGGGGS (SEQ ID NO: 661), GGGGS (SEQ ID NO: 662), GS, GGGGSGS (SEQ ID NO: 663), GGGGSGGGGSGGGGSGS (SEQ ID NO: 664), GGSLDPKGGGGS (SEQ ID NO: 665), PKSCDKTHTCPPCPAPELLG (SEQ ID NO: 666), SKYGPPCPPCPAPEFLG (SEQ ID NO: 667), GKSSGSGSESKS (SEQ ID NO: 668), GSTSGSGKSSEGKG (SEQ ID NO: 669), GSTSGSGKSSEGSGSTKG (SEQ ID NO: 670), GSTSGSGKPGSGEGSTKG (SEQ ID NO: 671), and GSTSGSGKPGSSEGST (SEQ ID NO: 672), where n is an integer of one or more.

Non-limiting examples of linkers can include a sequence that is at least 70% identical (e.g., at least 72%, at least 74%, at least 75%, at least 76%, at least 78%, at least 80%, at least 82%, at least 84%, at least 85%, at least 86%, at least 88%, at least 90%, at least 92%, at least 94%, at least 95%, at least 96%, at least 97%, at least 98%, at least 99%, or 100% identical) to the exemplary linker sequence described herein.

In some embodiments, an ACC can include at least 1, 2, 3, 4, 5, 6, 7, 8, 9, 10, 11, 12, 13, 14, 15, 16, 17, 18, 19, or 20 linker sequence(s) (e.g., the same or different linker sequences of any of the exemplary linker sequences described herein or known in the art). In some embodiments, a linker comprises sulfo-SIAB, SMPB, and sulfo-SMPB, wherein the linkers react with primary amines sulfhydryls.

Additional Exemplary linker sequences are listed in the following Table 2:

TABLE 2
Exemplary linker sequences

| | SEQ | | SEQ | | SEQ |
|---|---|---|---|---|---|
| | ID | | ID | | ID |
| Sequences | NO | Sequences | NO | Sequences | NO |
| (GSGGS)n | 645 | GKSSGSGSESKS | 668 | GGGS | 655 |
| n can be any integer | | | | | |
| that is 1 or greater | | | | | |
| (GGGS)n | 647 | GSTSGSGKSSEGKG | 669 | GSSGGSGGSGG | 654 |
| n can be any integer | | | | | |
| that is 1 or greater | | | | | |
| GGSG | 648 | GSTSGSGKSSEGSG | 670 | GGGSGGGS | 656 |
| | | STKG | | | |
| GGSGG | 649 | GSTSGSGKPGSGEG | 671 | GGGSGGGSGGG | 657 |
| | | STKG | | S | |
| GSGSG | 650 | GSTSGSGKPGSSEG | 672 | GGGGSGGGGSG | 658 |
| | | ST | | GGGS | |
| GSGGG | 651 | GGGGSGGGGSGGG | 660 | (GGGGS)n | 646 |
| | | GSGGGGSGGGGS | | n can be any | |
| | | | | integer that is 1 or | |
| | | | | greater | |
| GGGSG | 652 | GGGGSGGGGS | 661 | GGGGSGS | 663 |
| GSSSG | 653 | PKSCDKTHTCPPCP | 666 | GGGGSGGGGSG | 664 |
| | | APELLG | | GGGSGS | |
| GSSGGS | 673 | SKYGPPCPPCPAPE | 667 | GGSLDPKGGGG | 665 |
| | | FLG | | S | |
| ESKY | 674 | GPQGTAGQ | 676 | YGAGLGW | 677 |
| SGGG | 675 | SGGGG | 678 | | |

### Conjugation to Agents

The ACCs may be conjugated with one or more agents, for example, a targeting moiety to facilitate delivery to a cell or tissue of interest, an agent (e.g., a therapeutic agent, an antineoplastic agent), a toxin, or a fragment thereof.

In some embodiments, the ACC can be conjugated to a cytotoxic agent, including, without limitation, a toxin (e.g., an enzymatically active toxin of bacterial, fungal, plant, or animal origin, or fragments thereof) or a radioactive isotope. In some embodiments of, the activatable cytokine construct can be conjugated to a cytotoxic agent including, without limitation, a toxin (e.g., an enzymatically active toxin of bacterial, fungal, plant, or animal origin, or fragments thereof), or a radioactive isotope.

Non-limiting exemplary cytotoxic agents that can be conjugated to any of the ACCs described herein include: dolastatins and derivatives thereof (e.g., auristatin E, AFP, monomethyl auristatin D (MMAD), monomethyl auristatin F (MMAF), monomethyl auristatin E (MMAE), desmethyl auristatin E (DMAE), auristatin F, desmethyl auristatin F (DMAF), dolastatin 16 (DmJ), dolastatin 16 (Dpv), auristatin derivatives (e.g., auristatin tyramine, auristatin quinolone), maytansinoids (e.g., DM-1, DM-4), maytansinoid derivatives, duocarmycin, alpha-amanitin, turbostatin, phenstatin, hydroxyphenstatin, spongistatin 5, spongistatin 7, halistatin 1, halistatin 2, halistatin 3, halocomstatin, pyrrolobenzimidazoles (PBI), cibrostatin6, doxaliform, cemadotin analogue (CemCH2-SH), Pseudomonas toxin A (PES8) variant, Pseudomonase toxin A (ZZ-PE38) variant, ZJ-101, anthracycline, doxorubicin, daunorubicin, bryostatin, camptothecin, 7-substituted campothecin, 10, 11-difluoromethylenedioxycamptothecin, combretastatins, debromoaplysiatoxin, KahaMide-F, discodermolide, and Ecteinascidins.

Non-limiting exemplary enzymatically active toxins that can be conjugated to any of the ACCs described herein include: diphtheria toxin, exotoxin A chain from Pseudomonas aeruginosa, ricin A chain, abrin A chain, modeccin A chain, alpha-sarcin, Aleuriies fordii proteins, dianfhin proteins, Phytoiaca Americana proteins (e.g., PAPI, PAPII, and PAP-8), Momordica charantia inhibitor, curcin, crotirs, Sapaonaria officinalis inhibitor, geionin, mitogeliin, restrictocin, phenomycin, neomycin, and tricothecenes.

Non-limiting exemplary anti-neoplastics that can be conjugated to any of the ACCs described herein include: adriamycin, cerubidine, bleomycin, alkeran, velban, oncovin, fluorouracil, methotrexate, thiotepa, bisantrene, novantrone, thioguanine, procarabizine, and cytarabine.

Non-limiting exemplary antivirals that can be conjugated to any of the ACCs described herein include: acyclovir, vira A, and symmetrel.

Non-limiting exemplary antifungals that can be conjugated to any of the ACCs described herein include: nystatin.

Non-limiting exemplary conjugatable detection reagents that can be conjugated to any of the ACCs described herein include: fluorescein and derivatives thereof, fluorescein isothiocyanate (FITC).

Non-limiting exemplary antibacterials that can be conjugated to any of the activatable cytokine constructs described herein include: aminoglycosides, streptomycin, neomycin, kanamycin, amikacin, gentamicin, and tobramycin.

Non-limiting exemplary 3beta,16beta,17alpha-trihydroxycholest-5-en-22-one 16-O-(2-O-4-methoxybenzoyl-beta-D-xylopyranosyl)-(1→3)-(2-O-acetyl-alpha-L-arabinopyranoside) (OSW-1) that can be conjugated to any of the activatable cytokine constructs described herein include: s-nitrobenzyloxycarbonyl derivatives of 06-benzylguanine, toposisomerase inhibitors, hemiasterlin, cephalotaxine, homoharringionine, pyrrol obenzodiazepine dimers (PBDs), functionalized pyrrolobenzodiazepenes, calcicheamicins, podophyiitoxins, taxanes, and vinca alkoids.

Non-limiting exemplary radiopharmaceuticals that can be conjugated to any of the activatable cytokine constructs described herein include: ¹²³I, ⁸⁹Zr, ¹²⁵I, ¹³¹I, ^{99m}Tc, ²⁰¹Tl, ⁶²Cu, ¹⁸F, ⁶⁸Ga, ¹³N, ¹⁵O, ³⁸K, ⁸²Rb, ¹¹¹In, ¹³³Xe, ¹¹C, and ⁹⁹mTc (Technetium).

Non-limiting exemplary heavy metals that can be conjugated to any of the ACCs described herein include: barium, gold, and platinum.

Non-limiting exemplary anti-mycoplasmals that can be conjugated to any of the ACCs described herein include: tylosine, spectinomycin, streptomycin B, ampicillin, sulfanilamide, polymyxin, and chloramphenicol.

Those of ordinary skill in the art will recognize that a large variety of possible moieties can be conjugated to any of the activatable cytokine constructs described herein. Conjugation can include any chemical reaction that will bind the two molecules so long as the ACC and the other moiety retain their respective activities. Conjugation can include many chemical mechanisms, e.g., covalent binding, affinity binding, intercalation, coordinate binding, and complexation. In some embodiments, the preferred binding is covalent binding. Covalent binding can be achieved either by direct condensation of existing side chains or by the incorporation of external bridging molecules. Many bivalent or polyvalent linking agents are useful in conjugating any of the activatable cytokine constructs described herein. For example, conjugation can include organic compounds, such as thioesters, carbodiimides, succinimide esters, glutaraldehyde, diazobenzenes, and hexamethylene diamines. In some embodiments, the activatable cytokine construct can include, or otherwise introduce, one or more non-natural amino acid residues to provide suitable sites for conjugation.

In some embodiments of any of the ACCs described herein, an agent and/or conjugate is attached by disulfide bonds (e.g., disulfide bonds on a cysteine molecule) to the cytokine protein(s). Since many cancers naturally release high levels of glutathione, a reducing agent, glutathione present in the cancerous tissue microenvironment can reduce the disulfide bonds, and subsequently release the agent and/or the conjugate at the site of delivery.

In some embodiments of any of the ACCs described herein, when the conjugate binds to its target in the presence of complement within the target site (e.g., diseased tissue (e.g., cancerous tissue)), the amide or ester bond attaching the conjugate and/or agent to the linker is cleaved, resulting in the release of the conjugate and/or agent in its active form. These conjugates and/or agents when administered to a subject, will accomplish delivery and release of the conjugate and/or the agent at the target site (e.g., diseased tissue (e.g., cancerous tissue)). These conjugates and/or agents are particularly effective for the in vivo delivery of any of the conjugates and/or agents described herein.

In some embodiments, the linker is not cleavable by enzymes of the complement system. For example, the conjugate and/or agent is released without complement activation since complement activation ultimately lyses the target cell. In such embodiments, the conjugate and/or agent is to be delivered to the target cell (e.g., hormones, enzymes, corticosteroids, neurotransmitters, or genes). Furthermore, the linker is mildly susceptible to cleavage by serum proteases, and the conjugate and/or agent is released slowly at the target site.

In some embodiments of any of the ACCs described herein, the conjugate and/or agent is designed such that the conjugate and/or agent is delivered to the target site (e.g., disease tissue (e.g., cancerous tissue)) but the conjugate and/or agent is not released.

In some embodiments of any of the ACCs described herein, the conjugate and/or agent is attached to a cytokine protein either directly or via a non-cleavable linker. Exemplary non-cleavable linkers include amino acids (e.g., D-amino acids), peptides, or other organic compounds that may be modified to include functional groups that can subsequently be utilized in attachment to cytokines by methods described herein.

In some embodiments of any of the ACCs described herein, an ACC includes at least one point of conjugation for an agent. In some embodiments, all possible points of conjugation are available for conjugation to an agent. In some embodiments, the one or more points of conjugation include, without limitation, sulfur atoms involved in disulfide bonds, sulfur atoms involved in interchain disulfide bonds, sulfur atoms involved in interchain sulfide bonds but not sulfur atoms involved in intrachain disulfide bonds, and/or sulfur atoms of cysteine or other amino acid residues containing a sulfur atom. In such cases, residues may occur naturally in the protein construct structure or may be incorporated into the protein construct using methods including, without limitation, site-directed mutagenesis, chemical conversion, or mis-incorporation of non-natural amino acids.

This disclosure also provides methods and materials for preparing an ACC for conjugation. In some embodiments of any of the ACCs described herein, an ACC is modified to include one or more interchain disulfide bonds. For example, disulfide bonds in the ACC can undergo reduction following exposure to a reducing agent such as, without limitation, TCEP, DTT, or β-mercaptoethanol. In some cases, the reduction of the disulfide bonds is only partial. As used herein, the term partial reduction refers to situations where an ACC is contacted with a reducing agent and a fraction of all possible sites of conjugation undergo reduction (e.g., not all disulfide bonds are reduced). In some embodiments, an activatable cytokine construct is partially reduced following contact with a reducing agent if less than 99%, (e.g., less than 98%, 97%, 96%, 95%, 90%, 85%, 80%, 75%, 70%, 65%, 60%, 55%, 50% 45%, 40%, 35% 30%, 5% 20%, 15%, 10% or less than 5%) of all possible sites of conjugation are reduced. In some embodiments, the ACC having a reduction in one or more interchain disulfide bonds is conjugated to a drug reactive with free thiols.

This disclosure also provides methods and materials for conjugating a therapeutic agent to a particular location on an ACC. In some embodiments of any of the ACC described herein, an ACC is modified so that the therapeutic agents can be conjugated to the ACC at particular locations on the ACC. For example, an ACC can be partially reduced in a manner that facilitates conjugation to the ACC. In such cases, partial reduction of the ACC occurs in a manner that conjugation sites in the ACC are not reduced. In some embodiments, the conjugation site(s) on the ACC are selected to facilitate conjugation of an agent at a particular location on the protein construct. Various factors can influence the “level of reduction” of the ACC upon treatment with a reducing agent. For example, without limitation, the ratio of reducing agent to ACC, length of incubation, incubation temperature, and/or pH of the reducing reaction solution can require optimization in order to achieve partial reduction of the ACC with the methods and materials described herein. Any appropriate combination of factors (e.g., ratio of reducing agent to ACC, the length and temperature of incubation with reducing agent, and/or pH of reducing agent) can be used to achieve partial reduction of the ACC (e.g., general reduction of possible conjugation sites or reduction at specific conjugation sites).

An effective ratio of reducing agent to ACC can be any ratio that at least partially reduces the ACC in a manner that allows conjugation to an agent (e.g., general reduction of possible conjugation sites or reduction at specific conjugation sites). In some embodiments, the ratio of reducing agent to ACC will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

An effective incubation time and temperature for treating an ACC with a reducing agent can be any time and temperature that at least partially reduces the ACC in a manner that allows conjugation of an agent to an ACC (e.g., general reduction of possible conjugation sites or reduction at specific conjugation sites). In some embodiments, the incubation time and temperature for treating an ACC will be in a range from about 1 hour at 37° C. to about 12 hours at 37° C. (or any subranges therein).

An effective pH for a reduction reaction for treating an ACC with a reducing agent can be any pH that at least partially reduces the ACC in a manner that allows conjugation of the ACC to an agent (e.g., general reduction of possible conjugation sites or reduction at specific conjugation sites).

When a partially-reduced ACC is contacted with an agent containing thiols, the agent can conjugate to the interchain thiols in the ACC. An agent can be modified in a manner to include thiols using a thiol-containing reagent (e.g., cysteine or N-acetyl cysteine). For example, the ACC can be partially reduced following incubation with reducing agent (e.g., TEPC) for about 1 hour at about 37° C. at a desired ratio of reducing agent to ACC. An effective ratio of reducing agent to ACC can be any ratio that partially reduces at least two interchain disulfide bonds located in the ACC in a manner that allows conjugation of a thiol-containing agent (e.g., general reduction of possible conjugation sites or reduction at specific conjugation sites).

In some embodiments of any of the ACCs described herein, an ACC is reduced by a reducing agent in a manner that avoids reducing any intrachain disulfide bonds. In some embodiments of any of the ACCs described herein, an ACC is reduced by a reducing agent in a manner that avoids reducing any intrachain disulfide bonds and reduces at least one interchain disulfide bond.

In some embodiments of any of the ACCs described herein, the ACC can also include an agent conjugated to the ACC. In some embodiments, the conjugated agent is a therapeutic agent.

In some embodiments, the agent (e.g., agent conjugated to an activatable cytokine construct) is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

In some embodiments, the agent (e.g., cytotoxic agent conjugated to an activatable cytokine construct) is linked to the ACC using a carbohydrate moiety, sulfhydryl group, amino group, or carboxylate group.

In some embodiments, the agent (e.g., cytotoxic agent conjugated to an activatable cytokine construct) is conjugated to the ACC via a conjugating moiety. A conjugating moiety may comprise linker(s) and CM(s) described herein, as well as other type of molecules. In some embodiments, the agent (e.g., cytotoxic agent conjugated to an activatable cytokine construct) is conjugated to a cysteine or a lysine in the ACC. In some embodiments, the agent (e.g., cytotoxic agent conjugated to an activatable cytokine construct) is conjugated to another residue of the ACC, such as those residues disclosed herein. In some embodiments, the conjugating moiety is a thiol-containing conjugating moiety.

Those of ordinary skill in the art will recognize that a large variety of possible moieties can be coupled to the ACCs of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference). In general, an effective conjugation of an agent (e.g., cytotoxic agent) to an ACC can be accomplished by any chemical reaction that will bind the agent to the ACC while also allowing the agent and the ACC to retain functionality.

In some embodiments, a variety of bifunctional protein-coupling agents can be used to conjugate the agent to the ACC including, without limitation, N-succinimidyl-3-(2-pyridyldithiol) propionate (SPDP), iminothiolane (IT), bifunctional derivatives of imidoesters (e.g., dimethyl adipimidate HCL), active esters (e.g., disuccinimidyl suberate), aldehydes (e.g., glutareldehyde), bis-azido compounds (e.g., bis (p-azidobenzoyl) hexanediamine), bis-diazonium derivatives (e.g., bis-(p-diazoniumbenzoyl)-ethylenediamine), diisocyanates (e.g., tolyene 2,6-diisocyanate), and bis-active fluorine compounds (e.g., 1,5-difluoro-2,4-dinitrobenzene). For example, a ricin immunotoxin can be prepared as described in Vitetta et al., Science 238: 1098 (1987). In some embodiments, a carbon-14-labeled 1-isothiocyanatobenzyl-3-methyldiethylene triaminepentaacetic acid (MX-DTPA) chelating agent can be used to conjugate a radionucleotide to the ACC. (See, e.g., WO94/11026).

Examples of conjugating moieties are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an ACC by way of an oligopeptide linker. In some embodiments, suitable conjugating moieties include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. Additional conjugating moieties include SMCC, sulfo-SMCC, SPDB, or sulfo-SPDB.

The conjugating moieties described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing conjugating moieties are less soluble than sulfo-NHS esters. Further, the conjugating moieties SMPT contains a sterically-hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments of any of the ACCs, an agent can be conjugated to the ACC using a modified amino acid sequence included in the amino acid sequence of the ACC. By inserting conjugation-enabled amino acids at specific locations within the amino acid sequence of the ACC, the protein construct can be designed for controlled placement and/or dosage of the conjugated agent (e.g., cytotoxic agent). For example, the ACC can be modified to include a cysteine amino acid residue at positions on the first monomer, the second monomer, and/or the third monomer that provide reactive thiol groups and does not negatively impact protein folding and/or assembly and does not alter the binding of cytokine to its binding partners. In some embodiments, the ACC can be modified to include one or more non-natural amino acid residues within the amino acid sequence of the ACC to provide suitable sites for conjugation. In some embodiments, the ACC can be modified to include enzymatically activatable peptide sequences within the amino acid sequence of the ACC.

### Nucleic Acids

Provided herein are nucleic acids including sequences that encode the first monomer construct (or the protein portion of the first monomer construct) (e.g., any of the first monomers constructs described herein), the second monomer construct (or the protein portion of the second monomer construct) (e.g., any of the second monomer constructs described herein), and the third monomer construct (or the protein portion of the third monomer construct) (e.g., any of the third monomer constructs described herein) of any of the ACCs described herein. In some embodiments, a set of nucleic acids together encode the first monomer construct (or the protein portion of the first monomer construct), the second monomer construct (or the protein portion of the second monomer construct), and the third monomer construct (or the protein portion of the third monomer construct). In some embodiments, the nucleic acid sequence encoding the first monomer construct (or the protein portion of the first monomer construct) is at least 70% identical (e.g., at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical) to the nucleic acid sequence encoding the second monomer construct (or the protein portion of the second monomer construct). In some embodiments, the nucleic acid sequence encoding the first monomer construct (or the protein portion of the first monomer construct) is at least 70% identical (e.g., at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical) to the nucleic acid sequence encoding the third monomer construct (or the protein portion of the third monomer construct). In some embodiments, the nucleic acid sequence encoding the second monomer construct (or the protein portion of the second monomer construct) is at least 70% identical (e.g., at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical) to the nucleic acid sequence encoding the third monomer construct (or the protein portion of the third monomer construct).

### Vectors

Provided herein are vectors and sets of vectors including any of the nucleic acids described herein. One skilled in the art will be capable of selecting suitable vectors or sets of vectors (e.g., expression vectors) for making any of the ACCs described herein, and using the vectors or sets of vectors to express any of the ACCs described herein. For example, in selecting a vector or a set of vectors, the cell must be considered because the vector(s) may need to be able to integrate into a chromosome of the cell and/or replicate in it. Exemplary vectors that can be used to produce an ACC are also described below.

As used herein, the term “vector” refers to a polynucleotide capable of inducing the expression of a recombinant protein (e.g., a first or second monomer) in a cell (e.g., any of the cells described herein). A “vector” is able to deliver nucleic acids and fragments thereof into a host cell, and includes regulatory sequences (e.g., promoter, enhancer, poly(A) signal). Exogenous polynucleotides may be inserted into the expression vector in order to be expressed. The term “vector” also includes artificial chromosomes, plasmids, retroviruses, and baculovirus vectors.

Methods for constructing suitable vectors that include any of the nucleic acids described herein, and suitable for transforming cells (e.g., mammalian cells) are well-known in the art. See, e.g., Sambrook et al., Eds. “Molecular Cloning: A Laboratory Manual,” 2^{nd} Ed., Cold Spring Harbor Press, 1989 and Ausubel et al., Eds. “Current Protocols in Molecular Biology,” Current Protocols, 1993.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, for example, include sufficient cis-acting elements for expression; other elements for expression can be supplied by the host mammalian cell or in an in vitro expression system. Skilled practitioners will be capable of selecting suitable vectors and mammalian cells for making any of the ACCs described herein.

In some embodiments of any of the ACCs described herein, the ACC may be made biosynthetically using recombinant DNA technology and expression in eukaryotic or prokaryotic species.

In some embodiments, the vector includes a nucleic acid encoding the first monomer and the second monomer of any of the ACCs described herein. In some embodiments, the vector is an expression vector.

In some embodiments, a set of vectors together include a set of nucleic acids that together encode the first, the second, and the third monomer constructs of any of the ACCs described herein. In some embodiments, the pair of vectors is a set of expression vectors.

### Cells

Also provided herein are host cells including any of the vector or sets of vectors described herein including any of the nucleic acids described herein.

Any of the ACCs described herein can be produced by any cell (e.g., a mammalian cell). In some embodiments, a host cell is a mammalian cell (e.g., a human cell), a rodent cell (e.g., a mouse cell, a rat cell, a hamster cell, or a guinea pig cell), or a non-human primate cell.

Methods of introducing nucleic acids and vectors (e.g., any of the vectors or any of the sets of vectors described herein) into a cell are known in the art. Non-limiting examples of methods that can be used to introducing a nucleic acid into a cell include: lipofection, transfection, calcium phosphate transfection, cationic polymer transfection, viral transduction (e.g., adenoviral transduction, lentiviral transduction), nanoparticle transfection, and electroporation.

In some embodiments, the introducing step includes introducing into a cell a vector (e.g., any of the vectors or sets of vectors described herein) including a nucleic acid encoding the monomers that make up any of the ACCs described herein.

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary (CHO) cells and human embryonic kidney cells (e.g., HEK293 cells).

In some embodiments, the cell contains the nucleic acid encoding the first monomer and the second monomer of any one of the ACCs described herein. In some embodiments, the cell contains the pair of nucleic acids that together encode the first monomer and the second monomer of any of the ACCs described herein.

### Methods of Producing Activatable Cytokine Constructs

Provided herein are methods of producing any of the ACCs described herein that include: (a) culturing any of the recombinant host cells described herein in a liquid culture medium under conditions sufficient to produce the ACC; and (b) recovering the ACC from the host cell and/or the liquid culture medium.

Methods of culturing cells are well known in the art. Cells can be maintained in vitro under conditions that favor cell proliferation, cell differentiation and cell growth. For example, cells can be cultured by contacting a cell (e.g., any of the cells described herein) with a cell culture medium that includes the necessary growth factors and supplements sufficient to support cell viability and growth.

In some embodiments of any of the methods described herein, the method further includes isolating the recovered ACC. Non-limiting examples of methods of isolation include: ammonium sulfate precipitation, polyethylene glycol precipitation, size exclusion chromatography, ligand-affinity chromatography, ion-exchange chromatography (e.g., anion or cation), and hydrophobic interaction chromatography.

In some embodiments of any of the methods described herein, the method further includes formulating the isolated ACC into a pharmaceutical composition. Various formulations are known in the art and are described herein. Any of the isolated ACCs described herein can be formulated for any route of administration (e.g., intravenous, intratumoral, subcutaneous, intradermal, oral (e.g., inhalation), transdermal (e.g., topical), transmucosal, or intramuscular).

Also provided herein are ACCs produced by any of the methods described herein. Also provided are compositions (e.g., pharmaceutical compositions) that include any of the ACCs produced by any of the methods described herein. Also provided herein are kits that include at least one dose of any of the compositions (e.g., pharmaceutical compositions) described herein.

In some embodiments, the ACCs may comprise one or more tags that can be for purification, isolation, and/or detection of the ACC. Examples of such tags include affinity tags, such as His tag (e.g., 6×-His (hexahistidine) tag), FLAG tag, c-Myc tag, Glutathione-S-transferase (GST) tag, Maltose-Binding Protein (MBP) tag, Calmodulin-Binding Protein (CBP) tag, and Streptavidin/Biotin-Based tag. In these chases, the ACCs may be isolated or purified using the tag(s). In some examples, the tags may be removed from the ACCs.

In some embodiments, the cells used in the production process can produce a protein portion of the first, the second, and the third monomer constructs (e.g., with one or more affinity tags). The monomers may then associate non-covalently to form a trimer. In cases where the three monomers are the same, the cell may produce the monomer construct (e.g., with one or more affinity tags). The monomer construct may then associate non-covalently to form a homotrimer.

ACCs expressed in cells herein may be purified. The purification may be performed using an affinity column, e.g., an HSA-affinity column, or a streptavidin-affinity column, or other columns compatible with the tags described above. The sample from the affinity column may be further purified by other chromatography technology, e.g., size exclusion chromatography (SEC). The purified ACCs may have a purity of at least 80%, 90%, 95%, or 99%.

### Methods of Treatment

Provided herein are methods of treating a disease (e.g., a cancer (e.g., any of the cancers described herein)) in a subject including administering a therapeutically effective amount of any of the ACCs, the nucleic acids, vectors, compositions comprising the ACCs, nucleic acids, and/or the vectors described herein to the subject.

As used herein, the term “subject” refers to any organism such as a mammal. In some embodiments, the subject is a feline (e.g., a cat), a canine (e.g., a dog), an equine (e.g., a horse), a rabbit, a pig, a rodent (e.g., a mouse, a rat, a hamster or a guinea pig), a non-human primate (e.g., a simian (e.g., a monkey (e.g., a baboon, a marmoset), or an ape (e.g., a chimpanzee, a gorilla, an orangutan, or a gibbon)), or a human. In some embodiments, the subject is a human.

In some embodiments, the subject has been previously identified or diagnosed as having the disease (e.g., cancer (e.g., any of the cancers described herein)).

As used herein, the term “treat” includes reducing the severity, frequency or the number of one or more (e.g., 1, 2, 3, 4, or 5) symptoms or signs of a disease (e.g., a cancer (e.g., any of the cancers described herein)) in the subject (e.g., any of the subjects described herein). In some embodiments where the disease is cancer, treating results in reducing cancer growth, inhibiting cancer progression, inhibiting cancer metastasis, or reducing the risk of cancer recurrence in a subject having cancer.

In some embodiments of any of the methods described herein, the disease is a cancer. Also provided herein are methods of treating a subject in need thereof (e.g., any of the exemplary subjects described herein or known in the art) that include administering to the subject a therapeutically effective amount of any of the ACCs described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, a lymphoma (e.g., B-cell lymphoma, B-cell non-Hodgkin's lymphoma, Hodgkin's lymphoma, cutaneous T-cell lymphoma), a leukemia (e.g., hairy cell leukemia, chronic lymphocytic leukemia (CLL), acute myeloid leukemia (AML), chronic myeloid leukemia (CML), acute lymphocytic leukemia (ALL)), myelodysplastic syndromes (MDS), Kaposi sarcoma, retinoblastoma, stomach cancer, urothelial carcinoma, lung cancer, renal cell carcinoma, gastric and esophageal cancer, pancreatic cancer, prostate cancer, brain cancer, colon cancer, bone cancer, lung cancer, breast cancer, colorectal cancer, ovarian cancer, nasopharyngeal adenocarcimoa, non-small cell lung carcinoma (NSCLC), squamous cell head and neck carcinoma, endometrial cancer, bladder cancer, cervical cancer, liver cancer, and hepatocellular carcinoma. In some embodiments, the cancer is a lymphoma. In some embodiments, the lymphoma is Burkitt's lymphoma. In some aspects, the subject has been identified or diagnosed as having familial cancer syndromes such as L1 Fraumeni Syndrome, Familial Breast-Ovarian Cancer (BRCA1 or BRAC2 mutations) Syndromes, and others. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine.

Exemplary cancers described by the National Cancer Institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

Further exemplary cancers include diffuse large B-cell lymphoma (DLBCL) and mantle cell lymphoma (MCL).

Metastases of the aforementioned cancers can also be treated or prevented in accordance with the methods described herein.

In some embodiments, these methods can result in a reduction in the number, severity, or frequency of one or more symptoms of the cancer in the subject (e.g., as compared to the number, severity, or frequency of the one or more symptoms of the cancer in the subject prior to treatment).

In some embodiments, the methods of treating a disease (e.g., cancer) in a subject may comprise administering the ACC(s), nucleic acid(s), vector(s), composition (e.g., pharmaceutical composition) herein in combination with one or more immune checkpoint inhibitors (e.g., a PD-1 and/or PD-L1). The immune checkpoint inhibitors may be antibodies against PD-1 or PD-L1, e.g., those in Table 3 below.

In some embodiments of any of the methods described herein, the methods further include administering to a subject an additional therapeutic agent (e.g., one or more of the therapeutic agents listed in Table 3).

TABLE 3
Additional Therapeutic Agents

| Antibody Trade Name (antibody name) | Target |
|---|---|
| Raptiva ™ (efalizumab) | CD11a |
| Arzerra ™ (ofatumumab) | CD20 |
| Bexxar ™ (tositumomab) | CD20 |
| Gazyva ™ (obinutuzumab) | CD20 |
| Ocrevus ™ (ocrelizumab) | CD20 |
| Rituxan ™ (rituximab) | CD20 |
| Zevalin ™ (ibritumomab tiuxetan) | CD20 |
| Adcetris ™ (brentuximab vedotin) | CD30 |
| Myelotarg ™ (gemtuzumab) | CD33 |
| Mylotarg ™ (gemtuzumab ozogamicin) | CD33 |
| (vadastuximab) | CD33 |
| (vadastuximab talirine) | CD33 |
| Campath ™ (alemtuzumab) | CD52 |
| Lemtrada ™ (alemtuzumab) | CD52 |
| Tactress ™ (tamtuvetmab) | CD52 |
| Soliris ™ (eculizumab) | Complement C5 |
| Ultomiris ™ (ravulizumab) | Complement C5 |
| (olendalizumab) | Complement C5 |
| Yervoy ™ (ipilimumab) | CTLA-4 |
| (tremelimumab) | CTLA-4 |
| Orencia ™ (abatacept) | CTLA-4 |
| Hu5c8 | CD40L |
| (letolizumab) | CD40L |
| Rexomun ™ (ertumaxomab) | CD3/Her2 |
| Erbitux ™ (cetuximab) | EGFR |
| Portrazza ™ (necitumumab) | EGFR |
| Vectibix ™ (panitumumab) | EGFR |
| CH806 | EGFR |
| (depatuxizumab) | EGFR |
| (depatuxizumab mafodotin) | EGFR |
| (futuximab:modotuximab) | EGFR |
| ICR62 (imgatuzumab) | EGFR |
| (laprituximab) | EGFR |
| (losatuxizumab) | EGFR |
| (losatuxizumab vedotin) | EGFR |
| mAb 528 | EGFR |
| (matuzumab) | EGFR |
| (nimotuzumab) | EGFR |
| (tomuzotuximab) | EGFR |
| (zalutumumab) | EGFR |
| MDX-447 | EGFR/CD64 |
| (adecatumumab) | EpCAM |
| Panorex ™ (edrecolomab) | EpCAM |
| Vicinium ™ | EpCAM |
| Synagis ™ (palivizumab) | F protein of RSV |
| ReoPro ™ (abiciximab) | Glycoprotein receptor IIb/IIIa |
| Herceptin ™ (trastuzumab) | Her2 |
| Herceptin ™ Hylecta (trastuzumab; | Her2 |
| Hyaluronidase) | |
| (trastuzumab deruxtecan) | Her2 |
| (hertuzumab verdotin) | Her2 |
| Kadcyla ™ (trastuzumab emtansine) | Her2 |
| (margetuximab) | Her2 |
| (timigutuzumab) | Her2 |
| Xolair ™ (omalizumab) | IgE |
| (ligelizumab) | IgE |
| (figitumumab) | IGF1R |
| (teprotumumab) | IGF1R |
| Simulect ™ (basiliximab) | IL2R |
| Zenapax ™ (daclizumab) | IL2R |
| Zinbryta ™ (daclizumab) | IL2R |
| Actemra ™ (tocilizumab) | IL-6 receptor |
| Kevzara ™ (sarilumab) | IL-6 receptor |
| (vobarilizumab) | IL-6 receptor |
| Stelara ™ (ustekinumab) | IL-12/IL-23 |
| Tysabri ™ (natalizumab) | Integrinα4 |
| (abrilumab) | Integrinα4 |
| | Jagged 1 or Jagged 2 |
| (fasinumab) | NGF |
| (fulranumab) | NGF |
| (tanezumab) | NGF |
| | Notch, e.g., Notch 1 |
| Pidilizumab | Delta like-1 (PD-1 pathway |
| | inhibitor) |
| Opdivo ® (nivolumab) | PD1 |
| Keytruda ® (pembrolizumab) | PD1 |
| Libtayo ® (cemiplimab) | PD1 |
| BGB-A317 (tislelizumab) | PD1 |
| PDR001 (spartalizumab) | PD1 |
| JNJ-63723283 (cetrelimab) | PD1 |
| TSR042 (dostarlimab) | PD1 |
| AGEN2034 (balstilimab) | PD1 |
| JS001 (toripalimab) | PD1 |
| IOBI308 (sintilimab) | PD1 |
| BCD100 (prolgolimab) | PD1 |
| CBT-501 (genolimzumab | PD1 |
| ABBV181 (budigalimab) | PD1 |
| AK105 | PD1 |
| BI-754091 | PD1 |
| INCSHR-1210 | PD1 |
| MEDI0680 | PD1 |
| MGA012 | PD1 |
| SHR-1210 | PD1 |
| Imfinzi ™ (durvalumab) | PD-L1 |
| Tecentriq ® (atezolizumab) | PD-L1 |
| Bavencio ® (avelumab) | PD-L1 |
| KN035 (envafolimab) | PD-L1 |
| BMS936559 (MDX1105) | PD-L1 |
| BGBA 333 | PD-L1 |
| FAZ053 | PD-L1 |
| LY-3300054 | PD-L1 |
| SH-1316 | PD-L1 |
| AMP-224 | PD-L2 |
| (bavituximab) | Phosphatidylserine |
| huJ591 | PSMA |
| RAV12 | RAAG12 |
| Prolia ™ (denosumab) | RANKL |
| GC1008 (fresolimumab) | TGFbeta |
| Cimzia ™ (Certolizumab Pegol) | TNFα |
| Remicade ™ (infliximab) | TNFα |
| Humira ™ (adalimumab) | TNFα |
| Simponi ™ (golimumab) | TNFα |
| Enbrel ™ (etanercept) | TNF-R |
| (mapatumumab) | TRAIL-R1 |
| Avastin ™ (bevacizumab) | VEGF |
| Lucentis ™ (ranibizumab) | VEGF |
| (brolucizumab) | VEGF |
| (vanucizumab) | VEGF |

### Compositions/Kits

Also provided herein are compositions (e.g., pharmaceutical compositions) including any of the ACCs, nucleic acids, and/or vectors described herein and one or more (e.g., 1, 2, 3, 4, or 5) pharmaceutically acceptable carriers (e.g., any of the pharmaceutically acceptable carriers described herein), diluents, or excipients.

In some embodiments, the compositions (e.g. pharmaceutical compositions) that include any of the ACCs, nucleic acids, and/or vectors described herein can be disposed in a sterile vial or a pre-loaded syringe.

In some embodiments, the compositions (e.g. pharmaceutical compositions) that include any of the ACCs described herein can be formulated for different routes of administration (e.g., intravenous, subcutaneous, intramuscular, intraperitoneal, or intratumoral).

In some embodiments, any of the pharmaceutical compositions described herein can include one or more buffers (e.g., a neutral-buffered saline, a phosphate-buffered saline (PBS), amino acids (e.g., glycine), one or more carbohydrates (e.g., glucose, mannose, sucrose, dextran, or mannitol), one or more antioxidants, one or more chelating agents (e.g., EDTA or glutathione), one or more preservatives, and/or a pharmaceutically acceptable carrier (e.g., bacteriostatic water, PBS, or saline).

As used herein, the phrase “pharmaceutically acceptable carrier” refers to any and all solvents, dispersion media, coatings, antibacterial agents, antimicrobial agents, isotonic and absorption delaying agents, and the like, compatible with pharmaceutical administration. Suitable carriers include, but are not limited to: water, saline, ringer's solutions, dextrose solution, and about 5% human serum albumin.

In some embodiments of any of the pharmaceutical compositions described herein, any of the ACCs described herein are prepared with carriers that protect against rapid elimination from the body, e.g., sustained and controlled release formulations, including implants and microencapsulated delivery systems. Biodegradable, biocompatible polymers can be used, e.g., ethylene vinyl acetate, polyanhydrides, polyglycolic acid, collage, polyorthoesters, and polylactic acid. Methods for preparation of such pharmaceutical compositions and formulations are apparent to those skilled in the art.

Also provided herein are kits that include any of the ACCs described herein, any of the compositions that include any of the ACCs described herein, or any of the pharmaceutical compositions that include any of the ACCs described herein. Also provided are kits that include one or more second therapeutic agent(s) selected from Table 3 in addition to an ACC described herein. The second therapeutic agent(s) may be provided in a dosage administration form that is separate from the ACC. Alternatively, the second therapeutic agent(s) may be formulated together with the ACC.

Any of the kits described herein can include instructions for using any of the compositions (e.g., pharmaceutical compositions) and/or any of the ACCs described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

The present disclosure includes the following aspects including any combinations of any of the following aspects:

1. An activatable cytokine construct (ACC) comprising:
a first monomer construct comprising a first cytokine protein (CP1), a first cleavable moiety (CM1), and a first steric masking moiety (SMM1), wherein the CM1 is positioned between the CP1 and the SMM1;
a second monomer construct comprising a second cytokine protein (CP2), a second cleavable moiety (CM2), and a second steric masking moiety (SMM2), wherein the CM2 is positioned between the CP2 and the SMM2; and
a third monomer construct comprising a third cytokine protein (CP3), a third cleavable moiety (CM3), and a third steric masking moiety (SMM3), wherein the CM3 is positioned between the CP3 and the SMM3,
wherein:
the CP1, the CP2, and the CP3 bind to one another thereby forming a trimer of the first, the second, and the third monomer constructs; and
the SMM1, the SMM2, and the SMM3 are globular molecules.

2. The ACC of aspect 1, wherein the CP1, the CP2, and the CP3 are the same cytokine.

3. The ACC of aspect 2, wherein the cytokine is a member of tumor necrosis factor or tumor necrosis factor super family.

4. The ACC of aspect 2, wherein the CP1, the CP2 and the CP3 are tumor necrosis factor superfamily member 14 (TNFSF14).

5. The ACC of aspect 2, wherein each of the CP1, the CP2, and the CP3 comprises a sequence that is at least 80%, 90%, 95%, or 99% identical to SEQ ID NO: 54.

6. The ACC of any one or combination of aspects 1-5, wherein the SMM1, the SMM2, and the SMM3 are the same globular molecule.

7. The ACC of aspect 6, wherein the globular molecule is an albumin.

8. The ACC of aspect 7, wherein the albumin is a human serum albumin.

9. The ACC of aspect 7, wherein the albumin comprises a sequence that is at least 80%, 90%, 95%, or 99% identical to a human serum albumin.

10. The ACC of any one or combination of aspects 1-9, wherein the first monomer construct comprises at least one linker.

11. The ACC of aspect 10, wherein the at least one linker comprises a linker L1 disposed between the CP1 and the CM1, and/or a linker L2 between the CM1 and the SMM1.

12. The ACC any one or combination of aspects 1-11, wherein the second monomer construct comprises at least one linker.

13. The ACC of aspect 12, wherein the at least one linker comprises a linker L3 disposed between the CP2 and the CM2, and/or a linker L4 between the CM2 and the SMM2.

14. The ACC of any one or combination of aspects 1-13, wherein the third monomer construct comprises at least one linker.

15. The ACC of aspect 14, wherein the at least one linker comprises a linker L5 disposed between the CP3 and the CM3, and/or a linker L6 between the CM3 and the SMM3.

16. The ACC of any one or combination of aspects 1-15, wherein:
the first monomer construct further comprises a first affinity masking moiety (AMM1) and optionally a fourth cleavable moiety (CM4) positioned between the AMM1 and the CP1,
the second monomer construct further comprises a second affinity masking moiety (AMM2) and optionally a fifth cleavable moiety (CM5) positioned between the AMM2 and the CP2, and
the third monomer construct further comprises a third affinity masking moiety (AMM3) and optionally a sixth cleavable moiety (CM6) positioned between the AMM3 and the CP3.

17. The ACC of aspect 16, wherein the AMM1, the AMM2, and the AMM3 are the same.

18. The ACC of aspect 16, wherein the each of AMM1, the AMM2, and the AMM3 comprises a sequence of SEQ ID NO: 61.

19. The ACC of aspect 16, wherein the each of AMM1, the AMM2, and the AMM3 comprises a sequence that at least 80%, 90%, 95%, or 99% identical to SEQ ID NO:

61.

20. The ACC of any one or combination of aspects 1-19, wherein the CM1, the CM2, and the CM3 comprise a substrate of the same protease.

21. The ACC of any one or combination of aspects 1-19, wherein the CM1, the CM2, and the CM3 comprise substrates of different proteases.

22. The ACC of any one or combination of aspects 1-19, wherein each of the CM1, the CM2, and the CM3 comprises a sequence that is at least 95% identical to SEQ ID NO: 62 or 63.

23. The ACC of any one or combination of aspects 16-22, wherein the CM4, the CM5, and the CM6 comprise a substrate of the same protease.

24. The ACC of any one or combination of aspects 16-22, wherein the CM4, the CM5, and the CM6 comprise substrates of different proteases.

25. The ACC of any one or combination of aspects 16-22, wherein each of the CM4, the CM5, and the CM6 comprises a sequence that is at least 95% identical to SEQ ID NO. 62 or 63.

26. The ACC of any one or combination of aspects 20-25, wherein the protease(s) is/are produced by a tumor in a subject.

27. The ACC of any one or combination of aspects 20-25, wherein the protease(s) is/are selected from the group consisting of: ADAM8, ADAM9, ADAM10, ADAM12, ADAM15, ADAM17/TACE, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27, activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, and TMPRSS4.

28. The ACC of any one or combination of aspects 16-27, wherein the first monomer construct further comprises a linker L7 between the AMM1 and the CM4 and/or a linker L8 between the CM4 and the CP1.

29. The ACC of aspect any one or combination of aspects 16-28, wherein the second monomer construct further comprises a linker L9 between the AMM2 and the CM5 and/or a linker L10 between the CM5 and the CP2.

30. The ACC of any one or combination of aspects 16-29, wherein the third monomer construct further comprises a linker L11 between the AMM3 and the CM6 and/or a linker L12 between the CM6 and the CP3.

31. The ACC of any one or combination of aspects 11-30, wherein each of the linkers L1-L12 has a total length of 2 to 30 amino acids.

32. The ACC of any one or combination of aspects 11-31, wherein each of the linkers L1-L12 independently comprises a sequence of any one of SEQ ID NO: 64-69, 75-77, GGS, SGG, GSG, GS, or G.

33. The ACC of any one or combination of aspects 1-32, wherein in a N- to C-terminal direction:
the first monomer construct comprises the CP1, the CM1, and the SMM1,
the second monomer construct comprises the CP2, the CM2, and the SMM2, and
the third monomer construct comprises the CP3, the CM3, and the SMM3.

34. The ACC of any one or combination of aspects 1-32, wherein in a N- to C-terminal direction:
the first monomer construct comprises the SMM1, the CM1, and the CP1,
the second monomer construct comprises the SMM2, the CM2, and the CP2, and
the third monomer construct comprises the SMM3, the CM3, and the CP3.

35. The ACC of any one or combination of aspects 16-34, wherein in a N- to C-terminal direction:
the first monomer construct comprises the AMM1, the CM4, the CP1, the CM1, and the SMM1,
the second monomer construct comprises the AMM2, the CM5, the CP2, the CM2, and the SMM2, and
the third monomer construct comprises the AMM3, the CM6, the CP3, the CM3, and the SMM3.

36. The ACC of any one or combination of aspects 16-34, wherein in a N- to C-terminal direction:
the first monomer construct comprises the SMM1, the AMM1, the CM1, and the CP1;
the second monomer construct comprises the SMM2, the AMM2, the CM2, and the CP2, and
the third monomer construct comprises the SMM3, the AMM3, the CM3, and the CP3.

37. The ACC of any one or combination of aspects 16-34, wherein in a N- to C-terminal direction:
the first monomer construct comprises, the AMM1, the SMM1, the CM1, and the CP1;
the second monomer construct comprises the AMM2, the SMM2, the CM2, and the CP2, and
the third monomer construct comprises the AMM3, the SMM3, the CM3, and the CP3.

38. The ACC of any one or combination of aspects 16-34, wherein in a N- to C-terminal direction:
the first monomer construct comprises the SMM1, the CM1, the CP1, the CM4, and AMM1,
the second monomer construct comprises the SMM2, the CM2, the CP2, the CM5, and AMM2, and
the third monomer construct comprises the SMM3, the CM3, the CP3, the CM6, and the AMM3.

39. The ACC of any one or combination of aspects 16-34, wherein in a N- to C-terminal direction:
the first monomer construct comprises the CP1, the CM1, the AMM1, and the SMM1;
the second monomer construct comprises the CP2, the CM2, the AMM2, and the SMM2; and
the third monomer construct comprises the CP3, the CM3, the AMM3, and the SMM3.

40. The ACC of any one or combination of aspects 16-34, wherein in a N- to C-terminal direction:
the first monomer construct comprises the CP1, the CM1, the SMM1, and the AMM1;
the first monomer construct comprises the CP2, the CM2, the SMM2, and the AMM2; and
the first monomer construct comprises the CP3, the CM3, the SMM3, and the AMM3.

41. The ACC of any one or combination of aspects 1-40, wherein, in an inactive state, the ACC is characterized by having a reduced level of an activity of at least one of the CP1, the CP2, the CP3, or the trimer thereof as compared to a control level of the activity of at least one of the CP1, the CP2, the CP3, or the trimer thereof.

42. The ACC of aspect 41, wherein the ACC is characterized by at least a 2-fold, 5-fold, 10-fold, 500-fold, 10³-fold, 10⁴-fold, 10⁵-fold, or 10⁶-fold reduction in the activity of the trimer of CP1, CP2, and CP3 as compared to the activity of a control trimer of CP1, CP2, and CP3 that does not comprise a steric masking moiety or an affinity masking moiety.

43. The ACC of aspect 41 or 42, wherein the activity is activation of herpes virus entry mediator (HVEM).

44. The ACC of aspect 41 or 42, wherein the activity is activation of lymphotoxin beta receptor.

45. The ACC of aspect 41 or 42, wherein the activity is activation of herpes virus entry mediator (HVEM) and activation of lymphotoxin beta receptor.

46. The ACC of any one or combination of aspects 41-45, wherein the control trimer of CP1, CP2, and CP3 results from activation of the ACC.

47. An activatable cytokine construct (ACC) comprising:
a first monomer construct comprising a first cytokine protein (CP1), a first cleavable moiety (CM1), and a first affinity masking moiety (AMM1), wherein the CM1 is positioned between the CP1 and the AMM1;
a second monomer construct comprising a second cytokine protein (CP2), a second cleavable moiety (CM2), and a second affinity masking moiety (AMM2), wherein the CM2 is positioned between the CP2 and the AMM2; and
a third monomer construct comprising a third cytokine protein (CP3), a third cleavable moiety (CM3), and a third affinity steric masking moiety (AMM3), wherein the CM3 is positioned between the CP3 and the AMM3,
wherein the CP1, the CP2, and the CP3 bind to one another thereby forming a trimer of the first, the second, and the third monomer constructs.

48. An ACC of any one or combination of aspects 1-47, wherein the first monomer construct, the second monomer construct, and the third monomer construct are identical.

49. An ACC of any one or combination of aspects 1-48, wherein the ACC does not comprise any domain that facilitates formation of a trimer other than CP1, CP2, and CP3.

50. The ACC of aspect 49, wherein the ACC does not comprise any domain other than the CP1, the CP2, and the CP3 that covalently links the first, the second, and the third monomer constructs.

51. The ACC of aspect 49, wherein the ACC does not comprise a coil-coiled domain, an Fc domain, or a domain other than the CP1, the CP2, or the CP3 that is capable forming a disulfide bond.

52. The ACC of any one or combination of aspects 1-51, wherein the CP1, CP2, and CP3 are identical and each comprises the amino acid sequence of SEQ ID NO: 54.

53. The ACC of aspect 46, wherein the first monomer construct, the second monomer construct, and the third monomer construct are identical and each monomer comprises:
a. the amino acid sequence of SEQ ID NO: 54; and
b. an AMM comprising an amino acid sequence that is at least 95% identical to that of SEQ ID NO: 61; and
c. an SMM comprising an albumin.

54. A composition comprising the ACC of any one or combination of aspects 1-53.

55. The composition of aspect 54, wherein the composition is a pharmaceutical composition.

56. A container, vial, syringe, injector pen, or kit comprising at least one dose of the composition of aspect 54 or 55.

57. A nucleic acid encoding a polypeptide that comprises at least one of the first monomer construct, the second monomer construct, or the third monomer construct of the ACC of any one or combination of aspects 1-53.

58. The nucleic acid of aspect 57, comprising a sequence of any one of SEQ ID NOs: 9, 11, 13, 25, 31, 41, 43, 45, or 47.

59. A set of nucleic acids that together encode polypeptides that comprise the first monomer construct, the second monomer construct, and the third monomer construct in the ACC of any one or combination of aspects 1-53.

60. A vector comprising the nucleic acid or a set of nucleic acids of any of aspects 57-59.

61. A cell comprising the nucleic acid of any one or combination of aspects 57-59 or the vector of aspect 60.

62. A method of treating a subject in need thereof comprising administering to the subject a therapeutically effective amount of the ACC of any one or combination of aspects 1-53 or the composition of aspect 54 or 55.

63. The method of aspect 62, wherein the subject has been identified or diagnosed as having a cancer.

64. The method of aspect 62 or 63, further comprising administering an immune checkpoint inhibitor.

65. The method of aspect 64, wherein the immune checkpoint inhibitor is an anti-PD-1 antibody or anti-PD-L1 antibody.

66. A method of producing an ACC comprising:
culturing a cell of aspect 61 in a liquid culture medium under conditions sufficient to produce the ACC; and
recovering the ACC from the cell or the liquid culture medium.

67. The method of aspect 66, further comprising purifying the recovered ACC using affinity chromatography.

68. The method of aspect 66 or 67, further comprising formulating the recovered ACC into a pharmaceutical composition.

### EXAMPLES

The invention is further described in the following examples, which do not limit the scope of the invention described in the claims.

### Example 1: Activity of LIGHT Cytokine Constructs Engineered with a Non-Cleavable HSA Steric Mask

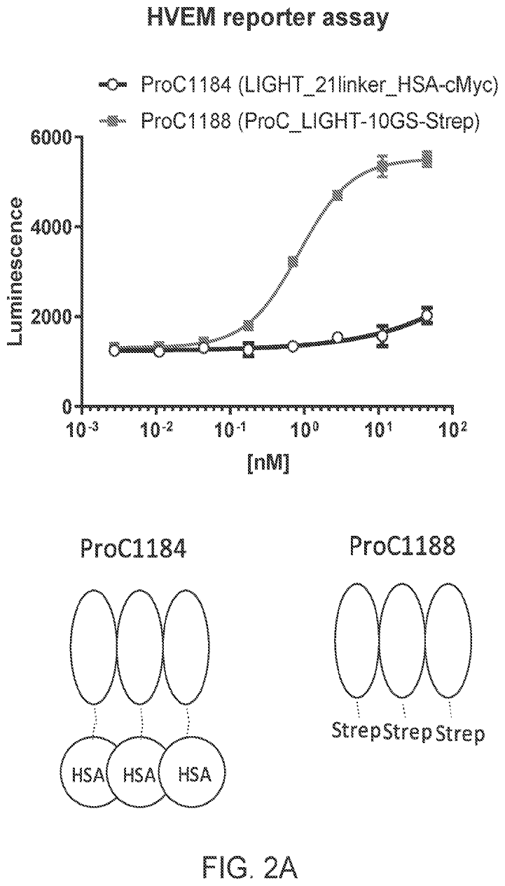

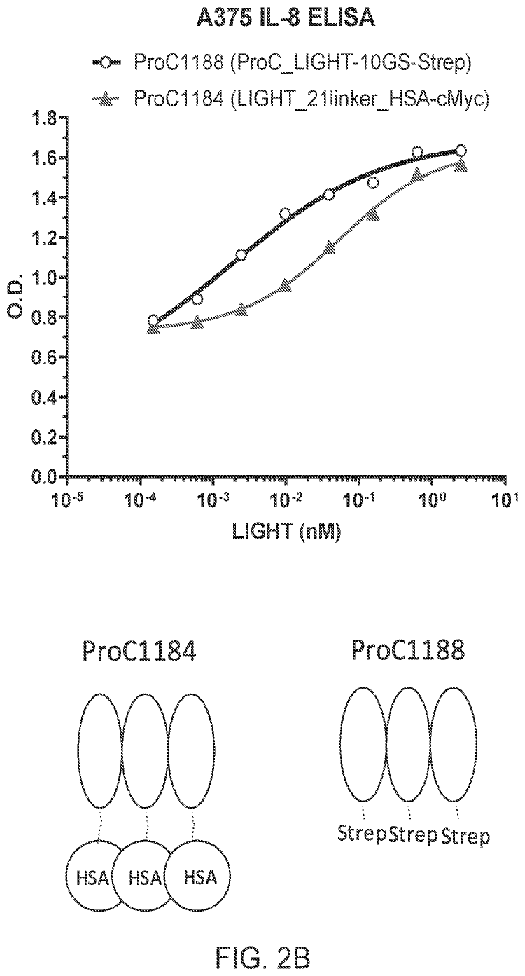

Cytokine construct LIGHT-21linker_HSA_Myc_cMyc (ProC1184) was prepared by recombinant methods. The 1^{st}, 2^{nd} and 3^{rd} monomer constructs of this construct were identical, with each being a polypeptide having the amino acid sequence shown in FIG. 2A (SEQ ID NO: 102). Each of the 1^{st}, 2^{nd} and 3^{rd} monomer constructs comprises, from N-terminus to C-terminus, a modified signal sequence from a mouse IgG kappa signal sequence (SEQ ID NO: 78), a mature cytokine protein that corresponds to a truncated human LIGHT polypeptide (SEQ ID NO: 54) (i.e., the ectodomain domain of human LIGHT, corresponding to residues 85-240 of SEQ ID NO: 79), a non-cleavable linker having the amino acid sequence of SEQ ID NO: 65, a Human Serum Albumin (HSA) sequence (SEQ ID NO: 56), a non-cleavable linker having the of SGG, and a Myc Tag sequence having the SEQ ID NO: 59. The polypeptide was prepared by transforming a host cell with a polynucleotide having the sequence of SEQ ID NO: 103, followed by cultivation of the resulting recombinant host cells. Trimerization of the resulting expressed polypeptides yielded cytokine construct, LIGHT-21linker_HSA_cMyc. The expressed trimeric polypeptides were purified using an HSA-affinity column (e.g., POROS CaptureSelect HSA resin), followed by size exclusion chromatography (SEC), if necessary, to obtain the trimeric protein in at least 95% purity. The final cytokine construct that is assayed did not include the signal sequence.

Cytokine construct LIGHT-10GS-Strep (Prod 188) was also prepared by recombinant methods. The 1^{st}, 2^{nd} and 3^{rd} monomer constructs of this CC were identical, with each being a polypeptide having the amino acid sequence shown in FIG. 2A (SEQ ID NO: 86). Each of the 1^{st}, 2^{nd} and 3^{rd} monomer constructs comprises, from N-terminus to C-terminus, a modified signal sequence from a mouse IgG kappa signal sequence (SEQ ID NO: 78), a mature cytokine protein that corresponds to a truncated human LIGHT polypeptide (SEQ ID NO: 54), a non-cleavable linker having the sequence of SEQ ID NO: 66, and a Strep Tag sequence (SEQ ID NO: 60). The polypeptide was prepared by transforming a host cell with a polynucleotide having the sequence of SEQ ID NO: 87, followed by cultivation of the resulting recombinant host cells. Trimerization of the resulting expressed polypeptides yielded cytokine construct, LIGHT-10GS-Strep. The expressed trimeric polypeptides were purified using a streptavidin-affinity column (e.g., streptavidin agarose resin), followed by size exclusion chromatography (SEC), if necessary, to obtain the trimeric protein in at least 95% purity. The final cytokine construct that is assayed did not have the signal sequence.

Activity of the LIGHT cytokine was evaluated using an HVEM cell-based assay and a Lymphotoxin beta receptor cell-based assay (A375 IL-8 ELISA), as described below.

The activity of each cytokine construct was tested in vitro using a recombinant human HVEM/NF-kB reporter Jurkat cell line expressing firefly luciferase gene (BPS Biosciences #79310). Cells were cultured in RPMI 1640 media supplemented with 10% HI FBS (heat inactivated fetal bovine serum) and 1% Pen/Strep (penicillin-streptomycin). The addition of LIGHT to these cells activates the HVEM receptor and subsequently signals NF-kB transcription factors to bind to the DNA elements required to induce transcription of the luciferase reporter gene. Expression of the luciferase reporter gene can be quantified using the ONE-Glo Luciferase Assay system (commercially available from Promega).

LIGHT-responsive Jurkat HVEM/NF-kB luciferase reporter cells were prepared at a concentration of 390,000 cells/mL in RPMI 1640 media (ThermoFisher Scientific, e.g., Catalog #11875093) supplemented with 10% HI FBS and 90 μL aliquots were pipetted into wells of a white flat-bottom 96-well plate (35,000 cells/well). The tested cytokines were diluted to a starting concentration of 450 nM in RPMI 1640 media supplemented with 10% HI FBS. Duplicates of four-fold serial dilutions were prepared from which 10 μL was added to each well. The plate was shaken for 1-2 minutes at 250 rpm then placed in a 37° C. incubator for 4 hours. Following the 4-hour incubation, the plate was removed from the incubator and allowed to equilibrate to room temperature. The ONE-Glo Luciferase reagent was prepared by transferring the contents of the ONE-Glo Assay Buffer to the lyophilized ONE-Glo Assay Substrate and inverting until the substrate was thoroughly dissolved. 15 mL aliquots of the reagent were stored at −20° C. and thawed to room temperature out of direct light on the day of the assay. Once the reagent and plate were equilibrated to room temperature, 100 μL aliquots of the ONE-Glo Luciferase reagent were pipetted into each well of the plate. The plate was placed on a plate shaker at 250 rpm covered from direct light for 1-2 minutes. After thorough mixing, the luciferase expression was measured using the Tecan Infinite M Plex multimode plate reader. Dose-response curves were generated and EC50 values were obtained by sigmoidal fit non-linear regression using GraphPad Prism software.

The activity of each cytokine construct was tested in vitro using A375 cells, a human melanoma cell line with a high expression of lymphotoxin beta receptor (LTbR). Cells were cultured in Dulbecco's Modified Eagle Medium (DMEM) media supplemented with 10% HI FBS and 1% Pen/Strep. The addition of LIGHT to these cells activates the LTbR which modulates a variety of inflammatory signals, including the secretion of IL-8. The IL-8 secretion by LTbR-activated A375 cells can be measured using the human IL-8/CXCL8 DuoSet ELISA kit (R&D Systems, Catalog #DY208).

A375 cells were prepared at a concentration of 400,000 cells/mL in DMEM media supplemented with 10% HI FBS and 100 μL aliquots were pipetted into the wells of a clear, flat-bottom 96-well plate (40,000 cells/well). The plate was incubated at 37° C. for 3-5 hours. Following incubation, the tested cytokines were diluted to a starting concentration of 5 nM or 400 nM in DMEM media supplemented with 10% HI FBS. Duplicates of four-fold serial dilutions were prepared from which 100 μL was added to each well. The plate was tapped lightly to mix then placed in a 37° C. incubator overnight. On the same day, another clear, flat-bottom 96-well plate was coated with 100 μL of the recommended dilution of IL-8 capture antibody provided in the R&D Systems IL-8 ELISA kit. The plate was then covered and incubated overnight at room temperature. The following day, IL-8 production was analyzed by following the protocol provided in the R&D IL-8 ELISA kit, Once complete, the IL-8 levels were measured using a spectrophotometer at 450 nm. Dose-response curves were generated and EC50 values were obtained by sigmoidal fit non-linear regression using GraphPad Prism software.

The data in FIGS. 2A and 2B show that LIGHT activity of the construct ProC1184 (engineered with a non-cleavable HSA moiety at its C-terminal extremity) was reduced as compared to the LIGHT construct ProC1188 (engineered with a short non-cleavable Strep Tag). This data indicates that the non-cleavable HSA moiety provides steric hindrance, blocking the engagement of LIGHT with its receptors, and activation of downstream signaling. EC50 values for ProC1184 and ProC1188 were computed from the HVEM assay results and Lymphotoxin beta receptor assay and are provided below in Table 4 and Table 5 respectively.

TABLE 4
EC50 [nM]: HVEM Reporter Assay

| | ProC1184 | ProC1188 |
|---|---|---|
| EC50 | >10e6 | 0.8433 |

TABLE 5
EC50 [pM]: Lymphotoxin beta receptor Reporter Assay

| | ProC1184 | ProC1188 |
|---|---|---|
| EC50 | 79.3 | 5.3 |

The data in Table 4 above indicates an extremely high (e.g., greater than 10⁶ fold (one million fold)) masking efficiency for ProC1184 as calculated by comparing the EC50 of the control ProC1188 trimer to the EC50 of the masked ProC1184 in the HVEM Reporter Assay.

### Example 2: Activity of LIGHT Cytokine Constructs Engineered with a Cleavable Affinity Peptide Mask

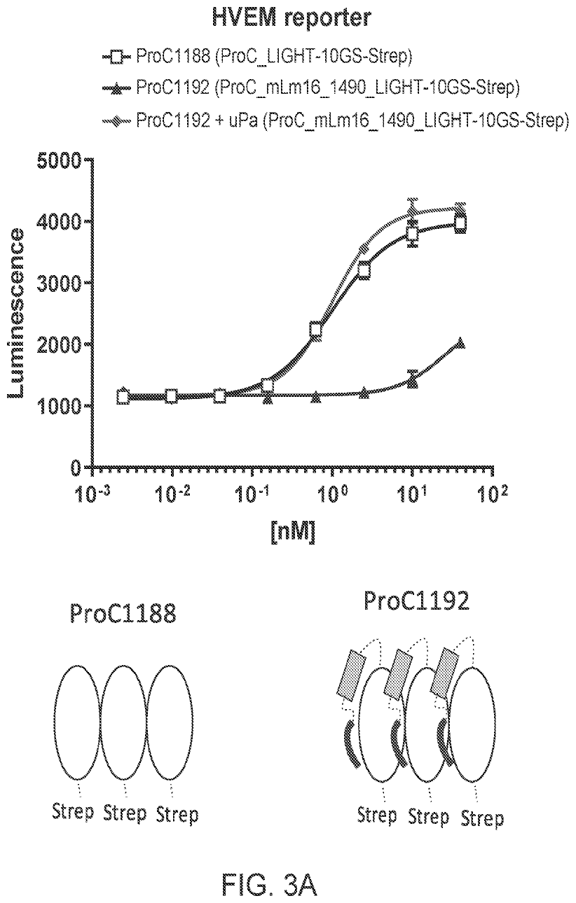

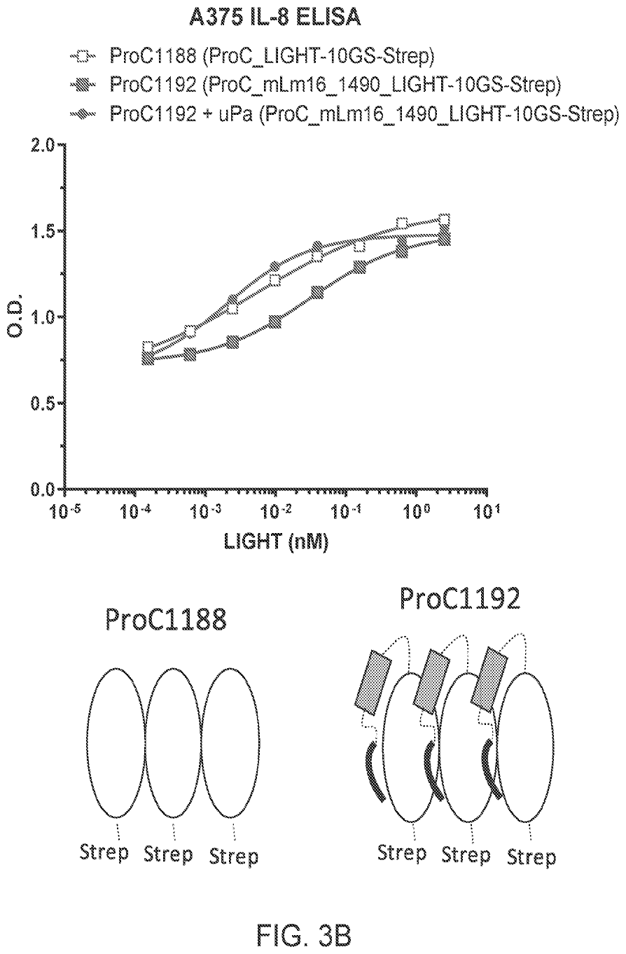

Cytokine construct ProC_mLm16_1490_LIGHT-10GS-Strep (ProC1192) was prepared by recombinant methods. The 1^{st}, 2^{nd} and 3^{rd} monomer constructs of this ACC were identical, with each being a polypeptide having the amino acid sequence shown in FIG. 3A (SEQ ID NO: 88). Each of the 1^{st}, 2^{nd} and 3^{rd} monomer constructs comprises, from N-terminus to C-terminus, a signal sequence from a modified mouse IgG kappa signal sequence (SEQ ID NO: 78), a header sequence (SEQ ID NO: 76), an affinity masking peptide having the sequence of SEQ ID NO: 61, a linker having the sequence of SEQ ID NO: 68, a cleavable moiety having the sequence of SEQ ID NO: 62, a linker having the sequence of GGS, a mature cytokine protein that corresponds to a truncated human LIGHT polypeptide (SEQ ID NO: 54) (i.e., the ectodomain domain of human LIGHT, corresponding to residues 85-240 of SEQ ID NO: 79), a non-cleavable linker having the sequence of SEQ ID NO: 66, and a Strep Tag sequence (SEQ ID NO: 60). The polypeptide was prepared by transforming a host cell with a polynucleotide having the sequence of SEQ ID NO: 89, followed by cultivation of the resulting recombinant host cells. Trimerization of the resulting expressed polypeptides yielded cytokine construct, ProC_mLm16_1490_LIGHT-10GS-Strep. The final cytokine construct that is assayed did not have the signal sequence.

The data in FIGS. 3A and 3B show that LIGHT activity of the ACC ProC1192 engineered with a cleavable affinity peptide mask at its N-terminal extremity was reduced as compared to the LIGHT construct ProC1188 engineered with a short non-cleavable Strep Tag. This data indicates that the cleavable affinity peptide mask provides effective masking by blocking the engagement of LIGHT with its receptors, and activation of downstream signaling.

To cleave the affinity peptide mask, LIGHT-containing ACCs were treated overnight at 37° C. with recombinant human proteases such as urokinase-type plasminogen activator (uPA). The results from these assays (FIGS. 3A and 3B) indicate that the treatment of LIGHT-containing ACCs with proteases could restore activity to a level that is comparable to the LIGHT cytokine engineered with no masking moiety (no affinity peptide mask or HSA moiety).

EC50 values for ProC1192 and ProC1188 were computed from the HVEM assay results and Lymphotoxin beta receptor assay and are provided below in Table 6 and Table 7 respectively.

TABLE 6
EC50 [nM]: HVEM Reporter Assay

| | ProC1192 | ProC1192 + uPa | ProC1188 |
|---|---|---|---|
| EC50 | 25.6 | 0.999 | 1.0 |

TABLE 7
EC50 [pM]: Lymphotoxin beta receptor Reporter Assay

| | ProC1192 | ProC1192 + uPa | ProC1188 |
|---|---|---|---|
| EC50 | 31.3 | 2.0 | 5.3 |

The data in Table 6 above indicates a masking efficiency for ProC1192 of greater than 20-fold as calculated by comparing the EC50 of either the control ProC1188 trimer or the protease-activated ProC1 192 to the EC50 of the masked ProC1 184 in the HVEM Reporter Assay.

### Example 3: Activity of LIGHT Cytokine Constructs Engineered with a Cleavable Affinity Peptide Mask and a Non-Cleavable HSA Steric Masking Moiety

Cytokine construct ProC_mLm-16_1490_LIGHT_21linker_HSA-cMyc (ProC1163) was prepared by recombinant methods. The 1^{st}, 2^{nd} and 3^{rd} monomer constructs of this CC were identical, with each being a polypeptide having the amino acid sequence shown SEQ ID NO: 130. Each of the 1^{st}, 2^{nd} and 3^{rd} monomer constructs comprises, from N-terminus to C-terminus, a signal sequence from a modified mouse IgG kappa signal sequence (SEQ ID NO: 78), a header sequence (SEQ ID NO: 76), a masking peptide having the sequence of SEQ ID NO: 61, a linker having the sequence of SEQ ID NO: 68, a cleavable moiety having the sequence of SEQ ID NO: 62, a linker having the sequence of GGS, a mature cytokine protein that corresponds to truncated human LIGHT polypeptide (SEQ ID NO: 54) (i.e., the ectodomain domain of human LIGHT, corresponding to residues 85-240 of SEQ ID NO: 79), a non-cleavable linker having the of SEQ ID NO: 65, a Human Serum Albumin (HSA) sequence (SEQ ID NO: 56), a non-cleavable linker having the sequence of SGG) and a Myc Tag sequence (SE ID NO: 59). The polypeptide was prepared by transforming a host cell with a polynucleotide having the sequence of SEQ ID NO: 131, followed by cultivation of the resulting recombinant host cells. Trimerization of the resulting expressed polypeptides yielded cytokine construct, ProC_mLm-16_1490_LIGHT-21linker_HSA_cMyc. The expressed trimeric polypeptides were purified using an HSA-affinity column (e.g., POROS CaptureSelect HSA resin), followed by size exclusion chromatography (SEC), if necessary, to obtain the trimeric protein in at least 95% purity. The final cytokine construct that is assayed did not have the signal sequence.

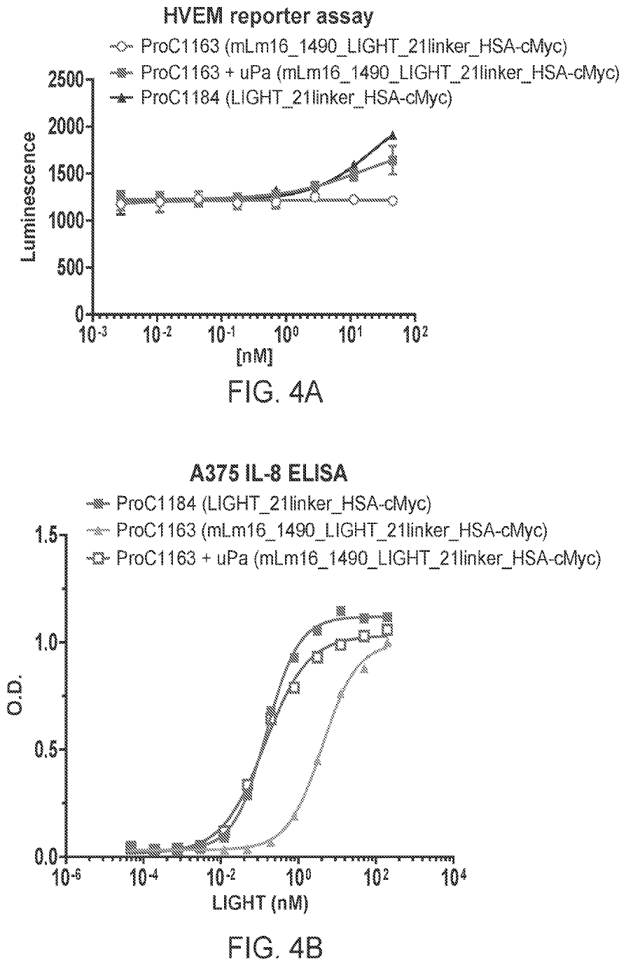

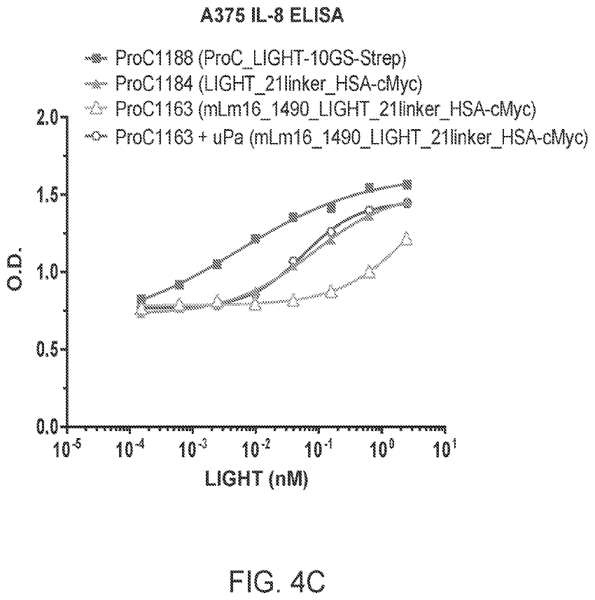

The data in FIGS. 4A and 4B show that LIGHT activity of the ACC ProC1163 engineered with a non-cleavable HSA moiety at its C-terminal extremity and a cleavable affinity peptide at its N-terminal extremity was reduced as compared to the LIGHT construct ProC1184 engineered with a non-cleavable HSA moiety. This data indicates that the non-cleavable HSA moiety and the cleavable affinity peptide can each reduce independently LIGHT activity. It also indicates that both approaches can be combined to further reduce LIGHT signaling activity. FIG. 4C similarly shows that the LIGHT activity of the ACC ProC1163 (with peptide mask in addition to non-cleavable HSA moiety) was reduced as compared to the LIGHT construct ProC1184 (non-cleavable HSA moiety), and that the LIGHT activity of the uPa protease-activated ProC1163 ACC was recovered to a level comparable to the ProC1184 construct. Both ProC1184 and ProC1163 display LIGHT activity that is lower than that of the control ProC1188 construct (engineered with a short non-cleavable Strep Tag).

### Example 4: Activity of LIGHT Activatable Cytokine Constructs Engineered with a Cleavable Affinity Peptide Mask and a Cleavable HSA Steric Masking Moiety

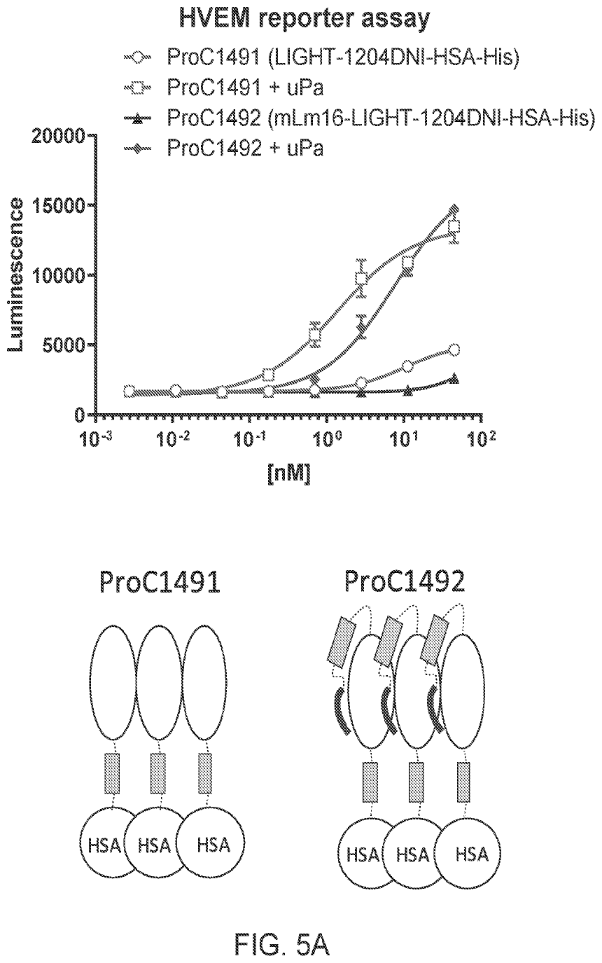

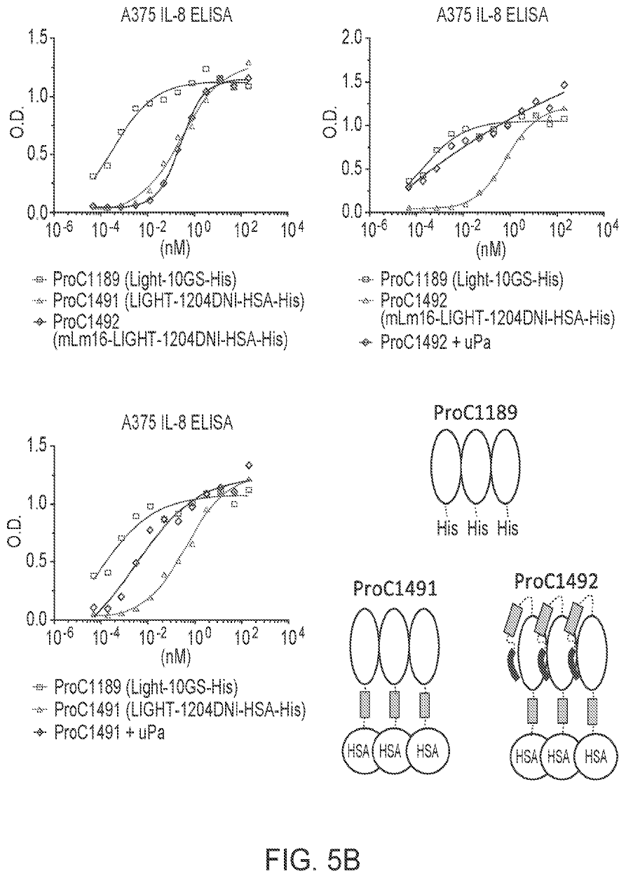

Cytokine construct ProC_LIGHT-1204DNI-HSA-His (ProC1491) was prepared by recombinant methods. The 1^{st}, 2^{nd} and 3^{rd} monomer constructs of this ACC were identical, with each being a polypeptide having the amino acid sequence shown in FIG. 5A (SEQ ID NO: 90). Each of the 1^{st}, 2^{nd} and 3^{rd} monomer constructs comprises, from N-terminus to C-terminus, a modified signal sequence from a mouse IgG kappa signal sequence (SEQ ID NO: 78), a mature cytokine protein that corresponds to a truncated human LIGHT polypeptide (SEQ ID NO: 54) (i.e., the ectodomain domain of human LIGHT, corresponding to residues 85-240 of SEQ ID NO: 79), a linker having the SEQ ID NO: 64, a cleavable moiety having the of SEQ ID NO: 63, a linker having the sequence of GS, a Human Serum Albumin (HSA) sequence (SEQ ID NO: 56), a linker having the sequence of S, and a His Tag having the of SEQ ID NO: 58. The polypeptide was prepared by transforming a host cell with a polynucleotide having the sequence of SEQ ID NO: 91, followed by cultivation of the resulting recombinant host cells. Trimerization of the resulting expressed polypeptides yielded cytokine construct, ProC_LIGHT-1204DNI-HSA-His. The final cytokine construct that is assayed did not have the signal sequence.

Cytokine construct ProC_mLm16-LIGHT-1204DNI-HSA-His (ProC1492) was prepared by recombinant methods. The 1^{st}, 2^{nd} and 3^{rd} monomer constructs of this ACC were identical, with each being a polypeptide having the amino acid sequence shown in FIG. 5A (SEQ ID NO: 92). Each of the 1^{st}, 2^{nd} and 3^{rd} monomer constructs comprises, from N-terminus to C-terminus, a modified signal sequence from a mouse IgG kappa signal sequence (SEQ ID NO: 78), a header sequence of SEQ ID NO: 76, a masking peptide having the sequence of SEQ ID NO: 61, a linker having the sequence of SEQ ID NO: 68, a cleavable moiety having the sequence of SEQ ID NO: 81, a linker having the sequence of GGS, a mature cytokine protein that corresponds to a truncated human LIGHT polypeptide (SEQ ID NO: 54) (i.e., the ectodomain domain of human LIGHT, corresponding to residues 85-240 of SEQ ID NO: 79), a linker having the SEQ ID NO: 64, a cleavable moiety having the of SEQ ID NO: 63, a linker of the sequence GS, a Human Serum Albumin (HSA) sequence (SEQ ID NO: 56), a linker having the sequence S, and a His Tag having the sequence of SEQ ID NO: 58. The polypeptide was prepared by transforming a host cell with a polynucleotide having the sequence of SEQ ID NO: 93, followed by cultivation of the resulting recombinant host cells. Trimerization of the resulting expressed polypeptides yielded cytokine construct, ProC_mLm16-LIGHT-1204DNI-HSA-His. The final cytokine construct that is assayed did not have the signal sequence.

The data in FIG. 5A show that, in the HVEM reporter assay, activity of LIGHT ProC1492 containing a cleavable peptide mask at its N-terminal extremity and a cleavable HSA moiety at its C-terminal extremity (dual mask) was further reduced as compared to the activity of LIGHT ProC1491 containing only a cleavable HSA moiety at its C-terminal extremity (single mask). Upon protease activation by uPa, ProC1491 and ProC1492 lost their masking potential and recovered LIGHT cytokine activity.

The data in FIG. 5B show that in the Lymphotoxin beta receptor assay (A375 ILL-8 ELISA), both ProC1491 and ProC1492 have reduced activity as compared to the unmasked LIGHT ProC1189. Upon protease activation by uPa, both ProC1491 and ProC1492 recovered activity similar to LIGHT ProC1189.

EC50 values for ProC1491 and ProC1492 were computed from the HVEM assay results and Lymphotoxin beta receptor assay and are provided below in Table 8 and Table 9 respectively.

TABLE 8
EC50 [nM]: HVEM Reporter Assay

| | ProC1491 | ProC1491 + uPa | ProC1492 | ProC1492 + uPa |
|---|---|---|---|---|
| EC50 | 10.51 | 1.3 | 67.5 | 7.7 |

TABLE 9
EC50 [pM]: Lymphotoxin beta receptor Reporter Assay

| | ProC1491 | ProC1491 + uPa | ProC1492 | ProC1492 + uPa |
|---|---|---|---|---|
| EC50 | 437.2 | 3.2 | 671.3 | ambiguous |

All-together, these data indicate that LIGHT activation of both HVEM and Lymphotoxin beta receptor pathway can be reduced by adding either or the combination of a cleavable peptide affinity mask (affinity masking moiety) and a cleavable HSA moiety (steric masking moiety) to LIGHT protein. Upon protease activation, LIGHT recovers its full signaling potential.

### Example 6. In Vitro Characterization of Additional Cytokine Constructs

Additional activatable cytokine constructs were also prepared by recombinant methods. The 1^{st}, 2^{nd} and 3^{rd} monomer constructs of these ACCs were identical.

In some ACCs, the HSA moiety was engineered at the N-terminal extremity of LIGHT, and the affinity peptide mask was engineered at the C-terminal extremity of LIGHT.

Cytokine construct ProC_cMyc-HSA-21GS-1204DNI-LIGHT (ProC1497) (SEQ ID NO: 120) comprises, from N-terminus to C-terminus, a modified signal sequence from a mouse IgG kappa signal sequence (SEQ ID NO: 78), a linker with sequence of GSG, a Myc Tag having the sequence of SEQ ID NO: 59, a linker having sequence of G, a Human Serum Albumin (HSA) sequence (SEQ ID NO: 56), a linker having the SEQ ID NO: 64, a cleavable moiety having the of SEQ ID NO: 63, a linker having the GS, and a mature cytokine protein that corresponds to a truncated human LIGHT polypeptide (SEQ ID NO: 54) (i.e., the ectodomain domain of human LIGHT, corresponding to residues 85-240 of SEQ ID NO: 79). The polypeptide was prepared by transforming a host cell with a polynucleotide having the sequence of SEQ ID NO: 121, followed by cultivation of the resulting recombinant host cells. Trimerization of the resulting expressed polypeptides yielded cytokine construct, ProC_cMyc-HSA-21GS-1204DNI-LIGHT. The final cytokine construct that is assayed did not have the signal sequence.

Cytokine construct ProC_cMyc-HSA-21GS-1204DNI-LIGHT-mLm16 (ProC1498) (SEQ ID NO: 126) comprises, from N-terminus to C-terminus, a signal sequence from a modified mouse IgG kappa signal sequence (SEQ ID NO: 78), a linker with sequence of GSG, a Myc Tag having the of SEQ ID NO: 59, a linker having sequence of G, a Human Serum Albumin (HSA) sequence (SEQ ID NO: 56), a linker having the SEQ ID NO: 64, a cleavable moiety having the of SEQ ID NO: 63, a linker having the GS, a mature cytokine protein that corresponds to a truncated human LIGHT polypeptide (SEQ ID NO: 54) (i.e., the ectodomain domain of human LIGHT, corresponding to residues 85-240 of SEQ ID NO: 79), a linker having the SEQ ID NO: 75, a cleavable moiety having the of SEQ ID NO: 62, a linker having the SEQ ID NO: 68, and a masking peptide having the sequence of SEQ ID NO: 61. The polypeptide was prepared by transforming a host cell with a polynucleotide having the sequence of SEQ ID NO: 127, followed by cultivation of the resulting recombinant host cells. Trimerization of the resulting expressed polypeptides yielded cytokine construct, ProC_cMyc-HSA-21GS-1204DNI-LIGHT-mLm16. The final cytokine construct that is assayed did not have the signal sequence.

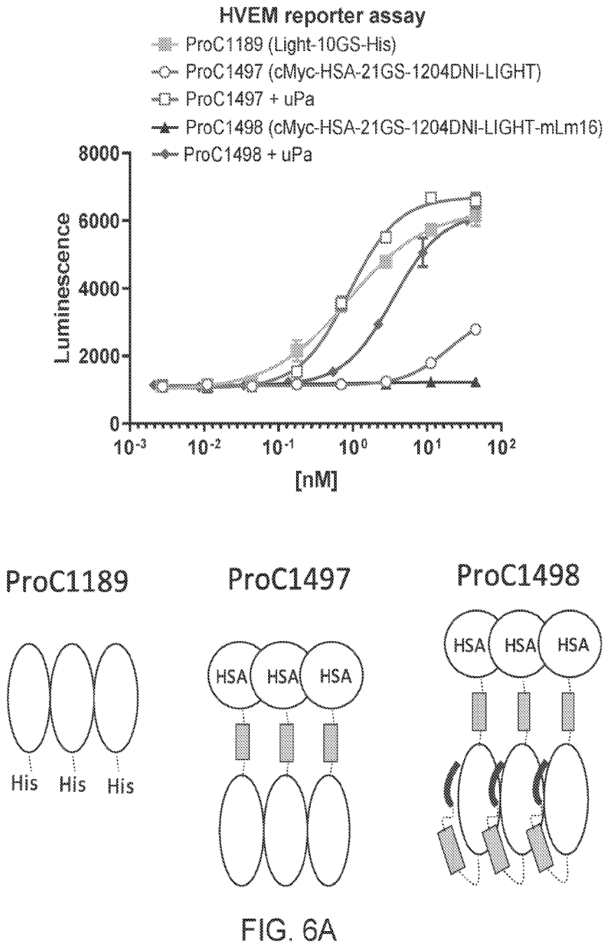

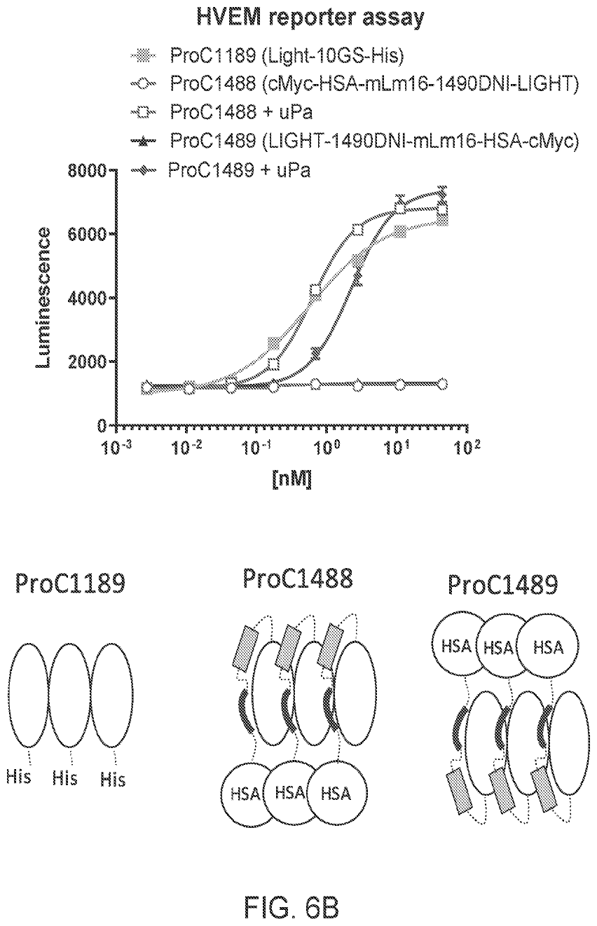

The data in FIG. 6A show that, in the HVEM reporter assay, activity of ACC ProC1497, engineered with a cleavable HSA moiety at its N-terminal extremity, or activity of ACC ProC1498, engineered with a cleavable HSA moiety at its N-terminal extremity and a cleavable affinity peptide mask at its C-terminal extremity is significantly reduced as compared to LIGHT ProC1189 engineered without a mask. Activity of ProC1498 was further reduced as compared to activity of ProC1497. It also indicates that the addition of a peptide affinity mask and an HSA moiety at either the C-terminal or N-Terminal extremity of LIGHT can be combined to further reduces LIGHT signaling activity. Upon protease activation by uPa, both ProC1497 and ProC1498 recovered activity similar to LIGHT ProC1189.

EC50 values for ProC1497 and ProC1498 were computed from the HVEM assay results are provided below in Table 10.

TABLE 10
EC50 [nM]: HVEM Reporter Assay

| | ProC1189 | ProC1497 | ProC1497 + uPa | ProC1498 | ProC1498 + uPa |
|---|---|---|---|---|---|
| EC50 | 0.86 | 19 | 0.89 | n.d. | 3.43 |

The data in Table 10 above indicates a masking efficiency for ProC1497 of 22-fold (22×) as calculated by comparing the EC50 of the intact ProC1497 ACC to the EC50 of the uPa protease-cleaved (activated) ProC1497 ACC in the HVEM Reporter Assay. The data in Table 10 indicates an extremely high (e.g., greater than 10⁶ fold (one million fold)) masking efficiency for ProC1498 as calculated by comparing the EC50 of the intact ProC1498 ACC to the EC50 of the uPa protease-cleaved (activated) ProC1498 ACC in the HVEM Reporter Assay. The EC50 values for the masked ACC ProC1498 was not detectable (n.d.) because no cytokine activity was detected at the concentrations tested in the HVEM reporter assay.

In some ACCs, the HSA moiety and affinity peptide mask were engineered on the same extremity of LIGHT, either N-terminal or C-terminal, such that a single cleavable moiety (CM) can be cleaved to remove both the HSA moiety and the affinity peptide mask.

Cytokine construct ProC_cMyc-HSA-mLm16-1490DNI-LIGHT (ProC1488) (SEQ ID NO: 124) comprises, from N-terminus to C-terminus, a signal sequence from a modified mouse IgG kappa signal sequence (SEQ ID NO: 78), a linker with sequence of GSG, a Myc Tag having the of SEQ ID NO: 59, a linker having sequence of G, a Human Serum Albumin (HSA) sequence (SEQ ID NO: 56), a linker having the SEQ ID NO: 67, a header having the sequence (SEQ ID NO: 76), a affinity masking peptide having the sequence of SEQ ID NO: 61, a linker having the SEQ ID NO: 68, a cleavable moiety having the amino acid sequence of SEQ ID NO: 62, a linker having the GGS, and a mature cytokine protein that corresponds to a truncated human LIGHT polypeptide (SEQ ID NO: 54). The polypeptide was prepared by transforming a host cell with a polynucleotide having the sequence of SEQ ID NO: 125, followed by cultivation of the resulting recombinant host cells. Trimerization of the resulting expressed polypeptides yielded cytokine construct, ProC_cMyc-HSA-mLm16-1490DNI-LIGHT. The final cytokine construct that is assayed did not have the signal sequence.

Cytokine construct ProC_LIGHT-1490DNI-mLm16-HSA-cMyc (ProC1489) comprises, from N-terminus to C-terminus, a modified signal sequence from a mouse IgG kappa signal sequence (SEQ ID NO: 122), a mature cytokine protein that corresponds to a truncated human LIGHT polypeptide (SEQ ID NO: 54) (i.e., the ectodomain domain of human LIGHT, corresponding to residues 85-240 of SEQ ID NO: 79), a linker having the SEQ ID NO: 75, a cleavable moiety having the of SEQ ID NO: 62, a linker having the SEQ ID NO: 68, a masking peptide having the sequence of SEQ ID NO: 61, a linker having the SEQ ID NO: 66, a Human Serum Albumin (HSA) sequence (SEQ ID NO: 56), a linker having sequence of S, and a Myc Tag having the of SEQ ID NO: 59. The polypeptide was prepared by transforming a host cell with a polynucleotide having the sequence of SEQ ID NO: 123, followed by cultivation of the resulting recombinant host cells. Trimerization of the resulting expressed polypeptides yielded cytokine construct, ProC_LIGHT-1490DNI-mLm16-HSA-cMyc. The final cytokine construct that is assayed did not have the signal sequence.

Activity of ACCs ProC1488 and ProC1489 was evaluated in the HVEM assay. Data in FIG. 6B show that when the HSA moiety and affinity peptide mask were engineered on the same extremity of LIGHT, either N-terminal or C-terminal, LIGHT activity is significantly reduced as compared to unmasked LIGHT ProC1189. Upon protease activation by uPa, both ProC1488 and ProC1489 recovered activity similar to LIGHT ProC1189.

EC50 values for ProC1488 and ProC1489 were computed from the HVEM assay results are provided below in Table 11.

TABLE 11
EC50 [nM]: HVEM Reporter Assay

| | ProC1189 | ProC1488 | ProC1488 + uPa | ProC1489 | ProC1489 + uPa |
|---|---|---|---|---|---|
| EC50 | 0.58 | n.d. | 0.63 | n.d. | 2.27 |

The EC50 values for the masked ACCs ProC1488 and ProC1489 were not detectable (n.d.) because no cytokine activity was detected at the concentrations tested in the HVEM reporter assay. This indicates that the masking of the LIGHT cytokine was very effective. The data in Table 11 above indicates an extremely high (e.g., greater than 10⁶ fold (one million fold)) masking efficiency for ProC1488 as calculated by comparing the EC50 of the intact ProC1488 ACC to the EC50 of the uPa protease-cleaved (activated) ProC1488 ACC in the HVEM Reporter Assay. The data in Table 11 indicates an extremely high (e.g., greater than 10⁶ fold (one million fold) masking efficiency for ProC1489 of greater than 10\hat{ }6 (one million-fold) as calculated by comparing the EC50 of the intact ProC1489 ACC to the EC50 of the uPa protease-cleaved (activated) ProC1489 ACC in the HVEM Reporter Assay.

### Example 7. In Vitro Characterization of a Human-Mouse Cross-Reactive LIGHT ACC

Tang et al. (Cancer Cell. 2016 Mar. 14; 29(3):285-296) have previously reported that 4 points mutations in human LIGHT (SEQ ID No: 55) retains binding to human HVEM and lymphotoxin beta receptor but confers binding to mouse HVEM and lymphotoxin beta receptor.

ACCs ProC1486 (ProC_mhLIGHT_1204DNI_HSA-cMyc) and ProC1487 (ProC_mLm-16_mhLIGHT_1204DNI_HSA-cMyc) have been engineered similarly to ProC1491 and ProC1492 respectively, with the differences that ProC1486 and ProC1487 contain a mature cytokine protein that corresponds to the ectodomain of human LIGHT with four points mutations (Tang et al.) and a C-terminal linker (SGG) and cMyc tag (SEQ ID NO: 59) in place of the linker and His tag present on ProC1491 and 1492. ProC1491 and 1492 contain a non-mutated a mature cytokine protein that corresponds to the ectodomain of human LIGHT.

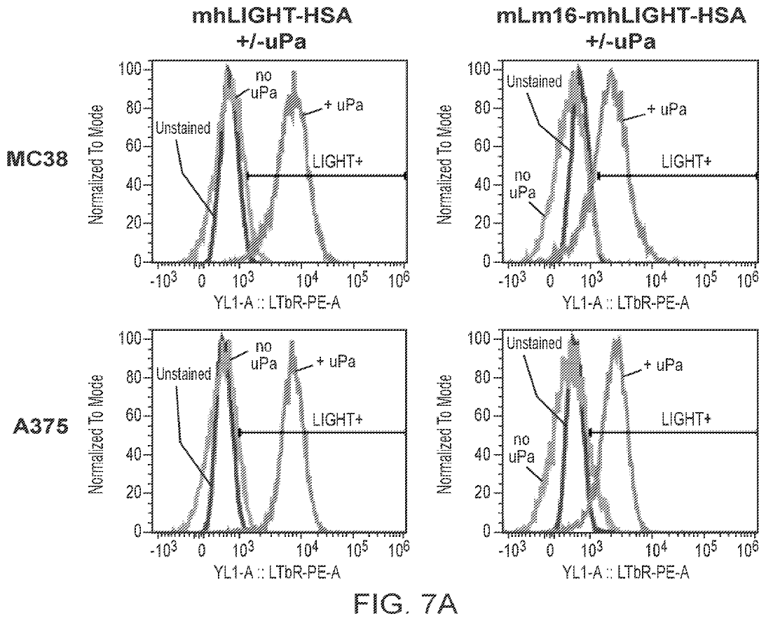

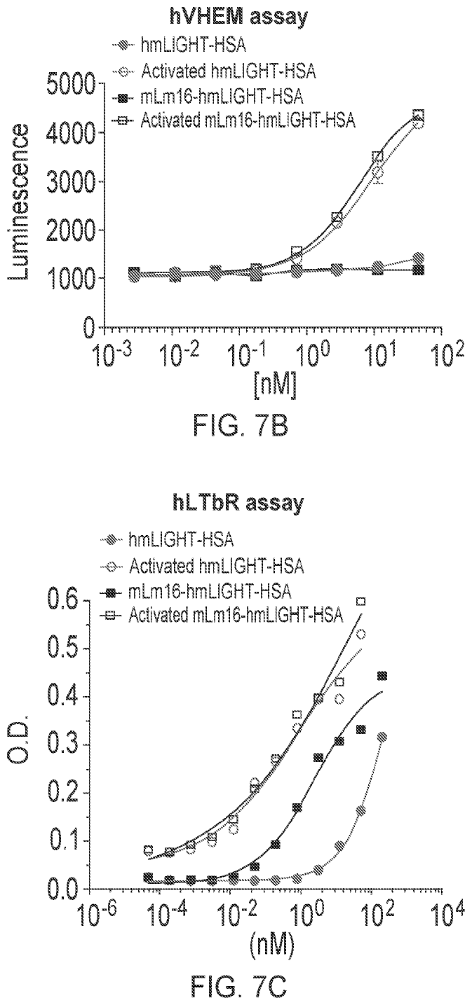

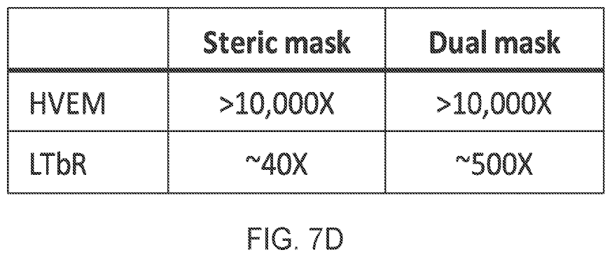

Binding of ProC1486 and ProC1487 to mouse and human lymphotoxin beta receptor was evaluated by flow cytometry. Binding to mouse receptor was evaluated using the MC38 cell line. Binding to human receptor was evaluated using the A375 cell line. The engagement of ProC1486 or ProC1487 to mouse or human receptor was detected with a PE-conjugated anti-human CD258 (LIGHT) Antibody [Biolegend Cat #: 318706 Clone: T5-39]. Data in FIG. 7A shows that ProC1486 and ProC1487 do not bind to A375 or MC38. Upon protease treatment with uPa, both ProC1486 and ProC1487 recovered binding to human and mouse lymphotoxin beta receptor. It indicates that human/mouse LIGHT ACCs engineered with a cleavable HSA moiety and/or a cleavable peptide affinity mask have reduced or no binding to mouse or human lymphotoxin beta receptor. Binding is recovered upon protease activation.

Activity of ACCs ProC1486 and ProC1487 was evaluated in the HVEM reporter assay and the A375-IL8 reporter assay as previously described.

Data in FIGS. 7B and 7C shows that activity of ProC1486 and ProC1487 was almost abolished in the HVEM assay and reduced in the Lymphotoxin beta receptor assay. Activity of ProC1487 engineered with both a peptide affinity mask and a HSA moiety was further reduced in the lymphotoxin beta receptor assay as compared to ProC1486 which is engineered with a HSA moiety only. This data indicated that the same peptide affinity mask and HSA moiety used to reduce human LIGHT activity can be used to reduce activity of a human-mouse cross-reactive LIGHT ACC. Upon protease activation with uPa, activity of ProC1486 and ProC1487 was significantly increased.

ACC ProC2076 (ProC_cMyc-HSA-mLm16-1490-mhLIGHT) has been engineered similarly to ProC1488 with the differences that ProC2076 contains a mature cytokine protein that corresponds to the ectodomain of human LIGHT with four points mutations (Tang et al., Cancer Cell. 2016 Mar. 14; 29(3):285-296).

Activity of ACCs ProC1486, ProC1487 and ProC2076 was evaluated in the Mouse HVEM reporter assay. The Mouse HVEM reporter cell line was generated by transfection of the NF-kB luc-reporter HEK-293s (BPS Bioscience, #60650) with a Mouse HVEM plasmind (Origene, #MC212911). Cells were transfected using Lipofectamine™ 2000 Transfection Reagent (ThermoFisher, #11668019) Cells expressing Mouse HVEM were selected using 0.8 mg/mL Geneticin (ThermoFisher, #10131035).

Cells were cultured in MEM media supplemented with 10% HI FBS (heat inactivated fetal bovine serum) 1% Pen/Strep (penicillin-streptomycin), Non Essential Amico Acid (ThermoFisher, #11140050), Sodium Pyruvate (ThermoFisher, #J61840.18), 50 ug/mL Hygromycin B (Thermofisher, #10687010) and 0.8 mg/mL Geneticin (ThermoFisher, #10131035). The addition of human-mouse cross-reactive LIGHT to these cells activated the Mouse HVEM receptor and subsequently signals NF-kB transcription factors to bind to the DNA elements required to induce transcription of the luciferase reporter gene. Expression of the luciferase reporter gene was quantified using the ONE-Glo Luciferase Assay system (commercially available from Promega).

LIGHT-responsive Jurkat HVEM/NF-kB luciferase reporter cells were plated at 20,000 cells per well in DMEM media (ThermoFisher Scientific, e.g., Catalog #10564011) supplemented with 10% HI FBS in a white flat-bottom 96-well plate. After overnight incubation, the media was aspirated. The tested cytokines were diluted to a starting concentration of 25 nM in DMEM media supplemented with 10% HI FBS. Duplicates of five-fold serial dilutions were prepared from which 100 μL was added to each well. The plate was shaken for 1-2 minutes at 250 rpm then placed in a 37° C. incubator for 4 hours. Following the 5-hour incubation, the plate was removed from the incubator and allowed to equilibrate to room temperature. The ONE-Glo Luciferase reagent was prepared by transferring the contents of the ONE-Glo Assay Buffer to the lyophilized ONE-Glo Assay Substrate and inverting until the substrate was thoroughly dissolved. 15 mL aliquots of the reagent were stored at −20° C. and thawed to room temperature out of direct light on the day of the assay. Once the reagent and plate were equilibrated to room temperature, 100 μL aliquots of the ONE-Glo Luciferase reagent were pipetted into each well of the plate. The plate was placed on a plate shaker at 250 rpm covered from direct light for 1-2 minutes. After thorough mixing, the luciferase expression was measured using the Tecan Infinite M Plex multimode plate reader. Dose-response curves were generated and EC50 values were obtained by sigmoidal fit non-linear regression using GraphPad Prism software.

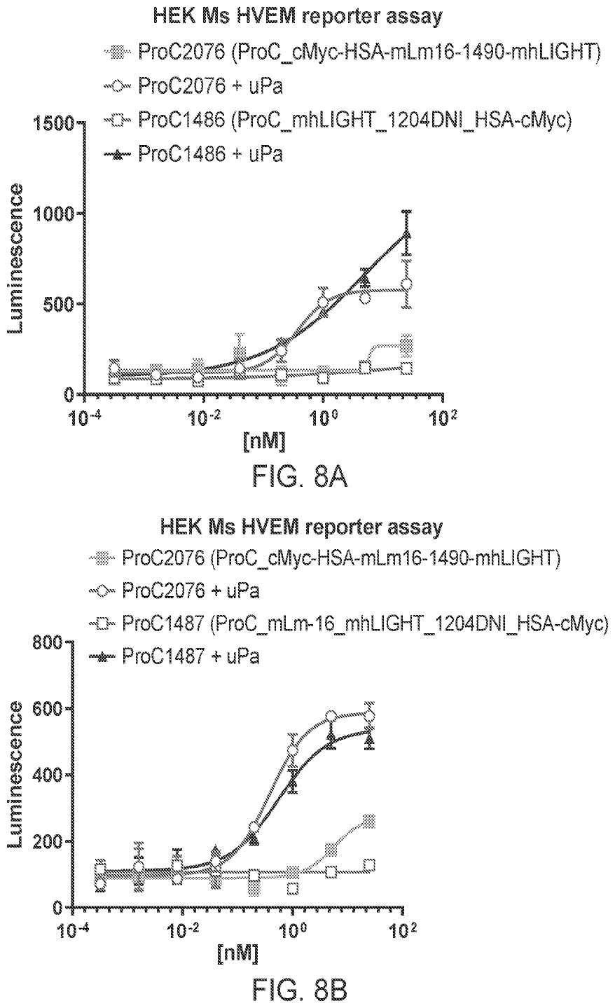

Data in FIGS. 8A and 8B shows that activity of ProC1486, ProC1487 and ProC2076 was significantly reduced in the HVEM assay. Activity of ProC2076, engineered with a cleavable HSA moiety at its C-terminal extremity (FIG. 8A), and activity of ProC1487 (FIG. 8.B), engineered with a cleavable HSA moiety at its C-terminal extremity and a cleavable affinity peptide mask at its N-terminal extremity, were further reduced as compared to ProC2076 which was engineered with a cleavable HSA moiety and a peptide affinity mask at it's N-terminal extremity. Upon protease activation with uPa, activity of ProC1486, ProC1487 and ProC2076 was significantly increased (FIGS. 8A and 8B).

### Example 8. In Vivo Characterization of a Human LIGHT ACC in HT-29 Xenograft Mice

The antitumor in vivo activity of human LIGHT ACC ProC1491 engineered with a cleavable HSA domain at the C-terminal extremity of LIGHT, and the antitumor activity of human LIGHT ProC1189 engineered with a His tag at the C-terminal extremity of LIGHT were evaluated in the HT-29 xenograft model. LTβR activation induced by its ligand lymphotoxin-α/β, LIGHT or an agonistic mAb triggers an IFNg-dependent tumor growth inhibition both in vitro and in vivo in HT29 and WiDr colon carcinoma models (Lukashev et al, Cancer Research, 2006).

To evaluate the in vivo antitumor activity of ProC1491 and ProC1189, on day 0, 2×10⁶ HT29-Luc2 tumor cells in 100 μL serum-free RPMI were injected SC in the flank of 7-8 weeks female nu/nu mice. When tumors reached ˜60-100 mm³ mice received an intraperitoneal injection of each test article dosed at 1 mg/kg. Each animal received a dose of test article on day 1, 5, 8, 12, 15, 19 and 22. Body weights and tumor measurements were recorded twice weekly for the duration of the study. Mice experiments were conducted according to the IACUC protocol AP303 (Use of Subcutaneous Mouse Tumor Models to Evaluate the Anti-Tumor Activity and IACUC Guideline G01: Management of Tumor Models).

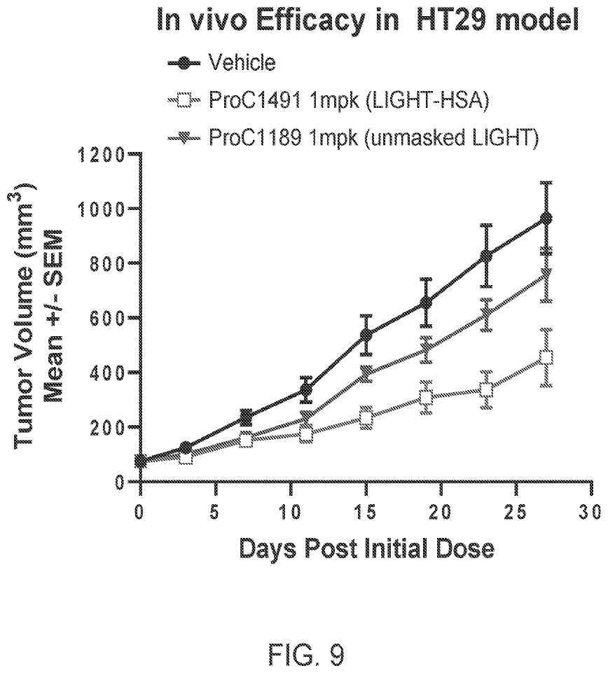

Percent tumor growth inhibition (% TGI) was calculated with the following formula: TGI (%) [1−(RTV of the treated group)/(RTV of the control group)]×100(%), were RTW=(tumor volume on measured day)/(tumor volume on day 0). Data in FIG. 9 shows that, in the HT-29 xenograft mice model, human LIGHT ACC ProC1491 engineered with a cleavable HSA domain at the C-terminal extremity of LIGHT was more potent at promoting tumor growth inhibition than human LIGHT ProC1189 engineered with a His tag at the C-terminal extremity of LIGHT. The relative percent TGI are indicated in Table 12. This indicates that human LIGHT ACC ProC1491 is more potent than human LIGHT ProC1189 at inducing tumor localized antitumor activity.

TABLE 12
Percent Tumor growth inhibition in HT-29 xenograft mice model:

| | ProC1491 | ProC1189 |
|---|---|---|
| % TGI | 52.5% | 21.1% |

### Example 9. In Vivo Characterization of a Human-Mouse Cross-Reactive LIGHT ACC in MC38 Syngeneic Mice

The antitumor activity of human-mouse cross-reactive LIGHT ACC ProC1486, ProC1487 and ProC2076 was evaluated using the MC38 colon adenocarcinoma syngeneic mice model.

Mice experiments were conducted according to the IACUC protocol AP303 (Use of Subcutaneous Mouse Tumor Models to Evaluate the Anti-Tumor Activity and IACUC Guideline G01: Management of Tumor Models).

Seven to nine weeks old female C57BL/6 mice were implanted with MC38 tumor cells in serum-free medium. The animals were dosed intra-peritoneally with LIGHT ACC as single agent or in combination with a mouse anti-PD-1 antibody (clone RPMI1-14; BioXCell, Cat #BP0146) and tumor measurements were recorded twice weekly for the duration of the study. Percent tumor growth inhibition (% TGI) was calculated with the following formula: TGI (%)=[1−(RTV of the treated group)/(RTV of the control group)], 100(%), were RTW=(tumor volume on measured day)/(tumor volume on day 0).

In some experiments, tumor and spleen were collected to evaluate PharmacoDymanic (PD) biomarkers of LIGHT ACC activity. Tumor samples were processed according to the Miltenyi tumor dissociation kit (Miltenyi, Cat #130-096-730). Spleens were processed mechanically using a syringe plunger and treated with ACK lysis buffer (ThermoFisher, cat #A1049201) to remove red blood cells. Immune cells were analyzed by Flow cytometry using an Attune NxT Flow cytometer (ThermoFisher). Flow cytometry data were plotted and analyzed using Prism Software.

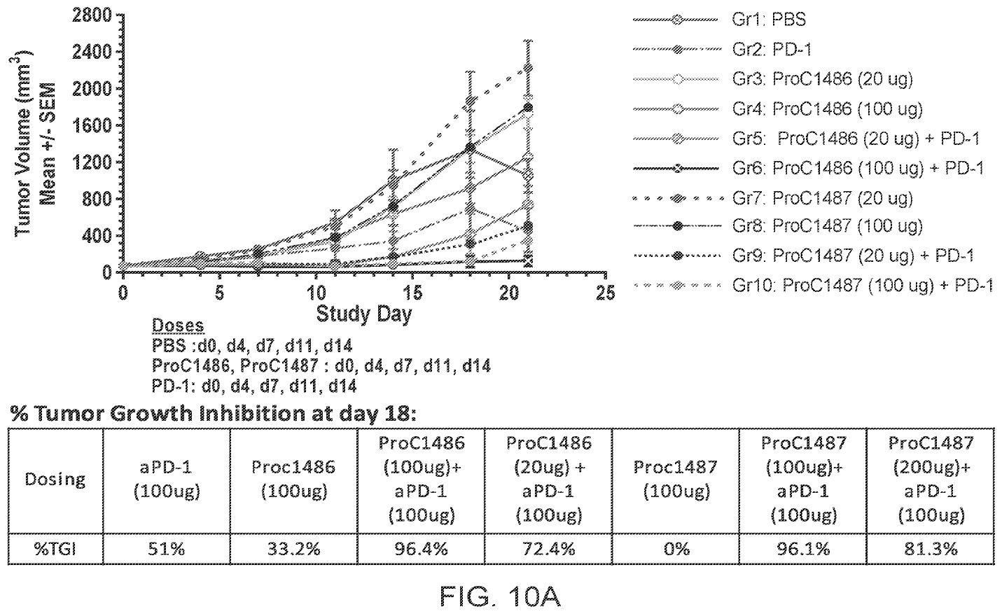

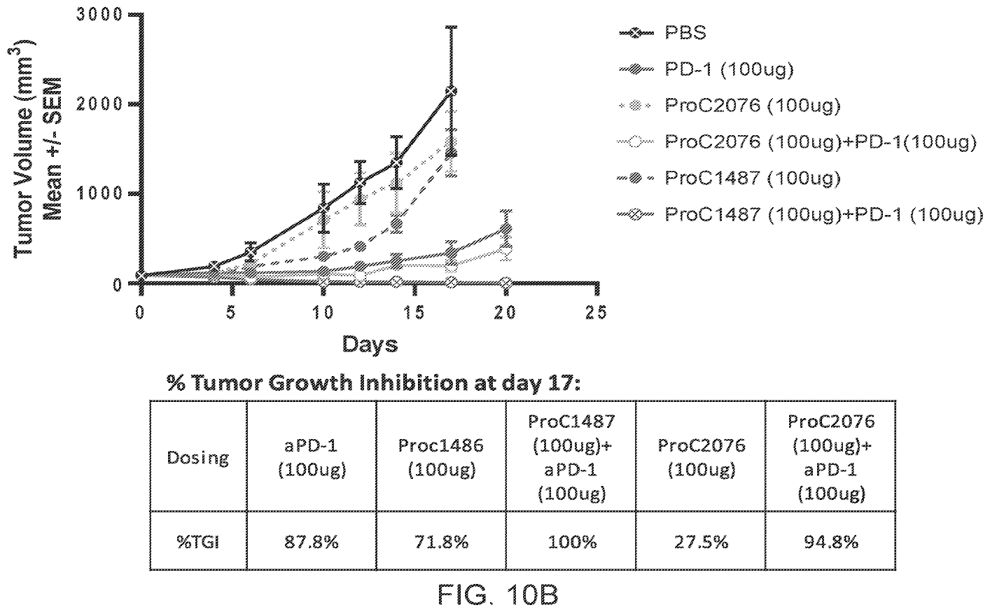

Data in FIGS. 10A and 10B show that ProC1486 and ProC1487 had single agent antitumor activity in the MC38 syngeneic mice model. The antitumor activity of ProC1486 and ProC1487 was further enhanced when dosed in combination with anti PD-1. ProC2076 had minimal single agent activity (FIG. 10B). Its antitumor activity was also increased in combination with an anti-PD-1 antibody in the MC38 syngeneic mice model (FIG. 10B).

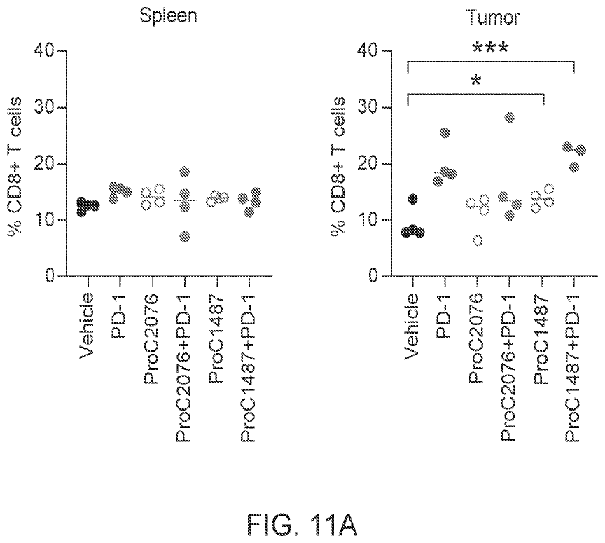

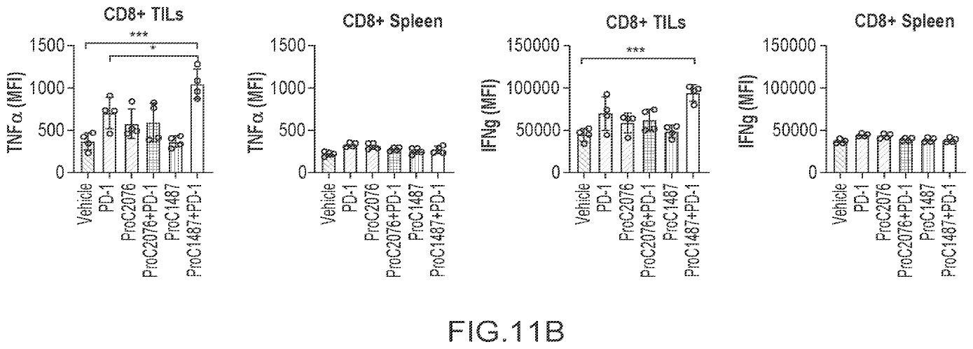

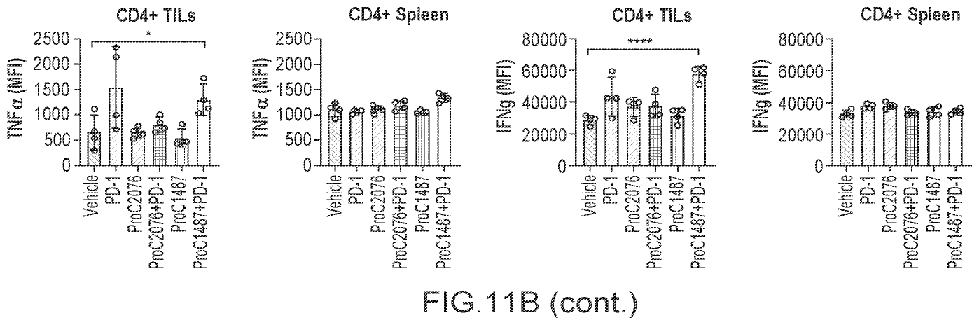

Data in FIGS. 11A-111B show that at day 6 post treatment initiation in the MC38 syngeneic mice model, ProC1487, dosed as a single agent or in combination with an anti-PD-1 antibody, was able to increase the level of CD8+ T cells in the tumor micro-environment (FIG. 11A) as well as to promote the production of Th1 cytokines (IFNγ, TNFα) by CD8+ T cells (FIG. 11B). ProC1487 had no or limited activity in the spleen, indicating that the activity of ProC1487 is only localized in the tumor. In some cases, the data were analyzed in Prism Software using a statistical t-test. The significance of the test is illustrated in FIGS. 11A and 11B with an asterix sign.

TABLE 12
Example Sequences

| SEQ ID | | |
|---|---|---|
| NO: | NOTES | SEQUENCE |
| 1 | | GQSGS |
| 2 | ProC1189/ | QERRSHEVNPAAHLTGANSSLTGSGGPLLWETQLGLAFLRG |
| | ProC_LIGHT-10GS- | LSYHDGALVVTKAGYYYIYSKVQLGGVGCPLGLASTITHGL |
| | His | YKRTPRYPEELELLVSQQSPCGRATSSSRVWWDSSFLGGVV |
| | Protein | HLEAGEEVVVRVLDERLVRLRDGTRSYFGAFMVSGGGSGG |
| | | GSGHHHHHH |
| 3 | ProC1189/ | CAGGAACGGCGCTCCCACGAGGTCAATCCGGCGGCTCAT |
| | ProC_LIGHT-10GS- | TTGACTGGAGCCAACTCTAGTCTTACCGGGAGCGGCGGT |
| | His | CCATTGCTGTGGGAGACGCAACTTGGATTGGCTTTTCTTC |
| | DNA | GGGGATTGTCTTACCACGATGGAGCACTCGTTGTTACGAA |
| | | AGCCGGTTACTACTACATTTATTCTAAGGTCCAGTTGGGT |
| | | GGAGTTGGCTGTCCCCTGGGACTTGCAAGCACTATCACTC |
| | | ACGGACTGTACAAGAGGACACCCCGATACCCCGAGGAAC |
| | | TCGAGCTGCTTGTGAGCCAACAGTCTCCATGTGGGCGCGC |
| | | AACTAGTTCTTCACGGGTGTGGTGGGACTCCAGTTTCCTG |
| | | GGAGGTGTTGTTCATCTCGAGGCAGGTGAAGAAGTAGTT |
| | | GTCAGGGTACTTGATGAACGGCTTGTTAGACTCCGGGAT |
| | | GGAACGAGGTCCTATTTCGGTGCGTTCATGGTAAGCGGG |
| | | GGCGGAAGTGGGGGCGGGTCAGGTCATCATCACCACCAT |
| | | CATTGA |
| 4 | ProC1193/ | QGQSGSRHPCRHDPHIICYKFGGGSSGGSISSGLLSGRSDNIG |
| | ProC_mLm- | GSQERRSHEVNPAAHLTGANSSLTGSGGPLLWETQLGLAFL |
| | 16_1490_LIGHT_10GS_ | RGLSYHDGALVVTKAGYYYIYSKVQLGGVGCPLGLASTITH |
| | His | GLYKRTPRYPEELELLVSQQSPCGRATSSSRVWWDSSFLGG |
| | Protein | VVHLEAGEEVVVRVLDERLVRLRDGTRSYFGAFMVSGGGS |
| | | GGGSGHHHHHH |
| 5 | ProC1193/ | CAAGGACAAAGTGGTTCCCGACACCCGTGCAGGCATGAC |
| | ProC_mLm- | CCACATATAATCTGTTATAAGTTTGGTGGAGGCTCCTCCG |
| | 16_1490_LIGHT_10GS_ | GCGGGTCTATCAGTTCCGGTCTCCTCAGCGGTCGCAGTGA |
| | His | TAATATCGGCGGTTCTCAGGAACGCAGGTCACACGAGGT |
| | DNA | AAATCCAGCCGCCCATTTGACTGGCGCTAATAGTTCTCTT |
| | | ACGGGTAGCGGTGGCCCTCTTCTGTGGGAAACGCAGTTG |
| | | GGCTTGGCTTTCCTTAGAGGTCTCAGCTATCACGACGGCG |
| | | CCCTCGTCGTTACAAAGGCCGGGTACTATTATATTTATTC |
| | | TAAGGTACAGCTTGGCGGGGGGGGTGCCCGCTGGGTCT |
| | | TGCATCTACGATCACTCATGGTCTCTACAAAAGAACACCA |
| | | AGGTATCCAGAAGAACTTGAGCTCCTCGTTAGCCAACAG |
| | | TCCCCCTGCGGACGGGCTACTTCCTCTAGCCGGGTATGGT |
| | | GGGATAGCAGTTTCCTTGGGGGGGTCGTGCATCTCGAGG |
| | | CTGGAGAGGAGGTAGTAGTCAGGGTCTTGGATGAGAGAT |
| | | TGGTAAGGCTTCGAGACGGTACAAGATCATACTTTGGGG |
| | | CCTTCATGGTGAGCGGGGGTGGCAGCGGCGGTGGCAGCG |
| | | GGCATCATCACCATCACCATTGA |
| 6 | ProC1188/ | QERRSHEVNPAAHLTGANSSLTGSGGPLLWETQLGLAFLRG |
| | ProC_LIGHT-10GS- | LSYHDGALVVTKAGYYYIYSKVQLGGVGCPLGLASTITHGL |
| | Strep | YKRTPRYPEELELLVSQQSPCGRATSSSRVWWDSSFLGGVV |
| | DNA | HLEAGEEVVVRVLDERLVRLRDGTRSYFGAFMVSGGGSGG |
| | | GSGSAWSHPQFEK |
| 7 | ProC1188/ | CAGGAACGCAGGTCCCATGAAGTAAACCCGGCGGCCCAT |
| | ProC_LIGHT-10GS- | CTTACGGGTGCAAATTCAAGCTTGACAGGCAGCGGAGGG |
| | Strep | CCGCTCCTGTGGGAGACGCAACTTGGGCTTGCGTTTCTCC |
| | DNA | GGGGCCTGTCATATCACGACGGTGCTCTTGTGGTAACGA |
| | | AAGCAGGGTACTATTATATATATAGCAAGGTTCAGTTGG |
| | | GTGGGGTTGGATGCCCGCTTGGACTCGCGTCCACTATTAC |
| | | TCACGGGTTGTATAAGAGGACACCGCGATACCCAGAGGA |
| | | GCTGGAACTCCTTGTTTCACAGCAGTCTCCTTGCGGGAGA |
| | | GCCACATCTTCATCCAGGGTCTGGTGGGACTCTTCTTTCT |
| | | TGGGCGGCGTTGTTCATCTGGAAGCGGGGGAAGAGGTTG |
| | | TAGTGAGGGTACTCGACGAGAGGCTGGTGAGGCTGCGAG |
| | | ACGGGACCCGCTCATATTTCGGAGCCTTCATGGTATCAGG |
| | | AGGGGGCTCCGGTGGGGGGTCAGGAAGCGCATGGTCACA |
| | | CCCTCAGTTCGAGAAATGA |
| 8 | ProC1192/ | QGQSGSRHPCRHDPHIICYKFGGGSSGGSISSGLLSGRSDNIG |
| | ProC_mLm16_1490_ | GSQERRSHEVNPAAHLTGANSSLTGSGGPLLWETQLGLAFL |
| | LIGHT-10GS-Strep | RGLSYHDGALVVTKAGYYYIYSKVQLGGVGCPLGLASTITH |
| | Protein | GLYKRTPRYPEELELLVSQQSPCGRATSSSRVWWDSSFLGG |
| | | VVHLEAGEEVVVRVLDERLVRLRDGTRSYFGAFMVSGGGS |
| | | GGGSGSAWSHPQFEK |
| 9 | ProC1192/ | CAAGGACAGAGTGGAAGTCGACACCCTTGTAGGCACGAC |
| | ProC_mLm16_1490_ | CCACATATAATCTGCTATAAATTTGGTGGGGGTTCCAGCG |
| | LIGHT-10GS-Strep | GTGGAAGTATCAGCTCCGGCCTTCTTAGTGGCAGGAGTG |
| | DNA | ACAATATTGGCGGATCTCAAGAGCGACGCAGTCACGAGG |
| | | TAAATCCTGCAGCACACTTGACAGGTGCTAACAGCTCACT |
| | | TACCGGAAGTGGGGGGCCGCTGTTGTGGGAGACGCAGCT |
| | | CGGGTTGGCCTTTCTTCGAGGGCTCTCTTATCACGACGGT |
| | | GCATTGGTGGTGACCAAAGCAGGGTATTACTACATATAC |
| | | TCTAAGGTGCAACTGGGTGGAGTAGGTTGTCCATTGGGG |
| | | CTCGCCTCTACAATAACTCATGGTCTGTATAAGCGAACCC |
| | | CGCGGTATCCCGAAGAGCTTGAACTCCTCGTCAGCCAGC |
| | | AATCTCCCTGTGGTCGGGCAACATCATCTAGCAGAGTGTG |
| | | GTGGGACTCCTCTTTTTTGGGTGGTGTAGTACATCTGGAA |
| | | GCGGGCGAAGAAGTGGTCGTCCGAGTGCTTGACGAACGA |
| | | CTCGTTAGACTGCGAGACGGAACTCGGAGCTACTTTGGA |
| | | GCTTTTATGGTTAGCGGTGGTGGTTCAGGAGGTGGGAGT |
| | | GGGAGTGCATGGTCACACCCGCAATTCGAGAAATGA |
| 10 | ProC1491/ | QERRSHEVNPAAHLTGANSSLTGSGGPLLWETQLGLAFLRG |
| | ProC_LIGHT- | LSYHDGALVVTKAGYYYIYSKVQLGGVGCPLGLASTITHGL |
| | 1204DNI-HSA-His | YKRTPRYPEELELLVSQQSPCGRATSSSRVWWDSSFLGGVV |
| | Protein | HLEAGEEVVVRVLDERLVRLRDGTRSYFGAFMVSSAGGGS |
| | | SGGSLSGRSDNIGSDAHKSEVAHRFKDLGEENFKALVLIAFA |
| | | QYLQQCPFEDHVKLVNEVTEFAKTCVADESAENCDKSLHT |
| | | LFGDKLCTVATLRETYGEMADCCAKQEPERNECFLQHKDD |
| | | NPNLPRLVRPEVDVMCTAFHDNEETFLKKYLYEIARRHPYF |
| | | YAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSHHHHHH |
| 11 | ProC1491/ | CAGGAGAGGAGATCACATGAAGTCAACCCGGCCGCCCAT |
| | ProC_LIGHT- | CTTACTGGTGCCAATAGTAGTCTTACAGGTTCTGGCGGAC |
| | 1204DNI-HSA-His | CCTTGCTTTGGGAGACCCAGCTGGGACTGGCTTTCCTTAG |
| | DNA | AGGCCTGAGTTATCACGACGGGGCGTTGGTCGTTACTAA |
| | | AGCCGGGTATTACTATATATACTCTAAGGTTCAGCTCGGC |
| | | GGTGTAGGATGCCCACTGGGTCTTGCGTCTACAATTACAC |
| | | ATGGTCTGTATAAGAGGACTCCCAGATATCCAGAAGAAC |
| | | TGGAATTGCTTGTTTCCCAGCAATCTCCGTGCGGACGCGC |
| | | AACGAGCTCCAGTAGGGTGTGGTGGGACTCCAGCTTCCT |
| | | GGGTGGCGTTGTCCATTTGGAGGCGGGAGAGGAAGTAGT |
| | | GGTGCGGGTCCTTGATGAACGCCTTGTACGACTTCGGGAC |
| | | GGTACAAGAAGCTACTTTGGGGCTTTCATGGTATCCAGCG |
| | | CCGGGGGCGGAAGCAGCGGCGGCAGCTTAAGCGGCAGA |
| | | AGCGACAACATCGGCAGCGATGCACATAAAAGTGAGGTG |
| | | GCCCACCGCTTTAAAGACCTCGGCGAGGAGAACTTCAAG |
| | | GCACTCGTACTTATTGCCTTTGCACAATACCTGCAGCAAT |
| | | GTCCGTTTGAAGATCATGTCAAGCTCGTAAATGAAGTGA |
| | | CAGAGTTCGCTAAGACCTGTGTAGCTGACGAAAGTGCCG |
| | | AAAACTGCGACAAATCATTGCATACATTGTTCGGCGACA |
| | | AGCTTTGTACAGTTGCCACGCTCCGCGAGACATACGGCG |
| | | AAATGGCCGATTGTTGTGCGAAACAGGAACCCGAAAGAA |
| | | ATGAGTGCTTCTTGCAACACAAAGACGATAATCCCAATCT |
| | | TCCGCGACTGGTGAGACCCGAAGTAGATGTAATGTGCAC |
| | | AGCTTTTCATGACAATGAGGAAACCTTTCTTAAAAAATAT |
| | | CTGTACGAAATCGCCCGCCGACACCCGTATTTCTATGCCC |
| | | CAGAGCTGTTGTTTTTCGCAAAGCGCTACAAGGCAGCGTT |
| | | TACCGAATGCTGCCAGGCCGCTGATAAAGCCGCTTGTCTG |
| | | CTGCCTAAGCTTGATGAGCTTCGCGACGAGGGTAAGGCT |
| | | TCCAGCGCTAAACAACGCCTGAAGTGTGCATCTTTGCAA |
| | | AAATTTGGTGAACGAGCCTTCAAGGCTTGGGCTGTTGCCA |
| | | GGCTGAGTCAACGGTTCCCGAAGGCGGAGTTTGCGGAGG |
| | | TTTCCAAGTTGGTAACGGACCTGACAAAGGTACATACCG |
| | | AATGCTGCCATGGCGACCTGTTGGAGTGCGCCGATGATC |
| | | GAGCGGATCTTGCCAAGTATATTTGCGAAAACCAAGACT |
| | | CCATTTCCAGTAAACTTAAGGAGTGTTGTGAGAAACCTCT |
| | | CCTTGAAAAGAGCCATTGTATAGCGGAGGTCGAAAACGA |
| | | TGAAATGCCCGCTGATCTGCCGTCACTCGCTGCTGACTTC |
| | | GTTGAATCCAAGGATGTATGTAAAAACTATGCCGAAGCT |
| | | AAGGACGTCTTTCTTGGAATGTTCTTGTATGAATACGCCC |
| | | GACGGCATCCTGACTATTCAGTTGTGCTCCTTCTTCGATT |
| | | GGCTAAAACTTATGAGACGACACTTGAAAAGTGTTGTGC |
| | | CGCCGCCGACCCACATGAGTGCTACGCTAAGGTTTTCGA |
| | | CGAATTTAAGCCCTTGGTTGAAGAACCGCAGAATCTCATT |
| | | AAGCAAAATTGTGAGCTCTTTGAACAACTCGGTGAATAC |
| | | AAATTTCAGAACGCGCTTCTGGTTAGATATACGAAGAAA |
| | | GTGCCACAGGTGAGCACCCCTACATTGGTTGAGGTCAGT |
| | | AGGAACCTCGGCAAGGTGGGATCTAAGTGCTGCAAACAC |
| | | CCGGAGGCAAAGAGAATGCCTTGCGCTGAGGATTATCTC |
| | | TCAGTGGTTCTGAATCAGCTTTGCGTTCTTCACGAAAAGA |
| | | CTCCCGTCAGCGACCGAGTCACGAAGTGCTGTACCGAGA |
| | | GCCTTGTTAATAGACGACCTTGCTTCTCCGCGCTTGAGGT |
| | | GGATGAGACCTACGTCCCAAAGGAATTCAACGCCGAGAC |
| | | GTTCACTTTCCATGCCGATATTTGCACCTTGTCAGAAAAG |
| | | GAGCGACAGATAAAAAAGCAAACTGCTTTGGTGGAGCTG |
| | | GTTAAGCATAAGCCTAAAGCGACGAAGGAACAGCTCAAA |
| | | GCCGTAATGGATGATTTCGCTGCCTTCGTGGAGAAATGTT |
| | | GTAAGGCCGATGACAAGGAAACCTGCTTCGCTGAAGAGG |
| | | GTAAAAAGCTCGTCGCCGCTAGTCAGGCGGCACTTGGTTT |
| | | GAGCCACCATCACCACCACCACtga |
| 12 | ProC1492/ | QGQSGSRHPCRHDPHIICYKFGGGSSGGSISSGLLSGRCDNIG |
| | ProC_mLm16- | GSQERRSHEVNPAAHLTGANSSLTGSGGPLLWETQLGLAFL |
| | LIGHT-1204DNI- | RGLSYHDGALVVTKAGYYYIYSKVQLGGVGCPLGLASTITH |
| | HSA-His (1490DNI | GLYKRTPRYPEELELLVSQQSPCGRATSSSRVWWDSSFLGG |
| | has S to C mutation) | VVHLEAGEEVVVRVLDERLVRLRDGTRSYFGAFMVSSAGG |
| | Protein | GSSGGSLSGRSDNIGSDAHKSEVAHRFKDLGEENFKALVLIA |
| | | FAQYLQQCPFEDHVKLVNEVTEFAKTCVADESAENCDKSL |
| | | HTLFGDKLCTVATLRETYGEMADCCAKQEPERNECFLQHK |
| | | DDNPNLPRLVRPEVDVMCTAFHDNEETFLKKYLYEIARRHP |
| | | YFYAPELLFFAKRYKAAFTECCQAADKAACLLPKLDELRDE |
| | | GKASSAKQRLKCASLQKFGERAFKAWAVARLSQRFPKAEF |
| | | AEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLA |
| | | KTYETTLEKCCAAADPHECYAKVFDEFKPLVEEPQNLIKQN |
| | | CELFEQLGEYKFQNALLVRYTKKVPQVSTPTLVEVSRNLGK |
| | | VGSKCCKHPEAKRMPCAEDYLSVVLNQLCVLHEKTPVSDR |
| | | VTKCCTESLVNRRPCFSALEVDETYVPKEFNAETFTFHADIC |
| | | TLSEKERQIKKQTALVELVKHKPKATKEQLKAVMDDFAAF |
| | | VEKCCKADDKETCFAEEGKKLVAASQAALGLSHHHHHH |
| 13 | ProC1492/ | CAGGGCCAGTCTGGATCCAGACACCCATGCAGACACGAC |
| | ProC_mLm16- | CCCCACATCATCTGTTACAAATTCGGCGGAGGCAGCTCC |
| | LIGHT-1204DNI- | GGCGGCAGCATCTCCAGCGGACTGCTGAGCGGCAGAtGC |
| | HSA-His (1490DNI | GATAACATCGGCGGAAGCCAGGAGAGGAGATCACATGA |
| | has S to C mutation) | AGTCAACCCGGCCGCCCATCTTACTGGTGCCAATAGTAGT |
| | DNA | CTTACAGGTTCTGGCGGACCCTTGCTTTGGGAGACCCAGC |
| | | TGGGACTGGCTTTCCTTAGAGGCCTGAGTTATCACGACGG |
| | | GGCGTTGGTCGTTACTAAAGCCGGGTATTACTATATATAC |
| | | TCTAAGGTTCAGCTCGGCGGTGTAGGATGCCCACTGGGTC |
| | | TTGCGTCTACAATTACACATGGTCTGTATAAGAGGACTCC |
| | | CAGATATCCAGAAGAACTGGAATTGCTTGTTTCCCAGCA |
| | | ATCTCCGTGCGGACGCGCAACGAGCTCCAGTAGGGTGTG |
| | | GTGGGACTCCAGCTTCCTGGGTGGCGTTGTCCATTTGGAG |
| | | GCGGGAGAGGAAGTAGTGGTGCGGGTCCTTGATGAACGC |
| | | CTTGTACGACTTCGGGACGGTACAAGAAGCTACTTTGGG |
| | | GCTTTCATGGTATCCAGCGCCGGGGGCGGAAGCAGCGGC |
| | | GGCAGCTTAAGCGGCAGAAGCGACAACATCGGCAGCGAT |
| | | GCACATAAAAGTGAGGTGGCCCACCGCTTTAAAGACCTC |
| | | GGCGAGGAGAACTTCAAGGCACTCGTACTTATTGCCTTTG |
| | | CACAATACCTGCAGCAATGTCCGTTTGAAGATCATGTCAA |
| | | GCTCGTAAATGAAGTGACAGAGTTCGCTAAGACCTGTGT |
| | | AGCTGACGAAAGTGCCGAAAACTGCGACAAATCATTGCA |
| | | TACATTGTTCGGCGACAAGCTTTGTACAGTTGCCACGCTC |
| | | CGCGAGACATACGGCGAAATGGCCGATTGTTGTGCGAAA |
| | | CAGGAACCCGAAAGAAATGAGTGCTTCTTGCAACACAAA |
| | | GACGATAATCCCAATCTTCCGCGACTGGTGAGACCCGAA |
| | | GTAGATGTAATGTGCACAGCTTTTCATGACAATGAGGAA |
| | | ACCTTTCTTAAAAAATATCTGTACGAAATCGCCCGCCGAC |
| | | ACCCGTATTTCTATGCCCCAGAGCTGTTGTTTTTCGCAAA |
| | | GCGCTACAAGGCAGCGTTTACCGAATGCTGCCAGGCCGC |
| | | TGATAAAGCCGCTTGTCTGCTGCCTAAGCTTGATGAGCTT |
| | | CGCGACGAGGGTAAGGCTTCCAGCGCTAAACAACGCCTG |
| | | AAGTGTGCATCTTTGCAAAAATTTGGTGAACGAGCCTTCA |
| | | AGGCTTGGGCTGTTGCCAGGCTGAGTCAACGGTTCCCGA |
| | | AGGCGGAGTTTGCGGAGGTTTCCAAGTTGGTAACGGACC |
| | | TGACAAAGGTACATACCGAATGtTGCCATGGCGACCTGTT |
| | | GGAGTGCGCCGATGATCGAGCGGATCTTGCCAAGTATAT |
| | | TTGCGAAAACCAAGACTCCATTTCCAGTAAACTTAAGGA |
| | | GTGTTGTGAGAAACCTCTCCTTGAAAAGAGCCATTGTATA |
| | | GCGGAGGTCGAAAACGATGAAATGCCCGCTGATCTGCCG |
| | | TCACTCGCTGCTGACTTCGTTGAATCCAAGGATGTATGTA |
| | | AAAACTATGCCGAAGCTAAGGACGTCTTTCTTGGAATGTT |
| | | CTTGTATGAATACGCCCGACGGCATCCTGACTATTCAGTT |
| | | GTGCTCCTTCTTCGATTGGCTAAAACTTATGAGACGACAC |
| | | TTGAAAAGTGTTGTGCCGCGGCGGACCCACATGAGTGCT |
| | | ACGCTAAGGTTTTCGACGAATTTAAGCCCTTGGTTGAAGA |
| | | ACCGCAGAATCTCATTAAGCAAAATTGTGAGCTCTTTGAA |
| | | CAACTCGGTGAATACAAATTTCAGAACGCGCTTCTGGTTA |
| | | GATATACGAAGAAAGTGCCACAGGTGAGCACCCCTACAT |
| | | TGGTTGAGGTCAGTAGGAACCTCGGCAAGGTGGGATCTA |
| | | AGTGCTGCAAACACCCGGAGGCAAAGAGAATGCCTTGCG |
| | | CTGAGGATTATCTCTCAGTGGTTCTGAATCAGCTTTGCGT |
| | | TCTTCACGAAAAGACTCCCGTCAGCGACCGAGTCACGAA |
| | | GTGCTGTACCGAGAGCCTTGTTAATAGACGACCTTGCTTC |
| | | TCCGCGCTTGAGGTGGATGAGACCTACGTCCCAAAGGAA |
| | | TTCAACGCCGAGACGTTCACTTTCCATGCCGATATTTGCA |
| | | CCTTGTCAGAAAAGGAGCGACAGATAAAAAAGCAAACTG |
| | | CTTTGGTGGAGCTGGTTAAGCATAAGCCTAAAGCGACGA |
| | | AGGAACAGCTCAAAGCCGTAATGGATGATTTCGCTGCCT |
| | | TCGTGGAGAAATGTTGTAAGGCCGATGACAAGGAAACCT |
| | | GCTTCGCTGAAGAGGGTAAAAAGCTCGTCGCCGCTAGTC |
| | | AGGCGGCACTTGGTTTGAGCCACCATCACCACCACCACtga |
| 14 | ProC1655/ | QGQSGSRHPCRHDPHIICYKFGGGSSGGSISSGLLSGRSDNIG |
| | ProC_mLm16- | GSQERRSHEVNPAAHLTGANSSLTGSGGPLLWETQLGLAFL |
| | LIGHT-1204DNI- | RGLSYHDGALVVTKAGYYYIYSKVQLGGVGCPLGLASTITH |
| | HSA-His 1490 | GLYKRTPRYPEELELLVSQQSPCGRATSSSRVWWDSSFLGG |
| | Protein | VVHLEAGEEVVVRVLDERLVRLRDGTRSYFGAFMVSSAGG |
| | | GSSGGSLSGRSDNIGSDAHKSEVAHRFKDLGEENFKALVLIA |
| | | FAQYLQQCPFEDHVKLVNEVTEFAKTCVADESAENCDKSL |
| | | HTLFGDKLCTVATLRETYGEMADCCAKQEPERNECFLQHK |
| | | DDNPNLPRLVRPEVDVMCTAFHDNEETFLKKYLYEIARRHP |
| | | YFYAPELLFFAKRYKAAFTECCQAADKAACLLPKLDELRDE |
| | | GKASSAKQRLKCASLQKFGERAFKAWAVARLSQRFPKAEF |
| | | AEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLA |
| | | KTYETTLEKCCAAADPHECYAKVFDEFKPLVEEPQNLIKQN |
| | | CELFEQLGEYKFQNALLVRYTKKVPQVSTPTLVEVSRNLGK |
| | | VGSKCCKHPEAKRMPCAEDYLSVVLNQLCVLHEKTPVSDR |
| | | VTKCCTESLVNRRPCFSALEVDETYVPKEFNAETFTFHADIC |
| | | TLSEKERQIKKQTALVELVKHKPKATKEQLKAVMDDFAAF |
| | | VEKCCKADDKETCFAEEGKKLVAASQAALGLSHHHHHH |
| 15 | ProC1655/ | CAGGGCCAGTCTGGATCCAGACACCCATGCAGACACGAC |
| | ProC_mLm16- | CCCCACATCATCTGTTACAAATTCGGCGGAGGCAGCTCC |
| | LIGHT-1204DNI- | GGCGGCAGCATCTCCAGCGGACTGCTGAGCGGCAGAAGC |
| | HSA-His 1490 | GATAACATCGGCGGAAGCCAGGAGAGGAGATCACATGA |
| | DNA | AGTCAACCCGGCCGCCCATCTTACTGGTGCCAATAGTAGT |
| | | CTTACAGGTTCTGGCGGACCCTTGCTTTGGGAGACCCAGC |
| | | TGGGACTGGCTTTCCTTAGAGGCCTGAGTTATCACGACGG |
| | | GGCGTTGGTCGTTACTAAAGCCGGGTATTACTATATATAC |
| | | TCTAAGGTTCAGCTCGGCGGTGTAGGATGCCCACTGGGTC |
| | | TTGCGTCTACAATTACACATGGTCTGTATAAGAGGACTCC |
| | | CAGATATCCAGAAGAACTGGAATTGCTTGTTTCCCAGCA |
| | | ATCTCCGTGCGGACGCGCAACGAGCTCCAGTAGGGTGTG |
| | | GTGGGACTCCAGCTTCCTGGGTGGCGTTGTCCATTTGGAG |
| | | GCGGGAGAGGAAGTAGTGGTGCGGGTCCTTGATGAACGC |
| | | CTTGTACGACTTCGGGACGGTACAAGAAGCTACTTTGGG |
| | | GCTTTCATGGTATCCAGCGCCGGGGGCGGAAGCAGCGGC |
| | | GGCAGCTTAAGCGGCAGAAGCGAtAACATCGGCAGCGAT |
| | | GCACATAAAAGTGAGGTGGCCCACCGCTTTAAAGACCTC |
| | | GGCGAGGAGAACTTCAAGGCACTCGTACTTATTGCCTTTG |
| | | CACAATACCTGCAGCAATGTCCGTTTGAAGATCATGTCAA |
| | | GCTCGTAAATGAAGTGACAGAGTTCGCTAAGACCTGTGT |
| | | AGCTGACGAAAGTGCCGAAAACTGCGACAAATCATTGCA |
| | | TACATTGTTCGGCGACAAGCTTTGTACAGTTGCCACGCTC |
| | | CGCGAGACATACGGCGAAATGGCCGATTGTTGTGCGAAA |
| | | CAGGAACCCGAAAGAAATGAGTGCTTCTTGCAACACAAA |
| | | GACGATAATCCCAATCTTCCGCGACTGGTGAGACCCGAA |
| | | GTAGATGTAATGTGCACAGCTTTTCATGACAATGAGGAA |
| | | ACCTTTCTTAAAAAATATCTGTACGAAATCGCCCGCCGAC |
| | | ACCCGTATTTCTATGCCCCAGAGCTGTTGTTTTTCGCAAA |
| | | GCGCTACAAGGCAGCGTTTACCGAATGCTGCCAGGCCGC |
| | | TGATAAAGCCGCTTGTCTGCTGCCTAAGCTTGATGAGCTT |
| | | CGCGACGAGGGTAAGGCTTCCAGCGCTAAACAACGCCTG |
| | | AAGTGTGCATCTTTGCAAAAATTTGGTGAACGAGCCTTCA |
| | | AGGCTTGGGCTGTTGCCAGGCTGAGTCAACGGTTCCCGA |
| | | AGGCGGAGTTTGCGGAGGTTTCCAAGTTGGTAACGGACC |
| | | TGACAAAGGTACATACCGAATGtTGCCATGGCGACCTGTT |
| | | GGAGTGCGCCGATGATCGAGCGGATCTTGCCAAGTATAT |
| | | TTGCGAAAACCAAGACTCCATTTCCAGTAAACTTAAGGA |
| | | GTGTTGTGAGAAACCTCTCCTTGAAAAGAGCCATTGTATA |
| | | GCGGAGGTCGAAAACGATGAAATGCCCGCTGATCTGCCG |
| | | TCACTCGCTGCTGACTTCGTTGAATCCAAGGATGTATGTA |
| | | AAAACTATGCCGAAGCTAAGGACGTCTTTCTTGGAATGTT |
| | | CTTGTATGAATACGCCCGACGGCATCCTGACTATTCAGTT |
| | | GTGCTCCTTCTTCGATTGGCTAAAACTTATGAGACGACAC |
| | | TTGAAAAGTGTTGTGCCGCGGCGGACCCACATGAGTGCT |
| | | ACGCTAAGGTTTTCGACGAATTTAAGCCCTTGGTTGAAGA |
| | | ACCGCAGAATCTCATTAAGCAAAATTGTGAGCTCTTTGAA |
| | | CAACTCGGTGAATACAAATTTCAGAACGCGCTTCTGGTTA |
| | | GATATACGAAGAAAGTGCCACAGGTGAGCACCCCTACAT |
| | | TGGTTGAGGTCAGTAGGAACCTCGGCAAGGTGGGATCTA |
| | | AGTGCTGCAAACACCCGGAGGCAAAGAGAATGCCTTGCG |
| | | CTGAGGATTATCTCTCAGTGGTTCTGAATCAGCTTTGCGT |
| | | TCTTCACGAAAAGACTCCCGTCAGCGACCGAGTCACGAA |
| | | GTGCTGTACCGAGAGCCTTGTTAATAGACGACCTTGCTTC |
| | | TCCGCGCTTGAGGTGGATGAGACCTACGTCCCAAAGGAA |
| | | TTCAACGCCGAGACGTTCACTTTCCATGCCGATATTTGCA |
| | | CCTTGTCAGAAAAGGAGCGACAGATAAAAAAGCAAACTG |
| | | CTTTGGTGGAGCTGGTTAAGCATAAGCCTAAAGCGACGA |
| | | AGGAACAGCTCAAAGCCGTAATGGATGATTTCGCTGCCT |
| | | TCGTGGAGAAATGTTGTAAGGCCGATGACAAGGAAACCT |
| | | GCTTCGCTGAAGAGGGTAAAAAGCTCGTCGCCGCTAGTC |
| | | AGGCGGCACTTGGTTTGAGCCACCATCACCACCACCACtga |
| 16 | ProC1499/ | DAHKSEVAHRFKDLGEENFKALVLIAFAQYLQQCPFEDHVK |
| | ProC_HSA-21GS- | LVNEVTEFAKTCVADESAENCDKSLHTLFGDKLCTVATLRE |
| | 1204DNI-LIGHT | TYGEMADCCAKQEPERNECFLQHKDDNPNLPRLVRPEVDV |
| | Protein | MCTAFHDNEETFLKKYLYEIARRHPYFYAPELLFFAKRYKA |
| | | AFTECCQAADKAACLLPKLDELRDEGKASSAKQRLKCASL |
| | | QKFGERAFKAWAVARLSQRFPKAEFAEVSKLVTDLTKVHT |
| | | ECCHGDLLECADDRADLAKYICENQDSISSKLKECCEKPLLE |
| | | KSHCIAEVENDEMPADLPSLAADFVESKDVCKNYAEAKDV |
| | | FLGMFLYEYARRHPDYSVVLLLRLAKTYETTLEKCCAAADP |
| | | HECYAKVFDEFKPLVEEPQNLIKQNCELFEQLGEYKFQNAL |
| | | LVRYTKKVPQVSTPTLVEVSRNLGKVGSKCCKHPEAKRMP |
| | | CAEDYLSVVLNQLCVLHEKTPVSDRVTKCCTESLVNRRPCF |
| | | SALEVDETYVPKEFNAETFTFHADICTLSEKERQIKKQTALV |
| | | ELVKHKPKATKEQLKAVMDDFAAFVEKCCKADDKETCFAE |
| | | EGKKLVAASQAALGLSSAGGGSSGGSLSGRSDNIGSQERRS |
| | | HEVNPAAHLTGANSSLTGSGGPLLWETQLGLAFLRGLSYHD |
| | | GALVVTKAGYYYIYSKVQLGGVGCPLGLASTITHGLYKRTP |
| | | RYPEELELLVSQQSPCGRATSSSRVWWDSSFLGGVVHLEAG |
| | | EEVVVRVLDERLVRLRDGTRSYFGAFMV |
| 17 | ProC1499/ | GATGCACATAAAAGTGAGGTGGCCCACCGCTTTAAAGAC |
| | ProC_HSA-21GS- | CTCGGCGAGGAGAACTTCAAGGCACTCGTACTTATTGCCT |
| | 1204DNI-LIGHT | TTGCACAATACCTGCAGCAATGTCCGTTTGAAGATCATGT |
| | DNA | CAAGCTCGTAAATGAAGTGACAGAGTTCGCTAAGACCTG |
| | | TGTAGCTGACGAAAGTGCCGAAAACTGCGACAAATCATT |
| | | GCATACATTGTTCGGCGACAAGCTTTGTACAGTTGCCACG |
| | | CTCCGCGAGACATACGGCGAAATGGCCGATTGTTGTGCG |
| | | AAACAGGAACCCGAAAGAAATGAGTGCTTCTTGCAACAC |
| | | AAAGACGATAATCCCAATCTTCCGCGACTGGTGAGACCC |
| | | GAAGTAGATGTAATGTGCACAGCTTTTCATGACAATGAG |
| | | GAAACCTTTCTTAAAAAATATCTGTACGAAATCGCCCGCC |
| | | GACACCCGTATTTCTATGCCCCAGAGCTGTTGTTTTTCGC |
| | | AAAGCGCTACAAGGCAGCGTTTACaGAATGCTGtCAGGCC |
| | | GCTGATAAAGCCGCTTGTCTGCTGCCTAAGCTTGATGAGC |
| | | TTCGCGACGAGGGTAAGGCTTCCAGCGCTAAACAACGCC |
| | | TGAAGTGTGCATCTTTGCAAAAATTTGGTGAACGAGCCTT |
| | | CAAGGCTTGGGCTGTTGCCAGGCTGAGTCAACGGTTCCC |
| | | GAAGGCGGAGTTTGCGGAGGTTTCCAAGTTGGTAACGGA |
| | | CCTGACAAAGGTACATACCGAATGCTGCCATGGCGACCT |
| | | GTTGGAGTGCGCCGATGATCGAGCGGATCTTGCCAAGTA |
| | | TATTTGCGAAAACCAAGACTCCATTTCCAGTAAACTTAAG |
| | | GAGTGTTGTGAGAAACCTCTCCTTGAAAAGAGCCATTGT |
| | | ATAGCGGAGGTCGAAAACGATGAAATGCCCGCTGATCTG |
| | | CCGTCACTCGCTGCTGACTTCGTTGAATCCAAGGATGTAT |
| | | GTAAAAACTATGCCGAAGCTAAGGACGTCTTTCTTGGAA |
| | | TGTTCTTGTATGAATACGCCCGACGGCATCCTGACTATTC |
| | | AGTTGTGCTCCTTCTTCGATTGGCTAAAACTTATGAGACG |
| | | ACACTTGAAAAGTGTTGTGCCGCCGCCGACCCACATGAG |
| | | TGCTACGCTAAGGTTTTCGACGAATTTAAGCCCTTGGTTG |
| | | AAGAACCGCAGAATCTCATTAAGCAAAATTGTGAGCTCT |
| | | TTGAACAACTCGGTGAATACAAATTTCAGAACGCGCTTCT |
| | | GGTTAGATATACGAAGAAAGTGCCACAGGTGAGCACCCC |
| | | TACATTGGTTGAGGTCAGTAGGAACCTCGGCAAGGTGGG |
| | | ATCTAAGTGCTGCAAACACCCGGAGGCAAAGAGAATGCC |
| | | TTGCGCTGAGGATTATCTCTCAGTGGTTCTGAATCAGCTT |
| | | TGCGTTCTTCACGAAAAGACTCCCGTCAGCGACCGAGTC |
| | | ACGAAGTGCTGTACCGAGAGCCTTGTTAATAGACGACCT |
| | | TGCTTCTCCGCGCTTGAGGTGGATGAGACCTACGTCCCAA |
| | | AGGAATTCAACGCCGAGACGTTCACTTTCCATGCCGATAT |
| | | TTGCACCTTGTCAGAAAAGGAGCGACAGATAAAAAAGCA |
| | | AACTGCTTTGGTGGAGCTGGTTAAGCATAAGCCTAAAGC |
| | | GACGAAGGAACAGCTCAAAGCCGTAATGGATGATTTCGC |
| | | TGCCTTCGTGGAGAAATGTTGTAAGGCCGATGACAAGGA |
| | | AACCTGCTTCGCTGAAGAGGGTAAAAAGCTCGTCGCCGC |
| | | TAGTCAGGCGGCACTTGGTTTGTCCAGCGCCGGGGGCGG |
| | | AAGCAGCGGCGGCAGCTTAAGCGGCAGAAGCGACAACA |
| | | TCGGCAGCCAGGAGAGACGGAGCCACGAGGTGAACCCC |
| | | GCTGCCCACCTGACCGGCGCCAACAGCAGTCTGACCGGC |
| | | AGCGGCGGCCCTCTGCTGTGGGAGACACAGCTGGGCCTG |
| | | GCCTTTCTGAGAGGCCTGTCCTACCACGACGGCGCCCTGG |
| | | TCGTGACCAAGGCCGGCTACTACTACATCTACAGCAAGG |
| | | TGCAGCTGGGAGGCGTGGGCTGCCCTCTGGGCCTGGCAT |
| | | CTACCATCACGCACGGCCTGTACAAGCGGACCCCTAGAT |
| | | ATCCTGAGGAACTGGAACTCCTGGTGTCCCAGCAGAGCC |
| | | CTTGCGGCAGAGCTACAAGCAGCTCAAGAGTGTGGTGGG |
| | | ACAGCAGCTTCCTGGGCGGAGTGGTCCACCTGGAAGCTG |
| | | GAGAAGAAGTGGTGGTCCGGGTGCTGGACGAGAGACTCG |
| | | TGAGGCTGAGAGACGGAACAAGAAGCTACTTCGGCGCTT |
| | | TTATGGTGtga |
| 18 | ProC1495/ | QERRSHEVNPAAHLTGANSSLTGSGGPLLWETQLGLAFLRG |
| | ProC_LIGHT- | LSYHDGALVVTKAGYYYIYSKVQLGGVGCPLGLASTITHGL |
| | 1204DNI-HSA-cMyc | YKRTPRYPEELELLVSQQSPCGRATSSSRVWWDSSFLGGVV |
| | Protein | HLEAGEEVVVRVLDERLVRLRDGTRSYFGAFMVSSAGGGS |
| | | SGGSLSGRSDNIGSDAHKSEVAHRFKDLGEENFKALVLIAFA |
| | | QYLQQCPFEDHVKLVNEVTEFAKTCVADESAENCDKSLHT |
| | | LFGDKLCTVATLRETYGEMADCCAKQEPERNECFLQHKDD |
| | | NPNLPRLVRPEVDVMCTAFHDNEETFLKKYLYEIARRHPYF |
| | | YAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSGGEQKLISEE |
| | | DL |
| 19 | ProC1495/ | CAGGAGAGGAGATCACATGAAGTCAACCCGGCCGCCCAT |
| | ProC_LIGHT- | CTTACTGGTGCCAATAGTAGTCTTACAGGTTCTGGCGGAC |
| | 1204DNI-HSA-cMyc | CCTTGCTTTGGGAGACCCAGCTGGGACTGGCTTTCCTTAG |
| | DNA | AGGCCTGAGTTATCACGACGGGGCGTTGGTCGTTACTAA |
| | | AGCCGGGTATTACTATATATACTCTAAGGTTCAGCTCGGC |
| | | GGTGTAGGATGCCCACTGGGTCTTGCGTCTACAATTACAC |
| | | ATGGTCTGTATAAGAGGACTCCCAGATATCCAGAAGAAC |
| | | TGGAATTGCTTGTTTCCCAGCAATCTCCGTGCGGACGCGC |
| | | AACGAGCTCCAGTAGGGTGTGGTGGGACTCCAGCTTCCT |
| | | GGGTGGCGTTGTCCATTTGGAGGCGGGAGAGGAAGTAGT |
| | | GGTGCGGGTCCTTGATGAACGCCTTGTACGACTTCGGGAC |
| | | GGTACAAGAAGCTACTTTGGGGCTTTCATGGTATCCAGCG |
| | | CCGGGGGCGGAAGCAGCGGCGGCAGCTTAAGCGGCAGA |
| | | AGCGACAACATCGGCAGCGATGCACATAAAAGTGAGGTG |
| | | GCCCACCGCTTTAAAGACCTCGGCGAGGAGAACTTCAAG |
| | | GCACTCGTACTTATTGCCTTTGCACAATACCTGCAGCAAT |
| | | GTCCGTTTGAAGATCATGTCAAGCTCGTAAATGAAGTGA |
| | | CAGAGTTCGCTAAGACCTGTGTAGCTGACGAAAGTGCCG |
| | | AAAACTGCGACAAATCATTGCATACATTGTTCGGCGACA |
| | | AGCTTTGTACAGTTGCCACGCTCCGCGAGACATACGGCG |
| | | AAATGGCCGATTGTTGTGCGAAACAGGAACCCGAAAGAA |
| | | ATGAGTGCTTCTTGCAACACAAAGACGATAATCCCAATCT |
| | | TCCGCGACTGGTGAGACCCGAAGTAGATGTAATGTGCAC |
| | | AGCTTTTCATGACAATGAGGAAACCTTTCTTAAAAAATAT |
| | | CTGTACGAAATCGCCCGCCGACACCCGTATTTCTATGCCC |
| | | CAGAGCTGTTGTTTTTCGCAAAGCGCTACAAGGCAGCGTT |
| | | TACCGAATGCTGCCAGGCCGCTGATAAAGCCGCTTGTCTG |
| | | CTGCCTAAGCTTGATGAGCTTCGCGACGAGGGTAAGGCT |
| | | TCCAGCGCTAAACAACGCCTGAAGTGTGCATCTTTGCAA |
| | | AAATTTGGTGAACGAGCCTTCAAGGCTTGGGCTGTTGCCA |
| | | GGCTGAGTCAACGGTTCCCGAAGGCGGAGTTTGCGGAGG |
| | | TTTCCAAGTTGGTAACGGACCTGACAAAGGTACATACCG |
| | | AATGCTGCCATGGCGACCTGTTGGAGTGCGCCGATGATC |
| | | GAGCGGATCTTGCCAAGTATATTTGCGAAAACCAAGACT |
| | | CCATTTCCAGTAAACTTAAGGAGTGTTGTGAGAAACCTCT |
| | | CCTTGAAAAGAGCCATTGTATAGCGGAGGTCGAAAACGA |
| | | TGAAATGCCCGCTGATCTGCCGTCACTCGCTGCTGACTTC |
| | | GTTGAATCCAAGGATGTATGTAAAAACTATGCCGAAGCT |
| | | AAGGACGTCTTTCTTGGAATGTTCTTGTATGAATACGCCC |
| | | GACGGCATCCTGACTATTCAGTTGTGCTCCTTCTTCGATT |
| | | GGCTAAAACTTATGAGACGACACTTGAAAAGTGTTGTGC |
| | | CGCCGCCGACCCACATGAGTGCTACGCTAAGGTTTTCGA |
| | | CGAATTTAAGCCCTTGGTTGAAGAACCGCAGAATCTCATT |
| | | AAGCAAAATTGTGAGCTCTTTGAACAACTCGGTGAATAC |
| | | AAATTTCAGAACGCGCTTCTGGTTAGATATACGAAGAAA |
| | | GTGCCACAGGTGAGCACCCCTACATTGGTTGAGGTCAGT |
| | | AGGAACCTCGGCAAGGTGGGATCTAAGTGCTGCAAACAC |
| | | CCGGAGGCAAAGAGAATGCCTTGCGCTGAGGATTATCTC |
| | | TCAGTGGTTCTGAATCAGCTTTGCGTTCTTCACGAAAAGA |
| | | CTCCCGTCAGCGACCGAGTCACGAAGTGCTGTACCGAGA |
| | | GCCTTGTTAATAGACGACCTTGCTTCTCCGCGCTTGAGGT |
| | | GGATGAGACCTACGTCCCAAAGGAATTCAACGCCGAGAC |
| | | GTTCACTTTCCATGCCGATATTTGCACCTTGTCAGAAAAG |
| | | GAGCGACAGATAAAAAAGCAAACTGCTTTGGTGGAGCTG |
| | | GTTAAGCATAAGCCTAAAGCGACGAAGGAACAGCTCAAA |
| | | GCCGTAATGGATGATTTCGCTGCCTTCGTGGAGAAATGTT |
| | | GTAAGGCCGATGACAAGGAAACCTGCTTCGCTGAAGAGG |
| | | GTAAAAAGCTCGTCGCCGCTAGTCAGGCGGCACTTGGTTT |
| | | GAGTGGTGGAGAACAAAAACTCATTTCCGAGGAAGATCT |
| | | Ttga |
| 20 | ProC1496/ | QGQSGSRHPCRHDPHIICYKFGGGSSGGSISSGLLSGRSDNIG |
| | ProC_mLm16- | GSQERRSHEVNPAAHLTGANSSLTGSGGPLLWETQLGLAFL |
| | LIGHT-1204DNI- | RGLSYHDGALVVTKAGYYYIYSKVQLGGVGCPLGLASTITH |
| | HSA-cMyc | GLYKRTPRYPEELELLVSQQSPCGRATSSSRVWWDSSFLGG |
| | Protein | VVHLEAGEEVVVRVLDERLVRLRDGTRSYFGAFMVSSAGG |
| | | GSSGGSLSGRSDNIGSDAHKSEVAHRFKDLGEENFKALVLIA |
| | | FAQYLQQCPFEDHVKLVNEVTEFAKTCVADESAENCDKSL |
| | | HTLFGDKLCTVATLRETYGEMADCCAKQEPERNECFLQHK |
| | | DDNPNLPRLVRPEVDVMCTAFHDNEETFLKKYLYEIARRHP |
| | | YFYAPELLFFAKRYKAAFTECCQAADKAACLLPKLDELRDE |
| | | GKASSAKQRLKCASLQKFGERAFKAWAVARLSQRFPKAEF |
| | | AEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLA |
| | | KTYETTLEKCCAAADPHECYAKVFDEFKPLVEEPQNLIKQN |
| | | CELFEQLGEYKFQNALLVRYTKKVPQVSTPTLVEVSRNLGK |
| | | VGSKCCKHPEAKRMPCAEDYLSVVLNQLCVLHEKTPVSDR |
| | | VTKCCTESLVNRRPCFSALEVDETYVPKEFNAETFTFHADIC |
| | | TLSEKERQIKKQTALVELVKHKPKATKEQLKAVMDDFAAF |
| | | VEKCCKADDKETCFAEEGKKLVAASQAALGLSGGEQKLISE |
| | | EDL |
| 21 | ProC1496/ | CAGGGACAGTCCGGATCTAGGCATCCATGTCGGCATGAC |
| | ProC_mLm16- | CCACATATCATATGCTACAAATTCGGTGGAGGATCTTCTG |
| | LIGHT-1204DNI- | GAGGCTCAATTTCATCAGGACTCCTGTCCGGGCGCTCCGA |
| | HSA-cMyc | CAATATTGGTGGCTCCCAGGAGAGGAGATCACATGAAGT |
| | DNA | CAACCCGGCCGCCCATCTTACTGGTGCCAATAGTAGTCTT |
| | | ACAGGTTCTGGCGGACCCTTGCTTTGGGAGACCCAGCTG |
| | | GGACTGGCTTTCCTTAGAGGCCTGAGTTATCACGACGGG |
| | | GCGTTGGTCGTTACTAAAGCCGGGTATTACTATATATACT |
| | | CTAAGGTTCAGCTCGGCGGTGTAGGATGCCCACTGGGTCT |
| | | TGCGTCTACAATTACACATGGTCTGTATAAGAGGACTCCC |
| | | AGATATCCAGAAGAACTGGAATTGCTTGTTTCCCAGCAAT |
| | | CTCCGTGCGGACGCGCAACGAGCTCCAGTAGGGTGTGGT |
| | | GGGACTCCAGCTTCCTGGGTGGCGTTGTCCATTTGGAGGC |
| | | GGGAGAGGAAGTAGTGGTGCGGGTCCTTGATGAACGCCT |
| | | TGTACGACTTCGGGACGGTACAAGAAGCTACTTTGGGGC |
| | | TTTCATGGTATCCAGCGCCGGGGGCGGAAGCAGCGGCGG |
| | | CAGCTTAAGCGGCAGAAGCGACAACATCGGCAGCGATGC |
| | | ACATAAAAGTGAGGTGGCCCACCGCTTTAAAGACCTCGG |
| | | CGAGGAGAACTTCAAGGCACTCGTACTTATTGCCTTTGCA |
| | | CAATACCTGCAGCAATGTCCGTTTGAAGATCATGTCAAGC |
| | | TCGTAAATGAAGTGACAGAGTTCGCTAAGACCTGTGTAG |
| | | CTGACGAAAGTGCCGAAAACTGCGACAAATCATTGCATA |
| | | CATTGTTCGGCGACAAGCTTTGTACAGTTGCCACGCTCCG |
| | | CGAGACATACGGCGAAATGGCCGATTGTTGTGCGAAACA |
| | | GGAACCCGAAAGAAATGAGTGCTTCTTGCAACACAAAGA |
| | | CGATAATCCCAATCTTCCGCGACTGGTGAGACCCGAAGT |
| | | AGATGTAATGTGCACAGCTTTTCATGACAATGAGGAAAC |
| | | CTTTCTTAAAAAATATCTGTACGAAATCGCCCGCCGACAC |
| | | CCGTATTTCTATGCCCCAGAGCTGTTGTTTTTCGCAAAGC |
| | | GCTACAAGGCAGCGTTTACCGAATGCTGCCAGGCCGCTG |
| | | ATAAAGCCGCTTGTCTGCTGCCTAAGCTTGATGAGCTTCG |
| | | CGACGAGGGTAAGGCTTCCAGCGCTAAACAACGCCTGAA |
| | | GTGTGCATCTTTGCAAAAATTTGGTGAACGAGCCTTCAAG |
| | | GCTTGGGCTGTTGCCAGGCTGAGTCAACGGTTCCCGAAG |
| | | GCGGAGTTTGCGGAGGTTTCCAAGTTGGTAACGGACCTG |
| | | ACAAAGGTACATACCGAATGCTGtCATGGCGACCTGTTGG |
| | | AGTGCGCCGATGATCGAGCGGATCTTGCCAAGTATATTTG |
| | | CGAAAACCAAGACTCCATTTCCAGTAAACTTAAGGAGTG |
| | | TTGTGAGAAACCTCTCCTTGAAAAGAGCCATTGTATAGCG |
| | | GAGGTCGAAAACGATGAAATGCCCGCTGATCTGCCGTCA |
| | | CTCGCTGCTGACTTCGTTGAATCCAAGGATGTATGTAAAA |
| | | ACTATGCCGAAGCTAAGGACGTCTTTCTTGGAATGTTCTT |
| | | GTATGAATACGCCCGACGGCATCCTGACTATTCAGTTGTG |
| | | CTCCTTCTTCGATTGGCTAAAACTTATGAGACGACACTTG |
| | | AAAAGTGTTGTGCCGCCGCCGACCCACATGAGTGCTACG |
| | | CTAAGGTTTTCGACGAATTTAAGCCCTTGGTTGAAGAACC |
| | | GCAGAATCTCATTAAGCAAAATTGTGAGCTCTTTGAACA |
| | | ACTCGGTGAATACAAATTTCAGAACGCGCTTCTGGTTAGA |
| | | TATACGAAGAAAGTGCCACAGGTGAGCACCCCTACATTG |
| | | GTTGAGGTCAGTAGGAACCTCGGCAAGGTGGGATCTAAG |
| | | TGCTGCAAACACCCGGAGGCAAAGAGAATGCCTTGCGCT |
| | | GAGGATTATCTCTCAGTGGTTCTGAATCAGCTTTGCGTTC |
| | | TTCACGAAAAGACTCCCGTCAGCGACCGAGTCACGAAGT |
| | | GCTGTACCGAGAGCCTTGTTAATAGACGACCTTGCTTCTC |
| | | CGCGCTTGAGGTGGATGAGACCTACGTCCCAAAGGAATT |
| | | CAACGCCGAGACGTTCACTTTCCATGCCGATATTTGCACC |
| | | TTGTCAGAAAAGGAGCGACAGATAAAAAAGCAAACTGCT |
| | | TTGGTGGAGCTGGTTAAGCATAAGCCTAAAGCGACGAAG |
| | | GAACAGCTCAAAGCCGTAATGGATGATTTCGCTGCCTTCG |
| | | TGGAGAAATGTTGTAAGGCCGATGACAAGGAAACCTGCT |
| | | TCGCTGAAGAGGGTAAAAAGCTCGTCGCCGCTAGTCAGG |
| | | CGGCACTTGGTTTGAGTGGTGGAGAACAAAAACTCATTT |
| | | CCGAGGAAGATCTTtga |
| 22 | ProC1184/ | QERRSHEVNPAAHLTGANSSLTGSGGPLLWETQLGLAFLRG |
| | ProC_LIGHT_ | LSYHDGALVVTKAGYYYIYSKVQLGGVGCPLGLASTITHGL |
| | 21linker_HSA-cMyc | YKRTPRYPEELELLVSQQSPCGRATSSSRVWWDSSFLGGVV |
| | Protein | HLEAGEEVVVRVLDERLVRLRDGTRSYFGAFMVSSAGGGS |
| | | SGGSSAGGGSSGGSDAHKSEVAHRFKDLGEENFKALVLIAF |
| | | AQYLQQCPFEDHVKLVNEVTEFAKTCVADESAENCDKSLH |
| | | TLFGDKLCTVATLRETYGEMADCCAKQEPERNECFLQHKD |
| | | DNPNLPRLVRPEVDVMCTAFHDNEETFLKKYLYEIARRHPY |
| | | FYAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSGGEQKLISEE |
| | | DL |
| 23 | ProC1184/ | CAGGAGAGGCGCTCTCATGAAGTAAACCCCGCCGCGCAC |
| | ProC_LIGHT_ | CTTACCGGTGCGAATTCCAGCCTTACGGGCTCCGGGGGC |
| | 21linker_HSA-cMyc | CCTCTTCTGTGGGAAACCCAACTCGGGCTGGCGTTTCTCA |
| | DNA | GGGGTCTCAGTTACCACGACGGCGCGCTTGTCGTTACTAA |
| | | AGCGGGCTACTACTATATCTACTCCAAGGTACAGCTCGGT |
| | | GGTGTAGGATGTCCGCTTGGGCTTGCCTCTACCATCACGC |
| | | ACGGTCTCTATAAAAGAACCCCAAGATACCCCGAAGAGT |
| | | TGGAACTGCTTGTTTCTCAACAGTCCCCTTGTGGTCGGGC |
| | | AACCAGTTCATCTCGGGTGTGGTGGGATAGTAGCTTTCTC |
| | | GGAGGAGTAGTCCACCTGGAAGCTGGGGAAGAGGTAGTC |
| | | GTCCGGGTACTCGATGAACGGCTCGTGCGCCTCCGAGAT |
| | | GGGACCCGGTCTTACTTTGGGGCTTTCATGGTTAGCTCTG |
| | | CGGGGGGAGGAAGCTCCGGGGGTAGTTCCGCCGGCGGCG |
| | | GATCTAGTGGCGGTTCTGATGCTCATAAAAGTGAGGTCG |
| | | CGCACAGATTCAAAGACCTCGGCGAGGAAAACTTTAAAG |
| | | CCCTCGTATTGATAGCTTTCGCCCAGTACCTCCAGCAGTG |
| | | CCCATTCGAGGATCACGTGAAACTCGTGAATGAAGTAAC |
| | | GGAATTTGCCAAAACGTGCGTGGCCGACGAGAGTGCCGA |
| | | AAATTGCGACAAGTCACTTCACACCCTTTTTGGTGACAAA |
| | | TTGTGTACTGTGGCGACGCTTAGGGAAACATACGGAGAA |
| | | ATGGCCGATTGCTGTGCCAAACAAGAGCCTGAAAGAAAC |
| | | GAATGCTTTCTTCAGCACAAAGACGATAACCCTAACTTGC |
| | | CGAGACTGGTGAGACCTGAAGTAGACGTCATGTGTACCG |
| | | CGTTCCATGACAACGAAGAGACTTTCTTGAAAAAATACC |
| | | TTTATGAAATAGCACGGAGGCACCCTTATTTTTACGCTCC |
| | | AGAGCTCCTCTTCTTTGCTAAACGGTACAAAGCGGCGTTT |
| | | ACCGAATGTTGCCAAGCTGCGGATAAAGCTGCATGTCTTC |
| | | TCCCGAAGTTGGATGAGCTGAGAGATGAGGGTAAAGCGT |
| | | CTTCCGCTAAACAACGACTTAAATGCGCTTCTCTGCAGAA |
| | | ATTTGGCGAACGAGCGTTCAAAGCCTGGGCCGTTGCGCG |
| | | CCTTTCACAGCGGTTTCCGAAAGCCGAGTTCGCCGAGGTT |
| | | TCAAAGCTGGTTACGGACTTGACAAAGGTGCATACAGAA |
| | | TGCTGCCACGGAGACCTGCTGGAGTGCGCCGATGATCGC |
| | | GCTGATTTGGCTAAATATATTTGCGAAAATCAGGACAGC |
| | | ATCAGCTCCAAGTTGAAGGAGTGTTGTGAAAAACCGCTG |
| | | CTGGAGAAGTCACACTGCATAGCCGAGGTGGAAAACGAC |
| | | GAGATGCCGGCAGACCTCCCCAGCCTTGCCGCGGATTTC |
| | | GTCGAATCAAAGGATGTATGCAAAAATTACGCGGAAGCA |
| | | AAAGATGTATTTCTGGGAATGTTCCTGTACGAGTACGCTA |
| | | GGCGACATCCCGACTACAGCGTTGTTCTGCTCTTGAGACT |
| | | TGCGAAGACGTACGAGACTACGCTCGAGAAGTGCTGTGC |
| | | AGCTGCGGACCCCCATGAGTGTTATGCTAAAGTCTTCGAC |
| | | GAATTTAAGCCTTTGGTGGAAGAGCCACAGAACCTGATC |
| | | AAACAAAATTGTGAACTGTTCGAACAATTGGGGGAGTAT |
| | | AAATTTCAGAACGCTCTCCTTGTTCGGTATACAAAAAAAG |
| | | TACCGCAGGTAAGTACGCCGACACTCGTGGAAGTAAGCA |
| | | GGAACCTCGGAAAGGTTGGATCAAAATGCTGCAAGCATC |
| | | CGGAGGCTAAGCGCATGCCATGCGCTGAGGATTATCTTTC |
| | | AGTGGTCCTGAACCAATTGTGCGTGTTGCACGAGAAAAC |
| | | TCCAGTAAGCGATAGAGTTACCAAATGTTGTACAGAGAG |
| | | CCTGGTTAATCGACGACCTTGCTTCAGCGCCTTGGAAGTC |
| | | GATGAGACGTATGTGCCGAAGGAATTTAACGCCGAAACT |
| | | TTCACTTTTCATGCAGATATCTGTACATTGAGCGAAAAGG |
| | | AAAGGCAAATTAAAAAACAAACTGCGCTTGTGGAATTGG |
| | | TCAAGCACAAGCCTAAAGCCACAAAGGAACAGCTCAAA |
| | | GCGGTAATGGACGATTTTGCGGCGTTCGTAGAGAAGTGT |
| | | TGCAAGGCGGACGATAAAGAAACGTGCTTTGCTGAGGAG |
| | | GGAAAAAAGCTTGTTGCTGCCAGTCAAGCGGCGCTTGGC |
| | | TTGAGTGGTGGAGAACAAAAACTCATTTCCGAGGAAGAT |
| | | CTTTGA |
| 24 | ProC1486/ | QERRSHEVNPAAHLTGANSSLTGSGGPLLWETQLGLAFLRG |
| | ProC_mhLIGHT_ | LSYHDGALVVTKTGYYYIYSKVQLGGVGCPLGLAGTITHGL |
| | 1204DNI_HSA-cMyc | YKRTPRYPEELELLVSQQSPCGRATSSSRVWWDSSFLGGVV |
| | Protein | HLEAGEEVVVRVLGKRLVRLRDGTRSYFGAFMVSSAGGGS |
| | | SGGSLSGRSDNIGSDAHKSEVAHRFKDLGEENFKALVLIAFA |
| | | QYLQQCPFEDHVKLVNEVTEFAKTCVADESAENCDKSLHT |
| | | LFGDKLCTVATLRETYGEMADCCAKQEPERNECFLQHKDD |
| | | NPNLPRLVRPEVDVMCTAFHDNEETFLKKYLYEIARRHPYF |
| | | YAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSGGEQKLISEE |
| | | DL |
| 25 | ProC1486/ | CAAGAACGCCGATCACACGAAGTGAACCCTGCTGCTCAC |
| | ProC_mhLIGHT_ | TTGACTGGAGCTAACTCCAGCCTTACGGGGAGCGGGGGT |
| | 1204DNI_HSA-cMyc | CCTTTGCTCTGGGAAACGCAGCTGGGTCTCGCGTTTCTTA |
| | DNA | GAGGGCTGAGTTATCACGATGGCGCCTTGGTCGTCACGA |
| | | AGACTGGGTACTACTATATTTATAGTAAGGTACAGTTGGG |
| | | GGGAGTGGGGTGCCCTCTCGGCCTGGCTGGGACGATCAC |
| | | GCATGGGCTCTACAAGCGCACGCCTAGGTATCCGGAGGA |
| | | GTTGGAACTGTTGGTCTCACAGCAGTCCCCGTGCGGGCG |
| | | AGCGACTTCCTCCAGTAGAGTGTGGTGGGACTCTAGCTTT |
| | | CTCGGGGGAGTGGTTCATCTTGAGGCAGGAGAGGAAGTG |
| | | GTGGTACGGGTTTTGGGCAAGAGATTGGTGAGACTGCGG |
| | | GATGGTACAAGATCTTATTTTGGTGCTTTCATGGTCTCCA |
| | | GCGCCGGGGGCGGAAGCAGCGGCGGCAGCTTAAGCGGC |
| | | AGAAGCGACAACATCGGCAGCGATGCTCATAAAAGTGAG |
| | | GTCGCGCACAGATTCAAAGACCTCGGCGAGGAAAACTTT |
| | | AAAGCCCTCGTATTGATAGCTTTCGCCCAGTACCTCCAGC |
| | | AGTGCCCATTCGAGGATCACGTGAAACTCGTGAATGAAG |
| | | TAACGGAATTTGCCAAAACGTGCGTGGCCGACGAGAGTG |
| | | CCGAAAATTGCGACAAGTCACTTCACACCCTTTTTGGTGA |
| | | CAAATTGTGTACTGTGGCGACGCTTAGGGAAACATACGG |
| | | AGAAATGGCCGATTGCTGTGCCAAACAAGAGCCTGAAAG |
| | | AAACGAATGCTTTCTTCAGCACAAAGACGATAACCCTAA |
| | | CTTGCCGAGACTGGTGAGACCTGAAGTAGACGTCATGTG |
| | | TACCGCGTTCCATGACAACGAAGAGACTTTCTTGAAAAA |
| | | ATACCTTTATGAAATAGCACGGAGGCACCCTTATTTTTAC |
| | | GCTCCAGAGCTCCTCTTCTTTGCTAAACGGTACAAAGCGG |
| | | CGTTTACCGAATGTTGCCAAGCTGCGGATAAAGCTGCAT |
| | | GTCTTCTCCCGAAGTTGGATGAGCTGAGAGATGAGGGTA |
| | | AAGCGTCTTCCGCTAAACAACGACTTAAATGCGCTTCTCT |
| | | GCAGAAATTTGGCGAACGAGCGTTCAAAGCCTGGGCCGT |
| | | TGCGCGCCTTTCACAGCGGTTTCCGAAAGCCGAGTTCGCC |
| | | GAGGTTTCAAAGCTGGTTACGGACTTGACAAAGGTGCAT |
| | | ACAGAATGCTGCCACGGAGACCTGCTGGAGTGCGCCGAT |
| | | GATCGCGCTGATTTGGCTAAATATATTTGCGAAAATCAGG |
| | | ACAGCATCAGCTCCAAGTTGAAGGAGTGTTGTGAAAAAC |
| | | CGCTGCTGGAGAAGTCACACTGCATAGCCGAGGTGGAAA |
| | | ACGACGAGATGCCGGCAGACCTCCCCAGCCTTGCCGCGG |
| | | ATTTCGTCGAATCAAAGGATGTATGCAAAAATTACGCGG |
| | | AAGCAAAAGATGTATTTCTGGGAATGTTCCTGTACGAGT |
| | | ACGCTAGGCGACATCCCGACTACAGCGTTGTTCTGCTCTT |
| | | GAGACTTGCGAAGACGTACGAGACTACGCTCGAGAAGTG |
| | | CTGTGCAGCTGCGGACCCCCATGAGTGTTATGCTAAAGTC |
| | | TTCGACGAATTTAAGCCTTTGGTGGAAGAGCCACAGAAC |
| | | CTGATCAAACAAAATTGTGAACTGTTCGAACAATTGGGG |
| | | GAGTATAAATTTCAGAACGCTCTCCTTGTTCGGTATACAA |
| | | AAAAAGTACCGCAGGTAAGTACGCCGACACTCGTGGAAG |
| | | TAAGCAGGAACCTCGGAAAGGTTGGATCAAAATGCTGCA |
| | | AGCATCCGGAGGCTAAGCGCATGCCATGCGCTGAGGATT |
| | | ATCTTTCAGTGGTCCTGAACCAATTGTGCGTGTTGCACGA |
| | | GAAAACTCCAGTAAGCGATAGAGTTACCAAATGTTGTAC |
| | | AGAGAGCCTGGTTAATCGACGACCTTGCTTCAGCGCCTTG |
| | | GAAGTCGATGAGACGTATGTGCCGAAGGAATTTAACGCC |
| | | GAAACTTTCACTTTTCATGCAGATATCTGTACATTGAGCG |
| | | AAAAGGAAAGGCAAATTAAAAAACAAACTGCGCTTGTGG |
| | | AATTGGTCAAGCACAAGCCTAAAGCCACAAAGGAACAGC |
| | | TCAAAGCGGTAATGGACGATTTTGCGGCGTTCGTAGAGA |
| | | AGTGTTGCAAGGCGGACGATAAAGAAACGTGCTTTGCTG |
| | | AGGAGGGAAAAAAGCTTGTTGCTGCCAGTCAAGCGGCGC |
| | | TTGGCTTGAGTGGTGGAGAACAAAAACTCATTTCCGAGG |
| | | AAGATCTTTGA |
| 26 | ProC1483/ | QERRSHEVNPAAHLTGANSSLTGSGGPLLWETQLGLAFLRG |
| | ProC_mhLIGHT_ | LSYHDGALVVTKTGYYYIYSKVQLGGVGCPLGLAGTITHGL |
| | 21linker_HSA-cMyc | YKRTPRYPEELELLVSQQSPCGRATSSSRVWWDSSFLGGVV |
| | Protein | HLEAGEEVVVRVLGKRLVRLRDGTRSYFGAFMVSSAGGGS |
| | | SGGSSAGGGSSGGSDAHKSEVAHRFKDLGEENFKALVLIAF |
| | | AQYLQQCPFEDHVKLVNEVTEFAKTCVADESAENCDKSLH |
| | | TLFGDKLCTVATLRETYGEMADCCAKQEPERNECFLQHKD |
| | | DNPNLPRLVRPEVDVMCTAFHDNEETFLKKYLYEIARRHPY |
| | | FYAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSGGEQKLISEE |
| | | DL |
| 27 | ProC1483/ | CAAGAACGCCGATCACACGAAGTGAACCCTGCTGCTCAC |
| | ProC_mhLIGHT_ | TTGACTGGAGCTAACTCCAGCCTTACGGGGAGCGGGGGT |
| | 21linker_HSA-cMyc | CCTTTGCTCTGGGAAACGCAGCTGGGTCTCGCGTTTCTTA |
| | DNA | GAGGGCTGAGTTATCACGATGGCGCCTTGGTCGTCACGA |
| | | AGACTGGGTACTACTATATTTATAGTAAGGTACAGTTGGG |
| | | GGGAGTGGGGTGCCCTCTCGGCCTGGCTGGGACGATCAC |
| | | GCATGGGCTCTACAAGCGCACGCCTAGGTATCCGGAGGA |
| | | GTTGGAACTGTTGGTCTCACAGCAGTCCCCGTGCGGGCG |
| | | AGCGACTTCCTCCAGTAGAGTGTGGTGGGACTCTAGCTTT |
| | | CTCGGGGGAGTGGTTCATCTTGAGGCAGGAGAGGAAGTG |
| | | GTGGTACGGGTTTTGGGCAAGAGATTGGTGAGACTGCGG |
| | | GATGGTACAAGATCTTATTTTGGTGCTTTCATGGTCAGCT |
| | | CTGCGGGGGGAGGAAGCTCCGGGGGTAGTTCCGCCGGCG |
| | | GCGGATCTAGTGGCGGTTCTGATGCTCATAAAAGTGAGG |
| | | TCGCGCACAGATTCAAAGACCTCGGCGAGGAAAACTTTA |
| | | AAGCCCTCGTATTGATAGCTTTCGCCCAGTACCTCCAGCA |
| | | GTGCCCATTCGAGGATCACGTGAAACTCGTGAATGAAGT |
| | | AACGGAATTTGCCAAAACGTGCGTGGCCGACGAGAGTGC |
| | | CGAAAATTGCGACAAGTCACTTCACACCCTTTTTGGTGAC |
| | | AAATTGTGTACTGTGGCGACGCTTAGGGAAACATACGGA |
| | | GAAATGGCCGATTGCTGTGCCAAACAAGAGCCTGAAAGA |
| | | AACGAATGCTTTCTTCAGCACAAAGACGATAACCCTAAC |
| | | TTGCCGAGACTGGTGAGACCTGAAGTAGACGTCATGTGT |
| | | ACCGCGTTCCATGACAACGAAGAGACTTTCTTGAAAAAA |
| | | TACCTTTATGAAATAGCACGGAGGCACCCTTATTTTTACG |
| | | CTCCAGAGCTCCTCTTCTTTGCTAAACGGTACAAAGCGGC |
| | | GTTTACCGAATGTTGCCAAGCTGCGGATAAAGCTGCATGT |
| | | CTTCTCCCGAAGTTGGATGAGCTGAGAGATGAGGGTAAA |
| | | GCGTCTTCCGCTAAACAACGACTTAAATGCGCTTCTCTGC |
| | | AGAAATTTGGCGAACGAGCGTTCAAAGCCTGGGCCGTTG |
| | | CGCGCCTTTCACAGCGGTTTCCGAAAGCCGAGTTCGCCG |
| | | AGGTTTCAAAGCTGGTTACGGACTTGACAAAGGTGCATA |
| | | CAGAATGCTGCCACGGAGACCTGCTGGAGTGCGCCGATG |
| | | ATCGCGCTGATTTGGCTAAATATATTTGCGAAAATCAGGA |
| | | CAGCATCAGCTCCAAGTTGAAGGAGTGTTGTGAAAAACC |
| | | GCTGCTGGAGAAGTCACACTGCATAGCCGAGGTGGAAAA |
| | | CGACGAGATGCCGGCAGACCTCCCCAGCCTTGCCGCGGA |
| | | TTTCGTCGAATCAAAGGATGTATGCAAAAATTACGCGGA |
| | | AGCAAAAGATGTATTTCTGGGAATGTTCCTGTACGAGTAC |
| | | GCTAGGCGACATCCCGACTACAGCGTTGTTCTGCTCTTGA |
| | | GACTTGCGAAGACGTACGAGACTACGCTCGAGAAGTGCT |
| | | GTGCAGCTGCGGACCCCCATGAGTGTTATGCTAAAGTCTT |
| | | CGACGAATTTAAGCCTTTGGTGGAAGAGCCACAGAACCT |
| | | GATCAAACAAAATTGTGAACTGTTCGAACAATTGGGGGA |
| | | GTATAAATTTCAGAACGCTCTCCTTGTTCGGTATACAAAA |
| | | AAAGTACCGCAGGTAAGTACGCCGACACTCGTGGAAGTA |
| | | AGCAGGAACCTCGGAAAGGTTGGATCAAAATGCTGCAAG |
| | | CATCCGGAGGCTAAGCGCATGCCATGCGCTGAGGATTAT |
| | | CTTTCAGTGGTCCTGAACCAATTGTGCGTGTTGCACGAGA |
| | | AAACTCCAGTAAGCGATAGAGTTACCAAATGTTGTACAG |
| | | AGAGCCTGGTTAATCGACGACCTTGCTTCAGCGCCTTGGA |
| | | AGTCGATGAGACGTATGTGCCGAAGGAATTTAACGCCGA |
| | | AACTTTCACTTTTCATGCAGATATCTGTACATTGAGCGAA |
| | | AAGGAAAGGCAAATTAAAAAACAAACTGCGCTTGTGGAA |
| | | TTGGTCAAGCACAAGCCTAAAGCCACAAAGGAACAGCTC |
| | | AAAGCGGTAATGGACGATTTTGCGGCGTTCGTAGAGAAG |
| | | TGTTGCAAGGCGGACGATAAAGAAACGTGCTTTGCTGAG |
| | | GAGGGAAAAAAGCTTGTTGCTGCCAGTCAAGCGGCGCTT |
| | | GGCTTGAGTGGTGGAGAACAAAAACTCATTTCCGAGGAA |
| | | GATCTTTGA |
| 28 | ProC1485/ | QGQSGSRHPCRHDPHIICYKFGGGSSGGSISSGLLSGRSDNIG |
| | ProC_mLm- | GSQERRSHEVNPAAHLTGANSSLTGSGGPLLWETQLGLAFL |
| | 16_mhLIGHT_ | RGLSYHDGALVVTKTGYYYIYSKVQLGGVGCPLGLAGTITH |
| | 21linker_HSA-cMyc | GLYKRTPRYPEELELLVSQQSPCGRATSSSRVWWDSSFLGG |
| | Protein | VVHLEAGEEVVVRVLGKRLVRLRDGTRSYFGAFMVSSAGG |
| | | GSSGGSSAGGGSSGGSDAHKSEVAHRFKDLGEENFKALVLI |
| | | AFAQYLQQCPFEDHVKLVNEVTEFAKTCVADESAENCDKS |
| | | LHTLFGDKLCTVATLRETYGEMADCCAKQEPERNECFLQH |
| | | KDDNPNLPRLVRPEVDVMCTAFHDNEETFLKKYLYEIARRH |
| | | PYFYAPELLFFAKRYKAAFTECCQAADKAACLLPKLDELRD |
| | | EGKASSAKQRLKCASLQKFGERAFKAWAVARLSQRFPKAE |
| | | FAEVSKLVTDLTKVHTECCHGDLLECADDRADLAKYICEN |
| | | QDSISSKLKECCEKPLLEKSHCIAEVENDEMPADLPSLAADF |
| | | VESKDVCKNYAEAKDVFLGMFLYEYARRHPDYSVVLLLRL |
| | | AKTYETTLEKCCAAADPHECYAKVFDEFKPLVEEPQNLIKQ |
| | | NCELFEQLGEYKFQNALLVRYTKKVPQVSTPTLVEVSRNLG |
| | | KVGSKCCKHPEAKRMPCAEDYLSVVLNQLCVLHEKTPVSD |
| | | RVTKCCTESLVNRRPCFSALEVDETYVPKEFNAETFTFHADI |
| | | CTLSEKERQIKKQTALVELVKHKPKATKEQLKAVMDDFAA |
| | | FVEKCCKADDKETCFAEEGKKLVAASQAALGLSGGEQKLIS |
| | | EEDL |
| 29 | ProC1485/ | CAGGGACAGTCCGGATCTAGGCATCCATGTCGGCATGAC |
| | ProC_mLm- | CCACATATCATATGCTACAAATTCGGTGGAGGATCTTCTG |
| | 16_mhLIGHT_ | GAGGCTCAATTTCATCAGGACTCCTGTCCGGGCGCTCCGA |
| | 21linker_HSA-cMyc | CAATATTGGTGGCTCCCAAGAACGCCGATCACACGAAGT |
| | DNA | GAACCCTGCTGCTCACTTGACTGGAGCTAACTCCAGCCTT |
| | | ACGGGGAGCGGGGGTCCTTTGCTCTGGGAAACGCAGCTG |
| | | GGTCTCGCGTTTCTTAGAGGGCTGAGTTATCACGATGGCG |
| | | CCTTGGTCGTCACGAAGACTGGGTACTACTATATTTATAG |
| | | TAAGGTACAGTTGGGGGGAGTGGGGTGCCCTCTCGGCCT |
| | | GGCTGGGACGATCACGCATGGGCTCTACAAGCGCACGCC |
| | | TAGGTATCCGGAGGAGTTGGAACTGTTGGTCTCACAGCA |
| | | GTCCCCGTGCGGGCGAGCGACTTCCTCCAGTAGAGTGTG |
| | | GTGGGACTCTAGCTTTCTCGGGGGAGTGGTTCATCTTGAG |
| | | GCAGGAGAGGAAGTGGTGGTACGGGTTTTGGGCAAGAGA |
| | | TTGGTGAGACTGCGGGATGGTACAAGATCTTATTTTGGTG |
| | | CTTTCATGGTCAGCTCTGCGGGGGGAGGAAGCTCCGGGG |
| | | GTAGTTCCGCCGGCGGCGGATCTAGTGGCGGTTCTGATGC |
| | | TCATAAAAGTGAGGTCGCGCACAGATTCAAAGACCTCGG |
| | | CGAGGAAAACTTTAAAGCCCTCGTATTGATAGCTTTCGCC |
| | | CAGTACCTCCAGCAGTGCCCATTCGAGGATCACGTGAAA |
| | | CTCGTGAATGAAGTAACGGAATTTGCCAAAACGTGCGTG |
| | | GCCGACGAGAGTGCCGAAAATTGCGACAAGTCACTTCAC |
| | | ACCCTTTTTGGTGACAAATTGTGTACTGTGGCGACGCTTA |
| | | GGGAAACATACGGAGAAATGGCCGATTGCTGTGCCAAAC |
| | | AAGAGCCTGAAAGAAACGAATGCTTTCTTCAGCACAAAG |
| | | ACGATAACCCTAACTTGCCGAGACTGGTGAGACCTGAAG |
| | | TAGACGTCATGTGTACCGCGTTCCATGACAACGAAGAGA |
| | | CTTTCTTGAAAAAATACCTTTATGAAATAGCACGGAGGC |
| | | ACCCTTATTTTTACGCTCCAGAGCTCCTCTTCTTTGCTAAA |
| | | CGGTACAAAGCGGCGTTTACCGAATGTTGCCAAGCTGCG |
| | | GATAAAGCTGCATGTCTTCTCCCGAAGTTGGATGAGCTGA |
| | | GAGATGAGGGTAAAGCGTCTTCCGCTAAACAACGACTTA |
| | | AATGCGCTTCTCTGCAGAAATTTGGCGAACGAGCGTTCA |
| | | AAGCCTGGGCCGTTGCGCGCCTTTCACAGCGGTTTCCGAA |
| | | AGCCGAGTTCGCCGAGGTTTCAAAGCTGGTTACGGACTT |
| | | GACAAAGGTGCATACAGAATGCTGCCACGGAGACCTGCT |
| | | GGAGTGCGCCGATGATCGCGCTGATTTGGCTAAATATATT |
| | | TGCGAAAATCAGGACAGCATCAGCTCCAAGTTGAAGGAG |
| | | TGTTGTGAAAAACCGCTGCTGGAGAAGTCACACTGCATA |
| | | GCCGAGGTGGAAAACGACGAGATGCCGGCAGACCTCCCC |
| | | AGCCTTGCCGCGGATTTCGTCGAATCAAAGGATGTATGC |
| | | AAAAATTACGCGGAAGCAAAAGATGTATTTCTGGGAATG |
| | | TTCCTGTACGAGTACGCTAGGCGACATCCCGACTACAGC |
| | | GTTGTTCTGCTCTTGAGACTTGCGAAGACGTACGAGACTA |
| | | CGCTCGAGAAGTGCTGTGCAGCTGCGGACCCCCATGAGT |
| | | GTTATGCTAAAGTCTTCGACGAATTTAAGCCTTTGGTGGA |
| | | AGAGCCACAGAACCTGATCAAACAAAATTGTGAACTGTT |
| | | CGAACAATTGGGGGAGTATAAATTTCAGAACGCTCTCCTT |
| | | GTTCGGTATACAAAAAAAGTACCGCAGGTAAGTACGCCG |
| | | ACACTCGTGGAAGTAAGCAGGAACCTCGGAAAGGTTGGA |
| | | TCAAAATGCTGCAAGCATCCGGAGGCTAAGCGCATGCCA |
| | | TGCGCTGAGGATTATCTTTCAGTGGTCCTGAACCAATTGT |
| | | GCGTGTTGCACGAGAAAACTCCAGTAAGCGATAGAGTTA |
| | | CCAAATGTTGTACAGAGAGCCTGGTTAATCGACGACCTT |
| | | GCTTCAGCGCCTTGGAAGTCGATGAGACGTATGTGCCGA |
| | | AGGAATTTAACGCCGAAACTTTCACTTTTCATGCAGATAT |
| | | CTGTACATTGAGCGAAAAGGAAAGGCAAATTAAAAAACA |
| | | AACTGCGCTTGTGGAATTGGTCAAGCACAAGCCTAAAGC |
| | | CACAAAGGAACAGCTCAAAGCGGTAATGGACGATTTTGC |
| | | GGCGTTCGTAGAGAAGTGTTGCAAGGCGGACGATAAAGA |
| | | AACGTGCTTTGCTGAGGAGGGAAAAAAGCTTGTTGCTGC |
| | | CAGTCAAGCGGCGCTTGGCTTGAGTGGTGGAGAACAAAA |
| | | ACTCATTTCCGAGGAAGATCTTTGA |
| 30 | ProC1487/ | QGQSGSRHPCRHDPHIICYKFGGGSSGGSISSGLLSGRSDNIG |
| | ProC_mLm- | GSQERRSHEVNPAAHLTGANSSLTGSGGPLLWETQLGLAFL |
| | 16_mhLIGHT_ | RGLSYHDGALVVTKTGYYYIYSKVQLGGVGCPLGLAGTITH |
| | 1204DNI_HSA-cMyc | GLYKRTPRYPEELELLVSQQSPCGRATSSSRVWWDSSFLGG |
| | Protein | VVHLEAGEEVVVRVLGKRLVRLRDGTRSYFGAFMVSSAGG |
| | | GSSGGSLSGRSDNIGSDAHKSEVAHRFKDLGEENFKALVLIA |
| | | FAQYLQQCPFEDHVKLVNEVTEFAKTCVADESAENCDKSL |
| | | HTLFGDKLCTVATLRETYGEMADCCAKQEPERNECFLQHK |
| | | DDNPNLPRLVRPEVDVMCTAFHDNEETFLKKYLYEIARRHP |
| | | YFYAPELLFFAKRYKAAFTECCQAADKAACLLPKLDELRDE |
| | | GKASSAKQRLKCASLQKFGERAFKAWAVARLSQRFPKAEF |
| | | AEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLA |
| | | KTYETTLEKCCAAADPHECYAKVFDEFKPLVEEPQNLIKQN |
| | | CELFEQLGEYKFQNALLVRYTKKVPQVSTPTLVEVSRNLGK |
| | | VGSKCCKHPEAKRMPCAEDYLSVVLNQLCVLHEKTPVSDR |
| | | VTKCCTESLVNRRPCFSALEVDETYVPKEFNAETFTFHADIC |
| | | TLSEKERQIKKQTALVELVKHKPKATKEQLKAVMDDFAAF |
| | | VEKCCKADDKETCFAEEGKKLVAASQAALGLSGGEQKLISE |
| | | EDL |
| 31 | ProC1487/ | CAGGGACAGTCCGGATCTAGGCATCCATGTCGGCATGAC |
| | ProC_mLm- | CCACATATCATATGCTACAAATTCGGTGGAGGATCTTCTG |
| | 16_mhLIGHT_ | GAGGCTCAATTTCATCAGGACTCCTGTCCGGGCGCTCCGA |
| | 1204DNI_HSA-cMyc | CAATATTGGTGGCTCCCAAGAACGCCGATCACACGAAGT |
| | DNA | GAACCCTGCTGCTCACTTGACTGGAGCTAACTCCAGCCTT |
| | | ACGGGGAGCGGGGGTCCTTTGCTCTGGGAAACGCAGCTG |
| | | GGTCTCGCGTTTCTTAGAGGGCTGAGTTATCACGATGGCG |
| | | CCTTGGTCGTCACGAAGACTGGGTACTACTATATTTATAG |
| | | TAAGGTACAGTTGGGGGGAGTGGGGTGCCCTCTCGGCCT |
| | | GGCTGGGACGATCACGCATGGGCTCTACAAGCGCACGCC |
| | | TAGGTATCCGGAGGAGTTGGAACTGTTGGTCTCACAGCA |
| | | GTCCCCGTGCGGGCGAGCGACTTCCTCCAGTAGAGTGTG |
| | | GTGGGACTCTAGCTTTCTCGGGGGAGTGGTTCATCTTGAG |
| | | GCAGGAGAGGAAGTGGTGGTACGGGTTTTGGGCAAGAGA |
| | | TTGGTGAGACTGCGGGATGGTACAAGATCTTATTTTGGTG |
| | | CTTTCATGGTCTCCAGCGCCGGGGGCGGAAGCAGCGGCG |
| | | GCAGCTTAAGCGGCAGAAGCGACAACATCGGCAGCGATG |
| | | CTCATAAAAGTGAGGTCGCGCACAGATTCAAAGACCTCG |
| | | GCGAGGAAAACTTTAAAGCCCTCGTATTGATAGCTTTCGC |
| | | CCAGTACCTCCAGCAGTGCCCATTCGAGGATCACGTGAA |
| | | ACTCGTGAATGAAGTAACGGAATTTGCCAAAACGTGCGT |
| | | GGCCGACGAGAGTGCCGAAAATTGCGACAAGTCACTTCA |
| | | CACCCTTTTTGGTGACAAATTGTGTACTGTGGCGACGCTT |
| | | AGGGAAACATACGGAGAAATGGCCGATTGCTGTGCCAAA |
| | | CAAGAGCCTGAAAGAAACGAATGCTTTCTTCAGCACAAA |
| | | GACGATAACCCTAACTTGCCGAGACTGGTGAGACCTGAA |
| | | GTAGACGTCATGTGTACCGCGTTCCATGACAACGAAGAG |
| | | ACTTTCTTGAAAAAATACCTTTATGAAATAGCACGGAGG |
| | | CACCCTTATTTTTACGCTCCAGAGCTCCTCTTCTTTGCTAA |
| | | ACGGTACAAAGCGGCGTTTACCGAATGTTGCCAAGCTGC |
| | | GGATAAAGCTGCATGTCTTCTCCCGAAGTTGGATGAGCTG |
| | | AGAGATGAGGGTAAAGCGTCTTCCGCTAAACAACGACTT |
| | | AAATGCGCTTCTCTGCAGAAATTTGGCGAACGAGCGTTC |
| | | AAAGCCTGGGCCGTTGCGCGCCTTTCACAGCGGTTTCCGA |
| | | AAGCCGAGTTCGCCGAGGTTTCAAAGCTGGTTACGGACT |
| | | TGACAAAGGTGCATACAGAATGCTGCCACGGAGACCTGC |
| | | TGGAGTGCGCCGATGATCGCGCTGATTTGGCTAAATATAT |
| | | TTGCGAAAATCAGGACAGCATCAGCTCCAAGTTGAAGGA |
| | | GTGTTGTGAAAAACCGCTGCTGGAGAAGTCACACTGCAT |
| | | AGCCGAGGTGGAAAACGACGAGATGCCGGCAGACCTCCC |
| | | CAGCCTTGCCGCGGATTTCGTCGAATCAAAGGATGTATGC |
| | | AAAAATTACGCGGAAGCAAAAGATGTATTTCTGGGAATG |
| | | TTCCTGTACGAGTACGCTAGGCGACATCCCGACTACAGC |
| | | GTTGTTCTGCTCTTGAGACTTGCGAAGACGTACGAGACTA |
| | | CGCTCGAGAAGTGCTGTGCAGCTGCGGACCCCCATGAGT |
| | | GTTATGCTAAAGTCTTCGACGAATTTAAGCCTTTGGTGGA |
| | | AGAGCCACAGAACCTGATCAAACAAAATTGTGAACTGTT |
| | | CGAACAATTGGGGGAGTATAAATTTCAGAACGCTCTCCTT |
| | | GTTCGGTATACAAAAAAAGTACCGCAGGTAAGTACGCCG |
| | | ACACTCGTGGAAGTAAGCAGGAACCTCGGAAAGGTTGGA |
| | | TCAAAATGCTGCAAGCATCCGGAGGCTAAGCGCATGCCA |
| | | TGCGCTGAGGATTATCTTTCAGTGGTCCTGAACCAATTGT |
| | | GCGTGTTGCACGAGAAAACTCCAGTAAGCGATAGAGTTA |
| | | CCAAATGTTGTACAGAGAGCCTGGTTAATCGACGACCTT |
| | | GCTTCAGCGCCTTGGAAGTCGATGAGACGTATGTGCCGA |
| | | AGGAATTTAACGCCGAAACTTTCACTTTTCATGCAGATAT |
| | | CTGTACATTGAGCGAAAAGGAAAGGCAAATTAAAAAACA |
| | | AACTGCGCTTGTGGAATTGGTCAAGCACAAGCCTAAAGC |
| | | CACAAAGGAACAGCTCAAAGCGGTAATGGACGATTTTGC |
| | | GGCGTTCGTAGAGAAGTGTTGCAAGGCGGACGATAAAGA |
| | | AACGTGCTTTGCTGAGGAGGGAAAAAAGCTTGTTGCTGC |
| | | CAGTCAAGCGGCGCTTGGCTTGAGTGGTGGAGAACAAAA |
| | | ACTCATTTCCGAGGAAGATCTTTGA |
| 32 | ProC1493/ | QERRSHEVNPAAHLTGANSSLTGSGGPLLWETQLGLAFLRG |
| | ProC_LIGHT-3HB- | LSYHDGALVVTKAGYYYIYSKVQLGGVGCPLGLASTITHGL |
| | 1204DNI-HSA-His | YKRTPRYPEELELLVSQQSPCGRATSSSRVWWDSSFLGGVV |
| | Protein | HLEAGEEVVVRVLDERLVRLRDGTRSYFGAFMVSSAGGGS |
| | | GGGSGGSGEIAALKQEIAALKKEIAALKWEIAALKQGYYGG |
| | | SGGSLSGRSDNIGSDAHKSEVAHRFKDLGEENFKALVLIAFA |
| | | QYLQQCPFEDHVKLVNEVTEFAKTCVADESAENCDKSLHT |
| | | LFGDKLCTVATLRETYGEMADCCAKQEPERNECFLQHKDD |
| | | NPNLPRLVRPEVDVMCTAFHDNEETFLKKYLYEIARRHPYF |
| | | YAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSHHHHHH |
| 33 | ProC1493/ | CAAGAGCGAAGATCCCATGAAGTAAACCCTGCAGCTCAC |
| | ProC_LIGHT-3HB- | CTTACAGGAGCCAACAGCAGTCTGACAGGTTCTGGGGGC |
| | 1204DNI-HSA-His | CCCTTGTTGTGGGAGACGCAACTGGGGCTTGCATTCCTCC |
| | DNA | GAGGGCTCAGTTACCACGACGGCGCGCTTGTTGTTACAA |
| | | AAGCAGGATACTACTATATTTACTCAAAGGTGCAGCTGG |
| | | GTGGAGTGGGATGTCCATTGGGCCTGGCCTCAACTATAA |
| | | CCCATGGCCTCTATAAAAGAACGCCCCGGTATCCTGAGG |
| | | AGCTGGAGCTGTTGGTCTCACAGCAGTCACCGTGCGGCA |
| | | GAGCCACATCATCCTCTCGCGTATGGTGGGACTCTTCCTT |
| | | CCTGGGAGGTGTAGTCCATCTCGAGGCAGGTGAAGAAGT |
| | | CGTAGTTCGCGTACTCGATGAACGCCTGGTTCGGCTGAGG |
| | | GATGGGACTAGGAGCTATTTCGGCGCATTTATGGTATCTT |
| | | CTGCAGGTGGAGGAAGTGGTGGCGGTTCCGGTGGTTCAG |
| | | GGGAAATCGCGGCACTCAAACAAGAGATAGCGGCTTTGA |
| | | AAAAGGAGATCGCAGCCCTGAAATGGGAAATAGCGGCCT |
| | | TGAAGCAGGGCTACTATGGCGGCAGCGGCGGCAGCCTAA |
| | | GCGGACGGTCCGACAATATCGGCAGCGATGCACATAAAA |
| | | GTGAGGTGGCCCACCGCTTTAAAGACCTCGGCGAGGAGA |
| | | ACTTCAAGGCACTCGTACTTATTGCCTTTGCACAATACCT |
| | | GCAGCAATGTCCGTTTGAAGATCATGTCAAGCTCGTAAAT |
| | | GAAGTGACAGAGTTCGCTAAGACCTGTGTAGCTGACGAA |
| | | AGTGCCGAAAACTGCGACAAATCATTGCATACATTGTTC |
| | | GGCGACAAGCTTTGTACAGTTGCCACGCTCCGCGAGACA |
| | | TACGGCGAAATGGCCGATTGTTGTGCGAAACAGGAACCC |
| | | GAAAGAAATGAGTGCTTCTTGCAACACAAAGACGATAAT |
| | | CCCAATCTTCCGCGACTGGTGAGACCCGAAGTAGATGTA |
| | | ATGTGCACAGCTTTTCATGACAATGAGGAAACCTTTCTTA |
| | | AAAAATATCTGTACGAAATCGCCCGCCGACACCCGTATTT |
| | | CTATGCCCCAGAGCTGTTGTTTTTCGCAAAGCGCTACAAG |
| | | GCAGCGTTTACCGAATGCTGCCAGGCCGCTGATAAAGCC |
| | | GCTTGTCTGCTGCCTAAGCTTGATGAGCTTCGCGACGAGG |
| | | GTAAGGCTTCCAGCGCTAAACAACGCCTGAAGTGTGCAT |
| | | CTTTGCAAAAATTTGGTGAACGAGCCTTCAAGGCTTGGGC |
| | | TGTTGCCAGGCTGAGTCAACGGTTCCCGAAGGCGGAGTT |
| | | TGCGGAGGTTTCCAAGTTGGTAACGGACCTGACAAAGGT |
| | | ACATACCGAATGCTGCCATGGCGACCTGTTGGAGTGCGC |
| | | CGATGATCGAGCGGATCTTGCCAAGTATATTTGCGAAAA |
| | | CCAAGACTCCATTTCCAGTAAACTTAAGGAGTGTTGTGAG |
| | | AAACCTCTCCTTGAAAAGAGCCATTGTATAGCGGAGGTC |
| | | GAAAACGATGAAATGCCCGCTGATCTGCCGTCACTCGCT |
| | | GCTGACTTCGTTGAATCCAAGGATGTATGTAAAAACTATG |
| | | CCGAAGCTAAGGACGTCTTTCTTGGAATGTTCTTGTATGA |
| | | ATACGCCCGACGGCATCCTGACTATTCAGTTGTGCTCCTT |
| | | CTTCGATTGGCTAAAACTTATGAGACGACACTTGAAAAG |
| | | TGTTGTGCCGCCGCCGACCCACATGAGTGCTACGCTAAG |
| | | GTTTTCGACGAATTTAAGCCCTTGGTTGAAGAACCGCAGA |
| | | ATCTCATTAAGCAAAATTGTGAGCTCTTTGAACAACTCGG |
| | | TGAATACAAATTTCAGAACGCGCTTCTGGTTAGATATACG |
| | | AAGAAAGTGCCACAGGTGAGCACCCCTACATTGGTTGAG |
| | | GTCAGTAGGAACCTCGGCAAGGTGGGATCTAAGTGCTGC |
| | | AAACACCCGGAGGCAAAGAGAATGCCTTGCGCTGAGGAT |
| | | TATCTCTCAGTGGTTCTGAATCAGCTTTGCGTTCTTCACG |
| | | AAAAGACTCCCGTCAGCGACCGAGTCACGAAGTGCTGTA |
| | | CCGAGAGCCTTGTTAATAGACGACCTTGCTTCTCCGCGCT |
| | | TGAGGTGGATGAGACCTACGTCCCAAAGGAATTCAACGC |
| | | CGAGACGTTCACTTTCCATGCCGATATTTGCACCTTGTCA |
| | | GAAAAGGAGCGACAGATAAAAAAGCAAACTGCTTTGGTG |
| | | GAGCTGGTTAAGCATAAGCCTAAAGCGACGAAGGAACAG |
| | | CTCAAAGCCGTAATGGATGATTTCGCTGCCTTCGTGGAGA |
| | | AATGTTGTAAGGCCGATGACAAGGAAACCTGCTTCGCTG |
| | | AAGAGGGTAAAAAGCTCGTCGCCGCTAGTCAGGCGGCAC |
| | | TTGGTTTGAGCCACCACCACCATCACCACtga |
| 34 | ProC1490/ | EIAALKQEIAALKKEIAALKWEIAALKQGYYGGSGGSQERR |
| | ProC_3HB-LIGHT- | SHEVNPAAHLTGANSSLTGSGGPLLWETQLGLAFLRGLSYH |
| | 1204DNI-HSA-cMyc | DGALVVTKAGYYYIYSKVQLGGVGCPLGLASTITHGLYKRT |
| | Protein | PRYPEELELLVSQQSPCGRATSSSRVWWDSSFLGGVVHLEA |
| | | GEEVVVRVLDERLVRLRDGTRSYFGAFMVSSAGGGSSGGS |
| | | LSGRSDNIGSDAHKSEVAHRFKDLGEENFKALVLIAFAQYL |
| | | QQCPFEDHVKLVNEVTEFAKTCVADESAENCDKSLHTLFGD |
| | | KLCTVATLRETYGEMADCCAKQEPERNECFLQHKDDNPNL |
| | | PRLVRPEVDVMCTAFHDNEETFLKKYLYEIARRHPYFYAPE |
| | | LLFFAKRYKAAFTECCQAADKAACLLPKLDELRDEGKASS |
| | | AKQRLKCASLQKFGERAFKAWAVARLSQRFPKAEFAEVSK |
| | | LVTDLTKVHTECCHGDLLECADDRADLAKYICENQDSISSK |
| | | LKECCEKPLLEKSHCIAEVENDEMPADLPSLAADFVESKDV |
| | | CKNYAEAKDVFLGMFLYEYARRHPDYSVVLLLRLAKTYET |
| | | TLEKCCAAADPHECYAKVFDEFKPLVEEPQNLIKQNCELFE |
| | | QLGEYKFQNALLVRYTKKVPQVSTPTLVEVSRNLGKVGSK |
| | | CCKHPEAKRMPCAEDYLSVVLNQLCVLHEKTPVSDRVTKC |
| | | CTESLVNRRPCFSALEVDETYVPKEFNAETFTFHADICTLSE |
| | | KERQIKKQTALVELVKHKPKATKEQLKAVMDDFAAFVEKC |
| | | CKADDKETCFAEEGKKLVAASQAALGLSGGEQKLISEEDL |
| 35 | ProC1490/ | GAAATCGCGGCACTCAAACAAGAGATAGCGGCTTTGAAA |
| | ProC_3HB-LIGHT- | AAGGAGATCGCAGCCCTGAAATGGGAAATAGCGGCCTTG |
| | 1204DNI-HSA-cMyc | AAGCAGGGCTACTATGGGGGATCCGGGGGTTCACAGGAG |
| | DNA | CGACGGTCCCATGAGGTGAATCCGGCAGCGCATTTGACG |
| | | GGGGCCAACTCATCCCTGACAGGTTCAGGTGGCCCTCTGT |
| | | TGTGGGAAACTCAGCTCGGACTGGCCTTCCTTAGAGGTTT |
| | | GTCATATCATGACGGAGCACTTGTAGTCACCAAAGCTGG |
| | | GTATTACTACATATACTCTAAGGTCCAGCTGGGTGGGGTG |
| | | GGCTGTCCACTTGGCTTGGCATCTACGATCACGCATGGGT |
| | | TGTACAAAAGAACTCCACGATATCCAGAAGAACTCGAAT |
| | | TGCTTGTCTCCCAACAATCTCCTTGTGGCAGGGCTACGTC |
| | | CAGTTCCCGAGTGTGGTGGGATTCAAGTTTTCTCGGGGGC |
| | | GTAGTCCATCTTGAAGCAGGGGAGGAAGTGGTCGTCCGA |
| | | GTGCTGGACGAACGGTTGGTTAGGCTTCGGGATGGGACA |
| | | AGAAGTTATTTTGGGGCCTTCATGGTATCCAGCGCCGGGG |
| | | GCGGAAGCAGCGGCGGCAGCTTAAGCGGCAGAAGCGAC |
| | | AACATCGGCAGCGATGCTCATAAAAGTGAGGTCGCGCAC |
| | | AGATTCAAAGACCTCGGCGAGGAAAACTTTAAAGCCCTC |
| | | GTATTGATAGCTTTCGCCCAGTACCTCCAGCAGTGCCCAT |
| | | TCGAGGATCACGTGAAACTCGTGAATGAAGTAACGGAAT |
| | | TTGCCAAAACGTGCGTGGCCGACGAGAGTGCCGAAAATT |
| | | GCGACAAGTCACTTCACACCCTTTTTGGTGACAAATTGTG |
| | | TACTGTGGCGACGCTTAGGGAAACATACGGAGAAATGGC |
| | | CGATTGCTGTGCCAAACAAGAGCCTGAAAGAAACGAATG |
| | | CTTTCTTCAGCACAAAGACGATAACCCTAACTTGCCGAGA |
| | | CTGGTGAGACCTGAAGTAGACGTCATGTGTACCGCGTTCC |
| | | ATGACAACGAAGAGACTTTCTTGAAAAAATACCTTTATG |
| | | AAATAGCACGGAGGCACCCTTATTTTTACGCTCCAGAGCT |
| | | CCTCTTCTTTGCTAAACGGTACAAAGCGGCGTTTACCGAA |
| | | TGTTGCCAAGCTGCGGATAAAGCTGCATGTCTTCTCCCGA |
| | | AGTTGGATGAGCTGAGAGATGAGGGTAAAGCGTCTTCCG |
| | | CTAAACAACGACTTAAATGCGCTTCTCTGCAGAAATTTGG |
| | | CGAACGAGCGTTCAAAGCCTGGGCCGTTGCGCGCCTTTC |
| | | ACAGCGGTTTCCGAAAGCCGAGTTCGCCGAGGTTTCAAA |
| | | GCTGGTTACGGACTTGACAAAGGTGCATACAGAATGCTG |
| | | CCACGGAGACCTGCTGGAGTGCGCCGATGATCGCGCTGA |
| | | TTTGGCTAAATATATTTGCGAAAATCAGGACAGCATCAG |
| | | CTCCAAGTTGAAGGAGTGTTGTGAAAAACCGCTGCTGGA |
| | | GAAGTCACACTGCATAGCCGAGGTGGAAAACGACGAGAT |
| | | GCCGGCAGACCTCCCCAGCCTTGCCGCGGATTTCGTCGA |
| | | ATCAAAGGATGTATGCAAAAATTACGCGGAAGCAAAAGA |
| | | TGTATTTCTGGGAATGTTCCTGTACGAGTACGCTAGGCGA |
| | | CATCCCGACTACAGCGTTGTTCTGCTCTTGAGACTTGCGA |
| | | AGACGTACGAGACTACGCTCGAGAAGTGCTGTGCAGCTG |
| | | CGGACCCCCATGAGTGTTATGCTAAAGTCTTCGACGAATT |
| | | TAAGCCTTTGGTGGAAGAGCCACAGAACCTGATCAAACA |
| | | AAATTGTGAACTGTTCGAACAATTGGGGGAGTATAAATT |
| | | TCAGAACGCTCTCCTTGTTCGGTATACAAAAAAAGTACCG |
| | | CAGGTAAGTACGCCGACACTCGTGGAAGTAAGCAGGAAC |
| | | CTCGGAAAGGTTGGATCAAAATGCTGCAAGCATCCGGAG |
| | | GCTAAGCGCATGCCATGCGCTGAGGATTATCTTTCAGTGG |
| | | TCCTGAACCAATTGTGCGTGTTGCACGAGAAAACTCCAGT |
| | | AAGCGATAGAGTTACCAAATGTTGTACAGAGAGCCTGGT |
| | | TAATCGACGACCTTGCTTCAGCGCCTTGGAAGTCGATGAG |
| | | ACGTATGTGCCGAAGGAATTTAACGCCGAAACTTTCACTT |
| | | TTCATGCAGATATCTGTACATTGAGCGAAAAGGAAAGGC |
| | | AAATTAAAAAACAAACTGCGCTTGTGGAATTGGTCAAGC |
| | | ACAAGCCTAAAGCCACAAAGGAACAGCTCAAAGCGGTA |
| | | ATGGACGATTTTGCGGCGTTCGTAGAGAAGTGTTGCAAG |
| | | GCGGACGATAAAGAAACGTGCTTTGCTGAGGAGGGAAAA |
| | | AAGCTTGTTGCTGCCAGTCAAGCGGCGCTTGGCTTGAGTG |
| | | GTGGAGAACAAAAACTCATTTCCGAGGAAGATCTTTGA |
| 36 | ProC1494/ | QGQSGSRHPCRHDPHIICYKFGGGSSGGSISSGLLSGRSDNIG |
| | ProC_mLm16- | GSQERRSHEVNPAAHLTGANSSLTGSGGPLLWETQLGLAFL |
| | LIGHT-3HB- | RGLSYHDGALVVTKAGYYYIYSKVQLGGVGCPLGLASTITH |
| | 1204DNI-HSA-His | GLYKRTPRYPEELELLVSQQSPCGRATSSSRVWWDSSFLGG |
| | Protein | VVHLEAGEEVVVRVLDERLVRLRDGTRSYFGAFMVSSAGG |
| | | GSGGGSGGSGEIAALKQEIAALKKEIAALKWEIAALKQGYY |
| | | GGSGGSLSGRSDNIGSDAHKSEVAHRFKDLGEENFKALVLI |
| | | AFAQYLQQCPFEDHVKLVNEVTEFAKTCVADESAENCDKS |
| | | LHTLFGDKLCTVATLRETYGEMADCCAKQEPERNECFLQH |
| | | KDDNPNLPRLVRPEVDVMCTAFHDNEETFLKKYLYEIARRH |
| | | PYFYAPELLFFAKRYKAAFTECCQAADKAACLLPKLDELRD |
| | | EGKASSAKQRLKCASLQKFGERAFKAWAVARLSQRFPKAE |
| | | FAEVSKLVTDLTKVHTECCHGDLLECADDRADLAKYICEN |
| | | QDSISSKLKECCEKPLLEKSHCIAEVENDEMPADLPSLAADF |
| | | VESKDVCKNYAEAKDVFLGMFLYEYARRHPDYSVVLLLRL |
| | | AKTYETTLEKCCAAADPHECYAKVFDEFKPLVEEPQNLIKQ |
| | | NCELFEQLGEYKFQNALLVRYTKKVPQVSTPTLVEVSRNLG |
| | | KVGSKCCKHPEAKRMPCAEDYLSVVLNQLCVLHEKTPVSD |
| | | RVTKCCTESLVNRRPCFSALEVDETYVPKEFNAETFTFHADI |
| | | CTLSEKERQIKKQTALVELVKHKPKATKEQLKAVMDDFAA |
| | | FVEKCCKADDKETCFAEEGKKLVAASQAALGLSHHHHHH |
| 37 | ProC1494/ | CAAGGACAGAGCGGCTCACGGCATCCTTGTAGACACGAC |
| | ProC_mLm16- | CCACACATCATCTGTTACAAGTTTGGAGGCGGCAGCAGC |
| | LIGHT-3HB- | GGCGGCTCTATCAGCTCTGGCCTTCTGAGCGGTCGGAGCG |
| | 1204DNI-HSA-His | ACAACATCGGAGGCAGCCAAGAGCGAAGATCCCATGAA |
| | DNA | GTAAACCCTGCAGCTCACCTTACAGGAGCCAACAGCAGT |
| | | CTGACAGGTTCTGGGGGCCCCTTGTTGTGGGAGACGCAA |
| | | CTGGGGCTTGCATTCCTCCGAGGGCTCAGTTACCACGACG |
| | | GCGCGCTTGTTGTTACAAAAGCAGGATACTACTATATTTA |
| | | CTCAAAGGTGCAGCTGGGTGGAGTGGGATGTCCATTGGG |
| | | CCTGGCCTCAACTATAACCCATGGCCTCTATAAAAGAAC |
| | | GCCCCGGTATCCTGAGGAGCTGGAGCTGTTGGTCTCACA |
| | | GCAGTCACCGTGCGGCAGAGCCACATCATCCTCTCGCGT |
| | | ATGGTGGGACTCTTCCTTCCTGGGAGGTGTAGTCCATCTC |
| | | GAGGCAGGTGAAGAAGTCGTAGTTCGCGTACTCGATGAA |
| | | CGCCTGGTTCGGCTGAGGGATGGGACTAGGAGCTATTTC |
| | | GGCGCATTTATGGTATCTTCTGCAGGTGGAGGAAGTGGT |
| | | GGCGGTTCCGGTGGTTCAGGGGAAATCGCGGCACTCAAA |
| | | CAAGAGATAGCGGCTTTGAAAAAGGAGATCGCAGCCCTG |
| | | AAATGGGAAATAGCGGCCTTGAAGCAGGGCTACTATGGC |
| | | GGCAGCGGCGGCAGCCTAAGCGGACGGTCCGACAATATC |
| | | GGCAGCGATGCACATAAAAGTGAGGTGGCCCACCGCTTT |
| | | AAAGACCTCGGCGAGGAGAACTTCAAGGCACTCGTACTT |
| | | ATTGCCTTTGCACAATACCTGCAGCAATGTCCGTTTGAAG |
| | | ATCATGTCAAGCTCGTAAATGAAGTGACAGAGTTCGCTA |
| | | AGACCTGTGTAGCTGACGAAAGTGCCGAAAACTGCGACA |
| | | AATCATTGCATACATTGTTCGGCGACAAGCTTTGTACAGT |
| | | TGCCACGCTCCGCGAGACATACGGCGAAATGGCCGATTG |
| | | TTGTGCGAAACAGGAACCCGAAAGAAATGAGTGCTTCTT |
| | | GCAACACAAAGACGATAATCCCAATCTTCCGCGACTGGT |
| | | GAGACCCGAAGTAGATGTAATGTGCACAGCTTTTCATGA |
| | | CAATGAGGAAACCTTTCTTAAAAAATATCTGTACGAAAT |
| | | CGCCCGCCGACACCCGTATTTCTATGCCCCAGAGCTGTTG |
| | | TTTTTCGCAAAGCGCTACAAGGCAGCGTTTACaGAATGCT |
| | | GCCAGGCCGCTGATAAAGCCGCTTGTCTGCTGCCTAAGCT |
| | | TGATGAGCTTCGCGACGAGGGTAAGGCTTCCAGCGCTAA |
| | | ACAACGCCTGAAGTGTGCATCTTTGCAAAAATTTGGTGA |
| | | ACGAGCCTTCAAGGCTTGGGCTGTTGCCAGGCTGAGTCA |
| | | ACGGTTCCCGAAGGCGGAGTTTGCGGAGGTTTCCAAGTT |
| | | GGTAACGGACCTGACAAAGGTACATACCGAATGCTGCCA |
| | | TGGCGACCTGTTGGAGTGCGCCGATGATCGAGCGGATCT |
| | | TGCCAAGTATATTTGCGAAAACCAAGACTCCATTTCCAGT |
| | | AAACTTAAGGAGTGTTGTGAGAAACCTCTCCTTGAAAAG |
| | | AGCCATTGTATAGCGGAGGTCGAAAACGATGAAATGCCC |
| | | GCTGATCTGCCGTCACTCGCTGCTGACTTCGTTGAATCCA |
| | | AGGATGTATGTAAAAACTATGCCGAAGCTAAGGACGTCT |
| | | TTCTTGGAATGTTCTTGTATGAATACGCCCGACGGCATCC |
| | | TGACTATTCAGTTGTGCTCCTTCTTCGATTGGCTAAAACTT |
| | | ATGAGACGACACTTGAAAAGTGTTGTGCCGCCGCCGACC |
| | | CACATGAGTGCTACGCTAAGGTTTTCGACGAATTTAAGCC |
| | | CTTGGTTGAAGAACCGCAGAATCTCATTAAGCAAAATTG |
| | | TGAGCTCTTTGAACAACTCGGTGAATACAAATTTCAGAAC |
| | | GCGCTTCTGGTTAGATATACGAAGAAAGTGCCACAGGTG |
| | | AGCACCCCTACATTGGTTGAGGTCAGTAGGAACCTCGGC |
| | | AAGGTGGGATCTAAGTGCTGCAAACACCCGGAGGCAAAG |
| | | AGAATGCCTTGCGCTGAGGATTATCTCTCAGTGGTTCTGA |
| | | ATCAGCTTTGCGTTCTTCACGAAAAGACTCCCGTCAGCGA |
| | | CCGAGTCACGAAGTGCTGTACCGAGAGCCTTGTTAATAG |
| | | ACGACCTTGCTTCTCCGCGCTTGAGGTGGATGAGACCTAC |
| | | GTCCCAAAGGAATTCAACGCCGAGACGTTCACTTTCCATG |
| | | CCGATATTTGCACCTTGTCAGAAAAGGAGCGACAGATAA |
| | | AAAAGCAAACTGCTTTGGTGGAGCTGGTTAAGCATAAGC |
| | | CTAAAGCGACGAAGGAACAGCTCAAAGCCGTAATGGATG |
| | | ATTTCGCTGCCTTCGTGGAGAAATGTTGTAAGGCCGATGA |
| | | CAAGGAAACCTGCTTCGCTGAAGAGGGTAAAAAGCTCGT |
| | | CGCCGCTAGTCAGGCGGCACTTGGTTTGAGCCACCACCA |
| | | CCATCACCACtga |
| 38 | ProC2006/ | GSGEQKLISEEDLGDAHKSEVAHRFKDLGEENFKALVLIAF |
| | ProC_cMyc-HSA- | AQYLQQCPFEDHVKLVNEVTEFAKTCVADESAENCDKSLH |
| | mLm16-1490DNI- | TLFGDKLCTVATLRETYGEMADCCAKQEPERNECFLQHKD |
| | LIGHT-3HB | DNPNLPRLVRPEVDVMCTAFHDNEETFLKKYLYEIARRHPY |
| | Protein | FYAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLGGGSSGGSGS |
| | | QGQSGSRHPCRHDPHIICYKFGGGSSGGSISSGLLSGRSDNIG |
| | | GSQERRSHEVNPAAHLTGANSSLTGSGGPLLWETQLGLAFL |
| | | RGLSYHDGALVVTKAGYYYIYSKVQLGGVGCPLGLASTITH |
| | | GLYKRTPRYPEELELLVSQQSPCGRATSSSRVWWDSSFLGG |
| | | VVHLEAGEEVVVRVLDERLVRLRDGTRSYFGAFMVSSAGG |
| | | GSGGGSGGSGEIAALKQEIAALKKEIAALKWEIAALKQGYY |
| | | GG |
| 39 | ProC2006/ | GGCTCTGGCGAACAAAAACTCATTTCCGAGGAAGATCTT |
| | ProC_cMyc-HSA- | GGCGATGCACATAAAAGTGAGGTGGCCCACCGCTTTAAA |
| | mLm16-1490DNI- | GACCTCGGCGAGGAGAACTTCAAGGCACTCGTACTTATT |
| | LIGHT-3HB | GCCTTTGCACAATACCTGCAGCAATGTCCGTTTGAAGATC |
| | DNA | ATGTCAAGCTCGTAAATGAAGTGACAGAGTTCGCTAAGA |
| | | CCTGTGTAGCTGACGAAAGTGCCGAAAACTGCGACAAAT |
| | | CATTGCATACATTGTTCGGCGACAAGCTTTGTACAGTTGC |
| | | CACGCTCCGCGAGACATACGGCGAAATGGCCGATTGTTG |
| | | TGCGAAACAGGAACCCGAAAGAAATGAGTGCTTCTTGCA |
| | | ACACAAAGACGATAATCCCAATCTTCCGCGACTGGTGAG |
| | | ACCCGAAGTAGATGTAATGTGCACAGCTTTTCATGACAAT |
| | | GAGGAAACCTTTCTTAAAAAATATCTGTACGAAATCGCC |
| | | CGCCGACACCCGTATTTCTATGCCCCAGAGCTGTTGTTTT |
| | | TCGCAAAGCGCTACAAGGCAGCGTTTACaGAATGCTGtCA |
| | | GGCCGCTGATAAAGCCGCTTGTCTGCTGCCTAAGCTTGAT |
| | | GAGCTTCGCGACGAGGGTAAGGCTTCCAGCGCTAAACAA |
| | | CGCCTGAAGTGTGCATCTTTGCAAAAATTTGGTGAACGA |
| | | GCCTTCAAGGCTTGGGCTGTTGCCAGGCTGAGTCAACGGT |
| | | TCCCGAAGGCGGAGTTTGCGGAGGTTTCCAAGTTGGTAA |
| | | CGGACCTGACAAAGGTACATACCGAATGCTGCCATGGCG |
| | | ACCTGTTGGAGTGCGCCGATGATCGAGCGGATCTTGCCA |
| | | AGTATATTTGCGAAAACCAAGACTCCATTTCCAGTAAACT |
| | | TAAGGAGTGTTGTGAGAAACCTCTCCTTGAAAAGAGCCA |
| | | TTGTATAGCGGAGGTCGAAAACGATGAAATGCCCGCTGA |
| | | TCTGCCGTCACTCGCTGCTGACTTCGTTGAATCCAAGGAT |
| | | GTATGTAAAAACTATGCCGAAGCTAAGGACGTCTTTCTTG |
| | | GAATGTTCTTGTATGAATACGCCCGACGGCATCCTGACTA |
| | | TTCAGTTGTGCTCCTTCTTCGATTGGCTAAAACTTATGAG |
| | | ACGACACTTGAAAAGTGTTGTGCCGCCGCCGACCCACAT |
| | | GAGTGCTACGCTAAGGTTTTCGACGAATTTAAGCCCTTGG |
| | | TTGAAGAACCGCAGAATCTCATTAAGCAAAATTGTGAGC |
| | | TCTTTGAACAACTCGGTGAATACAAATTTCAGAACGCGCT |
| | | TCTGGTTAGATATACGAAGAAAGTGCCACAGGTGAGCAC |
| | | CCCTACATTGGTTGAGGTCAGTAGGAACCTCGGCAAGGT |
| | | GGGATCTAAGTGCTGCAAACACCCGGAGGCAAAGAGAAT |
| | | GCCTTGCGCTGAGGATTATCTCTCAGTGGTTCTGAATCAG |
| | | CTTTGCGTTCTTCACGAAAAGACTCCCGTCAGCGACCGAG |
| | | TCACGAAGTGCTGTACCGAGAGCCTTGTTAATAGACGAC |
| | | CTTGCTTCTCCGCGCTTGAGGTGGATGAGACCTACGTCCC |
| | | AAAGGAATTCAACGCCGAGACGTTCACTTTCCATGCCGA |
| | | TATTTGCACCTTGTCAGAAAAGGAGCGACAGATAAAAAA |
| | | GCAAACTGCTTTGGTGGAGCTGGTTAAGCATAAGCCTAA |
| | | AGCGACGAAGGAACAGCTCAAAGCCGTAATGGATGATTT |
| | | CGCTGCCTTCGTGGAGAAATGTTGTAAGGCCGATGACAA |
| | | GGAAACCTGCTTCGCTGAAGAGGGTAAAAAGCTCGTCGC |
| | | CGCTAGTCAGGCGGCACTTGGTTTGGGGGGCGGAAGCAG |
| | | CGGCGGCAGCGGCAGCCAAGGCCAGAGCGGCAGCAGGC |
| | | ACCCTTGTAGACACGATCCTCACATCATCTGTTACAAGTT |
| | | TGGAGGAGGCAGTAGCGGTGGCAGTATTAGCAGCGGACT |
| | | GCTGAGTGGCAGAAGCGACAACATCGGCGGCAGCCAGG |
| | | AGAGACGGAGCCACGAGGTGAACCCCGCTGCCCACCTGA |
| | | CCGGCGCCAACAGCAGTCTGACCGGCAGCGGCGGCCCTC |
| | | TGCTGTGGGAGACACAGCTGGGCCTGGCCTTTCTGAGAG |
| | | GCCTGTCCTACCACGACGGCGCCCTGGTCGTGACCAAGG |
| | | CCGGCTACTACTACATCTACAGCAAGGTGCAGCTGGGAG |
| | | GCGTGGGCTGCCCTCTGGGCCTGGCATCTACCATCACGCA |
| | | CGGCCTGTACAAGCGGACCCCTAGATATCCTGAGGAACT |
| | | GGAACTCCTGGTGTCCCAGCAGAGCCCTTGCGGCAGAGC |
| | | TACAAGCAGCTCAAGAGTGTGGTGGGACAGCAGCTTCCT |
| | | GGGCGGAGTGGTCCACCTGGAAGCTGGAGAAGAAGTGGT |
| | | GGTCCGGGTGCTGGACGAGAGACTCGTGAGGCTGAGAGA |
| | | CGGAACAAGAAGCTACTTCGGCGCTTTTATGGTGTCTTCT |
| | | GCAGGTGGAGGAAGTGGTGGCGGTTCCGGTGGTTCAGGG |
| | | GAAATCGCGGCACTCAAACAAGAGATAGCGGCTTTGAAA |
| | | AAGGAGATCGCAGCCCTGAAATGGGAAATAGCGGCCTTG |
| | | AAGCAGGGCTACTATGGCGGCtga |
| 40 | ProC1497/ | GSGEQKLISEEDLGDAHKSEVAHRFKDLGEENFKALVLIAF |
| | ProC_cMyc-HSA- | AQYLQQCPFEDHVKLVNEVTEFAKTCVADESAENCDKSLH |
| | 21GS-1204DNI- | TLFGDKLCTVATLRETYGEMADCCAKQEPERNECFLQHKD |
| | LIGHT | DNPNLPRLVRPEVDVMCTAFHDNEETFLKKYLYEIARRHPY |
| | Protein | FYAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSSAGGGSSGG |
| | | SLSGRSDNIGSQERRSHEVNPAAHLTGANSSLTGSGGPLLW |
| | | ETQLGLAFLRGLSYHDGALVVTKAGYYYIYSKVQLGGVGC |
| | | PLGLASTITHGLYKRTPRYPEELELLVSQQSPCGRATSSSRV |
| | | WWDSSFLGGVVHLEAGEEVVVRVLDERLVRLRDGTRSYFG |
| | | AFMV |
| 41 | ProC1497/ | GGCTCTGGCGAACAAAAACTCATTTCCGAGGAAGATCTT |
| | ProC_cMyc-HSA- | GGCGATGCACATAAAAGTGAGGTGGCCCACCGCTTTAAA |
| | 21GS-1204DNI- | GACCTCGGCGAGGAGAACTTCAAGGCACTCGTACTTATT |
| | LIGHT | GCCTTTGCACAATACCTGCAGCAATGTCCGTTTGAAGATC |
| | DNA | ATGTCAAGCTCGTAAATGAAGTGACAGAGTTCGCTAAGA |
| | | CCTGTGTAGCTGACGAAAGTGCCGAAAACTGCGACAAAT |
| | | CATTGCATACATTGTTCGGCGACAAGCTTTGTACAGTTGC |
| | | CACGCTCCGCGAGACATACGGCGAAATGGCCGATTGTTG |
| | | TGCGAAACAGGAACCCGAAAGAAATGAGTGCTTCTTGCA |
| | | ACACAAAGACGATAATCCCAATCTTCCGCGACTGGTGAG |
| | | ACCCGAAGTAGATGTAATGTGCACAGCTTTTCATGACAAT |
| | | GAGGAAACCTTTCTTAAAAAATATCTGTACGAAATCGCC |
| | | CGCCGACACCCGTATTTCTATGCCCCAGAGCTGTTGTTTT |
| | | TCGCAAAGCGCTACAAGGCAGCGTTTACaGAATGCTGtCA |
| | | GGCCGCTGATAAAGCCGCTTGTCTGCTGCCTAAGCTTGAT |
| | | GAGCTTCGCGACGAGGGTAAGGCTTCCAGCGCTAAACAA |
| | | CGCCTGAAGTGTGCATCTTTGCAAAAATTTGGTGAACGA |
| | | GCCTTCAAGGCTTGGGCTGTTGCCAGGCTGAGTCAACGGT |
| | | TCCCGAAGGCGGAGTTTGCGGAGGTTTCCAAGTTGGTAA |
| | | CGGACCTGACAAAGGTACATACCGAATGCTGCCATGGCG |
| | | ACCTGTTGGAGTGCGCCGATGATCGAGCGGATCTTGCCA |
| | | AGTATATTTGCGAAAACCAAGACTCCATTTCCAGTAAACT |
| | | TAAGGAGTGTTGTGAGAAACCTCTCCTTGAAAAGAGCCA |
| | | TTGTATAGCGGAGGTCGAAAACGATGAAATGCCCGCTGA |
| | | TCTGCCGTCACTCGCTGCTGACTTCGTTGAATCCAAGGAT |
| | | GTATGTAAAAACTATGCCGAAGCTAAGGACGTCTTTCTTG |
| | | GAATGTTCTTGTATGAATACGCCCGACGGCATCCTGACTA |
| | | TTCAGTTGTGCTCCTTCTTCGATTGGCTAAAACTTATGAG |
| | | ACGACACTTGAAAAGTGTTGTGCCGCCGCCGACCCACAT |
| | | GAGTGCTACGCTAAGGTTTTCGACGAATTTAAGCCCTTGG |
| | | TTGAAGAACCGCAGAATCTCATTAAGCAAAATTGTGAGC |
| | | TCTTTGAACAACTCGGTGAATACAAATTTCAGAACGCGCT |
| | | TCTGGTTAGATATACGAAGAAAGTGCCACAGGTGAGCAC |
| | | CCCTACATTGGTTGAGGTCAGTAGGAACCTCGGCAAGGT |
| | | GGGATCTAAGTGCTGCAAACACCCGGAGGCAAAGAGAAT |
| | | GCCTTGCGCTGAGGATTATCTCTCAGTGGTTCTGAATCAG |
| | | CTTTGCGTTCTTCACGAAAAGACTCCCGTCAGCGACCGAG |
| | | TCACGAAGTGCTGTACCGAGAGCCTTGTTAATAGACGAC |
| | | CTTGCTTCTCCGCGCTTGAGGTGGATGAGACCTACGTCCC |
| | | AAAGGAATTCAACGCCGAGACGTTCACTTTCCATGCCGA |
| | | TATTTGCACCTTGTCAGAAAAGGAGCGACAGATAAAAAA |
| | | GCAAACTGCTTTGGTGGAGCTGGTTAAGCATAAGCCTAA |
| | | AGCGACGAAGGAACAGCTCAAAGCCGTAATGGATGATTT |
| | | CGCTGCCTTCGTGGAGAAATGTTGTAAGGCCGATGACAA |
| | | GGAAACCTGCTTCGCTGAAGAGGGTAAAAAGCTCGTCGC |
| | | CGCTAGTCAGGCGGCACTTGGTTTGTCCAGCGCCGGGGG |
| | | CGGAAGCAGCGGCGGCAGCTTAAGCGGCAGAAGCGACA |
| | | ACATCGGCAGCCAGGAGAGACGGAGCCACGAGGTGAAC |
| | | CCCGCTGCCCACCTGACCGGCGCCAACAGCAGTCTGACC |
| | | GGCAGCGGCGGCCCTCTGCTGTGGGAGACACAGCTGGGC |
| | | CTGGCCTTTCTGAGAGGCCTGTCCTACCACGACGGCGCCC |
| | | TGGTCGTGACCAAGGCCGGCTACTACTACATCTACAGCA |
| | | AGGTGCAGCTGGGAGGCGTGGGCTGCCCTCTGGGCCTGG |
| | | CATCTACCATCACGCACGGCCTGTACAAGCGGACCCCTA |
| | | GATATCCTGAGGAACTGGAACTCCTGGTGTCCCAGCAGA |
| | | GCCCTTGCGGCAGAGCTACAAGCAGCTCAAGAGTGTGGT |
| | | GGGACAGCAGCTTCCTGGGCGGAGTGGTCCACCTGGAAG |
| | | CTGGAGAAGAAGTGGTGGTCCGGGTGCTGGACGAGAGAC |
| | | TCGTGAGGCTGAGAGACGGAACAAGAAGCTACTTCGGCG |
| | | CTTTTATGGTGtga |
| 42 | ProC1489/ | QERRSHEVNPAAHLTGANSSLTGSGGPLLWETQLGLAFLRG |
| | ProC_LIGHT- | LSYHDGALVVTKAGYYYIYSKVQLGGVGCPLGLASTITHGL |
| | 1490DNI-mLm16- | YKRTPRYPEELELLVSQQSPCGRATSSSRVWWDSSFLGGVV |
| | HSA-cMyc | HLEAGEEVVVRVLDERLVRLRDGTRSYFGAFMVGGGSISSG |
| | Protein | LLSGRSDNIGGGSSGGSRHPCRHDPHIICYKFSGGGSGGGSG |
| | | DAHKSEVAHRFKDLGEENFKALVLIAFAQYLQQCPFEDHVK |
| | | LVNEVTEFAKTCVADESAENCDKSLHTLFGDKLCTVATLRE |
| | | TYGEMADCCAKQEPERNECFLQHKDDNPNLPRLVRPEVDV |
| | | MCTAFHDNEETFLKKYLYEIARRHPYFYAPELLFFAKRYKA |
| | | AFTECCQAADKAACLLPKLDELRDEGKASSAKQRLKCASL |
| | | QKFGERAFKAWAVARLSQRFPKAEFAEVSKLVTDLTKVHT |
| | | ECCHGDLLECADDRADLAKYICENQDSISSKLKECCEKPLLE |
| | | KSHCIAEVENDEMPADLPSLAADFVESKDVCKNYAEAKDV |
| | | FLGMFLYEYARRHPDYSVVLLLRLAKTYETTLEKCCAAADP |
| | | HECYAKVFDEFKPLVEEPQNLIKQNCELFEQLGEYKFQNAL |
| | | LVRYTKKVPQVSTPTLVEVSRNLGKVGSKCCKHPEAKRMP |
| | | CAEDYLSVVLNQLCVLHEKTPVSDRVTKCCTESLVNRRPCF |
| | | SALEVDETYVPKEFNAETFTFHADICTLSEKERQIKKQTALV |
| | | ELVKHKPKATKEQLKAVMDDFAAFVEKCCKADDKETCFAE |
| | | EGKKLVAASQAALGLSEQKLISEEDL |
| 43 | ProC1489/ | CAAGAGCGAAGATCCCATGAAGTAAACCCTGCAGCTCAC |
| | ProC_LIGHT- | CTTACAGGAGCCAACAGCAGTCTGACAGGTTCTGGGGGC |
| | 1490DNI-mLm16- | CCCTTGTTGTGGGAGACGCAACTGGGGCTTGCATTCCTCC |
| | HSA-cMyc | GAGGGCTCAGTTACCACGACGGCGCGCTTGTTGTTACAA |
| | DNA | AAGCAGGATACTACTATATTTACTCAAAGGTGCAGCTGG |
| | | GTGGAGTGGGATGTCCATTGGGCCTGGCCTCAACTATAA |
| | | CCCATGGCCTCTATAAAAGAACGCCCCGGTATCCTGAGG |
| | | AGCTGGAGCTGTTGGTCTCACAGCAGTCACCGTGCGGCA |
| | | GAGCCACATCATCCTCTCGCGTATGGTGGGACTCTTCCTT |
| | | CCTGGGAGGTGTAGTCCATCTCGAGGCAGGTGAAGAAGT |
| | | CGTAGTTCGCGTACTCGATGAACGCCTGGTTCGGCTGAGG |
| | | GATGGGACTAGGAGCTATTTCGGCGCATTTATGGTAGGC |
| | | GGCGGCTCTATCTCCTCCGGCCTGCTGAGCGGCAGAAGC |
| | | GACAACATCGGCGGAGGCAGCTCCGGCGGCTCTAGACAC |
| | | CCTTGTAGACACGATCCTCACATCATCTGTTACAAGTTTT |
| | | CTGGTGGAGGAAGTGGTGGCGGTTCCGGTGATGCACATA |
| | | AAAGTGAGGTGGCCCACCGCTTTAAAGACCTCGGCGAGG |
| | | AGAACTTCAAGGCACTCGTACTTATTGCCTTTGCACAATA |
| | | CCTGCAGCAATGTCCGTTTGAAGATCATGTCAAGCTCGTA |
| | | AATGAAGTGACAGAGTTCGCTAAGACCTGTGTAGCTGAC |
| | | GAAAGTGCCGAAAACTGCGACAAATCATTGCATACATTG |
| | | TTCGGCGACAAGCTTTGTACAGTTGCCACGCTCCGCGAGA |
| | | CATACGGCGAAATGGCCGATTGTTGTGCGAAACAGGAAC |
| | | CCGAAAGAAATGAGTGCTTCTTGCAACACAAAGACGATA |
| | | ATCCCAATCTTCCGCGACTGGTGAGACCCGAAGTAGATG |
| | | TAATGTGCACAGCTTTTCATGACAATGAGGAAACCTTTCT |
| | | TAAAAAATATCTGTACGAAATCGCCCGCCGACACCCGTA |
| | | TTTCTATGCCCCAGAGCTGTTGTTTTTCGCAAAGCGCTAC |
| | | AAGGCAGCGTTTACAGAATGCTGCCAGGCCGCTGATAAA |
| | | GCCGCTTGTCTGCTGCCTAAGCTTGATGAGCTTCGCGACG |
| | | AGGGTAAGGCTTCCAGCGCTAAACAACGCCTGAAGTGTG |
| | | CATCTTTGCAAAAATTTGGTGAACGAGCCTTCAAGGCTTG |
| | | GGCTGTTGCCAGGCTGAGTCAACGGTTCCCGAAGGCGGA |
| | | GTTTGCGGAGGTTTCCAAGTTGGTAACGGACCTGACAAA |
| | | GGTACATACCGAATGCTGCCATGGCGACCTGTTGGAGTG |
| | | CGCCGATGATCGAGCGGATCTTGCCAAGTATATTTGCGA |
| | | AAACCAAGACTCCATTTCCAGTAAACTTAAGGAGTGTTGT |
| | | GAGAAACCTCTCCTTGAAAAGAGCCATTGTATAGCGGAG |
| | | GTCGAAAACGATGAAATGCCCGCTGATCTGCCGTCACTC |
| | | GCTGCTGACTTCGTTGAATCCAAGGATGTATGTAAAAACT |
| | | ATGCCGAAGCTAAGGACGTCTTTCTTGGAATGTTCTTGTA |
| | | TGAATACGCCCGACGGCATCCTGACTATTCAGTTGTGCTC |
| | | CTTCTTCGATTGGCTAAAACTTATGAGACGACACTTGAAA |
| | | AGTGTTGTGCCGCCGCCGACCCACATGAGTGCTACGCTA |
| | | AGGTTTTCGACGAATTTAAGCCCTTGGTTGAAGAACCGCA |
| | | GAATCTCATTAAGCAAAATTGTGAGCTCTTTGAACAACTC |
| | | GGTGAATACAAATTTCAGAACGCGCTTCTGGTTAGATATA |
| | | CGAAGAAAGTGCCACAGGTGAGCACCCCTACATTGGTTG |
| | | AGGTCAGTAGGAACCTCGGCAAGGTGGGATCTAAGTGCT |
| | | GCAAACACCCGGAGGCAAAGAGAATGCCTTGCGCTGAGG |
| | | ATTATCTCTCAGTGGTTCTGAATCAGCTTTGCGTTCTTCAC |
| | | GAAAAGACTCCCGTCAGCGACCGAGTCACGAAGTGCTGT |
| | | ACCGAGAGCCTTGTTAATAGACGACCTTGCTTCTCCGCGC |
| | | TTGAGGTGGATGAGACCTACGTCCCAAAGGAATTCAACG |
| | | CCGAGACGTTCACTTTCCATGCCGATATTTGCACCTTGTC |
| | | AGAAAAGGAGCGACAGATAAAAAAGCAAACTGCTTTGGT |
| | | GGAGCTGGTTAAGCATAAGCCTAAAGCGACGAAGGAACA |
| | | GCTCAAAGCCGTAATGGATGATTTCGCTGCCTTCGTGGAG |
| | | AAATGTTGTAAGGCCGATGACAAGGAAACCTGCTTCGCT |
| | | GAAGAGGGTAAAAAGCTCGTCGCCGCTAGTCAGGCGGCA |
| | | CTTGGTTTGAGCGAACAAAAACTCATTTCCGAGGAAGAT |
| | | CTTtga |
| 44 | ProC1488/ | GSGEQKLISEEDLGDAHKSEVAHRFKDLGEENFKALVLIAF |
| | ProC_cMyc-HSA- | AQYLQQCPFEDHVKLVNEVTEFAKTCVADESAENCDKSLH |
| | mLm16-1490DNI- | TLFGDKLCTVATLRETYGEMADCCAKQEPERNECFLQHKD |
| | LIGHT | DNPNLPRLVRPEVDVMCTAFHDNEETFLKKYLYEIARRHPY |
| | Protein | FYAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLGGGSSGGSGS |
| | | QGQSGSRHPCRHDPHIICYKFGGGSSGGSISSGLLSGRSDNIG |
| | | GSQERRSHEVNPAAHLTGANSSLTGSGGPLLWETQLGLAFL |
| | | RGLSYHDGALVVTKAGYYYIYSKVQLGGVGCPLGLASTITH |
| | | GLYKRTPRYPEELELLVSQQSPCGRATSSSRVWWDSSFLGG |
| | | VVHLEAGEEVVVRVLDERLVRLRDGTRSYFGAFMV |
| 45 | ProC1488/ | GGCTCTGGCGAACAAAAACTCATTTCCGAGGAAGATCTT |
| | ProC_cMyc-HSA- | GGCGATGCACATAAAAGTGAGGTGGCCCACCGCTTTAAA |
| | mLm16-1490DNI- | GACCTCGGCGAGGAGAACTTCAAGGCACTCGTACTTATT |
| | LIGHT | GCCTTTGCACAATACCTGCAGCAATGTCCGTTTGAAGATC |
| | DNA | ATGTCAAGCTCGTAAATGAAGTGACAGAGTTCGCTAAGA |
| | | CCTGTGTAGCTGACGAAAGTGCCGAAAACTGCGACAAAT |
| | | CATTGCATACATTGTTCGGCGACAAGCTTTGTACAGTTGC |
| | | CACGCTCCGCGAGACATACGGCGAAATGGCCGATTGTTG |
| | | TGCGAAACAGGAACCCGAAAGAAATGAGTGCTTCTTGCA |
| | | ACACAAAGACGATAATCCCAATCTTCCGCGACTGGTGAG |
| | | ACCCGAAGTAGATGTAATGTGCACAGCTTTTCATGACAAT |
| | | GAGGAAACCTTTCTTAAAAAATATCTGTACGAAATCGCC |
| | | CGCCGACACCCGTATTTCTATGCCCCAGAGCTGTTGTTTT |
| | | TCGCAAAGCGCTACAAGGCAGCGTTTACaGAATGCTGtCA |
| | | GGCCGCTGATAAAGCCGCTTGTCTGCTGCCTAAGCTTGAT |
| | | GAGCTTCGCGACGAGGGTAAGGCTTCCAGCGCTAAACAA |
| | | CGCCTGAAGTGTGCATCTTTGCAAAAATTTGGTGAACGA |
| | | GCCTTCAAGGCTTGGGCTGTTGCCAGGCTGAGTCAACGGT |
| | | TCCCGAAGGCGGAGTTTGCGGAGGTTTCCAAGTTGGTAA |
| | | CGGACCTGACAAAGGTACATACCGAATGCTGCCATGGCG |
| | | ACCTGTTGGAGTGCGCCGATGATCGAGCGGATCTTGCCA |
| | | AGTATATTTGCGAAAACCAAGACTCCATTTCCAGTAAACT |
| | | TAAGGAGTGTTGTGAGAAACCTCTCCTTGAAAAGAGCCA |
| | | TTGTATAGCGGAGGTCGAAAACGATGAAATGCCCGCTGA |
| | | TCTGCCGTCACTCGCTGCTGACTTCGTTGAATCCAAGGAT |
| | | GTATGTAAAAACTATGCCGAAGCTAAGGACGTCTTTCTTG |
| | | GAATGTTCTTGTATGAATACGCCCGACGGCATCCTGACTA |
| | | TTCAGTTGTGCTCCTTCTTCGATTGGCTAAAACTTATGAG |
| | | ACGACACTTGAAAAGTGTTGTGCCGCCGCCGACCCACAT |
| | | GAGTGCTACGCTAAGGTTTTCGACGAATTTAAGCCCTTGG |
| | | TTGAAGAACCGCAGAATCTCATTAAGCAAAATTGTGAGC |
| | | TCTTTGAACAACTCGGTGAATACAAATTTCAGAACGCGCT |
| | | TCTGGTTAGATATACGAAGAAAGTGCCACAGGTGAGCAC |
| | | CCCTACATTGGTTGAGGTCAGTAGGAACCTCGGCAAGGT |
| | | GGGATCTAAGTGCTGCAAACACCCGGAGGCAAAGAGAAT |
| | | GCCTTGCGCTGAGGATTATCTCTCAGTGGTTCTGAATCAG |
| | | CTTTGCGTTCTTCACGAAAAGACTCCCGTCAGCGACCGAG |
| | | TCACGAAGTGCTGTACCGAGAGCCTTGTTAATAGACGAC |
| | | CTTGCTTCTCCGCGCTTGAGGTGGATGAGACCTACGTCCC |
| | | AAAGGAATTCAACGCCGAGACGTTCACTTTCCATGCCGA |
| | | TATTTGCACCTTGTCAGAAAAGGAGCGACAGATAAAAAA |
| | | GCAAACTGCTTTGGTGGAGCTGGTTAAGCATAAGCCTAA |
| | | AGCGACGAAGGAACAGCTCAAAGCCGTAATGGATGATTT |
| | | CGCTGCCTTCGTGGAGAAATGTTGTAAGGCCGATGACAA |
| | | GGAAACCTGCTTCGCTGAAGAGGGTAAAAAGCTCGTCGC |
| | | CGCTAGTCAGGCGGCACTTGGTTTGGGGGGCGGAAGCAG |
| | | CGGCGGCAGCGGCAGCCAAGGCCAGAGCGGCAGCAGGC |
| | | ACCCTTGTAGACACGATCCTCACATCATCTGTTACAAGTT |
| | | TGGAGGAGGCAGTAGCGGTGGCAGTATTAGCAGCGGACT |
| | | GCTGAGTGGCAGAAGCGACAACATCGGCGGCAGCCAGG |
| | | AGAGACGGAGCCACGAGGTGAACCCCGCTGCCCACCTGA |
| | | CCGGCGCCAACAGCAGTCTGACCGGCAGCGGCGGCCCTC |
| | | TGCTGTGGGAGACACAGCTGGGCCTGGCCTTTCTGAGAG |
| | | GCCTGTCCTACCACGACGGCGCCCTGGTCGTGACCAAGG |
| | | CCGGCTACTACTACATCTACAGCAAGGTGCAGCTGGGAG |
| | | GCGTGGGCTGCCCTCTGGGCCTGGCATCTACCATCACGCA |
| | | CGGCCTGTACAAGCGGACCCCTAGATATCCTGAGGAACT |
| | | GGAACTCCTGGTGTCCCAGCAGAGCCCTTGCGGCAGAGC |
| | | TACAAGCAGCTCAAGAGTGTGGTGGGACAGCAGCTTCCT |
| | | GGGCGGAGTGGTCCACCTGGAAGCTGGAGAAGAAGTGGT |
| | | GGTCCGGGTGCTGGACGAGAGACTCGTGAGGCTGAGAGA |
| | | CGGAACAAGAAGCTACTTCGGCGCTTTTATGGTGtga |
| 46 | ProC1498/ | GSGEQKLISEEDLGDAHKSEVAHRFKDLGEENFKALVLIAF |
| | ProC_cMyc-HSA- | AQYLQQCPFEDHVKLVNEVTEFAKTCVADESAENCDKSLH |
| | 21GS-1204DNI- | TLFGDKLCTVATLRETYGEMADCCAKQEPERNECFLQHKD |
| | LIGHT-mLm16 | DNPNLPRLVRPEVDVMCTAFHDNEETFLKKYLYEIARRHPY |
| | Protein | FYAPELLFFAKRYKAAFTECCQAADKAACLLPKLDELRDEG |
| | | KASSAKQRLKCASLQKFGERAFKAWAVARLSQRFPKAEFA |
| | | EVSKLVTDLTKVHTECCHGDLLECADDRADLAKYICENQD |
| | | SISSKLKECCEKPLLEKSHCIAEVENDEMPADLPSLAADFVE |
| | | SKDVCKNYAEAKDVFLGMFLYEYARRHPDYSVVLLLRLAK |
| | | TYETTLEKCCAAADPHECYAKVFDEFKPLVEEPQNLIKQNC |
| | | ELFEQLGEYKFQNALLVRYTKKVPQVSTPTLVEVSRNLGKV |
| | | GSKCCKHPEAKRMPCAEDYLSVVLNQLCVLHEKTPVSDRV |
| | | TKCCTESLVNRRPCFSALEVDETYVPKEFNAETFTFHADICT |
| | | LSEKERQIKKQTALVELVKHKPKATKEQLKAVMDDFAAFV |
| | | EKCCKADDKETCFAEEGKKLVAASQAALGLSSAGGGSSGG |
| | | SLSGRSDNIGSQERRSHEVNPAAHLTGANSSLTGSGGPLLW |
| | | ETQLGLAFLRGLSYHDGALVVTKAGYYYIYSKVQLGGVGC |
| | | PLGLASTITHGLYKRTPRYPEELELLVSQQSPCGRATSSSRV |
| | | WWDSSFLGGVVHLEAGEEVVVRVLDERLVRLRDGTRSYFG |
| | | AFMVGGGSISSGLLSGRSDNIGGGSSGGSRHPCRHDPHIICY |
| | | KF |
| 47 | ProC1498/ | GGCTCTGGCGAACAAAAACTCATTTCCGAGGAAGATCTT |
| | ProC_cMyc-HSA- | GGCGATGCACATAAAAGTGAGGTGGCCCACCGCTTTAAA |
| | 21GS-1204DNI- | GACCTCGGCGAGGAGAACTTCAAGGCACTCGTACTTATT |
| | LIGHT-mLm16 | GCCTTTGCACAATACCTGCAGCAATGTCCGTTTGAAGATC |
| | DNA | ATGTCAAGCTCGTAAATGAAGTGACAGAGTTCGCTAAGA |
| | | CCTGTGTAGCTGACGAAAGTGCCGAAAACTGCGACAAAT |
| | | CATTGCATACATTGTTCGGCGACAAGCTTTGTACAGTTGC |
| | | CACGCTCCGCGAGACATACGGCGAAATGGCCGATTGTTG |
| | | TGCGAAACAGGAACCCGAAAGAAATGAGTGCTTCTTGCA |
| | | ACACAAAGACGATAATCCCAATCTTCCGCGACTGGTGAG |
| | | ACCCGAAGTAGATGTAATGTGCACAGCTTTTCATGACAAT |
| | | GAGGAAACCTTTCTTAAAAAATATCTGTACGAAATCGCC |
| | | CGCCGACACCCGTATTTCTATGCCCCAGAGCTGTTGTTTT |
| | | TCGCAAAGCGCTACAAGGCAGCGTTTACaGAATGCTGtCA |
| | | GGCCGCTGATAAAGCCGCTTGTCTtCTGCCTAAGCTTGAT |
| | | GAGCTTCGCGACGAGGGTAAGGCTTCCAGCGCTAAACAA |
| | | CGCCTGAAGTGTGCATCTTTGCAAAAATTTGGTGAACGA |
| | | GCCTTCAAGGCTTGGGCTGTTGCCAGGCTGAGTCAACGGT |
| | | TCCCGAAGGCGGAGTTTGCGGAGGTTTCCAAGTTGGTAA |
| | | CGGACCTGACAAAGGTACATACCGAATGCTGCCATGGCG |
| | | ACCTGTTGGAGTGCGCCGATGATCGAGCGGATCTTGCCA |
| | | AGTATATTTGCGAAAACCAAGACTCCATTTCCAGTAAACT |
| | | TAAGGAGTGTTGTGAGAAACCTCTCCTTGAAAAGAGCCA |
| | | TTGTATAGCGGAGGTCGAAAACGATGAAATGCCCGCTGA |
| | | TCTGCCGTCACTCGCTGCTGACTTCGTTGAATCCAAGGAT |
| | | GTATGTAAAAACTATGCCGAAGCTAAGGACGTCTTTCTTG |
| | | GAATGTTCTTGTATGAATACGCCCGACGGCATCCTGACTA |
| | | TTCAGTTGTGCTCCTTCTTCGATTGGCTAAAACTTATGAG |
| | | ACGACACTTGAAAAGTGTTGTGCCGCtGCtGACCCACATG |
| | | AGTGCTACGCTAAGGTTTTCGACGAATTTAAGCCaTTGGT |
| | | TGAAGAACCGCAGAATCTCATTAAGCAAAATTGTGAGCT |
| | | CTTTGAACAACTCGGTGAATACAAATTTCAGAACGCGCTT |
| | | CTGGTTAGATATACGAAGAAAGTGCCACAGGTGAGCACC |
| | | CCTACATTGGTTGAGGTCAGTAGGAACCTCGGCAAGGTG |
| | | GGATCTAAGTGCTGCAAACACCCGGAGGCAAAGAGAATG |
| | | CCTTGCGCTGAGGATTATCTCTCAGTGGTTCTGAATCAGC |
| | | TTTGCGTTCTTCACGAAAAGACTCCCGTCAGCGACCGAGT |
| | | CACGAAGTGCTGTACCGAGAGCCTTGTTAATAGACGACC |
| | | TTGCTTCTCCGCGCTTGAGGTGGATGAGACCTACGTCCCA |
| | | AAGGAATTCAACGCCGAGACGTTCACTTTCCATGCCGAT |
| | | ATTTGCACCTTGTCAGAAAAGGAGCGACAGATAAAAAAG |
| | | CAAACTGCTTTGGTGGAGCTGGTTAAGCATAAGCCTAAA |
| | | GCGACGAAGGAACAGCTCAAAGCCGTAATGGATGATTTC |
| | | GCTGCCTTCGTGGAGAAATGTTGTAAGGCCGATGACAAG |
| | | GAAACCTGCTTCGCTGAAGAGGGTAAAAAGCTCGTCGCC |
| | | GCTAGTCAGGCGGCACTTGGTTTGTCCAGCGCCGGGGGC |
| | | GGAAGCAGCGGCGGCAGCTTAAGCGGCAGAAGCGACAA |
| | | CATCGGCAGCCAGGAGAGACGGAGCCACGAGGTGAACC |
| | | CCGCTGCCCACCTGACCGGCGCCAACAGCAGTCTGACCG |
| | | GCAGCGGCGGCCCTCTGCTGTGGGAGACACAGCTGGGCC |
| | | TGGCCTTTCTGAGAGGCCTGTCCTACCACGACGGCGCCCT |
| | | GGTCGTGACCAAGGCCGGCTACTACTACATCTACAGCAA |
| | | GGTGCAGCTGGGAGGCGTGGGCTGCCCTCTGGGCCTGGC |
| | | ATCTACCATCACGCACGGCCTGTACAAGCGGACCCCTAG |
| | | ATATCCTGAGGAACTGGAACTCCTGGTGTCCCAGCAGAG |
| | | CCCTTGCGGCAGAGCTACAAGCAGCTCAAGAGTGTGGTG |
| | | GGACAGCAGCTTCCTGGGCGGAGTGGTCCACCTGGAAGC |
| | | TGGAGAAGAAGTGGTGGTCCGGGTGCTGGACGAGAGACT |
| | | CGTGAGGCTGAGAGACGGAACAAGAAGCTACTTCGGCGC |
| | | TTTTATGGTGGGCGGAGGCTCCATTTCTAGCGGCCTGCTG |
| | | AGCGGCAGAAGCGATAACATCGGCGGaGGAAGCAGCGGA |
| | | GGCAGCAGACACCCCTGCAGACACGATCCTCACATCATC |
| | | TGCTACAAGTTCtga |
| 48 | ProC1162/ | QGQSGSRHPCRHDPHIICYKFGGGSSGGSISSGLLSGRSDNIG |
| | ProC_mLm- | GSQERRSHEVNPAAHLTGANSSLTGSGGPLLWETQLGLAFL |
| | 16_1490_LIGHT_ | RGLSYHDGALVVTKAGYYYIYSKVQLGGVGCPLGLASTITH |
| | 11linker_HSA-cMyc | GLYKRTPRYPEELELLVSQQSPCGRATSSSRVWWDSSFLGG |
| | Protein | VVHLEAGEEVVVRVLDERLVRLRDGTRSYFGAFMVSSAGG |
| | | GSSGGSDAHKSEVAHRFKDLGEENFKALVLIAFAQYLQQCP |
| | | FEDHVKLVNEVTEFAKTCVADESAENCDKSLHTLFGDKLCT |
| | | VATLRETYGEMADCCAKQEPERNECFLQHKDDNPNLPRLV |
| | | RPEVDVMCTAFHDNEETFLKKYLYEIARRHPYFYAPELLFF |
| | | AKRYKAAFTECCQAADKAACLLPKLDELRDEGKASSAKQR |
| | | LKCASLQKFGERAFKAWAVARLSQRFPKAEFAEVSKLVTD |
| | | LTKVHTECCHGDLLECADDRADLAKYICENQDSISSKLKEC |
| | | CEKPLLEKSHCIAEVENDEMPADLPSLAADFVESKDVCKNY |
| | | AEAKDVFLGMFLYEYARRHPDYSVVLLLRLAKTYETTLEK |
| | | CCAAADPHECYAKVFDEFKPLVEEPQNLIKQNCELFEQLGE |
| | | YKFQNALLVRYTKKVPQVSTPTLVEVSRNLGKVGSKCCKH |
| | | PEAKRMPCAEDYLSVVLNQLCVLHEKTPVSDRVTKCCTESL |
| | | VNRRPCFSALEVDETYVPKEFNAETFTFHADICTLSEKERQI |
| | | KKQTALVELVKHKPKATKEQLKAVMDDFAAFVEKCCKAD |
| | | DKETCFAEEGKKLVAASQAALGLSGGEQKLISEEDL |
| 49 | ProC1162/ | CAAGGGCAATCCGGATCTCGGCATCCGTGTCGGCATGAC |
| | ProC_mLm- | CCTCACATCATATGCTATAAGTTTGGGGGGGGATCCTCAG |
| | 16_1490_LIGHT_ | GTGGTTCCATTTCTTCTGGTTTGTTGTCTGGAAGATCAGA |
| | 11linker_HSA-cMyc | CAACATCGGCGGATCTCAAGAAAGACGCTCTCATGAGGT |
| | DNA | GAATCCTGCCGCGCACCTTACTGGGGCAAACTCCAGTCT |
| | | GACCGGATCAGGTGGGCCATTGCTTTGGGAGACACAGTT |
| | | GGGACTGGCATTCCTTCGAGGCCTCAGCTACCATGATGGC |
| | | GCCCTGGTCGTAACGAAAGCTGGGTATTACTATATATATA |
| | | GCAAAGTCCAACTGGGGGGGGTTGGGTGTCCCCTGGGGC |
| | | TCGCGTCAACCATCACCCACGGACTCTATAAGAGAACAC |
| | | CAAGATACCCAGAAGAGTTGGAACTTCTCGTGTCTCAAC |
| | | AAAGTCCATGTGGGAGGGCAACATCTTCTTCACGCGTAT |
| | | GGTGGGATTCATCCTTTCTCGGTGGCGTTGTCCATCTTGA |
| | | AGCAGGGGAGGAAGTTGTTGTCAGAGTCTTGGATGAAAG |
| | | ACTCGTGAGATTGCGGGATGGTACTAGATCCTACTTCGGC |
| | | GCGTTCATGGTCTCCTCAGCGGGAGGTGGGAGTAGTGGC |
| | | GGCTCAGACGCCCATAAATCCGAAGTGGCACATCGATTT |
| | | AAGGACCTCGGGGAGGAGAACTTCAAAGCTCTGGTACTC |
| | | ATCGCCTTTGCTCAGTACTTGCAGCAATGCCCCTTCGAGG |
| | | ATCATGTCAAACTCGTGAACGAGGTTACGGAGTTCGCTA |
| | | AAACATGTGTTGCTGACGAGTCTGCAGAGAACTGTGACA |
| | | AATCCCTCCACACGCTGTTCGGTGATAAACTGTGTACGGT |
| | | GGCTACCCTCAGGGAAACCTACGGAGAGATGGCCGATTG |
| | | TTGCGCCAAACAGGAGCCTGAGAGGAACGAATGCTTTCT |
| | | TCAACACAAGGATGACAATCCTAACTTGCCTAGGCTGGTT |
| | | CGGCCCGAGGTCGATGTGATGTGCACAGCGTTTCACGAC |
| | | AACGAAGAAACATTCCTGAAGAAATACTTGTACGAGATT |
| | | GCTAGGCGACACCCATATTTCTACGCGCCGGAGCTTCTCT |
| | | TCTTTGCGAAGCGCTACAAGGCTGCATTTACAGAGTGCTG |
| | | CCAAGCCGCTGATAAGGCGGCCTGTCTTCTCCCCAAGCTC |
| | | GATGAACTCCGAGATGAAGGGAAAGCTTCATCAGCGAAA |
| | | CAAAGATTGAAATGTGCTTCCCTCCAAAAGTTTGGAGAA |
| | | CGAGCCTTTAAGGCTTGGGCAGTGGCACGGCTCAGTCAG |
| | | CGCTTTCCTAAGGCTGAATTTGCCGAAGTGTCCAAGCTTG |
| | | TAACGGATCTCACTAAAGTTCATACTGAATGCTGCCACGG |
| | | AGACCTTCTCGAATGCGCGGACGATCGCGCGGACTTGGC |
| | | GAAATATATATGCGAGAATCAAGATAGTATCAGCAGTAA |
| | | ACTCAAAGAGTGCTGCGAGAAGCCTCTCCTCGAAAAGAG |
| | | CCACTGTATCGCCGAGGTGGAAAATGATGAGATGCCTGC |
| | | GGACTTGCCATCCCTTGCCGCAGACTTTGTCGAATCAAAA |
| | | GACGTTTGCAAGAATTACGCGGAGGCAAAAGATGTATTC |
| | | CTTGGCATGTTCTTGTACGAATACGCACGGCGCCACCCTG |
| | | ACTATTCAGTAGTGTTGCTCTTGAGACTCGCTAAAACATA |
| | | CGAAACGACGCTTGAGAAATGTTGCGCAGCAGCCGATCC |
| | | CCACGAGTGTTACGCAAAGGTGTTCGACGAGTTTAAACC |
| | | CCTCGTTGAAGAACCTCAAAACCTGATAAAACAAAATTG |
| | | TGAGTTGTTCGAGCAGTTGGGAGAGTACAAGTTTCAGAA |
| | | TGCTCTCCTGGTTCGGTACACCAAGAAGGTCCCACAAGTG |
| | | TCCACGCCCACCCTCGTAGAGGTATCACGGAACCTTGGC |
| | | AAGGTCGGTAGCAAGTGCTGCAAACACCCAGAAGCTAAG |
| | | CGCATGCCATGCGCTGAAGACTATCTGTCTGTGGTGCTTA |
| | | ATCAATTGTGTGTACTGCATGAGAAAACTCCTGTGTCCGA |
| | | CCGGGTTACCAAGTGCTGTACCGAGTCACTCGTCAACCG |
| | | GCGACCTTGTTTTTCTGCGCTGGAGGTTGATGAGACGTAT |
| | | GTTCCGAAAGAATTCAACGCCGAGACTTTCACCTTCCATG |
| | | CTGATATATGCACACTCAGTGAAAAAGAACGACAAATAA |
| | | AGAAGCAAACCGCATTGGTCGAGCTGGTCAAGCATAAAC |
| | | CCAAAGCTACAAAAGAACAATTGAAGGCTGTTATGGATG |
| | | ACTTTGCCGCGTTCGTAGAGAAATGCTGCAAGGCAGATG |
| | | ATAAAGAGACATGTTTCGCCGAAGAGGGCAAGAAACTGG |
| | | TGGCCGCCTCTCAAGCTGCACTTGGGCTCTCTGGAGGCGA |
| | | ACAAAAGCTTATAAGCGAAGAGGACTTGTGA |
| 50 | ProC1163/ | QGQSGSRHPCRHDPHIICYKFGGGSSGGSISSGLLSGRSDNIG |
| | ProC_mLm- | GSQERRSHEVNPAAHLTGANSSLTGSGGPLLWETQLGLAFL |
| | 16_1490_LIGHT_ | RGLSYHDGALVVTKAGYYYIYSKVQLGGVGCPLGLASTITH |
| | 21linker_HSA-cMyc | GLYKRTPRYPEELELLVSQQSPCGRATSSSRVWWDSSFLGG |
| | Protein | VVHLEAGEEVVVRVLDERLVRLRDGTRSYFGAFMVSSAGG |
| | | GSSGGSSAGGGSSGGSDAHKSEVAHRFKDLGEENFKALVLI |
| | | AFAQYLQQCPFEDHVKLVNEVTEFAKTCVADESAENCDKS |
| | | LHTLFGDKLCTVATLRETYGEMADCCAKQEPERNECFLQH |
| | | KDDNPNLPRLVRPEVDVMCTAFHDNEETFLKKYLYEIARRH |
| | | PYFYAPELLFFAKRYKAAFTECCQAADKAACLLPKLDELRD |
| | | EGKASSAKQRLKCASLQKFGERAFKAWAVARLSQRFPKAE |
| | | FAEVSKLVTDLTKVHTECCHGDLLECADDRADLAKYICEN |
| | | QDSISSKLKECCEKPLLEKSHCIAEVENDEMPADLPSLAADF |
| | | VESKDVCKNYAEAKDVFLGMFLYEYARRHPDYSVVLLLRL |
| | | AKTYETTLEKCCAAADPHECYAKVFDEFKPLVEEPQNLIKQ |
| | | NCELFEQLGEYKFQNALLVRYTKKVPQVSTPTLVEVSRNLG |
| | | KVGSKCCKHPEAKRMPCAEDYLSVVLNQLCVLHEKTPVSD |
| | | RVTKCCTESLVNRRPCFSALEVDETYVPKEFNAETFTFHADI |
| | | CTLSEKERQIKKQTALVELVKHKPKATKEQLKAVMDDFAA |
| | | FVEKCCKADDKETCFAEEGKKLVAASQAALGLSGGEQKLIS |
| | | EEDL |
| 51 | ProC1163/ | CAGGGACAGTCCGGATCTAGGCATCCATGTCGGCATGAC |
| | ProC_mLm- | CCACATATCATATGCTACAAATTCGGTGGAGGATCTTCTG |
| | 16_1490_LIGHT_ | GAGGCTCAATTTCATCAGGACTCCTGTCCGGGCGCTCCGA |
| | 21linker_HSA-cMyc | CAATATTGGTGGCTCCCAGGAGAGGAGATCACATGAAGT |
| | DNA | CAACCCGGCCGCCCATCTTACTGGTGCCAATAGTAGTCTT |
| | | ACAGGTTCTGGCGGACCCTTGCTTTGGGAGACCCAGCTG |
| | | GGACTGGCTTTCCTTAGAGGCCTGAGTTATCACGACGGG |
| | | GCGTTGGTCGTTACTAAAGCCGGGTATTACTATATATACT |
| | | CTAAGGTTCAGCTCGGCGGTGTAGGATGCCCACTGGGTCT |
| | | TGCGTCTACAATTACACATGGTCTGTATAAGAGGACTCCC |
| | | AGATATCCAGAAGAACTGGAATTGCTTGTTTCCCAGCAAT |
| | | CTCCGTGCGGACGCGCAACGAGCTCCAGTAGGGTGTGGT |
| | | GGGACTCCAGCTTCCTGGGTGGCGTTGTCCATTTGGAGGC |
| | | GGGAGAGGAAGTAGTGGTGCGGGTCCTTGATGAACGCCT |
| | | TGTACGACTTCGGGACGGTACAAGAAGCTACTTTGGGGC |
| | | TTTCATGGTATCTTCTGCAGGGGGGGGGTCATCCGGCGGA |
| | | AGTTCAGCGGGGGGGGGATCCTCCGGTGGCAGTGATGCA |
| | | CATAAAAGTGAGGTGGCCCACCGCTTTAAAGACCTCGGC |
| | | GAGGAGAACTTCAAGGCACTCGTACTTATTGCCTTTGCAC |
| | | AATACCTGCAGCAATGTCCGTTTGAAGATCATGTCAAGCT |
| | | CGTAAATGAAGTGACAGAGTTCGCTAAGACCTGTGTAGC |
| | | TGACGAAAGTGCCGAAAACTGCGACAAATCATTGCATAC |
| | | ATTGTTCGGCGACAAGCTTTGTACAGTTGCCACGCTCCGC |
| | | GAGACATACGGCGAAATGGCCGATTGTTGTGCGAAACAG |
| | | GAACCCGAAAGAAATGAGTGCTTCTTGCAACACAAAGAC |
| | | GATAATCCCAATCTTCCGCGACTGGTGAGACCCGAAGTA |
| | | GATGTAATGTGCACAGCTTTTCATGACAATGAGGAAACC |
| | | TTTCTTAAAAAATATCTGTACGAAATCGCCCGCCGACACC |
| | | CGTATTTCTATGCCCCAGAGCTGTTGTTTTTCGCAAAGCG |
| | | CTACAAGGCAGCGTTTACCGAATGCTGCCAGGCCGCTGA |
| | | TAAAGCCGCTTGTCTGCTGCCTAAGCTTGATGAGCTTCGC |
| | | GACGAGGGTAAGGCTTCCAGCGCTAAACAACGCCTGAAG |
| | | TGTGCATCTTTGCAAAAATTTGGTGAACGAGCCTTCAAGG |
| | | CTTGGGCTGTTGCCAGGCTGAGTCAACGGTTCCCGAAGG |
| | | CGGAGTTTGCGGAGGTTTCCAAGTTGGTAACGGACCTGA |
| | | CAAAGGTACATACCGAATGCTGCCATGGCGACCTGTTGG |
| | | AGTGCGCCGATGATCGAGCGGATCTTGCCAAGTATATTTG |
| | | CGAAAACCAAGACTCCATTTCCAGTAAACTTAAGGAGTG |
| | | TTGTGAGAAACCTCTCCTTGAAAAGAGCCATTGTATAGCG |
| | | GAGGTCGAAAACGATGAAATGCCCGCTGATCTGCCGTCA |
| | | CTCGCTGCTGACTTCGTTGAATCCAAGGATGTATGTAAAA |
| | | ACTATGCCGAAGCTAAGGACGTCTTTCTTGGAATGTTCTT |
| | | GTATGAATACGCCCGACGGCATCCTGACTATTCAGTTGTG |
| | | CTCCTTCTTCGATTGGCTAAAACTTATGAGACGACACTTG |
| | | AAAAGTGTTGTGCCGCCGCCGACCCACATGAGTGCTACG |
| | | CTAAGGTTTTCGACGAATTTAAGCCCTTGGTTGAAGAACC |
| | | GCAGAATCTCATTAAGCAAAATTGTGAGCTCTTTGAACA |
| | | ACTCGGTGAATACAAATTTCAGAACGCGCTTCTGGTTAGA |
| | | TATACGAAGAAAGTGCCACAGGTGAGCACCCCTACATTG |
| | | GTTGAGGTCAGTAGGAACCTCGGCAAGGTGGGATCTAAG |
| | | TGCTGCAAACACCCGGAGGCAAAGAGAATGCCTTGCGCT |
| | | GAGGATTATCTCTCAGTGGTTCTGAATCAGCTTTGCGTTC |
| | | TTCACGAAAAGACTCCCGTCAGCGACCGAGTCACGAAGT |
| | | GCTGTACCGAGAGCCTTGTTAATAGACGACCTTGCTTCTC |
| | | CGCGCTTGAGGTGGATGAGACCTACGTCCCAAAGGAATT |
| | | CAACGCCGAGACGTTCACTTTCCATGCCGATATTTGCACC |
| | | TTGTCAGAAAAGGAGCGACAGATAAAAAAGCAAACTGCT |
| | | TTGGTGGAGCTGGTTAAGCATAAGCCTAAAGCGACGAAG |
| | | GAACAGCTCAAAGCCGTAATGGATGATTTCGCTGCCTTCG |
| | | TGGAGAAATGTTGTAAGGCCGATGACAAGGAAACCTGCT |
| | | TCGCTGAAGAGGGTAAAAAGCTCGTCGCCGCTAGTCAGG |
| | | CGGCACTTGGTTTGTCAGGTGGGGAGCAGAAACTTATCTC |
| | | CGAAGAGGATTTGTAA |
| 52 | ProC1164/ | QGQSGSRHPCRHDPHIICYKFGGGSSGGSISSGLLSGRSDNIG |
| | ProC_mLm- | GSQERRSHEVNPAAHLTGANSSLTGSGGPLLWETQLGLAFL |
| | 16_1490_LIGHT_ | RGLSYHDGALVVTKAGYYYIYSKVQLGGVGCPLGLASTITH |
| | IgG4 WT hinge_HSA- | GLYKRTPRYPEELELLVSQQSPCGRATSSSRVWWDSSFLGG |
| | cMyc | VVHLEAGEEVVVRVLDERLVRLRDGTRSYFGAFMVESKYG |
| | Protein | PPCPSCPAPEFLGGPSDAHKSEVAHRFKDLGEENFKALVLIA |
| | | FAQYLQQCPFEDHVKLVNEVTEFAKTCVADESAENCDKSL |
| | | HTLFGDKLCTVATLRETYGEMADCCAKQEPERNECFLQHK |
| | | DDNPNLPRLVRPEVDVMCTAFHDNEETFLKKYLYEIARRHP |
| | | YFYAPELLFFAKRYKAAFTECCQAADKAACLLPKLDELRDE |
| | | GKASSAKQRLKCASLQKFGERAFKAWAVARLSQRFPKAEF |
| | | AEVSKLVTDLTKVHTECCHGDLLECADDRADLAKYICENQ |
| | | DSISSKLKECCEKPLLEKSHCIAEVENDEMPADLPSLAADFV |
| | | ESKDVCKNYAEAKDVFLGMFLYEYARRHPDYSVVLLLRLA |
| | | KTYETTLEKCCAAADPHECYAKVFDEFKPLVEEPQNLIKQN |
| | | CELFEQLGEYKFQNALLVRYTKKVPQVSTPTLVEVSRNLGK |
| | | VGSKCCKHPEAKRMPCAEDYLSVVLNQLCVLHEKTPVSDR |
| | | VTKCCTESLVNRRPCFSALEVDETYVPKEFNAETFTFHADIC |
| | | TLSEKERQIKKQTALVELVKHKPKATKEQLKAVMDDFAAF |
| | | VEKCCKADDKETCFAEEGKKLVAASQAALGLSGGEQKLISE |
| | | EDL |
| 53 | ProC1164/ | CAAGGCCAGAGTGGGAGTAGGCATCCATGTCGCCATGAT |
| | ProC_mLm- | CCTCACATTATATGCTATAAATTTGGTGGGGGAAGCAGTG |
| | 16_1490_LIGHT_ | GTGGGTCCATCAGTAGTGGGCTGTTGTCCGGTCGGAGTG |
| | IgG4 WT hinge_HSA- | ATAATATTGGAGGCTCCCAGGAACGCAGGAGTCATGAAG |
| | cMyc | TTAACCCGGCGGCACATCTTACGGGTGCGAACTCTAGTCT |
| | DNA | TACCGGGAGTGGGGGGCCCCTGCTTTGGGAGACACAGCT |
| | | TGGGCTCGCTTTCCTCAGGGGACTCTCTTACCATGATGGC |
| | | GCACTGGTAGTAACCAAAGCTGGATACTATTATATTTACT |
| | | CTAAAGTTCAATTGGGGGGCGTTGGCTGCCCACTCGGCCT |
| | | CGCATCTACTATCACCCATGGGTTGTATAAGCGGACCCCT |
| | | AGATACCCTGAGGAACTTGAGCTTTTGGTTTCTCAACAGT |
| | | CACCTTGCGGTAGGGCTACCTCATCAAGCCGCGTTTGGTG |
| | | GGACAGTTCATTTCTCGGGGGCGTAGTCCATCTGGAGGC |
| | | AGGTGAGGAAGTAGTGGTTCGAGTGCTCGACGAGCGCCT |
| | | CGTACGACTCCGGGATGGGACGAGAAGTTATTTCGGCGC |
| | | CTTTATGGTAGAGTCAAAATACGGGCCTCCCTGTCCCTCT |
| | | TGTCCGGCACCCGAATTTCTTGGCGGGCCCTCTGATGCCC |
| | | ACAAGTCTGAAGTTGCGCATAGATTTAAAGACCTCGGAG |
| | | AGGAGAATTTTAAAGCACTCGTGCTTATCGCATTTGCGCA |
| | | GTACCTGCAGCAGTGCCCTTTCGAGGACCACGTCAAGCTT |
| | | GTCAACGAGGTGACAGAATTCGCCAAAACATGTGTCGCC |
| | | GATGAGTCTGCCGAGAACTGTGACAAAAGTTTGCATACC |
| | | CTGTTCGGCGACAAACTCTGCACTGTAGCAACTCTTAGGG |
| | | AAACATACGGGGAAATGGCAGACTGTTGCGCGAAACAGG |
| | | AGCCAGAACGAAACGAATGTTTCTTGCAGCACAAGGATG |
| | | ATAACCCTAATCTCCCGAGGTTGGTCAGGCCCGAAGTCG |
| | | ACGTAATGTGCACGGCTTTCCATGACAATGAGGAAACCT |
| | | TCCTCAAGAAGTACCTCTACGAAATAGCACGAAGACATC |
| | | CGTATTTCTATGCTCCTGAGCTGCTCTTTTTCGCTAAACGC |
| | | TATAAGGCCGCATTCACGGAGTGTTGTCAAGCCGCCGAT |
| | | AAGGCTGCTTGCCTTCTGCCCAAGTTGGACGAACTGCGCG |
| | | ATGAGGGGAAAGCATCATCAGCAAAGCAACGCCTGAAAT |
| | | GCGCTAGTTTGCAAAAATTCGGGGAACGCGCCTTCAAAG |
| | | CTTGGGCTGTCGCGCGGCTCTCACAGAGGTTCCCCAAGG |
| | | CTGAGTTCGCCGAAGTTTCCAAGCTGGTTACTGATTTGAC |
| | | CAAAGTGCACACAGAATGTTGCCACGGCGACCTGCTTGA |
| | | GTGTGCGGATGACCGCGCAGATCTCGCGAAGTATATATG |
| | | CGAAAACCAGGATTCAATTAGCTCTAAATTGAAAGAATG |
| | | TTGTGAGAAACCTCTGCTTGAAAAGTCACACTGCATTGCG |
| | | GAGGTGGAAAATGATGAAATGCCCGCAGATCTCCCCTCT |
| | | TTGGCAGCGGACTTCGTGGAGAGTAAAGACGTCTGTAAG |
| | | AATTACGCCGAGGCGAAGGATGTATTCCTGGGGATGTTT |
| | | CTCTATGAATACGCTCGCAGACATCCTGACTACTCTGTGG |
| | | TGCTGCTGTTGCGCCTCGCTAAGACCTACGAAACAACCCT |
| | | GGAAAAATGTTGTGCCGCAGCTGATCCACACGAATGCTA |
| | | TGCTAAAGTCTTTGATGAATTTAAGCCTTTGGTTGAGGAG |
| | | CCCCAGAACCTGATAAAACAGAACTGTGAATTGTTCGAG |
| | | CAACTTGGAGAGTATAAATTCCAAAACGCGCTTCTCGTGC |
| | | GGTACACCAAGAAGGTGCCTCAAGTCAGTACTCCAACCC |
| | | TTGTGGAGGTAAGTCGCAATCTCGGTAAAGTTGGCAGTA |
| | | AATGTTGTAAGCATCCTGAAGCGAAGCGCATGCCTTGTG |
| | | CAGAGGACTATCTGTCAGTAGTTCTTAACCAGCTGTGTGT |
| | | GCTTCATGAGAAAACACCCGTGTCCGACAGGGTGACAAA |
| | | GTGTTGCACTGAGAGTCTCGTGAACCGGAGACCTTGTTTC |
| | | TCCGCCCTGGAAGTGGACGAAACCTATGTGCCGAAGGAA |
| | | TTCAATGCTGAAACGTTTACGTTCCATGCCGACATCTGCA |
| | | CGCTTAGCGAGAAAGAGAGACAGATAAAGAAGCAAACC |
| | | GCCTTGGTGGAACTCGTTAAGCACAAGCCAAAAGCAACC |
| | | AAAGAACAACTCAAAGCGGTCATGGACGATTTCGCTGCA |
| | | TTTGTAGAGAAATGCTGCAAAGCGGATGACAAGGAGACG |
| | | TGTTTCGCTGAAGAGGGAAAAAAGCTCGTGGCAGCATCC |
| | | CAGGCTGCACTTGGTCTGTCTGGTGGTGAGCAAAAACTG |
| | | ATTTCAGAGGAGGATTTGTGA |
| 54 | human LIGHT | QERRSHEVNPAAHLTGANSSLTGSGGPLLWETQLGLAFLRG |
| | protomer | LSYHDGALVVTKAGYYYIYSKVQLGGVGCPLGLASTITHGL |
| | | YKRTPRYPEELELLVSQQSPCGRATSSSRVWWDSSFLGGVV |
| | | HLEAGEEVVVRVLDERLVRLRDGTRSYFGAFMV |
| 55 | mouse-human | QERRSHEVNPAAHLTGANSSLTGSGGPLLWETQLGLAFLRG |
| | LIGHT protomer | LSYHDGALVVTKTGYYYIYSKVQLGGVGCPLGLAGTITHGL |
| | | YKRTPRYPEELELLVSQQSPCGRATSSSRVWWDSSFLGGVV |
| | | HLEAGEEVVVRVLGKRLVRLRDGTRSYFGAFMV |
| 56 | HSA | DAHKSEVAHRFKDLGEENFKALVLIAFAQYLQQCPFEDHVK |
| | | LVNEVTEFAKTCVADESAENCDKSLHTLFGDKLCTVATLRE |
| | | TYGEMADCCAKQEPERNECFLQHKDDNPNLPRLVRPEVDV |
| | | MCTAFHDNEETFLKKYLYEIARRHPYFYAPELLFFAKRYKA |
| | | AFTECCQAADKAACLLPKLDELRDEGKASSAKQRLKCASL |
| | | QKFGERAFKAWAVARLSQRFPKAEFAEVSKLVTDLTKVHT |
| | | ECCHGDLLECADDRADLAKYICENQDSISSKLKECCEKPLLE |
| | | KSHCIAEVENDEMPADLPSLAADFVESKDVCKNYAEAKDV |
| | | FLGMFLYEYARRHPDYSVVLLLRLAKTYETTLEKCCAAADP |
| | | HECYAKVFDEFKPLVEEPQNLIKQNCELFEQLGEYKFQNAL |
| | | LVRYTKKVPQVSTPTLVEVSRNLGKVGSKCCKHPEAKRMP |
| | | CAEDYLSVVLNQLCVLHEKTPVSDRVTKCCTESLVNRRPCF |
| | | SALEVDETYVPKEFNAETFTFHADICTLSEKERQIKKQTALV |
| | | ELVKHKPKATKEQLKAVMDDFAAFVEKCCKADDKETCFAE |
| | | EGKKLVAASQAALGL |
| 57 | 3HB-Trimerization | EIAALKQEIAALKKEIAALKWEIAALKQGYY |
| | three helix bundle | |
| 58 | His | HHHHHH |
| 59 | cMyc | EQKLISEEDL |
| 60 | Strep | SAWSHPQFEK |
| 61 | mLm16 | RHPCRHDPHIICYKF |
| 62 | 1490DNI | ISSGLLSGRSDNI |
| 63 | 1204DNI | LSGRSDNI |
| 64 | Linker (11) | SSAGGGSSGGS |
| 65 | Linker (21) | SSAGGGSSGGSSAGGGSSGGS |
| 66 | Linker (10) | SGGGSGGGSG |
| 67 | Linker (11c) | GGGSSGGSGS |
| 68 | Linker (8) | GGGSSGGS |
| 69 | Linker (11b) | SGGGSGGGSGS |
| | Linker (3a) | GGS |
| | Linker (3b) | SGG |
| | Linker (3c) | GSG |
| | Linker(2) | GS |
| | Linker(1b) | G |
| 75 | Linker(4) | GGGS |
| 76 | Header | QGQSGS |
| 77 | Linker (IgG4 WT | ESKYGPPCPSCPAPEFLGGPS |
| | hinge) | |
| 78 | signal peptide | METDTLLLWVLLLWVPGSTG |
| 79 | Full length human | MEESVVRPSVFVVDGQTDIPFTRLGRSHRRQSCSVARVGLG |
| | LIGHT | LLLLLMGAGLAVQGWFLLQLHWRLGEMVTRLPDGPAGSW |
| | | EQLIQERRSHEVNPAAHLTGANSSLTGSGGPLLWETQLGLA |
| | | FLRGLSYHDGALVVTKAGYYYIYSKVQLGGVGCPLGLASTI |
| | | THGLYKRTPRYPEELELLVSQQSPCGRATSSSRVWWDSSFL |
| | | GGVVHLEAGEEVVVRVLDERLVRLRDGTRSYFGAFMV |
| | Linker (1a) | S |
| 81 | 1490DNI-SER to | ISSGLLSGRCDNI |
| | CYS | |
| 82 | ProC1189/ | METDTLLLWVLLLWVPGSTGQERRSHEVNPAAHLTGANSS |
| | ProC_LIGHT-10GS- | LTGSGGPLLWETQLGLAFLRGLSYHDGALVVTKAGYYYIYS |
| | His | KVQLGGVGCPLGLASTITHGLYKRTPRYPEELELLVSQQSPC |
| | Protein with signal | GRATSSSRVWWDSSFLGGVVHLEAGEEVVVRVLDERLVRL |
| | sequence | RDGTRSYFGAFMVSGGGSGGGSGHHHHHH |
| 83 | ProC1189/ | atggaaaccgacacactgctgctgtgggtgctgcttttgtgggtgccaggatccacaggcCA |
| | ProC_LIGHT-10GS- | GGAACGGCGCTCCCACGAGGTCAATCCGGCGGCTCATTT |
| | His | GACTGGAGCCAACTCTAGTCTTACCGGGAGCGGCGGTCC |
| | DNA with coding | ATTGCTGTGGGAGACGCAACTTGGATTGGCTTTTCTTCGG |
| | sequence for signal | GGATTGTCTTACCACGATGGAGCACTCGTTGTTACGAAAG |
| | peptide | CCGGTTACTACTACATTTATTCTAAGGTCCAGTTGGGTGG |
| | | AGTTGGCTGTCCCCTGGGACTTGCAAGCACTATCACTCAC |
| | | GGACTGTACAAGAGGACACCCCGATACCCCGAGGAACTC |
| | | GAGCTGCTTGTGAGCCAACAGTCTCCATGTGGGCGCGCA |
| | | ACTAGTTCTTCACGGGTGTGGTGGGACTCCAGTTTCCTGG |
| | | GAGGTGTTGTTCATCTCGAGGCAGGTGAAGAAGTAGTTG |
| | | TCAGGGTACTTGATGAACGGCTTGTTAGACTCCGGGATG |
| | | GAACGAGGTCCTATTTCGGTGCGTTCATGGTAAGCGGGG |
| | | GCGGAAGTGGGGGCGGGTCAGGTCATCATCACCACCATC |
| | | ATTGA |
| 84 | ProC1193/ | METDTLLLWVLLLWVPGSTGQGQSGSRHPCRHDPHIICYKF |
| | ProC_mLm- | GGGSSGGSISSGLLSGRSDNIGGSQERRSHEVNPAAHLTGAN |
| | 16_1490_LIGHT_ | SSLTGSGGPLLWETQLGLAFLRGLSYHDGALVVTKAGYYYI |
| | 10GS His | YSKVQLGGVGCPLGLASTITHGLYKRTPRYPEELELLVSQQS |
| | Protein with signal | PCGRATSSSRVWWDSSFLGGVVHLEAGEEVVVRVLDERLV |
| | sequence | RLRDGTRSYFGAFMVSGGGSGGGSGHHHHHH |
| 85 | ProC1193/ | atggaaaccgacacactgctgctgtgggtgctgcttttgtgggtgccaggatccacaggcCA |
| | ProC_mLm- | AGGACAAAGTGGTTCCCGACACCCGTGCAGGCATGACCC |
| | 16_1490_LIGHT_ | ACATATAATCTGTTATAAGTTTGGTGGAGGCTCCTCCGGC |
| | 10GS His | GGGTCTATCAGTTCCGGTCTCCTCAGCGGTCGCAGTGATA |
| | DNA with coding | ATATCGGCGGTTCTCAGGAACGCAGGTCACACGAGGTAA |
| | sequence for signal | ATCCAGCCGCCCATTTGACTGGCGCTAATAGTTCTCTTAC |
| | peptide | GGGTAGCGGTGGCCCTCTTCTGTGGGAAACGCAGTTGGG |
| | | CTTGGCTTTCCTTAGAGGTCTCAGCTATCACGACGGCGCC |
| | | CTCGTCGTTACAAAGGCCGGGTACTATTATATTTATTCTA |
| | | AGGTACAGCTTGGCGGGGTGGGGTGCCCGCTGGGTCTTG |
| | | CATCTACGATCACTCATGGTCTCTACAAAAGAACACCAA |
| | | GGTATCCAGAAGAACTTGAGCTCCTCGTTAGCCAACAGT |
| | | CCCCCTGCGGACGGGCTACTTCCTCTAGCCGGGTATGGTG |
| | | GGATAGCAGTTTCCTTGGGGGGGTCGTGCATCTCGAGGCT |
| | | GGAGAGGAGGTAGTAGTCAGGGTCTTGGATGAGAGATTG |
| | | GTAAGGCTTCGAGACGGTACAAGATCATACTTTGGGGCC |
| | | TTCATGGTGAGCGGGGGTGGCAGCGGCGGTGGCAGCGGG |
| | | CATCATCACCATCACCATTGA |
| 86 | ProC1188/ | METDTLLLWVLLLWVPGSTGQERRSHEVNPAAHLTGANSS |
| | ProC_LIGHT-10GS- | LTGSGGPLLWETQLGLAFLRGLSYHDGALVVTKAGYYYIYS |
| | Strep | KVQLGGVGCPLGLASTITHGLYKRTPRYPEELELLVSQQSPC |
| | Protein with signal | GRATSSSRVWWDSSFLGGVVHLEAGEEVVVRVLDERLVRL |
| | sequence | RDGTRSYFGAFMVSGGGSGGGSGSAWSHPQFEK |
| 87 | ProC1188/ | atggaaaccgacacactgctgctgtgggtgctgcttttgtgggtgccaggatccacaggcCA |
| | ProC_LIGHT-10GS- | GGAACGCAGGTCCCATGAAGTAAACCCGGCGGCCCATCT |
| | Strep | TACGGGTGCAAATTCAAGCTTGACAGGCAGCGGAGGGCC |
| | DNA with coding | GCTCCTGTGGGAGACGCAACTTGGGCTTGCGTTTCTCCGG |
| | sequence for signal | GGCCTGTCATATCACGACGGTGCTCTTGTGGTAACGAAA |
| | peptide | GCAGGGTACTATTATATATATAGCAAGGTTCAGTTGGGTG |
| | | GGGTTGGATGCCCGCTTGGACTCGCGTCCACTATTACTCA |
| | | CGGGTTGTATAAGAGGACACCGCGATACCCAGAGGAGCT |
| | | GGAACTCCTTGTTTCACAGCAGTCTCCTTGCGGGAGAGCC |
| | | ACATCTTCATCCAGGGTCTGGTGGGACTCTTCTTTCTTGG |
| | | GCGGCGTTGTTCATCTGGAAGCGGGGGAAGAGGTTGTAG |
| | | TGAGGGTACTCGACGAGAGGCTGGTGAGGCTGCGAGACG |
| | | GGACCCGCTCATATTTCGGAGCCTTCATGGTATCAGGAGG |
| | | GGGCTCCGGTGGGGGGTCAGGAAGCGCATGGTCACACCC |
| | | TCAGTTCGAGAAATGA |
| 88 | ProC1192/ | METDTLLLWVLLLWVPGSTGQGQSGSRHPCRHDPHIICYKF |
| | ProC_mLm16_1490_ | GGGSSGGSISSGLLSGRSDNIGGSQERRSHEVNPAAHLTGAN |
| | LIGHT-10GS-Strep | SSLTGSGGPLLWETQLGLAFLRGLSYHDGALVVTKAGYYYI |
| | Protein with signal | YSKVQLGGVGCPLGLASTITHGLYKRTPRYPEELELLVSQQS |
| | sequence | PCGRATSSSRVWWDSSFLGGVVHLEAGEEVVVRVLDERLV |
| | | RLRDGTRSYFGAFMVSGGGSGGGSGSAWSHPQFEK |
| 89 | ProC1192/ | atggaaaccgacacactgctgctgtgggtgctgcttttgtgggtgccaggatccacaggcCA |
| | ProC_mLm16_1490_ | AGGACAGAGTGGAAGTCGACACCCTTGTAGGCACGACCC |
| | LIGHT-10GS-Strep | ACATATAATCTGCTATAAATTTGGTGGGGGTTCCAGCGGT |
| | DNA with coding | GGAAGTATCAGCTCCGGCCTTCTTAGTGGCAGGAGTGAC |
| | sequence for signal | AATATTGGCGGATCTCAAGAGCGACGCAGTCACGAGGTA |
| | peptide | AATCCTGCAGCACACTTGACAGGTGCTAACAGCTCACTTA |
| | | CCGGAAGTGGGGGGCCGCTGTTGTGGGAGACGCAGCTCG |
| | | GGTTGGCCTTTCTTCGAGGGCTCTCTTATCACGACGGTGC |
| | | ATTGGTGGTGACCAAAGCAGGGTATTACTACATATACTCT |
| | | AAGGTGCAACTGGGTGGAGTAGGTTGTCCATTGGGGCTC |
| | | GCCTCTACAATAACTCATGGTCTGTATAAGCGAACCCCGC |
| | | GGTATCCCGAAGAGCTTGAACTCCTCGTCAGCCAGCAAT |
| | | CTCCCTGTGGTCGGGCAACATCATCTAGCAGAGTGTGGTG |
| | | GGACTCCTCTTTTTTGGGTGGTGTAGTACATCTGGAAGCG |
| | | GGCGAAGAAGTGGTCGTCCGAGTGCTTGACGAACGACTC |
| | | GTTAGACTGCGAGACGGAACTCGGAGCTACTTTGGAGCT |
| | | TTTATGGTTAGCGGTGGTGGTTCAGGAGGTGGGAGTGGG |
| | | AGTGCATGGTCACACCCGCAATTCGAGAAATGA |
| 90 | ProC1491/ | METDTLLLWVLLLWVPGSTGQERRSHEVNPAAHLTGANSS |
| | ProC_LIGHT- | LTGSGGPLLWETQLGLAFLRGLSYHDGALVVTKAGYYYIYS |
| | 1204DNI-HSA-His | KVQLGGVGCPLGLASTITHGLYKRTPRYPEELELLVSQQSPC |
| | Protein with signal | GRATSSSRVWWDSSFLGGVVHLEAGEEVVVRVLDERLVRL |
| | sequence | RDGTRSYFGAFMVSSAGGGSSGGSLSGRSDNIGSDAHKSEV |
| | | AHRFKDLGEENFKALVLIAFAQYLQQCPFEDHVKLVNEVTE |
| | | FAKTCVADESAENCDKSLHTLFGDKLCTVATLRETYGEMA |
| | | DCCAKQEPERNECFLQHKDDNPNLPRLVRPEVDVMCTAFH |
| | | DNEETFLKKYLYEIARRHPYFYAPELLFFAKRYKAAFTECC |
| | | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLSHHHHHH |
| 91 | ProC1491/ | atggaaaccgacacactgctgctgtgggtgctgcttttgtgggtgccaggatccacaggcCA |
| | ProC_LIGHT- | GGAGAGGAGATCACATGAAGTCAACCCGGCCGCCCATCT |
| | 1204DNI-HSA-His | TACTGGTGCCAATAGTAGTCTTACAGGTTCTGGCGGACCC |
| | DNA with coding | TTGCTTTGGGAGACCCAGCTGGGACTGGCTTTCCTTAGAG |
| | sequence for signal | GCCTGAGTTATCACGACGGGGCGTTGGTCGTTACTAAAG |
| | peptide | CCGGGTATTACTATATATACTCTAAGGTTCAGCTCGGCGG |
| | | TGTAGGATGCCCACTGGGTCTTGCGTCTACAATTACACAT |
| | | GGTCTGTATAAGAGGACTCCCAGATATCCAGAAGAACTG |
| | | GAATTGCTTGTTTCCCAGCAATCTCCGTGCGGACGCGCAA |
| | | CGAGCTCCAGTAGGGTGTGGTGGGACTCCAGCTTCCTGG |
| | | GTGGCGTTGTCCATTTGGAGGCGGGAGAGGAAGTAGTGG |
| | | TGCGGGTCCTTGATGAACGCCTTGTACGACTTCGGGACGG |
| | | TACAAGAAGCTACTTTGGGGCTTTCATGGTATCCAGCGCC |
| | | GGGGGCGGAAGCAGCGGCGGCAGCTTAAGCGGCAGAAG |
| | | CGACAACATCGGCAGCGATGCACATAAAAGTGAGGTGGC |
| | | CCACCGCTTTAAAGACCTCGGCGAGGAGAACTTCAAGGC |
| | | ACTCGTACTTATTGCCTTTGCACAATACCTGCAGCAATGT |
| | | CCGTTTGAAGATCATGTCAAGCTCGTAAATGAAGTGACA |
| | | GAGTTCGCTAAGACCTGTGTAGCTGACGAAAGTGCCGAA |
| | | AACTGCGACAAATCATTGCATACATTGTTCGGCGACAAG |
| | | CTTTGTACAGTTGCCACGCTCCGCGAGACATACGGCGAA |
| | | ATGGCCGATTGTTGTGCGAAACAGGAACCCGAAAGAAAT |
| | | GAGTGCTTCTTGCAACACAAAGACGATAATCCCAATCTTC |
| | | CGCGACTGGTGAGACCCGAAGTAGATGTAATGTGCACAG |
| | | CTTTTCATGACAATGAGGAAACCTTTCTTAAAAAATATCT |
| | | GTACGAAATCGCCCGCCGACACCCGTATTTCTATGCCCCA |
| | | GAGCTGTTGTTTTTCGCAAAGCGCTACAAGGCAGCGTTTA |
| | | CCGAATGCTGCCAGGCCGCTGATAAAGCCGCTTGTCTGCT |
| | | GCCTAAGCTTGATGAGCTTCGCGACGAGGGTAAGGCTTC |
| | | CAGCGCTAAACAACGCCTGAAGTGTGCATCTTTGCAAAA |
| | | ATTTGGTGAACGAGCCTTCAAGGCTTGGGCTGTTGCCAGG |
| | | CTGAGTCAACGGTTCCCGAAGGCGGAGTTTGCGGAGGTT |
| | | TCCAAGTTGGTAACGGACCTGACAAAGGTACATACCGAA |
| | | TGCTGCCATGGCGACCTGTTGGAGTGCGCCGATGATCGA |
| | | GCGGATCTTGCCAAGTATATTTGCGAAAACCAAGACTCC |
| | | ATTTCCAGTAAACTTAAGGAGTGTTGTGAGAAACCTCTCC |
| | | TTGAAAAGAGCCATTGTATAGCGGAGGTCGAAAACGATG |
| | | AAATGCCCGCTGATCTGCCGTCACTCGCTGCTGACTTCGT |
| | | TGAATCCAAGGATGTATGTAAAAACTATGCCGAAGCTAA |
| | | GGACGTCTTTCTTGGAATGTTCTTGTATGAATACGCCCGA |
| | | CGGCATCCTGACTATTCAGTTGTGCTCCTTCTTCGATTGG |
| | | CTAAAACTTATGAGACGACACTTGAAAAGTGTTGTGCCG |
| | | CCGCCGACCCACATGAGTGCTACGCTAAGGTTTTCGACG |
| | | AATTTAAGCCCTTGGTTGAAGAACCGCAGAATCTCATTAA |
| | | GCAAAATTGTGAGCTCTTTGAACAACTCGGTGAATACAA |
| | | ATTTCAGAACGCGCTTCTGGTTAGATATACGAAGAAAGT |
| | | GCCACAGGTGAGCACCCCTACATTGGTTGAGGTCAGTAG |
| | | GAACCTCGGCAAGGTGGGATCTAAGTGCTGCAAACACCC |
| | | GGAGGCAAAGAGAATGCCTTGCGCTGAGGATTATCTCTC |
| | | AGTGGTTCTGAATCAGCTTTGCGTTCTTCACGAAAAGACT |
| | | CCCGTCAGCGACCGAGTCACGAAGTGCTGTACCGAGAGC |
| | | CTTGTTAATAGACGACCTTGCTTCTCCGCGCTTGAGGTGG |
| | | ATGAGACCTACGTCCCAAAGGAATTCAACGCCGAGACGT |
| | | TCACTTTCCATGCCGATATTTGCACCTTGTCAGAAAAGGA |
| | | GCGACAGATAAAAAAGCAAACTGCTTTGGTGGAGCTGGT |
| | | TAAGCATAAGCCTAAAGCGACGAAGGAACAGCTCAAAGC |
| | | CGTAATGGATGATTTCGCTGCCTTCGTGGAGAAATGTTGT |
| | | AAGGCCGATGACAAGGAAACCTGCTTCGCTGAAGAGGGT |
| | | AAAAAGCTCGTCGCCGCTAGTCAGGCGGCACTTGGTTTG |
| | | AGCCACCATCACCACCACCACtga |
| 92 | ProC1492/ | METDTLLLWVLLLWVPGSTGQGQSGSRHPCRHDPHIICYKF |
| | ProC_mLm16- | GGGSSGGSISSGLLSGRCDNIGGSQERRSHEVNPAAHLTGAN |
| | LIGHT-1204DNI- | SSLTGSGGPLLWETQLGLAFLRGLSYHDGALVVTKAGYYYI |
| | HSA-His (1490DNI | YSKVQLGGVGCPLGLASTITHGLYKRTPRYPEELELLVSQQS |
| | has S to C mutation) | PCGRATSSSRVWWDSSFLGGVVHLEAGEEVVVRVLDERLV |
| | Protein with signal | RLRDGTRSYFGAFMVSSAGGGSSGGSLSGRSDNIGSDAHKS |
| | sequence | EVAHRFKDLGEENFKALVLIAFAQYLQQCPFEDHVKLVNEV |
| | | TEFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSHHHHHH |
| 93 | ProC1492/ | atggaaaccgacacactgctgctgtgggtgctgcttttgtgggtgccaggatccacaggcCA |
| | ProC_mLm16- | GGGCCAGTCTGGATCCAGACACCCATGCAGACACGACCC |
| | LIGHT-1204DNI- | CCACATCATCTGTTACAAATTCGGCGGAGGCAGCTCCGG |
| | HSA-His (1490DNI | CGGCAGCATCTCCAGCGGACTGCTGAGCGGCAGAtGCGA |
| | has S to C mutation) | TAACATCGGCGGAAGCCAGGAGAGGAGATCACATGAAGT |
| | DNA with coding | CAACCCGGCCGCCCATCTTACTGGTGCCAATAGTAGTCTT |
| | sequence for signal | ACAGGTTCTGGCGGACCCTTGCTTTGGGAGACCCAGCTG |
| | peptide | GGACTGGCTTTCCTTAGAGGCCTGAGTTATCACGACGGG |
| | | GCGTTGGTCGTTACTAAAGCCGGGTATTACTATATATACT |
| | | CTAAGGTTCAGCTCGGCGGTGTAGGATGCCCACTGGGTCT |
| | | TGCGTCTACAATTACACATGGTCTGTATAAGAGGACTCCC |
| | | AGATATCCAGAAGAACTGGAATTGCTTGTTTCCCAGCAAT |
| | | CTCCGTGCGGACGCGCAACGAGCTCCAGTAGGGTGTGGT |
| | | GGGACTCCAGCTTCCTGGGTGGCGTTGTCCATTTGGAGGC |
| | | GGGAGAGGAAGTAGTGGTGCGGGTCCTTGATGAACGCCT |
| | | TGTACGACTTCGGGACGGTACAAGAAGCTACTTTGGGGC |
| | | TTTCATGGTATCCAGCGCCGGGGGCGGAAGCAGCGGCGG |
| | | CAGCTTAAGCGGCAGAAGCGACAACATCGGCAGCGATGC |
| | | ACATAAAAGTGAGGTGGCCCACCGCTTTAAAGACCTCGG |
| | | CGAGGAGAACTTCAAGGCACTCGTACTTATTGCCTTTGCA |
| | | CAATACCTGCAGCAATGTCCGTTTGAAGATCATGTCAAGC |
| | | TCGTAAATGAAGTGACAGAGTTCGCTAAGACCTGTGTAG |
| | | CTGACGAAAGTGCCGAAAACTGCGACAAATCATTGCATA |
| | | CATTGTTCGGCGACAAGCTTTGTACAGTTGCCACGCTCCG |
| | | CGAGACATACGGCGAAATGGCCGATTGTTGTGCGAAACA |
| | | GGAACCCGAAAGAAATGAGTGCTTCTTGCAACACAAAGA |
| | | CGATAATCCCAATCTTCCGCGACTGGTGAGACCCGAAGT |
| | | AGATGTAATGTGCACAGCTTTTCATGACAATGAGGAAAC |
| | | CTTTCTTAAAAAATATCTGTACGAAATCGCCCGCCGACAC |
| | | CCGTATTTCTATGCCCCAGAGCTGTTGTTTTTCGCAAAGC |
| | | GCTACAAGGCAGCGTTTACCGAATGCTGCCAGGCCGCTG |
| | | ATAAAGCCGCTTGTCTGCTGCCTAAGCTTGATGAGCTTCG |
| | | CGACGAGGGTAAGGCTTCCAGCGCTAAACAACGCCTGAA |
| | | GTGTGCATCTTTGCAAAAATTTGGTGAACGAGCCTTCAAG |
| | | GCTTGGGCTGTTGCCAGGCTGAGTCAACGGTTCCCGAAG |
| | | GCGGAGTTTGCGGAGGTTTCCAAGTTGGTAACGGACCTG |
| | | ACAAAGGTACATACCGAATGtTGCCATGGCGACCTGTTGG |
| | | AGTGCGCCGATGATCGAGCGGATCTTGCCAAGTATATTTG |
| | | CGAAAACCAAGACTCCATTTCCAGTAAACTTAAGGAGTG |
| | | TTGTGAGAAACCTCTCCTTGAAAAGAGCCATTGTATAGCG |
| | | GAGGTCGAAAACGATGAAATGCCCGCTGATCTGCCGTCA |
| | | CTCGCTGCTGACTTCGTTGAATCCAAGGATGTATGTAAAA |
| | | ACTATGCCGAAGCTAAGGACGTCTTTCTTGGAATGTTCTT |
| | | GTATGAATACGCCCGACGGCATCCTGACTATTCAGTTGTG |
| | | CTCCTTCTTCGATTGGCTAAAACTTATGAGACGACACTTG |
| | | AAAAGTGTTGTGCCGCGGCGGACCCACATGAGTGCTACG |
| | | CTAAGGTTTTCGACGAATTTAAGCCCTTGGTTGAAGAACC |
| | | GCAGAATCTCATTAAGCAAAATTGTGAGCTCTTTGAACA |
| | | ACTCGGTGAATACAAATTTCAGAACGCGCTTCTGGTTAGA |
| | | TATACGAAGAAAGTGCCACAGGTGAGCACCCCTACATTG |
| | | GTTGAGGTCAGTAGGAACCTCGGCAAGGTGGGATCTAAG |
| | | TGCTGCAAACACCCGGAGGCAAAGAGAATGCCTTGCGCT |
| | | GAGGATTATCTCTCAGTGGTTCTGAATCAGCTTTGCGTTC |
| | | TTCACGAAAAGACTCCCGTCAGCGACCGAGTCACGAAGT |
| | | GCTGTACCGAGAGCCTTGTTAATAGACGACCTTGCTTCTC |
| | | CGCGCTTGAGGTGGATGAGACCTACGTCCCAAAGGAATT |
| | | CAACGCCGAGACGTTCACTTTCCATGCCGATATTTGCACC |
| | | TTGTCAGAAAAGGAGCGACAGATAAAAAAGCAAACTGCT |
| | | TTGGTGGAGCTGGTTAAGCATAAGCCTAAAGCGACGAAG |
| | | GAACAGCTCAAAGCCGTAATGGATGATTTCGCTGCCTTCG |
| | | TGGAGAAATGTTGTAAGGCCGATGACAAGGAAACCTGCT |
| | | TCGCTGAAGAGGGTAAAAAGCTCGTCGCCGCTAGTCAGG |
| | | CGGCACTTGGTTTGAGCCACCATCACCACCACCACtga |
| 94 | ProC1655/ | METDTLLLWVLLLWVPGSTGQGQSGSRHPCRHDPHIICYKF |
| | ProC_mLm16- | GGGSSGGSISSGLLSGRSDNIGGSQERRSHEVNPAAHLTGAN |
| | LIGHT-1204DNI- | SSLTGSGGPLLWETQLGLAFLRGLSYHDGALVVTKAGYYYI |
| | HSA-His 1490 | YSKVQLGGVGCPLGLASTITHGLYKRTPRYPEELELLVSQQS |
| | Protein with signal | PCGRATSSSRVWWDSSFLGGVVHLEAGEEVVVRVLDERLV |
| | sequence | RLRDGTRSYFGAFMVSSAGGGSSGGSLSGRSDNIGSDAHKS |
| | | EVAHRFKDLGEENFKALVLIAFAQYLQQCPFEDHVKLVNEV |
| | | TEFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSHHHHHH |
| 95 | ProC1655/ | atggaaaccgacacactgctgctgtgggtgctgcttttgtgggtgccaggatccacaggcCA |
| | ProC_mLm16- | GGGCCAGTCTGGATCCAGACACCCATGCAGACACGACCC |
| | LIGHT-1204DNI- | CCACATCATCTGTTACAAATTCGGCGGAGGCAGCTCCGG |
| | HSA-His 1490 | CGGCAGCATCTCCAGCGGACTGCTGAGCGGCAGAAGCGA |
| | DNA with coding | TAACATCGGCGGAAGCCAGGAGAGGAGATCACATGAAGT |
| | sequence for signal | CAACCCGGCCGCCCATCTTACTGGTGCCAATAGTAGTCTT |
| | peptide | ACAGGTTCTGGCGGACCCTTGCTTTGGGAGACCCAGCTG |
| | | GGACTGGCTTTCCTTAGAGGCCTGAGTTATCACGACGGG |
| | | GCGTTGGTCGTTACTAAAGCCGGGTATTACTATATATACT |
| | | CTAAGGTTCAGCTCGGCGGTGTAGGATGCCCACTGGGTCT |
| | | TGCGTCTACAATTACACATGGTCTGTATAAGAGGACTCCC |
| | | AGATATCCAGAAGAACTGGAATTGCTTGTTTCCCAGCAAT |
| | | CTCCGTGCGGACGCGCAACGAGCTCCAGTAGGGTGTGGT |
| | | GGGACTCCAGCTTCCTGGGTGGCGTTGTCCATTTGGAGGC |
| | | GGGAGAGGAAGTAGTGGTGCGGGTCCTTGATGAACGCCT |
| | | TGTACGACTTCGGGACGGTACAAGAAGCTACTTTGGGGC |
| | | TTTCATGGTATCCAGCGCCGGGGGCGGAAGCAGCGGCGG |
| | | CAGCTTAAGCGGCAGAAGCGAtAACATCGGCAGCGATGC |
| | | ACATAAAAGTGAGGTGGCCCACCGCTTTAAAGACCTCGG |
| | | CGAGGAGAACTTCAAGGCACTCGTACTTATTGCCTTTGCA |
| | | CAATACCTGCAGCAATGTCCGTTTGAAGATCATGTCAAGC |
| | | TCGTAAATGAAGTGACAGAGTTCGCTAAGACCTGTGTAG |
| | | CTGACGAAAGTGCCGAAAACTGCGACAAATCATTGCATA |
| | | CATTGTTCGGCGACAAGCTTTGTACAGTTGCCACGCTCCG |
| | | CGAGACATACGGCGAAATGGCCGATTGTTGTGCGAAACA |
| | | GGAACCCGAAAGAAATGAGTGCTTCTTGCAACACAAAGA |
| | | CGATAATCCCAATCTTCCGCGACTGGTGAGACCCGAAGT |
| | | AGATGTAATGTGCACAGCTTTTCATGACAATGAGGAAAC |
| | | CTTTCTTAAAAAATATCTGTACGAAATCGCCCGCCGACAC |
| | | CCGTATTTCTATGCCCCAGAGCTGTTGTTTTTCGCAAAGC |
| | | GCTACAAGGCAGCGTTTACCGAATGCTGCCAGGCCGCTG |
| | | ATAAAGCCGCTTGTCTGCTGCCTAAGCTTGATGAGCTTCG |
| | | CGACGAGGGTAAGGCTTCCAGCGCTAAACAACGCCTGAA |
| | | GTGTGCATCTTTGCAAAAATTTGGTGAACGAGCCTTCAAG |
| | | GCTTGGGCTGTTGCCAGGCTGAGTCAACGGTTCCCGAAG |
| | | GCGGAGTTTGCGGAGGTTTCCAAGTTGGTAACGGACCTG |
| | | ACAAAGGTACATACCGAATGUTGCCATGGCGACCTGTTGG |
| | | AGTGCGCCGATGATCGAGCGGATCTTGCCAAGTATATTTG |
| | | CGAAAACCAAGACTCCATTTCCAGTAAACTTAAGGAGTG |
| | | TTGTGAGAAACCTCTCCTTGAAAAGAGCCATTGTATAGCG |
| | | GAGGTCGAAAACGATGAAATGCCCGCTGATCTGCCGTCA |
| | | CTCGCTGCTGACTTCGTTGAATCCAAGGATGTATGTAAAA |
| | | ACTATGCCGAAGCTAAGGACGTCTTTCTTGGAATGTTCTT |
| | | GTATGAATACGCCCGACGGCATCCTGACTATTCAGTTGTG |
| | | CTCCTTCTTCGATTGGCTAAAACTTATGAGACGACACTTG |
| | | AAAAGTGTTGTGCCGCGGCGGACCCACATGAGTGCTACG |
| | | CTAAGGTTTTCGACGAATTTAAGCCCTTGGTTGAAGAACC |
| | | GCAGAATCTCATTAAGCAAAATTGTGAGCTCTTTGAACA |
| | | ACTCGGTGAATACAAATTTCAGAACGCGCTTCTGGTTAGA |
| | | TATACGAAGAAAGTGCCACAGGTGAGCACCCCTACATTG |
| | | GTTGAGGTCAGTAGGAACCTCGGCAAGGTGGGATCTAAG |
| | | TGCTGCAAACACCCGGAGGCAAAGAGAATGCCTTGCGCT |
| | | GAGGATTATCTCTCAGTGGTTCTGAATCAGCTTTGCGTTC |
| | | TTCACGAAAAGACTCCCGTCAGCGACCGAGTCACGAAGT |
| | | GCTGTACCGAGAGCCTTGTTAATAGACGACCTTGCTTCTC |
| | | CGCGCTTGAGGTGGATGAGACCTACGTCCCAAAGGAATT |
| | | CAACGCCGAGACGTTCACTTTCCATGCCGATATTTGCACC |
| | | TTGTCAGAAAAGGAGCGACAGATAAAAAAGCAAACTGCT |
| | | TTGGTGGAGCTGGTTAAGCATAAGCCTAAAGCGACGAAG |
| | | GAACAGCTCAAAGCCGTAATGGATGATTTCGCTGCCTTCG |
| | | TGGAGAAATGTTGTAAGGCCGATGACAAGGAAACCTGCT |
| | | TCGCTGAAGAGGGTAAAAAGCTCGTCGCCGCTAGTCAGG |
| | | CGGCACTTGGTTTGAGCCACCATCACCACCACCACtga |
| 96 | ProC1499/ | METDTLLLWVLLLWVPGSTGDAHKSEVAHRFKDLGEENFK |
| | ProC_HSA-21GS- | ALVLIAFAQYLQQCPFEDHVKLVNEVTEFAKTCVADESAEN |
| | 1204DNI-LIGHT | CDKSLHTLFGDKLCTVATLRETYGEMADCCAKQEPERNEC |
| | Protein with signal | FLQHKDDNPNLPRLVRPEVDVMCTAFHDNEETFLKKYLYEI |
| | sequence | ARRHPYFYAPELLFFAKRYKAAFTECCQAADKAACLLPKL |
| | | DELRDEGKASSAKQRLKCASLQKFGERAFKAWAVARLSQR |
| | | FPKAEFAEVSKLVTDLTKVHTECCHGDLLECADDRADLAK |
| | | YICENQDSISSKLKECCEKPLLEKSHCIAEVENDEMPADLPSL |
| | | AADFVESKDVCKNYAEAKDVFLGMFLYEYARRHPDYSVV |
| | | LLLRLAKTYETTLEKCCAAADPHECYAKVFDEFKPLVEEPQ |
| | | NLIKQNCELFEQLGEYKFQNALLVRYTKKVPQVSTPTLVEV |
| | | SRNLGKVGSKCCKHPEAKRMPCAEDYLSVVLNQLCVLHEK |
| | | TPVSDRVTKCCTESLVNRRPCFSALEVDETYVPKEFNAETFT |
| | | FHADICTLSEKERQIKKQTALVELVKHKPKATKEQLKAVMD |
| | | DFAAFVEKCCKADDKETCFAEEGKKLVAASQAALGLSSAG |
| | | GGSSGGSLSGRSDNIGSQERRSHEVNPAAHLTGANSSLTGSG |
| | | GPLLWETQLGLAFLRGLSYHDGALVVTKAGYYYIYSKVQL |
| | | GGVGCPLGLASTITHGLYKRTPRYPEELELLVSQQSPCGRAT |
| | | SSSRVWWDSSFLGGVVHLEAGEEVVVRVLDERLVRLRDGT |
| | | RSYFGAFMV |
| 97 | ProC1499/ | atggaaaccgacacactgctgctgtgggtgctgcttttgtgggtgccaggatccacaggcGA |
| | ProC_HSA-21GS- | TGCACATAAAAGTGAGGTGGCCCACCGCTTTAAAGACCT |
| | 1204DNI-LIGHT | CGGCGAGGAGAACTTCAAGGCACTCGTACTTATTGCCTTT |
| | DNA with coding | GCACAATACCTGCAGCAATGTCCGTTTGAAGATCATGTCA |
| | sequence for signal | AGCTCGTAAATGAAGTGACAGAGTTCGCTAAGACCTGTG |
| | peptide | TAGCTGACGAAAGTGCCGAAAACTGCGACAAATCATTGC |
| | | ATACATTGTTCGGCGACAAGCTTTGTACAGTTGCCACGCT |
| | | CCGCGAGACATACGGCGAAATGGCCGATTGTTGTGCGAA |
| | | ACAGGAACCCGAAAGAAATGAGTGCTTCTTGCAACACAA |
| | | AGACGATAATCCCAATCTTCCGCGACTGGTGAGACCCGA |
| | | AGTAGATGTAATGTGCACAGCTTTTCATGACAATGAGGA |
| | | AACCTTTCTTAAAAAATATCTGTACGAAATCGCCCGCCGA |
| | | CACCCGTATTTCTATGCCCCAGAGCTGTTGTTTTTCGCAA |
| | | AGCGCTACAAGGCAGCGTTTACaGAATGCTGtCAGGCCGC |
| | | TGATAAAGCCGCTTGTCTGCTGCCTAAGCTTGATGAGCTT |
| | | CGCGACGAGGGTAAGGCTTCCAGCGCTAAACAACGCCTG |
| | | AAGTGTGCATCTTTGCAAAAATTTGGTGAACGAGCCTTCA |
| | | AGGCTTGGGCTGTTGCCAGGCTGAGTCAACGGTTCCCGA |
| | | AGGCGGAGTTTGCGGAGGTTTCCAAGTTGGTAACGGACC |
| | | TGACAAAGGTACATACCGAATGCTGCCATGGCGACCTGT |
| | | TGGAGTGCGCCGATGATCGAGCGGATCTTGCCAAGTATA |
| | | TTTGCGAAAACCAAGACTCCATTTCCAGTAAACTTAAGG |
| | | AGTGTTGTGAGAAACCTCTCCTTGAAAAGAGCCATTGTAT |
| | | AGCGGAGGTCGAAAACGATGAAATGCCCGCTGATCTGCC |
| | | GTCACTCGCTGCTGACTTCGTTGAATCCAAGGATGTATGT |
| | | AAAAACTATGCCGAAGCTAAGGACGTCTTTCTTGGAATG |
| | | TTCTTGTATGAATACGCCCGACGGCATCCTGACTATTCAG |
| | | TTGTGCTCCTTCTTCGATTGGCTAAAACTTATGAGACGAC |
| | | ACTTGAAAAGTGTTGTGCCGCCGCCGACCCACATGAGTG |
| | | CTACGCTAAGGTTTTCGACGAATTTAAGCCCTTGGTTGAA |
| | | GAACCGCAGAATCTCATTAAGCAAAATTGTGAGCTCTTTG |
| | | AACAACTCGGTGAATACAAATTTCAGAACGCGCTTCTGG |
| | | TTAGATATACGAAGAAAGTGCCACAGGTGAGCACCCCTA |
| | | CATTGGTTGAGGTCAGTAGGAACCTCGGCAAGGTGGGAT |
| | | CTAAGTGCTGCAAACACCCGGAGGCAAAGAGAATGCCTT |
| | | GCGCTGAGGATTATCTCTCAGTGGTTCTGAATCAGCTTTG |
| | | CGTTCTTCACGAAAAGACTCCCGTCAGCGACCGAGTCAC |
| | | GAAGTGCTGTACCGAGAGCCTTGTTAATAGACGACCTTG |
| | | CTTCTCCGCGCTTGAGGTGGATGAGACCTACGTCCCAAAG |
| | | GAATTCAACGCCGAGACGTTCACTTTCCATGCCGATATTT |
| | | GCACCTTGTCAGAAAAGGAGCGACAGATAAAAAAGCAA |
| | | ACTGCTTTGGTGGAGCTGGTTAAGCATAAGCCTAAAGCG |
| | | ACGAAGGAACAGCTCAAAGCCGTAATGGATGATTTCGCT |
| | | GCCTTCGTGGAGAAATGTTGTAAGGCCGATGACAAGGAA |
| | | ACCTGCTTCGCTGAAGAGGGTAAAAAGCTCGTCGCCGCT |
| | | AGTCAGGCGGCACTTGGTTTGTCCAGCGCCGGGGGCGGA |
| | | AGCAGCGGCGGCAGCTTAAGCGGCAGAAGCGACAACAT |
| | | CGGCAGCCAGGAGAGACGGAGCCACGAGGTGAACCCCG |
| | | CTGCCCACCTGACCGGCGCCAACAGCAGTCTGACCGGCA |
| | | GCGGCGGCCCTCTGCTGTGGGAGACACAGCTGGGCCTGG |
| | | CCTTTCTGAGAGGCCTGTCCTACCACGACGGCGCCCTGGT |
| | | CGTGACCAAGGCCGGCTACTACTACATCTACAGCAAGGT |
| | | GCAGCTGGGAGGCGTGGGCTGCCCTCTGGGCCTGGCATC |
| | | TACCATCACGCACGGCCTGTACAAGCGGACCCCTAGATA |
| | | TCCTGAGGAACTGGAACTCCTGGTGTCCCAGCAGAGCCC |
| | | TTGCGGCAGAGCTACAAGCAGCTCAAGAGTGTGGTGGGA |
| | | CAGCAGCTTCCTGGGCGGAGTGGTCCACCTGGAAGCTGG |
| | | AGAAGAAGTGGTGGTCCGGGTGCTGGACGAGAGACTCGT |
| | | GAGGCTGAGAGACGGAACAAGAAGCTACTTCGGCGCTTT |
| | | TATGGTGtga |
| 98 | ProC1495/ | METDTLLLWVLLLWVPGSTGQERRSHEVNPAAHLTGANSS |
| | ProC_LIGHT- | LTGSGGPLLWETQLGLAFLRGLSYHDGALVVTKAGYYYIYS |
| | 1204DNI-HSA-cmyc | KVQLGGVGCPLGLASTITHGLYKRTPRYPEELELLVSQQSPC |
| | Protein with signal | GRATSSSRVWWDSSFLGGVVHLEAGEEVVVRVLDERLVRL |
| | sequence | RDGTRSYFGAFMVSSAGGGSSGGSLSGRSDNIGSDAHKSEV |
| | | AHRFKDLGEENFKALVLIAFAQYLQQCPFEDHVKLVNEVTE |
| | | FAKTCVADESAENCDKSLHTLFGDKLCTVATLRETYGEMA |
| | | DCCAKQEPERNECFLQHKDDNPNLPRLVRPEVDVMCTAFH |
| | | DNEETFLKKYLYEIARRHPYFYAPELLFFAKRYKAAFTECC |
| | | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLSGGEQKLISEEDL |
| 99 | ProC1495/ | atggaaaccgacacactgctgctgtgggtgctgcttttgtgggtgccaggatccacaggcCA |
| | ProC_LIGHT- | GGAGAGGAGATCACATGAAGTCAACCCGGCCGCCCATCT |
| | 1204DNI-HSA-cmyc | TACTGGTGCCAATAGTAGTCTTACAGGTTCTGGCGGACCC |
| | DNA with coding | TTGCTTTGGGAGACCCAGCTGGGACTGGCTTTCCTTAGAG |
| | sequence for signal | GCCTGAGTTATCACGACGGGGCGTTGGTCGTTACTAAAG |
| | peptide | CCGGGTATTACTATATATACTCTAAGGTTCAGCTCGGCGG |
| | | TGTAGGATGCCCACTGGGTCTTGCGTCTACAATTACACAT |
| | | GGTCTGTATAAGAGGACTCCCAGATATCCAGAAGAACTG |
| | | GAATTGCTTGTTTCCCAGCAATCTCCGTGCGGACGCGCAA |
| | | CGAGCTCCAGTAGGGTGTGGTGGGACTCCAGCTTCCTGG |
| | | GTGGCGTTGTCCATTTGGAGGCGGGAGAGGAAGTAGTGG |
| | | TGCGGGTCCTTGATGAACGCCTTGTACGACTTCGGGACGG |
| | | TACAAGAAGCTACTTTGGGGCTTTCATGGTATCCAGCGCC |
| | | GGGGGCGGAAGCAGCGGCGGCAGCTTAAGCGGCAGAAG |
| | | CGACAACATCGGCAGCGATGCACATAAAAGTGAGGTGGC |
| | | CCACCGCTTTAAAGACCTCGGCGAGGAGAACTTCAAGGC |
| | | ACTCGTACTTATTGCCTTTGCACAATACCTGCAGCAATGT |
| | | CCGTTTGAAGATCATGTCAAGCTCGTAAATGAAGTGACA |
| | | GAGTTCGCTAAGACCTGTGTAGCTGACGAAAGTGCCGAA |
| | | AACTGCGACAAATCATTGCATACATTGTTCGGCGACAAG |
| | | CTTTGTACAGTTGCCACGCTCCGCGAGACATACGGCGAA |
| | | ATGGCCGATTGTTGTGCGAAACAGGAACCCGAAAGAAAT |
| | | GAGTGCTTCTTGCAACACAAAGACGATAATCCCAATCTTC |
| | | CGCGACTGGTGAGACCCGAAGTAGATGTAATGTGCACAG |
| | | CTTTTCATGACAATGAGGAAACCTTTCTTAAAAAATATCT |
| | | GTACGAAATCGCCCGCCGACACCCGTATTTCTATGCCCCA |
| | | GAGCTGTTGTTTTTCGCAAAGCGCTACAAGGCAGCGTTTA |
| | | CCGAATGCTGCCAGGCCGCTGATAAAGCCGCTTGTCTGCT |
| | | GCCTAAGCTTGATGAGCTTCGCGACGAGGGTAAGGCTTC |
| | | CAGCGCTAAACAACGCCTGAAGTGTGCATCTTTGCAAAA |
| | | ATTTGGTGAACGAGCCTTCAAGGCTTGGGCTGTTGCCAGG |
| | | CTGAGTCAACGGTTCCCGAAGGCGGAGTTTGCGGAGGTT |
| | | TCCAAGTTGGTAACGGACCTGACAAAGGTACATACCGAA |
| | | TGCTGCCATGGCGACCTGTTGGAGTGCGCCGATGATCGA |
| | | GCGGATCTTGCCAAGTATATTTGCGAAAACCAAGACTCC |
| | | ATTTCCAGTAAACTTAAGGAGTGTTGTGAGAAACCTCTCC |
| | | TTGAAAAGAGCCATTGTATAGCGGAGGTCGAAAACGATG |
| | | AAATGCCCGCTGATCTGCCGTCACTCGCTGCTGACTTCGT |
| | | TGAATCCAAGGATGTATGTAAAAACTATGCCGAAGCTAA |
| | | GGACGTCTTTCTTGGAATGTTCTTGTATGAATACGCCCGA |
| | | CGGCATCCTGACTATTCAGTTGTGCTCCTTCTTCGATTGG |
| | | CTAAAACTTATGAGACGACACTTGAAAAGTGTTGTGCCG |
| | | CCGCCGACCCACATGAGTGCTACGCTAAGGTTTTCGACG |
| | | AATTTAAGCCCTTGGTTGAAGAACCGCAGAATCTCATTAA |
| | | GCAAAATTGTGAGCTCTTTGAACAACTCGGTGAATACAA |
| | | ATTTCAGAACGCGCTTCTGGTTAGATATACGAAGAAAGT |
| | | GCCACAGGTGAGCACCCCTACATTGGTTGAGGTCAGTAG |
| | | GAACCTCGGCAAGGTGGGATCTAAGTGCTGCAAACACCC |
| | | GGAGGCAAAGAGAATGCCTTGCGCTGAGGATTATCTCTC |
| | | AGTGGTTCTGAATCAGCTTTGCGTTCTTCACGAAAAGACT |
| | | CCCGTCAGCGACCGAGTCACGAAGTGCTGTACCGAGAGC |
| | | CTTGTTAATAGACGACCTTGCTTCTCCGCGCTTGAGGTGG |
| | | ATGAGACCTACGTCCCAAAGGAATTCAACGCCGAGACGT |
| | | TCACTTTCCATGCCGATATTTGCACCTTGTCAGAAAAGGA |
| | | GCGACAGATAAAAAAGCAAACTGCTTTGGTGGAGCTGGT |
| | | TAAGCATAAGCCTAAAGCGACGAAGGAACAGCTCAAAGC |
| | | CGTAATGGATGATTTCGCTGCCTTCGTGGAGAAATGTTGT |
| | | AAGGCCGATGACAAGGAAACCTGCTTCGCTGAAGAGGGT |
| | | AAAAAGCTCGTCGCCGCTAGTCAGGCGGCACTTGGTTTG |
| | | AGTGGTGGAGAACAAAAACTCATTTCCGAGGAAGATCTTt |
| | | ga |
| 100 | ProC1496/ | METDTLLLWVLLLWVPGSTGQGQSGSRHPCRHDPHIICYKF |
| | ProC_mLm16- | GGGSSGGSISSGLLSGRSDNIGGSQERRSHEVNPAAHLTGAN |
| | LIGHT-1204DNI- | SSLTGSGGPLLWETQLGLAFLRGLSYHDGALVVTKAGYYYI |
| | HSA-cMyc | YSKVQLGGVGCPLGLASTITHGLYKRTPRYPEELELLVSQQS |
| | Protein with signal | PCGRATSSSRVWWDSSFLGGVVHLEAGEEVVVRVLDERLV |
| | sequence | RLRDGTRSYFGAFMVSSAGGGSSGGSLSGRSDNIGSDAHKS |
| | | EVAHRFKDLGEENFKALVLIAFAQYLQQCPFEDHVKLVNEV |
| | | TEFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 101 | ProC1496/ | atggaaaccgacacactgctgctgtgggtgctgcttttgtgggtgccaggatccacaggcCA |
| | ProC_mLm16- | GGGACAGTCCGGATCTAGGCATCCATGTCGGCATGACCC |
| | LIGHT-1204DNI- | ACATATCATATGCTACAAATTCGGTGGAGGATCTTCTGGA |
| | HSA-cMyc | GGCTCAATTTCATCAGGACTCCTGTCCGGGCGCTCCGACA |
| | DNA with coding | ATATTGGTGGCTCCCAGGAGAGGAGATCACATGAAGTCA |
| | sequence for signal | ACCCGGCCGCCCATCTTACTGGTGCCAATAGTAGTCTTAC |
| | peptide | AGGTTCTGGCGGACCCTTGCTTTGGGAGACCCAGCTGGG |
| | | ACTGGCTTTCCTTAGAGGCCTGAGTTATCACGACGGGGCG |
| | | TTGGTCGTTACTAAAGCCGGGTATTACTATATATACTCTA |
| | | AGGTTCAGCTCGGCGGTGTAGGATGCCCACTGGGTCTTGC |
| | | GTCTACAATTACACATGGTCTGTATAAGAGGACTCCCAG |
| | | ATATCCAGAAGAACTGGAATTGCTTGTTTCCCAGCAATCT |
| | | CCGTGCGGACGCGCAACGAGCTCCAGTAGGGTGTGGTGG |
| | | GACTCCAGCTTCCTGGGTGGCGTTGTCCATTTGGAGGCGG |
| | | GAGAGGAAGTAGTGGTGCGGGTCCTTGATGAACGCCTTG |
| | | TACGACTTCGGGACGGTACAAGAAGCTACTTTGGGGCTTT |
| | | CATGGTATCCAGCGCCGGGGGCGGAAGCAGCGGCGGCA |
| | | GCTTAAGCGGCAGAAGCGACAACATCGGCAGCGATGCAC |
| | | ATAAAAGTGAGGTGGCCCACCGCTTTAAAGACCTCGGCG |
| | | AGGAGAACTTCAAGGCACTCGTACTTATTGCCTTTGCACA |
| | | ATACCTGCAGCAATGTCCGTTTGAAGATCATGTCAAGCTC |
| | | GTAAATGAAGTGACAGAGTTCGCTAAGACCTGTGTAGCT |
| | | GACGAAAGTGCCGAAAACTGCGACAAATCATTGCATACA |
| | | TTGTTCGGCGACAAGCTTTGTACAGTTGCCACGCTCCGCG |
| | | AGACATACGGCGAAATGGCCGATTGTTGTGCGAAACAGG |
| | | AACCCGAAAGAAATGAGTGCTTCTTGCAACACAAAGACG |
| | | ATAATCCCAATCTTCCGCGACTGGTGAGACCCGAAGTAG |
| | | ATGTAATGTGCACAGCTTTTCATGACAATGAGGAAACCTT |
| | | TCTTAAAAAATATCTGTACGAAATCGCCCGCCGACACCC |
| | | GTATTTCTATGCCCCAGAGCTGTTGTTTTTCGCAAAGCGC |
| | | TACAAGGCAGCGTTTACCGAATGCTGCCAGGCCGCTGAT |
| | | AAAGCCGCTTGTCTGCTGCCTAAGCTTGATGAGCTTCGCG |
| | | ACGAGGGTAAGGCTTCCAGCGCTAAACAACGCCTGAAGT |
| | | GTGCATCTTTGCAAAAATTTGGTGAACGAGCCTTCAAGGC |
| | | TTGGGCTGTTGCCAGGCTGAGTCAACGGTTCCCGAAGGC |
| | | GGAGTTTGCGGAGGTTTCCAAGTTGGTAACGGACCTGAC |
| | | AAAGGTACATACCGAATGCTGtCATGGCGACCTGTTGGAG |
| | | TGCGCCGATGATCGAGCGGATCTTGCCAAGTATATTTGCG |
| | | AAAACCAAGACTCCATTTCCAGTAAACTTAAGGAGTGTT |
| | | GTGAGAAACCTCTCCTTGAAAAGAGCCATTGTATAGCGG |
| | | AGGTCGAAAACGATGAAATGCCCGCTGATCTGCCGTCAC |
| | | TCGCTGCTGACTTCGTTGAATCCAAGGATGTATGTAAAAA |
| | | CTATGCCGAAGCTAAGGACGTCTTTCTTGGAATGTTCTTG |
| | | TATGAATACGCCCGACGGCATCCTGACTATTCAGTTGTGC |
| | | TCCTTCTTCGATTGGCTAAAACTTATGAGACGACACTTGA |
| | | AAAGTGTTGTGCCGCCGCCGACCCACATGAGTGCTACGC |
| | | TAAGGTTTTCGACGAATTTAAGCCCTTGGTTGAAGAACCG |
| | | CAGAATCTCATTAAGCAAAATTGTGAGCTCTTTGAACAAC |
| | | TCGGTGAATACAAATTTCAGAACGCGCTTCTGGTTAGATA |
| | | TACGAAGAAAGTGCCACAGGTGAGCACCCCTACATTGGT |
| | | TGAGGTCAGTAGGAACCTCGGCAAGGTGGGATCTAAGTG |
| | | CTGCAAACACCCGGAGGCAAAGAGAATGCCTTGCGCTGA |
| | | GGATTATCTCTCAGTGGTTCTGAATCAGCTTTGCGTTCTTC |
| | | ACGAAAAGACTCCCGTCAGCGACCGAGTCACGAAGTGCT |
| | | GTACCGAGAGCCTTGTTAATAGACGACCTTGCTTCTCCGC |
| | | GCTTGAGGTGGATGAGACCTACGTCCCAAAGGAATTCAA |
| | | CGCCGAGACGTTCACTTTCCATGCCGATATTTGCACCTTG |
| | | TCAGAAAAGGAGCGACAGATAAAAAAGCAAACTGCTTTG |
| | | GTGGAGCTGGTTAAGCATAAGCCTAAAGCGACGAAGGAA |
| | | CAGCTCAAAGCCGTAATGGATGATTTCGCTGCCTTCGTGG |
| | | AGAAATGTTGTAAGGCCGATGACAAGGAAACCTGCTTCG |
| | | CTGAAGAGGGTAAAAAGCTCGTCGCCGCTAGTCAGGCGG |
| | | CACTTGGTTTGAGTGGTGGAGAACAAAAACTCATTTCCG |
| | | AGGAAGATCTTtga |
| 102 | ProC1184/ | METDTLLLWVLLLWVPGSTGQERRSHEVNPAAHLTGANSS |
| | ProC_LIGHT_ | LTGSGGPLLWETQLGLAFLRGLSYHDGALVVTKAGYYYIYS |
| | 21linker_HSA-cMyc | KVQLGGVGCPLGLASTITHGLYKRTPRYPEELELLVSQQSPC |
| | Protein with signal | GRATSSSRVWWDSSFLGGVVHLEAGEEVVVRVLDERLVRL |
| | sequence | RDGTRSYFGAFMVSSAGGGSSGGSSAGGGSSGGSDAHKSE |
| | | VAHRFKDLGEENFKALVLIAFAQYLQQCPFEDHVKLVNEVT |
| | | EFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 103 | ProC1184/ | atggaaaccgacacactgctgctgtgggtgctgcttttgtgggtgccaggatccacaggcCA |
| | ProC_LIGHT_ | GGAGAGGCGCTCTCATGAAGTAAACCCCGCCGCGCACCT |
| | 21linker_HSA-cMyc | TACCGGTGCGAATTCCAGCCTTACGGGCTCCGGGGGCCC |
| | DNA with coding | TCTTCTGTGGGAAACCCAACTCGGGCTGGCGTTTCTCAGG |
| | sequence for signal | GGTCTCAGTTACCACGACGGCGCGCTTGTCGTTACTAAAG |
| | peptide | CGGGCTACTACTATATCTACTCCAAGGTACAGCTCGGTGG |
| | | TGTAGGATGTCCGCTTGGGCTTGCCTCTACCATCACGCAC |
| | | GGTCTCTATAAAAGAACCCCAAGATACCCCGAAGAGTTG |
| | | GAACTGCTTGTTTCTCAACAGTCCCCTTGTGGTCGGGCAA |
| | | CCAGTTCATCTCGGGTGTGGTGGGATAGTAGCTTTCTCGG |
| | | AGGAGTAGTCCACCTGGAAGCTGGGGAAGAGGTAGTCGT |
| | | CCGGGTACTCGATGAACGGCTCGTGCGCCTCCGAGATGG |
| | | GACCCGGTCTTACTTTGGGGCTTTCATGGTTAGCTCTGCG |
| | | GGGGGAGGAAGCTCCGGGGGTAGTTCCGCCGGCGGCGGA |
| | | TCTAGTGGCGGTTCTGATGCTCATAAAAGTGAGGTCGCGC |
| | | ACAGATTCAAAGACCTCGGCGAGGAAAACTTTAAAGCCC |
| | | TCGTATTGATAGCTTTCGCCCAGTACCTCCAGCAGTGCCC |
| | | ATTCGAGGATCACGTGAAACTCGTGAATGAAGTAACGGA |
| | | ATTTGCCAAAACGTGCGTGGCCGACGAGAGTGCCGAAAA |
| | | TTGCGACAAGTCACTTCACACCCTTTTTGGTGACAAATTG |
| | | TGTACTGTGGCGACGCTTAGGGAAACATACGGAGAAATG |
| | | GCCGATTGCTGTGCCAAACAAGAGCCTGAAAGAAACGAA |
| | | TGCTTTCTTCAGCACAAAGACGATAACCCTAACTTGCCGA |
| | | GACTGGTGAGACCTGAAGTAGACGTCATGTGTACCGCGT |
| | | TCCATGACAACGAAGAGACTTTCTTGAAAAAATACCTTTA |
| | | TGAAATAGCACGGAGGCACCCTTATTTTTACGCTCCAGAG |
| | | CTCCTCTTCTTTGCTAAACGGTACAAAGCGGCGTTTACCG |
| | | AATGTTGCCAAGCTGCGGATAAAGCTGCATGTCTTCTCCC |
| | | GAAGTTGGATGAGCTGAGAGATGAGGGTAAAGCGTCTTC |
| | | CGCTAAACAACGACTTAAATGCGCTTCTCTGCAGAAATTT |
| | | GGCGAACGAGCGTTCAAAGCCTGGGCCGTTGCGCGCCTT |
| | | TCACAGCGGTTTCCGAAAGCCGAGTTCGCCGAGGTTTCA |
| | | AAGCTGGTTACGGACTTGACAAAGGTGCATACAGAATGC |
| | | TGCCACGGAGACCTGCTGGAGTGCGCCGATGATCGCGCT |
| | | GATTTGGCTAAATATATTTGCGAAAATCAGGACAGCATC |
| | | AGCTCCAAGTTGAAGGAGTGTTGTGAAAAACCGCTGCTG |
| | | GAGAAGTCACACTGCATAGCCGAGGTGGAAAACGACGA |
| | | GATGCCGGCAGACCTCCCCAGCCTTGCCGCGGATTTCGTC |
| | | GAATCAAAGGATGTATGCAAAAATTACGCGGAAGCAAAA |
| | | GATGTATTTCTGGGAATGTTCCTGTACGAGTACGCTAGGC |
| | | GACATCCCGACTACAGCGTTGTTCTGCTCTTGAGACTTGC |
| | | GAAGACGTACGAGACTACGCTCGAGAAGTGCTGTGCAGC |
| | | TGCGGACCCCCATGAGTGTTATGCTAAAGTCTTCGACGAA |
| | | TTTAAGCCTTTGGTGGAAGAGCCACAGAACCTGATCAAA |
| | | CAAAATTGTGAACTGTTCGAACAATTGGGGGAGTATAAA |
| | | TTTCAGAACGCTCTCCTTGTTCGGTATACAAAAAAAGTAC |
| | | CGCAGGTAAGTACGCCGACACTCGTGGAAGTAAGCAGGA |
| | | ACCTCGGAAAGGTTGGATCAAAATGCTGCAAGCATCCGG |
| | | AGGCTAAGCGCATGCCATGCGCTGAGGATTATCTTTCAGT |
| | | GGTCCTGAACCAATTGTGCGTGTTGCACGAGAAAACTCC |
| | | AGTAAGCGATAGAGTTACCAAATGTTGTACAGAGAGCCT |
| | | GGTTAATCGACGACCTTGCTTCAGCGCCTTGGAAGTCGAT |
| | | GAGACGTATGTGCCGAAGGAATTTAACGCCGAAACTTTC |
| | | ACTTTTCATGCAGATATCTGTACATTGAGCGAAAAGGAA |
| | | AGGCAAATTAAAAAACAAACTGCGCTTGTGGAATTGGTC |
| | | AAGCACAAGCCTAAAGCCACAAAGGAACAGCTCAAAGC |
| | | GGTAATGGACGATTTTGCGGCGTTCGTAGAGAAGTGTTG |
| | | CAAGGCGGACGATAAAGAAACGTGCTTTGCTGAGGAGGG |
| | | AAAAAAGCTTGTTGCTGCCAGTCAAGCGGCGCTTGGCTT |
| | | GAGTGGTGGAGAACAAAAACTCATTTCCGAGGAAGATCT |
| | | TTGA |
| 104 | ProC1486/ | METDTLLLWVLLLWVPGSTGQERRSHEVNPAAHLTGANSS |
| | ProC_mhLIGHT_ | LTGSGGPLLWETQLGLAFLRGLSYHDGALVVTKTGYYYIYS |
| | 1204DNI HSA-cMyc | KVQLGGVGCPLGLAGTITHGLYKRTPRYPEELELLVSQQSP |
| | Protein with signal | CGRATSSSRVWWDSSFLGGVVHLEAGEEVVVRVLGKRLVR |
| | sequence | LRDGTRSYFGAFMVSSAGGGSSGGSLSGRSDNIGSDAHKSE |
| | | VAHRFKDLGEENFKALVLIAFAQYLQQCPFEDHVKLVNEVT |
| | | EFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 105 | ProC1486/ | atggaaaccgacacactgctgctgtgggtgctgcttttgtgggtgccaggatccacaggcCA |
| | ProC_mhLIGHT_ | AGAACGCCGATCACACGAAGTGAACCCTGCTGCTCACTT |
| | 1204DNI HSA-cMyc | GACTGGAGCTAACTCCAGCCTTACGGGGAGCGGGGGTCC |
| | DNA with coding | TTTGCTCTGGGAAACGCAGCTGGGTCTCGCGTTTCTTAGA |
| | sequence for signal | GGGCTGAGTTATCACGATGGCGCCTTGGTCGTCACGAAG |
| | peptide | ACTGGGTACTACTATATTTATAGTAAGGTACAGTTGGGGG |
| | | GAGTGGGGTGCCCTCTCGGCCTGGCTGGGACGATCACGC |
| | | ATGGGCTCTACAAGCGCACGCCTAGGTATCCGGAGGAGT |
| | | TGGAACTGTTGGTCTCACAGCAGTCCCCGTGCGGGCGAG |
| | | CGACTTCCTCCAGTAGAGTGTGGTGGGACTCTAGCTTTCT |
| | | CGGGGGAGTGGTTCATCTTGAGGCAGGAGAGGAAGTGGT |
| | | GGTACGGGTTTTGGGCAAGAGATTGGTGAGACTGCGGGA |
| | | TGGTACAAGATCTTATTTTGGTGCTTTCATGGTCTCCAGC |
| | | GCCGGGGGCGGAAGCAGCGGCGGCAGCTTAAGCGGCAG |
| | | AAGCGACAACATCGGCAGCGATGCTCATAAAAGTGAGGT |
| | | CGCGCACAGATTCAAAGACCTCGGCGAGGAAAACTTTAA |
| | | AGCCCTCGTATTGATAGCTTTCGCCCAGTACCTCCAGCAG |
| | | TGCCCATTCGAGGATCACGTGAAACTCGTGAATGAAGTA |
| | | ACGGAATTTGCCAAAACGTGCGTGGCCGACGAGAGTGCC |
| | | GAAAATTGCGACAAGTCACTTCACACCCTTTTTGGTGACA |
| | | AATTGTGTACTGTGGCGACGCTTAGGGAAACATACGGAG |
| | | AAATGGCCGATTGCTGTGCCAAACAAGAGCCTGAAAGAA |
| | | ACGAATGCTTTCTTCAGCACAAAGACGATAACCCTAACTT |
| | | GCCGAGACTGGTGAGACCTGAAGTAGACGTCATGTGTAC |
| | | CGCGTTCCATGACAACGAAGAGACTTTCTTGAAAAAATA |
| | | CCTTTATGAAATAGCACGGAGGCACCCTTATTTTTACGCT |
| | | CCAGAGCTCCTCTTCTTTGCTAAACGGTACAAAGCGGCGT |
| | | TTACCGAATGTTGCCAAGCTGCGGATAAAGCTGCATGTCT |
| | | TCTCCCGAAGTTGGATGAGCTGAGAGATGAGGGTAAAGC |
| | | GTCTTCCGCTAAACAACGACTTAAATGCGCTTCTCTGCAG |
| | | AAATTTGGCGAACGAGCGTTCAAAGCCTGGGCCGTTGCG |
| | | CGCCTTTCACAGCGGTTTCCGAAAGCCGAGTTCGCCGAG |
| | | GTTTCAAAGCTGGTTACGGACTTGACAAAGGTGCATACA |
| | | GAATGCTGCCACGGAGACCTGCTGGAGTGCGCCGATGAT |
| | | CGCGCTGATTTGGCTAAATATATTTGCGAAAATCAGGAC |
| | | AGCATCAGCTCCAAGTTGAAGGAGTGTTGTGAAAAACCG |
| | | CTGCTGGAGAAGTCACACTGCATAGCCGAGGTGGAAAAC |
| | | GACGAGATGCCGGCAGACCTCCCCAGCCTTGCCGCGGAT |
| | | TTCGTCGAATCAAAGGATGTATGCAAAAATTACGCGGAA |
| | | GCAAAAGATGTATTTCTGGGAATGTTCCTGTACGAGTACG |
| | | CTAGGCGACATCCCGACTACAGCGTTGTTCTGCTCTTGAG |
| | | ACTTGCGAAGACGTACGAGACTACGCTCGAGAAGTGCTG |
| | | TGCAGCTGCGGACCCCCATGAGTGTTATGCTAAAGTCTTC |
| | | GACGAATTTAAGCCTTTGGTGGAAGAGCCACAGAACCTG |
| | | ATCAAACAAAATTGTGAACTGTTCGAACAATTGGGGGAG |
| | | TATAAATTTCAGAACGCTCTCCTTGTTCGGTATACAAAAA |
| | | AAGTACCGCAGGTAAGTACGCCGACACTCGTGGAAGTAA |
| | | GCAGGAACCTCGGAAAGGTTGGATCAAAATGCTGCAAGC |
| | | ATCCGGAGGCTAAGCGCATGCCATGCGCTGAGGATTATC |
| | | TTTCAGTGGTCCTGAACCAATTGTGCGTGTTGCACGAGAA |
| | | AACTCCAGTAAGCGATAGAGTTACCAAATGTTGTACAGA |
| | | GAGCCTGGTTAATCGACGACCTTGCTTCAGCGCCTTGGAA |
| | | GTCGATGAGACGTATGTGCCGAAGGAATTTAACGCCGAA |
| | | ACTTTCACTTTTCATGCAGATATCTGTACATTGAGCGAAA |
| | | AGGAAAGGCAAATTAAAAAACAAACTGCGCTTGTGGAAT |
| | | TGGTCAAGCACAAGCCTAAAGCCACAAAGGAACAGCTCA |
| | | AAGCGGTAATGGACGATTTTGCGGCGTTCGTAGAGAAGT |
| | | GTTGCAAGGCGGACGATAAAGAAACGTGCTTTGCTGAGG |
| | | AGGGAAAAAAGCTTGTTGCTGCCAGTCAAGCGGCGCTTG |
| | | GCTTGAGTGGTGGAGAACAAAAACTCATTTCCGAGGAAG |
| | | ATCTTTGA |
| 106 | ProC1483/ | METDTLLLWVLLLWVPGSTGQERRSHEVNPAAHLTGANSS |
| | ProC_mhLIGHT_ | LTGSGGPLLWETQLGLAFLRGLSYHDGALVVTKTGYYYIYS |
| | 21linker_HSA-cMyc | KVQLGGVGCPLGLAGTITHGLYKRTPRYPEELELLVSQQSP |
| | Protein with signal | CGRATSSSRVWWDSSFLGGVVHLEAGEEVVVRVLGKRLVR |
| | sequence | LRDGTRSYFGAFMVSSAGGGSSGGSSAGGGSSGGSDAHKSE |
| | | VAHRFKDLGEENFKALVLIAFAQYLQQCPFEDHVKLVNEVT |
| | | EFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 107 | ProC1483/ | atggaaaccgacacactgctgctgtgggtgctgcttttgtgggtgccaggatccacaggcCA |
| | ProC_mhLIGHT_ | AGAACGCCGATCACACGAAGTGAACCCTGCTGCTCACTT |
| | 21linker_HSA-cMyc | GACTGGAGCTAACTCCAGCCTTACGGGGAGCGGGGGTCC |
| | DNA with coding | TTTGCTCTGGGAAACGCAGCTGGGTCTCGCGTTTCTTAGA |
| | sequence for signal | GGGCTGAGTTATCACGATGGCGCCTTGGTCGTCACGAAG |
| | peptide | ACTGGGTACTACTATATTTATAGTAAGGTACAGTTGGGGG |
| | | GAGTGGGGTGCCCTCTCGGCCTGGCTGGGACGATCACGC |
| | | ATGGGCTCTACAAGCGCACGCCTAGGTATCCGGAGGAGT |
| | | TGGAACTGTTGGTCTCACAGCAGTCCCCGTGCGGGCGAG |
| | | CGACTTCCTCCAGTAGAGTGTGGTGGGACTCTAGCTTTCT |
| | | CGGGGGAGTGGTTCATCTTGAGGCAGGAGAGGAAGTGGT |
| | | GGTACGGGTTTTGGGCAAGAGATTGGTGAGACTGCGGGA |
| | | TGGTACAAGATCTTATTTTGGTGCTTTCATGGTCAGCTCT |
| | | GCGGGGGGAGGAAGCTCCGGGGGTAGTTCCGCCGGCGGC |
| | | GGATCTAGTGGCGGTTCTGATGCTCATAAAAGTGAGGTC |
| | | GCGCACAGATTCAAAGACCTCGGCGAGGAAAACTTTAAA |
| | | GCCCTCGTATTGATAGCTTTCGCCCAGTACCTCCAGCAGT |
| | | GCCCATTCGAGGATCACGTGAAACTCGTGAATGAAGTAA |
| | | CGGAATTTGCCAAAACGTGCGTGGCCGACGAGAGTGCCG |
| | | AAAATTGCGACAAGTCACTTCACACCCTTTTTGGTGACAA |
| | | ATTGTGTACTGTGGCGACGCTTAGGGAAACATACGGAGA |
| | | AATGGCCGATTGCTGTGCCAAACAAGAGCCTGAAAGAAA |
| | | CGAATGCTTTCTTCAGCACAAAGACGATAACCCTAACTTG |
| | | CCGAGACTGGTGAGACCTGAAGTAGACGTCATGTGTACC |
| | | GCGTTCCATGACAACGAAGAGACTTTCTTGAAAAAATAC |
| | | CTTTATGAAATAGCACGGAGGCACCCTTATTTTTACGCTC |
| | | CAGAGCTCCTCTTCTTTGCTAAACGGTACAAAGCGGCGTT |
| | | TACCGAATGTTGCCAAGCTGCGGATAAAGCTGCATGTCTT |
| | | CTCCCGAAGTTGGATGAGCTGAGAGATGAGGGTAAAGCG |
| | | TCTTCCGCTAAACAACGACTTAAATGCGCTTCTCTGCAGA |
| | | AATTTGGCGAACGAGCGTTCAAAGCCTGGGCCGTTGCGC |
| | | GCCTTTCACAGCGGTTTCCGAAAGCCGAGTTCGCCGAGGT |
| | | TTCAAAGCTGGTTACGGACTTGACAAAGGTGCATACAGA |
| | | ATGCTGCCACGGAGACCTGCTGGAGTGCGCCGATGATCG |
| | | CGCTGATTTGGCTAAATATATTTGCGAAAATCAGGACAG |
| | | CATCAGCTCCAAGTTGAAGGAGTGTTGTGAAAAACCGCT |
| | | GCTGGAGAAGTCACACTGCATAGCCGAGGTGGAAAACGA |
| | | CGAGATGCCGGCAGACCTCCCCAGCCTTGCCGCGGATTT |
| | | CGTCGAATCAAAGGATGTATGCAAAAATTACGCGGAAGC |
| | | AAAAGATGTATTTCTGGGAATGTTCCTGTACGAGTACGCT |
| | | AGGCGACATCCCGACTACAGCGTTGTTCTGCTCTTGAGAC |
| | | TTGCGAAGACGTACGAGACTACGCTCGAGAAGTGCTGTG |
| | | CAGCTGCGGACCCCCATGAGTGTTATGCTAAAGTCTTCGA |
| | | CGAATTTAAGCCTTTGGTGGAAGAGCCACAGAACCTGAT |
| | | CAAACAAAATTGTGAACTGTTCGAACAATTGGGGGAGTA |
| | | TAAATTTCAGAACGCTCTCCTTGTTCGGTATACAAAAAAA |
| | | GTACCGCAGGTAAGTACGCCGACACTCGTGGAAGTAAGC |
| | | AGGAACCTCGGAAAGGTTGGATCAAAATGCTGCAAGCAT |
| | | CCGGAGGCTAAGCGCATGCCATGCGCTGAGGATTATCTTT |
| | | CAGTGGTCCTGAACCAATTGTGCGTGTTGCACGAGAAAA |
| | | CTCCAGTAAGCGATAGAGTTACCAAATGTTGTACAGAGA |
| | | GCCTGGTTAATCGACGACCTTGCTTCAGCGCCTTGGAAGT |
| | | CGATGAGACGTATGTGCCGAAGGAATTTAACGCCGAAAC |
| | | TTTCACTTTTCATGCAGATATCTGTACATTGAGCGAAAAG |
| | | GAAAGGCAAATTAAAAAACAAACTGCGCTTGTGGAATTG |
| | | GTCAAGCACAAGCCTAAAGCCACAAAGGAACAGCTCAA |
| | | AGCGGTAATGGACGATTTTGCGGCGTTCGTAGAGAAGTG |
| | | TTGCAAGGCGGACGATAAAGAAACGTGCTTTGCTGAGGA |
| | | GGGAAAAAAGCTTGTTGCTGCCAGTCAAGCGGCGCTTGG |
| | | CTTGAGTGGTGGAGAACAAAAACTCATTTCCGAGGAAGA |
| | | TCTTTGA |
| 108 | ProC1485/ | METDTLLLWVLLLWVPGSTGQGQSGSRHPCRHDPHIICYKF |
| | ProC_mLm- | GGGSSGGSISSGLLSGRSDNIGGSQERRSHEVNPAAHLTGAN |
| | 16_mhLIGHT_ | SSLTGSGGPLLWETQLGLAFLRGLSYHDGALVVTKTGYYYI |
| | 21linker_HSA-cMyc | YSKVQLGGVGCPLGLAGTITHGLYKRTPRYPEELELLVSQQ |
| | Protein with signal | SPCGRATSSSRVWWDSSFLGGVVHLEAGEEVVVRVLGKRL |
| | sequence | VRLRDGTRSYFGAFMVSSAGGGSSGGSSAGGGSSGGSDAH |
| | | KSEVAHRFKDLGEENFKALVLIAFAQYLQQCPFEDHVKLVN |
| | | EVTEFAKTCVADESAENCDKSLHTLFGDKLCTVATLRETYG |
| | | EMADCCAKQEPERNECFLQHKDDNPNLPRLVRPEVDVMCT |
| | | AFHDNEETFLKKYLYEIARRHPYFYAPELLFFAKRYKAAFT |
| | | ECCQAADKAACLLPKLDELRDEGKASSAKQRLKCASLQKF |
| | | GERAFKAWAVARLSQRFPKAEFAEVSKLVTDLTKVHTECC |
| | | HGDLLECADDRADLAKYICENQDSISSKLKECCEKPLLEKSH |
| | | CIAEVENDEMPADLPSLAADFVESKDVCKNYAEAKDVFLG |
| | | MFLYEYARRHPDYSVVLLLRLAKTYETTLEKCCAAADPHE |
| | | CYAKVFDEFKPLVEEPQNLIKQNCELFEQLGEYKFQNALLV |
| | | RYTKKVPQVSTPTLVEVSRNLGKVGSKCCKHPEAKRMPCA |
| | | EDYLSVVLNQLCVLHEKTPVSDRVTKCCTESLVNRRPCFSA |
| | | LEVDETYVPKEFNAETFTFHADICTLSEKERQIKKQTALVEL |
| | | VKHKPKATKEQLKAVMDDFAAFVEKCCKADDKETCFAEE |
| | | GKKLVAASQAALGLSGGEQKLISEEDL |
| 109 | ProC1485/ | atggaaaccgacacactgctgctgtgggtgctgcttttgtgggtgccaggatccacaggcCA |
| | ProC_mLm- | GGGACAGTCCGGATCTAGGCATCCATGTCGGCATGACCC |
| | 16_mhLIGHT_ | ACATATCATATGCTACAAATTCGGTGGAGGATCTTCTGGA |
| | 21linker_HSA-cMyc | GGCTCAATTTCATCAGGACTCCTGTCCGGGCGCTCCGACA |
| | DNA with coding | ATATTGGTGGCTCCCAAGAACGCCGATCACACGAAGTGA |
| | sequence for signal | ACCCTGCTGCTCACTTGACTGGAGCTAACTCCAGCCTTAC |
| | peptide | GGGGAGCGGGGGTCCTTTGCTCTGGGAAACGCAGCTGGG |
| | | TCTCGCGTTTCTTAGAGGGCTGAGTTATCACGATGGCGCC |
| | | TTGGTCGTCACGAAGACTGGGTACTACTATATTTATAGTA |
| | | AGGTACAGTTGGGGGGAGTGGGGTGCCCTCTCGGCCTGG |
| | | CTGGGACGATCACGCATGGGCTCTACAAGCGCACGCCTA |
| | | GGTATCCGGAGGAGTTGGAACTGTTGGTCTCACAGCAGT |
| | | CCCCGTGCGGGCGAGCGACTTCCTCCAGTAGAGTGTGGT |
| | | GGGACTCTAGCTTTCTCGGGGGAGTGGTTCATCTTGAGGC |
| | | AGGAGAGGAAGTGGTGGTACGGGTTTTGGGCAAGAGATT |
| | | GGTGAGACTGCGGGATGGTACAAGATCTTATTTTGGTGCT |
| | | TTCATGGTCAGCTCTGCGGGGGGAGGAAGCTCCGGGGGT |
| | | AGTTCCGCCGGCGGCGGATCTAGTGGCGGTTCTGATGCTC |
| | | ATAAAAGTGAGGTCGCGCACAGATTCAAAGACCTCGGCG |
| | | AGGAAAACTTTAAAGCCCTCGTATTGATAGCTTTCGCCCA |
| | | GTACCTCCAGCAGTGCCCATTCGAGGATCACGTGAAACT |
| | | CGTGAATGAAGTAACGGAATTTGCCAAAACGTGCGTGGC |
| | | CGACGAGAGTGCCGAAAATTGCGACAAGTCACTTCACAC |
| | | CCTTTTTGGTGACAAATTGTGTACTGTGGCGACGCTTAGG |
| | | GAAACATACGGAGAAATGGCCGATTGCTGTGCCAAACAA |
| | | GAGCCTGAAAGAAACGAATGCTTTCTTCAGCACAAAGAC |
| | | GATAACCCTAACTTGCCGAGACTGGTGAGACCTGAAGTA |
| | | GACGTCATGTGTACCGCGTTCCATGACAACGAAGAGACT |
| | | TTCTTGAAAAAATACCTTTATGAAATAGCACGGAGGCAC |
| | | CCTTATTTTTACGCTCCAGAGCTCCTCTTCTTTGCTAAACG |
| | | GTACAAAGCGGCGTTTACCGAATGTTGCCAAGCTGCGGA |
| | | TAAAGCTGCATGTCTTCTCCCGAAGTTGGATGAGCTGAGA |
| | | GATGAGGGTAAAGCGTCTTCCGCTAAACAACGACTTAAA |
| | | TGCGCTTCTCTGCAGAAATTTGGCGAACGAGCGTTCAAA |
| | | GCCTGGGCCGTTGCGCGCCTTTCACAGCGGTTTCCGAAAG |
| | | CCGAGTTCGCCGAGGTTTCAAAGCTGGTTACGGACTTGAC |
| | | AAAGGTGCATACAGAATGCTGCCACGGAGACCTGCTGGA |
| | | GTGCGCCGATGATCGCGCTGATTTGGCTAAATATATTTGC |
| | | GAAAATCAGGACAGCATCAGCTCCAAGTTGAAGGAGTGT |
| | | TGTGAAAAACCGCTGCTGGAGAAGTCACACTGCATAGCC |
| | | GAGGTGGAAAACGACGAGATGCCGGCAGACCTCCCCAGC |
| | | CTTGCCGCGGATTTCGTCGAATCAAAGGATGTATGCAAA |
| | | AATTACGCGGAAGCAAAAGATGTATTTCTGGGAATGTTC |
| | | CTGTACGAGTACGCTAGGCGACATCCCGACTACAGCGTT |
| | | GTTCTGCTCTTGAGACTTGCGAAGACGTACGAGACTACGC |
| | | TCGAGAAGTGCTGTGCAGCTGCGGACCCCCATGAGTGTT |
| | | ATGCTAAAGTCTTCGACGAATTTAAGCCTTTGGTGGAAGA |
| | | GCCACAGAACCTGATCAAACAAAATTGTGAACTGTTCGA |
| | | ACAATTGGGGGAGTATAAATTTCAGAACGCTCTCCTTGTT |
| | | CGGTATACAAAAAAAGTACCGCAGGTAAGTACGCCGACA |
| | | CTCGTGGAAGTAAGCAGGAACCTCGGAAAGGTTGGATCA |
| | | AAATGCTGCAAGCATCCGGAGGCTAAGCGCATGCCATGC |
| | | GCTGAGGATTATCTTTCAGTGGTCCTGAACCAATTGTGCG |
| | | TGTTGCACGAGAAAACTCCAGTAAGCGATAGAGTTACCA |
| | | AATGTTGTACAGAGAGCCTGGTTAATCGACGACCTTGCTT |
| | | CAGCGCCTTGGAAGTCGATGAGACGTATGTGCCGAAGGA |
| | | ATTTAACGCCGAAACTTTCACTTTTCATGCAGATATCTGT |
| | | ACATTGAGCGAAAAGGAAAGGCAAATTAAAAAACAAAC |
| | | TGCGCTTGTGGAATTGGTCAAGCACAAGCCTAAAGCCAC |
| | | AAAGGAACAGCTCAAAGCGGTAATGGACGATTTTGCGGC |
| | | GTTCGTAGAGAAGTGTTGCAAGGCGGACGATAAAGAAAC |
| | | GTGCTTTGCTGAGGAGGGAAAAAAGCTTGTTGCTGCCAG |
| | | TCAAGCGGCGCTTGGCTTGAGTGGTGGAGAACAAAAACT |
| | | CATTTCCGAGGAAGATCTTTGA |
| 110 | ProC1487/ | METDTLLLWVLLLWVPGSTGQGQSGSRHPCRHDPHIICYKF |
| | ProC_mLm- | GGGSSGGSISSGLLSGRSDNIGGSQERRSHEVNPAAHLTGAN |
| | 16_mhLIGHT_ | SSLTGSGGPLLWETQLGLAFLRGLSYHDGALVVTKTGYYYI |
| | 1204DNI_HSA-cMyc | YSKVQLGGVGCPLGLAGTITHGLYKRTPRYPEELELLVSQQ |
| | Protein with signal | SPCGRATSSSRVWWDSSFLGGVVHLEAGEEVVVRVLGKRL |
| | sequence | VRLRDGTRSYFGAFMVSSAGGGSSGGSLSGRSDNIGSDAHK |
| | | SEVAHRFKDLGEENFKALVLIAFAQYLQQCPFEDHVKLVNE |
| | | VTEFAKTCVADESAENCDKSLHTLFGDKLCTVATLRETYGE |
| | | MADCCAKQEPERNECFLQHKDDNPNLPRLVRPEVDVMCTA |
| | | FHDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 111 | ProC1487/ | atggaaaccgacacactgctgctgtgggtgctgcttttgtgggtgccaggatccacaggcCA |
| | ProC_mLm- | GGGACAGTCCGGATCTAGGCATCCATGTCGGCATGACCC |
| | 16_mhLIGHT_ | ACATATCATATGCTACAAATTCGGTGGAGGATCTTCTGGA |
| | 1204DNI_HSA-cMyc | GGCTCAATTTCATCAGGACTCCTGTCCGGGCGCTCCGACA |
| | DNA with coding | ATATTGGTGGCTCCCAAGAACGCCGATCACACGAAGTGA |
| | sequence for signal | ACCCTGCTGCTCACTTGACTGGAGCTAACTCCAGCCTTAC |
| | peptide | GGGGAGCGGGGGTCCTTTGCTCTGGGAAACGCAGCTGGG |
| | | TCTCGCGTTTCTTAGAGGGCTGAGTTATCACGATGGCGCC |
| | | TTGGTCGTCACGAAGACTGGGTACTACTATATTTATAGTA |
| | | AGGTACAGTTGGGGGGAGTGGGGTGCCCTCTCGGCCTGG |
| | | CTGGGACGATCACGCATGGGCTCTACAAGCGCACGCCTA |
| | | GGTATCCGGAGGAGTTGGAACTGTTGGTCTCACAGCAGT |
| | | CCCCGTGCGGGCGAGCGACTTCCTCCAGTAGAGTGTGGT |
| | | GGGACTCTAGCTTTCTCGGGGGAGTGGTTCATCTTGAGGC |
| | | AGGAGAGGAAGTGGTGGTACGGGTTTTGGGCAAGAGATT |
| | | GGTGAGACTGCGGGATGGTACAAGATCTTATTTTGGTGCT |
| | | TTCATGGTCTCCAGCGCCGGGGGCGGAAGCAGCGGCGGC |
| | | AGCTTAAGCGGCAGAAGCGACAACATCGGCAGCGATGCT |
| | | CATAAAAGTGAGGTCGCGCACAGATTCAAAGACCTCGGC |
| | | GAGGAAAACTTTAAAGCCCTCGTATTGATAGCTTTCGCCC |
| | | AGTACCTCCAGCAGTGCCCATTCGAGGATCACGTGAAAC |
| | | TCGTGAATGAAGTAACGGAATTTGCCAAAACGTGCGTGG |
| | | CCGACGAGAGTGCCGAAAATTGCGACAAGTCACTTCACA |
| | | CCCTTTTTGGTGACAAATTGTGTACTGTGGCGACGCTTAG |
| | | GGAAACATACGGAGAAATGGCCGATTGCTGTGCCAAACA |
| | | AGAGCCTGAAAGAAACGAATGCTTTCTTCAGCACAAAGA |
| | | CGATAACCCTAACTTGCCGAGACTGGTGAGACCTGAAGT |
| | | AGACGTCATGTGTACCGCGTTCCATGACAACGAAGAGAC |
| | | TTTCTTGAAAAAATACCTTTATGAAATAGCACGGAGGCA |
| | | CCCTTATTTTTACGCTCCAGAGCTCCTCTTCTTTGCTAAAC |
| | | GGTACAAAGCGGCGTTTACCGAATGTTGCCAAGCTGCGG |
| | | ATAAAGCTGCATGTCTTCTCCCGAAGTTGGATGAGCTGAG |
| | | AGATGAGGGTAAAGCGTCTTCCGCTAAACAACGACTTAA |
| | | ATGCGCTTCTCTGCAGAAATTTGGCGAACGAGCGTTCAA |
| | | AGCCTGGGCCGTTGCGCGCCTTTCACAGCGGTTTCCGAAA |
| | | GCCGAGTTCGCCGAGGTTTCAAAGCTGGTTACGGACTTG |
| | | ACAAAGGTGCATACAGAATGCTGCCACGGAGACCTGCTG |
| | | GAGTGCGCCGATGATCGCGCTGATTTGGCTAAATATATTT |
| | | GCGAAAATCAGGACAGCATCAGCTCCAAGTTGAAGGAGT |
| | | GTTGTGAAAAACCGCTGCTGGAGAAGTCACACTGCATAG |
| | | CCGAGGTGGAAAACGACGAGATGCCGGCAGACCTCCCCA |
| | | GCCTTGCCGCGGATTTCGTCGAATCAAAGGATGTATGCA |
| | | AAAATTACGCGGAAGCAAAAGATGTATTTCTGGGAATGT |
| | | TCCTGTACGAGTACGCTAGGCGACATCCCGACTACAGCG |
| | | TTGTTCTGCTCTTGAGACTTGCGAAGACGTACGAGACTAC |
| | | GCTCGAGAAGTGCTGTGCAGCTGCGGACCCCCATGAGTG |
| | | TTATGCTAAAGTCTTCGACGAATTTAAGCCTTTGGTGGAA |
| | | GAGCCACAGAACCTGATCAAACAAAATTGTGAACTGTTC |
| | | GAACAATTGGGGGAGTATAAATTTCAGAACGCTCTCCTT |
| | | GTTCGGTATACAAAAAAAGTACCGCAGGTAAGTACGCCG |
| | | ACACTCGTGGAAGTAAGCAGGAACCTCGGAAAGGTTGGA |
| | | TCAAAATGCTGCAAGCATCCGGAGGCTAAGCGCATGCCA |
| | | TGCGCTGAGGATTATCTTTCAGTGGTCCTGAACCAATTGT |
| | | GCGTGTTGCACGAGAAAACTCCAGTAAGCGATAGAGTTA |
| | | CCAAATGTTGTACAGAGAGCCTGGTTAATCGACGACCTT |
| | | GCTTCAGCGCCTTGGAAGTCGATGAGACGTATGTGCCGA |
| | | AGGAATTTAACGCCGAAACTTTCACTTTTCATGCAGATAT |
| | | CTGTACATTGAGCGAAAAGGAAAGGCAAATTAAAAAACA |
| | | AACTGCGCTTGTGGAATTGGTCAAGCACAAGCCTAAAGC |
| | | CACAAAGGAACAGCTCAAAGCGGTAATGGACGATTTTGC |
| | | GGCGTTCGTAGAGAAGTGTTGCAAGGCGGACGATAAAGA |
| | | AACGTGCTTTGCTGAGGAGGGAAAAAAGCTTGTTGCTGC |
| | | CAGTCAAGCGGCGCTTGGCTTGAGTGGTGGAGAACAAAA |
| | | ACTCATTTCCGAGGAAGATCTTTGA |
| 112 | ProC1493/ | METDTLLLWVLLLWVPGSTGQERRSHEVNPAAHLTGANSS |
| | ProC_LIGHT-3HB- | LTGSGGPLLWETQLGLAFLRGLSYHDGALVVTKAGYYYIYS |
| | 1204DNI-HSA-His | KVQLGGVGCPLGLASTITHGLYKRTPRYPEELELLVSQQSPC |
| | Protein with signal | GRATSSSRVWWDSSFLGGVVHLEAGEEVVVRVLDERLVRL |
| | sequence | RDGTRSYFGAFMVSSAGGGSGGGSGGSGEIAALKQEIAALK |
| | | KEIAALKWEIAALKQGYYGGSGGSLSGRSDNIGSDAHKSEV |
| | | AHRFKDLGEENFKALVLIAFAQYLQQCPFEDHVKLVNEVTE |
| | | FAKTCVADESAENCDKSLHTLFGDKLCTVATLRETYGEMA |
| | | DCCAKQEPERNECFLQHKDDNPNLPRLVRPEVDVMCTAFH |
| | | DNEETFLKKYLYEIARRHPYFYAPELLFFAKRYKAAFTECC |
| | | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLSHHHHHH |
| 113 | ProC1493/ | atggaaaccgacacactgctgctgtgggtgctgcttttgtgggtgccaggatccacaggcCA |
| | ProC_LIGHT-3HB- | AGAGCGAAGATCCCATGAAGTAAACCCTGCAGCTCACCT |
| | 1204DNI-HSA-His | TACAGGAGCCAACAGCAGTCTGACAGGTTCTGGGGGCCC |
| | DNA with coding | CTTGTTGTGGGAGACGCAACTGGGGCTTGCATTCCTCCGA |
| | sequence for signal | GGGCTCAGTTACCACGACGGCGCGCTTGTTGTTACAAAA |
| | peptide | GCAGGATACTACTATATTTACTCAAAGGTGCAGCTGGGT |
| | | GGAGTGGGATGTCCATTGGGCCTGGCCTCAACTATAACC |
| | | CATGGCCTCTATAAAAGAACGCCCCGGTATCCTGAGGAG |
| | | CTGGAGCTGTTGGTCTCACAGCAGTCACCGTGCGGCAGA |
| | | GCCACATCATCCTCTCGCGTATGGTGGGACTCTTCCTTCC |
| | | TGGGAGGTGTAGTCCATCTCGAGGCAGGTGAAGAAGTCG |
| | | TAGTTCGCGTACTCGATGAACGCCTGGTTCGGCTGAGGG |
| | | ATGGGACTAGGAGCTATTTCGGCGCATTTATGGTATCTTC |
| | | TGCAGGTGGAGGAAGTGGTGGCGGTTCCGGTGGTTCAGG |
| | | GGAAATCGCGGCACTCAAACAAGAGATAGCGGCTTTGAA |
| | | AAAGGAGATCGCAGCCCTGAAATGGGAAATAGCGGCCTT |
| | | GAAGCAGGGCTACTATGGCGGCAGCGGCGGCAGCCTAAG |
| | | CGGACGGTCCGACAATATCGGCAGCGATGCACATAAAAG |
| | | TGAGGTGGCCCACCGCTTTAAAGACCTCGGCGAGGAGAA |
| | | CTTCAAGGCACTCGTACTTATTGCCTTTGCACAATACCTG |
| | | CAGCAATGTCCGTTTGAAGATCATGTCAAGCTCGTAAATG |
| | | AAGTGACAGAGTTCGCTAAGACCTGTGTAGCTGACGAAA |
| | | GTGCCGAAAACTGCGACAAATCATTGCATACATTGTTCG |
| | | GCGACAAGCTTTGTACAGTTGCCACGCTCCGCGAGACAT |
| | | ACGGCGAAATGGCCGATTGTTGTGCGAAACAGGAACCCG |
| | | AAAGAAATGAGTGCTTCTTGCAACACAAAGACGATAATC |
| | | CCAATCTTCCGCGACTGGTGAGACCCGAAGTAGATGTAA |
| | | TGTGCACAGCTTTTCATGACAATGAGGAAACCTTTCTTAA |
| | | AAAATATCTGTACGAAATCGCCCGCCGACACCCGTATTTC |
| | | TATGCCCCAGAGCTGTTGTTTTTCGCAAAGCGCTACAAGG |
| | | CAGCGTTTACCGAATGCTGCCAGGCCGCTGATAAAGCCG |
| | | CTTGTCTGCTGCCTAAGCTTGATGAGCTTCGCGACGAGGG |
| | | TAAGGCTTCCAGCGCTAAACAACGCCTGAAGTGTGCATC |
| | | TTTGCAAAAATTTGGTGAACGAGCCTTCAAGGCTTGGGCT |
| | | GTTGCCAGGCTGAGTCAACGGTTCCCGAAGGCGGAGTTT |
| | | GCGGAGGTTTCCAAGTTGGTAACGGACCTGACAAAGGTA |
| | | CATACCGAATGCTGCCATGGCGACCTGTTGGAGTGCGCC |
| | | GATGATCGAGCGGATCTTGCCAAGTATATTTGCGAAAAC |
| | | CAAGACTCCATTTCCAGTAAACTTAAGGAGTGTTGTGAG |
| | | AAACCTCTCCTTGAAAAGAGCCATTGTATAGCGGAGGTC |
| | | GAAAACGATGAAATGCCCGCTGATCTGCCGTCACTCGCT |
| | | GCTGACTTCGTTGAATCCAAGGATGTATGTAAAAACTATG |
| | | CCGAAGCTAAGGACGTCTTTCTTGGAATGTTCTTGTATGA |
| | | ATACGCCCGACGGCATCCTGACTATTCAGTTGTGCTCCTT |
| | | CTTCGATTGGCTAAAACTTATGAGACGACACTTGAAAAG |
| | | TGTTGTGCCGCCGCCGACCCACATGAGTGCTACGCTAAG |
| | | GTTTTCGACGAATTTAAGCCCTTGGTTGAAGAACCGCAGA |
| | | ATCTCATTAAGCAAAATTGTGAGCTCTTTGAACAACTCGG |
| | | TGAATACAAATTTCAGAACGCGCTTCTGGTTAGATATACG |
| | | AAGAAAGTGCCACAGGTGAGCACCCCTACATTGGTTGAG |
| | | GTCAGTAGGAACCTCGGCAAGGTGGGATCTAAGTGCTGC |
| | | AAACACCCGGAGGCAAAGAGAATGCCTTGCGCTGAGGAT |
| | | TATCTCTCAGTGGTTCTGAATCAGCTTTGCGTTCTTCACG |
| | | AAAAGACTCCCGTCAGCGACCGAGTCACGAAGTGCTGTA |
| | | CCGAGAGCCTTGTTAATAGACGACCTTGCTTCTCCGCGCT |
| | | TGAGGTGGATGAGACCTACGTCCCAAAGGAATTCAACGC |
| | | CGAGACGTTCACTTTCCATGCCGATATTTGCACCTTGTCA |
| | | GAAAAGGAGCGACAGATAAAAAAGCAAACTGCTTTGGTG |
| | | GAGCTGGTTAAGCATAAGCCTAAAGCGACGAAGGAACAG |
| | | CTCAAAGCCGTAATGGATGATTTCGCTGCCTTCGTGGAGA |
| | | AATGTTGTAAGGCCGATGACAAGGAAACCTGCTTCGCTG |
| | | AAGAGGGTAAAAAGCTCGTCGCCGCTAGTCAGGCGGCAC |
| | | TTGGTTTGAGCCACCACCACCATCACCACtga |
| 114 | ProC1490/ | METDTLLLWVLLLWVPGSTGEIAALKQEIAALKKEIAALKW |
| | ProC_3HB-LIGHT- | EIAALKQGYYGGSGGSQERRSHEVNPAAHLTGANSSLTGSG |
| | 1204DNI-HSA-cMyc | GPLLWETQLGLAFLRGLSYHDGALVVTKAGYYYIYSKVQL |
| | Protein with signal | GGVGCPLGLASTITHGLYKRTPRYPEELELLVSQQSPCGRAT |
| | sequence | SSSRVWWDSSFLGGVVHLEAGEEVVVRVLDERLVRLRDGT |
| | | RSYFGAFMVSSAGGGSSGGSLSGRSDNIGSDAHKSEVAHRF |
| | | KDLGEENFKALVLIAFAQYLQQCPFEDHVKLVNEVTEFAKT |
| | | CVADESAENCDKSLHTLFGDKLCTVATLRETYGEMADCCA |
| | | KQEPERNECFLQHKDDNPNLPRLVRPEVDVMCTAFHDNEE |
| | | TFLKKYLYEIARRHPYFYAPELLFFAKRYKAAFTECCQAAD |
| | | KAACLLPKLDELRDEGKASSAKQRLKCASLQKFGERAFKA |
| | | WAVARLSQRFPKAEFAEVSKLVTDLTKVHTECCHGDLLEC |
| | | ADDRADLAKYICENQDSISSKLKECCEKPLLEKSHCIAEVEN |
| | | DEMPADLPSLAADFVESKDVCKNYAEAKDVFLGMFLYEYA |
| | | RRHPDYSVVLLLRLAKTYETTLEKCCAAADPHECYAKVFD |
| | | EFKPLVEEPQNLIKQNCELFEQLGEYKFQNALLVRYTKKVP |
| | | QVSTPTLVEVSRNLGKVGSKCCKHPEAKRMPCAEDYLSVV |
| | | LNQLCVLHEKTPVSDRVTKCCTESLVNRRPCFSALEVDETY |
| | | VPKEFNAETFTFHADICTLSEKERQIKKQTALVELVKHKPKA |
| | | TKEQLKAVMDDFAAFVEKCCKADDKETCFAEEGKKLVAAS |
| | | QAALGLSGGEQKLISEEDL |
| 115 | ProC1490/ | atggaaaccgacacactgctgctgtgggtgctgcttttgtgggtgccaggatccacaggcGA |
| | ProC_3HB-LIGHT- | AATCGCGGCACTCAAACAAGAGATAGCGGCTTTGAAAAA |
| | 1204DNI-HSA-cMyc | GGAGATCGCAGCCCTGAAATGGGAAATAGCGGCCTTGAA |
| | DNA with coding | GCAGGGCTACTATGGGGGATCCGGGGGTTCACAGGAGCG |
| | sequence for signal | ACGGTCCCATGAGGTGAATCCGGCAGCGCATTTGACGGG |
| | peptide | GGCCAACTCATCCCTGACAGGTTCAGGTGGCCCTCTGTTG |
| | | TGGGAAACTCAGCTCGGACTGGCCTTCCTTAGAGGTTTGT |
| | | CATATCATGACGGAGCACTTGTAGTCACCAAAGCTGGGT |
| | | ATTACTACATATACTCTAAGGTCCAGCTGGGTGGGGTGG |
| | | GCTGTCCACTTGGCTTGGCATCTACGATCACGCATGGGTT |
| | | GTACAAAAGAACTCCACGATATCCAGAAGAACTCGAATT |
| | | GCTTGTCTCCCAACAATCTCCTTGTGGCAGGGCTACGTCC |
| | | AGTTCCCGAGTGTGGTGGGATTCAAGTTTTCTCGGGGGCG |
| | | TAGTCCATCTTGAAGCAGGGGAGGAAGTGGTCGTCCGAG |
| | | TGCTGGACGAACGGTTGGTTAGGCTTCGGGATGGGACAA |
| | | GAAGTTATTTTGGGGCCTTCATGGTATCCAGCGCCGGGGG |
| | | CGGAAGCAGCGGCGGCAGCTTAAGCGGCAGAAGCGACA |
| | | ACATCGGCAGCGATGCTCATAAAAGTGAGGTCGCGCACA |
| | | GATTCAAAGACCTCGGCGAGGAAAACTTTAAAGCCCTCG |
| | | TATTGATAGCTTTCGCCCAGTACCTCCAGCAGTGCCCATT |
| | | CGAGGATCACGTGAAACTCGTGAATGAAGTAACGGAATT |
| | | TGCCAAAACGTGCGTGGCCGACGAGAGTGCCGAAAATTG |
| | | CGACAAGTCACTTCACACCCTTTTTGGTGACAAATTGTGT |
| | | ACTGTGGCGACGCTTAGGGAAACATACGGAGAAATGGCC |
| | | GATTGCTGTGCCAAACAAGAGCCTGAAAGAAACGAATGC |
| | | TTTCTTCAGCACAAAGACGATAACCCTAACTTGCCGAGAC |
| | | TGGTGAGACCTGAAGTAGACGTCATGTGTACCGCGTTCC |
| | | ATGACAACGAAGAGACTTTCTTGAAAAAATACCTTTATG |
| | | AAATAGCACGGAGGCACCCTTATTTTTACGCTCCAGAGCT |
| | | CCTCTTCTTTGCTAAACGGTACAAAGCGGCGTTTACCGAA |
| | | TGTTGCCAAGCTGCGGATAAAGCTGCATGTCTTCTCCCGA |
| | | AGTTGGATGAGCTGAGAGATGAGGGTAAAGCGTCTTCCG |
| | | CTAAACAACGACTTAAATGCGCTTCTCTGCAGAAATTTGG |
| | | CGAACGAGCGTTCAAAGCCTGGGCCGTTGCGCGCCTTTC |
| | | ACAGCGGTTTCCGAAAGCCGAGTTCGCCGAGGTTTCAAA |
| | | GCTGGTTACGGACTTGACAAAGGTGCATACAGAATGCTG |
| | | CCACGGAGACCTGCTGGAGTGCGCCGATGATCGCGCTGA |
| | | TTTGGCTAAATATATTTGCGAAAATCAGGACAGCATCAG |
| | | CTCCAAGTTGAAGGAGTGTTGTGAAAAACCGCTGCTGGA |
| | | GAAGTCACACTGCATAGCCGAGGTGGAAAACGACGAGAT |
| | | GCCGGCAGACCTCCCCAGCCTTGCCGCGGATTTCGTCGA |
| | | ATCAAAGGATGTATGCAAAAATTACGCGGAAGCAAAAGA |
| | | TGTATTTCTGGGAATGTTCCTGTACGAGTACGCTAGGCGA |
| | | CATCCCGACTACAGCGTTGTTCTGCTCTTGAGACTTGCGA |
| | | AGACGTACGAGACTACGCTCGAGAAGTGCTGTGCAGCTG |
| | | CGGACCCCCATGAGTGTTATGCTAAAGTCTTCGACGAATT |
| | | TAAGCCTTTGGTGGAAGAGCCACAGAACCTGATCAAACA |
| | | AAATTGTGAACTGTTCGAACAATTGGGGGAGTATAAATT |
| | | TCAGAACGCTCTCCTTGTTCGGTATACAAAAAAAGTACCG |
| | | CAGGTAAGTACGCCGACACTCGTGGAAGTAAGCAGGAAC |
| | | CTCGGAAAGGTTGGATCAAAATGCTGCAAGCATCCGGAG |
| | | GCTAAGCGCATGCCATGCGCTGAGGATTATCTTTCAGTGG |
| | | TCCTGAACCAATTGTGCGTGTTGCACGAGAAAACTCCAGT |
| | | AAGCGATAGAGTTACCAAATGTTGTACAGAGAGCCTGGT |
| | | TAATCGACGACCTTGCTTCAGCGCCTTGGAAGTCGATGAG |
| | | ACGTATGTGCCGAAGGAATTTAACGCCGAAACTTTCACTT |
| | | TTCATGCAGATATCTGTACATTGAGCGAAAAGGAAAGGC |
| | | AAATTAAAAAACAAACTGCGCTTGTGGAATTGGTCAAGC |
| | | ACAAGCCTAAAGCCACAAAGGAACAGCTCAAAGCGGTA |
| | | ATGGACGATTTTGCGGCGTTCGTAGAGAAGTGTTGCAAG |
| | | GCGGACGATAAAGAAACGTGCTTTGCTGAGGAGGGAAAA |
| | | AAGCTTGTTGCTGCCAGTCAAGCGGCGCTTGGCTTGAGTG |
| | | GTGGAGAACAAAAACTCATTTCCGAGGAAGATCTTTGA |
| 116 | ProC1494/ | METDTLLLWVLLLWVPGSTGQGQSGSRHPCRHDPHIICYKF |
| | ProC_mLm16- | GGGSSGGSISSGLLSGRSDNIGGSQERRSHEVNPAAHLTGAN |
| | LIGHT-3HB- | SSLTGSGGPLLWETQLGLAFLRGLSYHDGALVVTKAGYYYI |
| | 1204DNI-HSA-His | YSKVQLGGVGCPLGLASTITHGLYKRTPRYPEELELLVSQQS |
| | Protein with signal | PCGRATSSSRVWWDSSFLGGVVHLEAGEEVVVRVLDERLV |
| | sequence | RLRDGTRSYFGAFMVSSAGGGSGGGSGGSGEIAALKQEIAA |
| | | LKKEIAALKWEIAALKQGYYGGSGGSLSGRSDNIGSDAHKS |
| | | EVAHRFKDLGEENFKALVLIAFAQYLQQCPFEDHVKLVNEV |
| | | TEFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSHHHHHH |
| 117 | ProC1494/ | atggaaaccgacacactgctgctgtgggtgctgcttttgtgggtgccaggatccacaggcCA |
| | ProC_mLm16- | AGGACAGAGCGGCTCACGGCATCCTTGTAGACACGACCC |
| | LIGHT-3HB- | ACACATCATCTGTTACAAGTTTGGAGGCGGCAGCAGCGG |
| | 1204DNI-HSA-His | CGGCTCTATCAGCTCTGGCCTTCTGAGCGGTCGGAGCGAC |
| | DNA with coding | AACATCGGAGGCAGCCAAGAGCGAAGATCCCATGAAGTA |
| | sequence for signal | AACCCTGCAGCTCACCTTACAGGAGCCAACAGCAGTCTG |
| | peptide | ACAGGTTCTGGGGGCCCCTTGTTGTGGGAGACGCAACTG |
| | | GGGCTTGCATTCCTCCGAGGGCTCAGTTACCACGACGGC |
| | | GCGCTTGTTGTTACAAAAGCAGGATACTACTATATTTACT |
| | | CAAAGGTGCAGCTGGGTGGAGTGGGATGTCCATTGGGCC |
| | | TGGCCTCAACTATAACCCATGGCCTCTATAAAAGAACGC |
| | | CCCGGTATCCTGAGGAGCTGGAGCTGTTGGTCTCACAGC |
| | | AGTCACCGTGCGGCAGAGCCACATCATCCTCTCGCGTAT |
| | | GGTGGGACTCTTCCTTCCTGGGAGGTGTAGTCCATCTCGA |
| | | GGCAGGTGAAGAAGTCGTAGTTCGCGTACTCGATGAACG |
| | | CCTGGTTCGGCTGAGGGATGGGACTAGGAGCTATTTCGG |
| | | CGCATTTATGGTATCTTCTGCAGGTGGAGGAAGTGGTGGC |
| | | GGTTCCGGTGGTTCAGGGGAAATCGCGGCACTCAAACAA |
| | | GAGATAGCGGCTTTGAAAAAGGAGATCGCAGCCCTGAAA |
| | | TGGGAAATAGCGGCCTTGAAGCAGGGCTACTATGGCGGC |
| | | AGCGGCGGCAGCCTAAGCGGACGGTCCGACAATATCGGC |
| | | AGCGATGCACATAAAAGTGAGGTGGCCCACCGCTTTAAA |
| | | GACCTCGGCGAGGAGAACTTCAAGGCACTCGTACTTATT |
| | | GCCTTTGCACAATACCTGCAGCAATGTCCGTTTGAAGATC |
| | | ATGTCAAGCTCGTAAATGAAGTGACAGAGTTCGCTAAGA |
| | | CCTGTGTAGCTGACGAAAGTGCCGAAAACTGCGACAAAT |
| | | CATTGCATACATTGTTCGGCGACAAGCTTTGTACAGTTGC |
| | | CACGCTCCGCGAGACATACGGCGAAATGGCCGATTGTTG |
| | | TGCGAAACAGGAACCCGAAAGAAATGAGTGCTTCTTGCA |
| | | ACACAAAGACGATAATCCCAATCTTCCGCGACTGGTGAG |
| | | ACCCGAAGTAGATGTAATGTGCACAGCTTTTCATGACAAT |
| | | GAGGAAACCTTTCTTAAAAAATATCTGTACGAAATCGCC |
| | | CGCCGACACCCGTATTTCTATGCCCCAGAGCTGTTGTTTT |
| | | TCGCAAAGCGCTACAAGGCAGCGTTTACaGAATGCTGCCA |
| | | GGCCGCTGATAAAGCCGCTTGTCTGCTGCCTAAGCTTGAT |
| | | GAGCTTCGCGACGAGGGTAAGGCTTCCAGCGCTAAACAA |
| | | CGCCTGAAGTGTGCATCTTTGCAAAAATTTGGTGAACGA |
| | | GCCTTCAAGGCTTGGGCTGTTGCCAGGCTGAGTCAACGGT |
| | | TCCCGAAGGCGGAGTTTGCGGAGGTTTCCAAGTTGGTAA |
| | | CGGACCTGACAAAGGTACATACCGAATGCTGCCATGGCG |
| | | ACCTGTTGGAGTGCGCCGATGATCGAGCGGATCTTGCCA |
| | | AGTATATTTGCGAAAACCAAGACTCCATTTCCAGTAAACT |
| | | TAAGGAGTGTTGTGAGAAACCTCTCCTTGAAAAGAGCCA |
| | | TTGTATAGCGGAGGTCGAAAACGATGAAATGCCCGCTGA |
| | | TCTGCCGTCACTCGCTGCTGACTTCGTTGAATCCAAGGAT |
| | | GTATGTAAAAACTATGCCGAAGCTAAGGACGTCTTTCTTG |
| | | GAATGTTCTTGTATGAATACGCCCGACGGCATCCTGACTA |
| | | TTCAGTTGTGCTCCTTCTTCGATTGGCTAAAACTTATGAG |
| | | ACGACACTTGAAAAGTGTTGTGCCGCCGCCGACCCACAT |
| | | GAGTGCTACGCTAAGGTTTTCGACGAATTTAAGCCCTTGG |
| | | TTGAAGAACCGCAGAATCTCATTAAGCAAAATTGTGAGC |
| | | TCTTTGAACAACTCGGTGAATACAAATTTCAGAACGCGCT |
| | | TCTGGTTAGATATACGAAGAAAGTGCCACAGGTGAGCAC |
| | | CCCTACATTGGTTGAGGTCAGTAGGAACCTCGGCAAGGT |
| | | GGGATCTAAGTGCTGCAAACACCCGGAGGCAAAGAGAAT |
| | | GCCTTGCGCTGAGGATTATCTCTCAGTGGTTCTGAATCAG |
| | | CTTTGCGTTCTTCACGAAAAGACTCCCGTCAGCGACCGAG |
| | | TCACGAAGTGCTGTACCGAGAGCCTTGTTAATAGACGAC |
| | | CTTGCTTCTCCGCGCTTGAGGTGGATGAGACCTACGTCCC |
| | | AAAGGAATTCAACGCCGAGACGTTCACTTTCCATGCCGA |
| | | TATTTGCACCTTGTCAGAAAAGGAGCGACAGATAAAAAA |
| | | GCAAACTGCTTTGGTGGAGCTGGTTAAGCATAAGCCTAA |
| | | AGCGACGAAGGAACAGCTCAAAGCCGTAATGGATGATTT |
| | | CGCTGCCTTCGTGGAGAAATGTTGTAAGGCCGATGACAA |
| | | GGAAACCTGCTTCGCTGAAGAGGGTAAAAAGCTCGTCGC |
| | | CGCTAGTCAGGCGGCACTTGGTTTGAGCCACCACCACCA |
| | | TCACCACtga |
| 118 | ProC2006/ | METDTLLLWVLLLWVPGSTGGSGEQKLISEEDLGDAHKSEV |
| | ProC_cMyc-HSA- | AHRFKDLGEENFKALVLIAFAQYLQQCPFEDHVKLVNEVTE |
| | mLm16-1490DNI- | FAKTCVADESAENCDKSLHTLFGDKLCTVATLRETYGEMA |
| | LIGHT-3HB | DCCAKQEPERNECFLQHKDDNPNLPRLVRPEVDVMCTAFH |
| | Protein with signal | DNEETFLKKYLYEIARRHPYFYAPELLFFAKRYKAAFTECC |
| | sequence | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLGGGSSGGSGSQGQSGSRHPCRHDPHIICYKF |
| | | GGGSSGGSISSGLLSGRSDNIGGSQERRSHEVNPAAHLTGAN |
| | | SSLTGSGGPLLWETQLGLAFLRGLSYHDGALVVTKAGYYYI |
| | | YSKVQLGGVGCPLGLASTITHGLYKRTPRYPEELELLVSQQS |
| | | PCGRATSSSRVWWDSSFLGGVVHLEAGEEVVVRVLDERLV |
| | | RLRDGTRSYFGAFMVSSAGGGSGGGSGGSGEIAALKQEIAA |
| | | LKKEIAALKWEIAALKQGYYGG |
| 119 | ProC2006/ | atggaaaccgacacactgctgctgtgggtgctgcttttgtgggtgccaggatccacaggcGG |
| | ProC_cMyc-HSA- | CTCTGGCGAACAAAAACTCATTTCCGAGGAAGATCTTGG |
| | mLm16-1490DNI- | CGATGCACATAAAAGTGAGGTGGCCCACCGCTTTAAAGA |
| | LIGHT-3HB | CCTCGGCGAGGAGAACTTCAAGGCACTCGTACTTATTGCC |
| | DNA with coding | TTTGCACAATACCTGCAGCAATGTCCGTTTGAAGATCATG |
| | sequence for signal | TCAAGCTCGTAAATGAAGTGACAGAGTTCGCTAAGACCT |
| | peptide | GTGTAGCTGACGAAAGTGCCGAAAACTGCGACAAATCAT |
| | | TGCATACATTGTTCGGCGACAAGCTTTGTACAGTTGCCAC |
| | | GCTCCGCGAGACATACGGCGAAATGGCCGATTGTTGTGC |
| | | GAAACAGGAACCCGAAAGAAATGAGTGCTTCTTGCAACA |
| | | CAAAGACGATAATCCCAATCTTCCGCGACTGGTGAGACC |
| | | CGAAGTAGATGTAATGTGCACAGCTTTTCATGACAATGA |
| | | GGAAACCTTTCTTAAAAAATATCTGTACGAAATCGCCCGC |
| | | CGACACCCGTATTTCTATGCCCCAGAGCTGTTGTTTTTCG |
| | | CAAAGCGCTACAAGGCAGCGTTTACaGAATGCTGtCAGGC |
| | | CGCTGATAAAGCCGCTTGTCTGCTGCCTAAGCTTGATGAG |
| | | CTTCGCGACGAGGGTAAGGCTTCCAGCGCTAAACAACGC |
| | | CTGAAGTGTGCATCTTTGCAAAAATTTGGTGAACGAGCCT |
| | | TCAAGGCTTGGGCTGTTGCCAGGCTGAGTCAACGGTTCCC |
| | | GAAGGCGGAGTTTGCGGAGGTTTCCAAGTTGGTAACGGA |
| | | CCTGACAAAGGTACATACCGAATGCTGCCATGGCGACCT |
| | | GTTGGAGTGCGCCGATGATCGAGCGGATCTTGCCAAGTA |
| | | TATTTGCGAAAACCAAGACTCCATTTCCAGTAAACTTAAG |
| | | GAGTGTTGTGAGAAACCTCTCCTTGAAAAGAGCCATTGT |
| | | ATAGCGGAGGTCGAAAACGATGAAATGCCCGCTGATCTG |
| | | CCGTCACTCGCTGCTGACTTCGTTGAATCCAAGGATGTAT |
| | | GTAAAAACTATGCCGAAGCTAAGGACGTCTTTCTTGGAA |
| | | TGTTCTTGTATGAATACGCCCGACGGCATCCTGACTATTC |
| | | AGTTGTGCTCCTTCTTCGATTGGCTAAAACTTATGAGACG |
| | | ACACTTGAAAAGTGTTGTGCCGCCGCCGACCCACATGAG |
| | | TGCTACGCTAAGGTTTTCGACGAATTTAAGCCCTTGGTTG |
| | | AAGAACCGCAGAATCTCATTAAGCAAAATTGTGAGCTCT |
| | | TTGAACAACTCGGTGAATACAAATTTCAGAACGCGCTTCT |
| | | GGTTAGATATACGAAGAAAGTGCCACAGGTGAGCACCCC |
| | | TACATTGGTTGAGGTCAGTAGGAACCTCGGCAAGGTGGG |
| | | ATCTAAGTGCTGCAAACACCCGGAGGCAAAGAGAATGCC |
| | | TTGCGCTGAGGATTATCTCTCAGTGGTTCTGAATCAGCTT |
| | | TGCGTTCTTCACGAAAAGACTCCCGTCAGCGACCGAGTC |
| | | ACGAAGTGCTGTACCGAGAGCCTTGTTAATAGACGACCT |
| | | TGCTTCTCCGCGCTTGAGGTGGATGAGACCTACGTCCCAA |
| | | AGGAATTCAACGCCGAGACGTTCACTTTCCATGCCGATAT |
| | | TTGCACCTTGTCAGAAAAGGAGCGACAGATAAAAAAGCA |
| | | AACTGCTTTGGTGGAGCTGGTTAAGCATAAGCCTAAAGC |
| | | GACGAAGGAACAGCTCAAAGCCGTAATGGATGATTTCGC |
| | | TGCCTTCGTGGAGAAATGTTGTAAGGCCGATGACAAGGA |
| | | AACCTGCTTCGCTGAAGAGGGTAAAAAGCTCGTCGCCGC |
| | | TAGTCAGGCGGCACTTGGTTTGGGGGGCGGAAGCAGCGG |
| | | CGGCAGCGGCAGCCAAGGCCAGAGCGGCAGCAGGCACC |
| | | CTTGTAGACACGATCCTCACATCATCTGTTACAAGTTTGG |
| | | AGGAGGCAGTAGCGGTGGCAGTATTAGCAGCGGACTGCT |
| | | GAGTGGCAGAAGCGACAACATCGGCGGCAGCCAGGAGA |
| | | GACGGAGCCACGAGGTGAACCCCGCTGCCCACCTGACCG |
| | | GCGCCAACAGCAGTCTGACCGGCAGCGGCGGCCCTCTGC |
| | | TGTGGGAGACACAGCTGGGCCTGGCCTTTCTGAGAGGCC |
| | | TGTCCTACCACGACGGCGCCCTGGTCGTGACCAAGGCCG |
| | | GCTACTACTACATCTACAGCAAGGTGCAGCTGGGAGGCG |
| | | TGGGCTGCCCTCTGGGCCTGGCATCTACCATCACGCACGG |
| | | CCTGTACAAGCGGACCCCTAGATATCCTGAGGAACTGGA |
| | | ACTCCTGGTGTCCCAGCAGAGCCCTTGCGGCAGAGCTAC |
| | | AAGCAGCTCAAGAGTGTGGTGGGACAGCAGCTTCCTGGG |
| | | CGGAGTGGTCCACCTGGAAGCTGGAGAAGAAGTGGTGGT |
| | | CCGGGTGCTGGACGAGAGACTCGTGAGGCTGAGAGACGG |
| | | AACAAGAAGCTACTTCGGCGCTTTTATGGTGTCTTCTGCA |
| | | GGTGGAGGAAGTGGTGGCGGTTCCGGTGGTTCAGGGGAA |
| | | ATCGCGGCACTCAAACAAGAGATAGCGGCTTTGAAAAAG |
| | | GAGATCGCAGCCCTGAAATGGGAAATAGCGGCCTTGAAG |
| | | CAGGGCTACTATGGCGGCtga |
| 120 | ProC1497/ | METDTLLLWVLLLWVPGSTGGSGEQKLISEEDLGDAHKSEV |
| | ProC_cMyc-HSA- | AHRFKDLGEENFKALVLIAFAQYLQQCPFEDHVKLVNEVTE |
| | 21GS-1204DNI- | FAKTCVADESAENCDKSLHTLFGDKLCTVATLRETYGEMA |
| | LIGHT | DCCAKQEPERNECFLQHKDDNPNLPRLVRPEVDVMCTAFH |
| | Protein with signal | DNEETFLKKYLYEIARRHPYFYAPELLFFAKRYKAAFTECC |
| | sequence | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLSSAGGGSSGGSLSGRSDNIGSQERRSHEVNP |
| | | AAHLTGANSSLTGSGGPLLWETQLGLAFLRGLSYHDGALV |
| | | VTKAGYYYIYSKVQLGGVGCPLGLASTITHGLYKRTPRYPE |
| | | ELELLVSQQSPCGRATSSSRVWWDSSFLGGVVHLEAGEEVV |
| | | VRVLDERLVRLRDGTRSYFGAFMV |
| 121 | ProC1497/ | atggaaaccgacacactgctgctgtgggtgctgcttttgtgggtgccaggatccacaggcGG |
| | ProC_cMyc-HSA- | CTCTGGCGAACAAAAACTCATTTCCGAGGAAGATCTTGG |
| | 21GS-1204DNI- | CGATGCACATAAAAGTGAGGTGGCCCACCGCTTTAAAGA |
| | LIGHT | CCTCGGCGAGGAGAACTTCAAGGCACTCGTACTTATTGCC |
| | DNA with coding | TTTGCACAATACCTGCAGCAATGTCCGTTTGAAGATCATG |
| | sequence for signal | TCAAGCTCGTAAATGAAGTGACAGAGTTCGCTAAGACCT |
| | peptide | GTGTAGCTGACGAAAGTGCCGAAAACTGCGACAAATCAT |
| | | TGCATACATTGTTCGGCGACAAGCTTTGTACAGTTGCCAC |
| | | GCTCCGCGAGACATACGGCGAAATGGCCGATTGTTGTGC |
| | | GAAACAGGAACCCGAAAGAAATGAGTGCTTCTTGCAACA |
| | | CAAAGACGATAATCCCAATCTTCCGCGACTGGTGAGACC |
| | | CGAAGTAGATGTAATGTGCACAGCTTTTCATGACAATGA |
| | | GGAAACCTTTCTTAAAAAATATCTGTACGAAATCGCCCGC |
| | | CGACACCCGTATTTCTATGCCCCAGAGCTGTTGTTTTTCG |
| | | CAAAGCGCTACAAGGCAGCGTTTACaGAATGCTGtCAGGC |
| | | CGCTGATAAAGCCGCTTGTCTGCTGCCTAAGCTTGATGAG |
| | | CTTCGCGACGAGGGTAAGGCTTCCAGCGCTAAACAACGC |
| | | CTGAAGTGTGCATCTTTGCAAAAATTTGGTGAACGAGCCT |
| | | TCAAGGCTTGGGCTGTTGCCAGGCTGAGTCAACGGTTCCC |
| | | GAAGGCGGAGTTTGCGGAGGTTTCCAAGTTGGTAACGGA |
| | | CCTGACAAAGGTACATACCGAATGCTGCCATGGCGACCT |
| | | GTTGGAGTGCGCCGATGATCGAGCGGATCTTGCCAAGTA |
| | | TATTTGCGAAAACCAAGACTCCATTTCCAGTAAACTTAAG |
| | | GAGTGTTGTGAGAAACCTCTCCTTGAAAAGAGCCATTGT |
| | | ATAGCGGAGGTCGAAAACGATGAAATGCCCGCTGATCTG |
| | | CCGTCACTCGCTGCTGACTTCGTTGAATCCAAGGATGTAT |
| | | GTAAAAACTATGCCGAAGCTAAGGACGTCTTTCTTGGAA |
| | | TGTTCTTGTATGAATACGCCCGACGGCATCCTGACTATTC |
| | | AGTTGTGCTCCTTCTTCGATTGGCTAAAACTTATGAGACG |
| | | ACACTTGAAAAGTGTTGTGCCGCCGCCGACCCACATGAG |
| | | TGCTACGCTAAGGTTTTCGACGAATTTAAGCCCTTGGTTG |
| | | AAGAACCGCAGAATCTCATTAAGCAAAATTGTGAGCTCT |
| | | TTGAACAACTCGGTGAATACAAATTTCAGAACGCGCTTCT |
| | | GGTTAGATATACGAAGAAAGTGCCACAGGTGAGCACCCC |
| | | TACATTGGTTGAGGTCAGTAGGAACCTCGGCAAGGTGGG |
| | | ATCTAAGTGCTGCAAACACCCGGAGGCAAAGAGAATGCC |
| | | TTGCGCTGAGGATTATCTCTCAGTGGTTCTGAATCAGCTT |
| | | TGCGTTCTTCACGAAAAGACTCCCGTCAGCGACCGAGTC |
| | | ACGAAGTGCTGTACCGAGAGCCTTGTTAATAGACGACCT |
| | | TGCTTCTCCGCGCTTGAGGTGGATGAGACCTACGTCCCAA |
| | | AGGAATTCAACGCCGAGACGTTCACTTTCCATGCCGATAT |
| | | TTGCACCTTGTCAGAAAAGGAGCGACAGATAAAAAAGCA |
| | | AACTGCTTTGGTGGAGCTGGTTAAGCATAAGCCTAAAGC |
| | | GACGAAGGAACAGCTCAAAGCCGTAATGGATGATTTCGC |
| | | TGCCTTCGTGGAGAAATGTTGTAAGGCCGATGACAAGGA |
| | | AACCTGCTTCGCTGAAGAGGGTAAAAAGCTCGTCGCCGC |
| | | TAGTCAGGCGGCACTTGGTTTGTCCAGCGCCGGGGGCGG |
| | | AAGCAGCGGCGGCAGCTTAAGCGGCAGAAGCGACAACA |
| | | TCGGCAGCCAGGAGAGACGGAGCCACGAGGTGAACCCC |
| | | GCTGCCCACCTGACCGGCGCCAACAGCAGTCTGACCGGC |
| | | AGCGGCGGCCCTCTGCTGTGGGAGACACAGCTGGGCCTG |
| | | GCCTTTCTGAGAGGCCTGTCCTACCACGACGGCGCCCTGG |
| | | TCGTGACCAAGGCCGGCTACTACTACATCTACAGCAAGG |
| | | TGCAGCTGGGAGGCGTGGGCTGCCCTCTGGGCCTGGCAT |
| | | CTACCATCACGCACGGCCTGTACAAGCGGACCCCTAGAT |
| | | ATCCTGAGGAACTGGAACTCCTGGTGTCCCAGCAGAGCC |
| | | CTTGCGGCAGAGCTACAAGCAGCTCAAGAGTGTGGTGGG |
| | | ACAGCAGCTTCCTGGGCGGAGTGGTCCACCTGGAAGCTG |
| | | GAGAAGAAGTGGTGGTCCGGGTGCTGGACGAGAGACTCG |
| | | TGAGGCTGAGAGACGGAACAAGAAGCTACTTCGGCGCTT |
| | | TTATGGTGtga |
| 122 | ProC1489/ | METDTLLLWVLLLWVPGSTGQERRSHEVNPAAHLTGANSS |
| | ProC_LIGHT- | LTGSGGPLLWETQLGLAFLRGLSYHDGALVVTKAGYYYIYS |
| | 1490DNI-mLm16- | KVQLGGVGCPLGLASTITHGLYKRTPRYPEELELLVSQQSPC |
| | HSA-cMyc | GRATSSSRVWWDSSFLGGVVHLEAGEEVVVRVLDERLVRL |
| | Protein with signal | RDGTRSYFGAFMVGGGSISSGLLSGRSDNIGGGSSGGSRHPC |
| | sequence | RHDPHIICYKFSGGGSGGGSGDAHKSEVAHRFKDLGEENFK |
| | | ALVLIAFAQYLQQCPFEDHVKLVNEVTEFAKTCVADESAEN |
| | | CDKSLHTLFGDKLCTVATLRETYGEMADCCAKQEPERNEC |
| | | FLQHKDDNPNLPRLVRPEVDVMCTAFHDNEETFLKKYLYEI |
| | | ARRHPYFYAPELLFFAKRYKAAFTECCQAADKAACLLPKL |
| | | DELRDEGKASSAKQRLKCASLQKFGERAFKAWAVARLSQR |
| | | FPKAEFAEVSKLVTDLTKVHTECCHGDLLECADDRADLAK |
| | | YICENQDSISSKLKECCEKPLLEKSHCIAEVENDEMPADLPSL |
| | | AADFVESKDVCKNYAEAKDVFLGMFLYEYARRHPDYSVV |
| | | LLLRLAKTYETTLEKCCAAADPHECYAKVFDEFKPLVEEPQ |
| | | NLIKQNCELFEQLGEYKFQNALLVRYTKKVPQVSTPTLVEV |
| | | SRNLGKVGSKCCKHPEAKRMPCAEDYLSVVLNQLCVLHEK |
| | | TPVSDRVTKCCTESLVNRRPCFSALEVDETYVPKEFNAETFT |
| | | FHADICTLSEKERQIKKQTALVELVKHKPKATKEQLKAVMD |
| | | DFAAFVEKCCKADDKETCFAEEGKKLVAASQAALGLSEQK |
| | | LISEEDL |
| 123 | ProC1489/ | atggaaaccgacacactgctgctgtgggtgctgcttttgtgggtgccaggatccacaggcCA |
| | ProC_LIGHT- | AGAGCGAAGATCCCATGAAGTAAACCCTGCAGCTCACCT |
| | 1490DNI-mLm16- | TACAGGAGCCAACAGCAGTCTGACAGGTTCTGGGGGCCC |
| | HSA-cMyc | CTTGTTGTGGGAGACGCAACTGGGGCTTGCATTCCTCCGA |
| | DNA with coding | GGGCTCAGTTACCACGACGGCGCGCTTGTTGTTACAAAA |
| | sequence for signal | GCAGGATACTACTATATTTACTCAAAGGTGCAGCTGGGT |
| | peptide | GGAGTGGGATGTCCATTGGGCCTGGCCTCAACTATAACC |
| | | CATGGCCTCTATAAAAGAACGCCCCGGTATCCTGAGGAG |
| | | CTGGAGCTGTTGGTCTCACAGCAGTCACCGTGCGGCAGA |
| | | GCCACATCATCCTCTCGCGTATGGTGGGACTCTTCCTTCC |
| | | TGGGAGGTGTAGTCCATCTCGAGGCAGGTGAAGAAGTCG |
| | | TAGTTCGCGTACTCGATGAACGCCTGGTTCGGCTGAGGG |
| | | ATGGGACTAGGAGCTATTTCGGCGCATTTATGGTAGGCG |
| | | GCGGCTCTATCTCCTCCGGCCTGCTGAGCGGCAGAAGCG |
| | | ACAACATCGGCGGAGGCAGCTCCGGCGGCTCTAGACACC |
| | | CTTGTAGACACGATCCTCACATCATCTGTTACAAGTTTTC |
| | | TGGTGGAGGAAGTGGTGGCGGTTCCGGTGATGCACATAA |
| | | AAGTGAGGTGGCCCACCGCTTTAAAGACCTCGGCGAGGA |
| | | GAACTTCAAGGCACTCGTACTTATTGCCTTTGCACAATAC |
| | | CTGCAGCAATGTCCGTTTGAAGATCATGTCAAGCTCGTAA |
| | | ATGAAGTGACAGAGTTCGCTAAGACCTGTGTAGCTGACG |
| | | AAAGTGCCGAAAACTGCGACAAATCATTGCATACATTGT |
| | | TCGGCGACAAGCTTTGTACAGTTGCCACGCTCCGCGAGA |
| | | CATACGGCGAAATGGCCGATTGTTGTGCGAAACAGGAAC |
| | | CCGAAAGAAATGAGTGCTTCTTGCAACACAAAGACGATA |
| | | ATCCCAATCTTCCGCGACTGGTGAGACCCGAAGTAGATG |
| | | TAATGTGCACAGCTTTTCATGACAATGAGGAAACCTTTCT |
| | | TAAAAAATATCTGTACGAAATCGCCCGCCGACACCCGTA |
| | | TTTCTATGCCCCAGAGCTGTTGTTTTTCGCAAAGCGCTAC |
| | | AAGGCAGCGTTTACAGAATGCTGCCAGGCCGCTGATAAA |
| | | GCCGCTTGTCTGCTGCCTAAGCTTGATGAGCTTCGCGACG |
| | | AGGGTAAGGCTTCCAGCGCTAAACAACGCCTGAAGTGTG |
| | | CATCTTTGCAAAAATTTGGTGAACGAGCCTTCAAGGCTTG |
| | | GGCTGTTGCCAGGCTGAGTCAACGGTTCCCGAAGGCGGA |
| | | GTTTGCGGAGGTTTCCAAGTTGGTAACGGACCTGACAAA |
| | | GGTACATACCGAATGCTGCCATGGCGACCTGTTGGAGTG |
| | | CGCCGATGATCGAGCGGATCTTGCCAAGTATATTTGCGA |
| | | AAACCAAGACTCCATTTCCAGTAAACTTAAGGAGTGTTGT |
| | | GAGAAACCTCTCCTTGAAAAGAGCCATTGTATAGCGGAG |
| | | GTCGAAAACGATGAAATGCCCGCTGATCTGCCGTCACTC |
| | | GCTGCTGACTTCGTTGAATCCAAGGATGTATGTAAAAACT |
| | | ATGCCGAAGCTAAGGACGTCTTTCTTGGAATGTTCTTGTA |
| | | TGAATACGCCCGACGGCATCCTGACTATTCAGTTGTGCTC |
| | | CTTCTTCGATTGGCTAAAACTTATGAGACGACACTTGAAA |
| | | AGTGTTGTGCCGCCGCCGACCCACATGAGTGCTACGCTA |
| | | AGGTTTTCGACGAATTTAAGCCCTTGGTTGAAGAACCGCA |
| | | GAATCTCATTAAGCAAAATTGTGAGCTCTTTGAACAACTC |
| | | GGTGAATACAAATTTCAGAACGCGCTTCTGGTTAGATATA |
| | | CGAAGAAAGTGCCACAGGTGAGCACCCCTACATTGGTTG |
| | | AGGTCAGTAGGAACCTCGGCAAGGTGGGATCTAAGTGCT |
| | | GCAAACACCCGGAGGCAAAGAGAATGCCTTGCGCTGAGG |
| | | ATTATCTCTCAGTGGTTCTGAATCAGCTTTGCGTTCTTCAC |
| | | GAAAAGACTCCCGTCAGCGACCGAGTCACGAAGTGCTGT |
| | | ACCGAGAGCCTTGTTAATAGACGACCTTGCTTCTCCGCGC |
| | | TTGAGGTGGATGAGACCTACGTCCCAAAGGAATTCAACG |
| | | CCGAGACGTTCACTTTCCATGCCGATATTTGCACCTTGTC |
| | | AGAAAAGGAGCGACAGATAAAAAAGCAAACTGCTTTGGT |
| | | GGAGCTGGTTAAGCATAAGCCTAAAGCGACGAAGGAACA |
| | | GCTCAAAGCCGTAATGGATGATTTCGCTGCCTTCGTGGAG |
| | | AAATGTTGTAAGGCCGATGACAAGGAAACCTGCTTCGCT |
| | | GAAGAGGGTAAAAAGCTCGTCGCCGCTAGTCAGGCGGCA |
| | | CTTGGTTTGAGCGAACAAAAACTCATTTCCGAGGAAGAT |
| | | CTTtga |
| 124 | ProC1488/ | METDTLLLWVLLLWVPGSTGGSGEQKLISEEDLGDAHKSEV |
| | ProC_cMyc-HSA- | AHRFKDLGEENFKALVLIAFAQYLQQCPFEDHVKLVNEVTE |
| | mLm16-1490DNI- | FAKTCVADESAENCDKSLHTLFGDKLCTVATLRETYGEMA |
| | LIGHT | DCCAKQEPERNECFLQHKDDNPNLPRLVRPEVDVMCTAFH |
| | Protein with signal | DNEETFLKKYLYEIARRHPYFYAPELLFFAKRYKAAFTECC |
| | sequence | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLGGGSSGGSGSQGQSGSRHPCRHDPHIICYKF |
| | | GGGSSGGSISSGLLSGRSDNIGGSQERRSHEVNPAAHLTGAN |
| | | SSLTGSGGPLLWETQLGLAFLRGLSYHDGALVVTKAGYYYI |
| | | YSKVQLGGVGCPLGLASTITHGLYKRTPRYPEELELLVSQQS |
| | | PCGRATSSSRVWWDSSFLGGVVHLEAGEEVVVRVLDERLV |
| | | RLRDGTRSYFGAFMV |
| 125 | ProC1488/ | atggaaaccgacacactgctgctgtgggtgctgcttttgtgggtgccaggatccacaggcGG |
| | ProC_cMyc-HSA- | CTCTGGCGAACAAAAACTCATTTCCGAGGAAGATCTTGG |
| | mLm16-1490DNI- | CGATGCACATAAAAGTGAGGTGGCCCACCGCTTTAAAGA |
| | LIGHT | CCTCGGCGAGGAGAACTTCAAGGCACTCGTACTTATTGCC |
| | DNA with coding | TTTGCACAATACCTGCAGCAATGTCCGTTTGAAGATCATG |
| | sequence for signal | TCAAGCTCGTAAATGAAGTGACAGAGTTCGCTAAGACCT |
| | peptide | GTGTAGCTGACGAAAGTGCCGAAAACTGCGACAAATCAT |
| | | TGCATACATTGTTCGGCGACAAGCTTTGTACAGTTGCCAC |
| | | GCTCCGCGAGACATACGGCGAAATGGCCGATTGTTGTGC |
| | | GAAACAGGAACCCGAAAGAAATGAGTGCTTCTTGCAACA |
| | | CAAAGACGATAATCCCAATCTTCCGCGACTGGTGAGACC |
| | | CGAAGTAGATGTAATGTGCACAGCTTTTCATGACAATGA |
| | | GGAAACCTTTCTTAAAAAATATCTGTACGAAATCGCCCGC |
| | | CGACACCCGTATTTCTATGCCCCAGAGCTGTTGTTTTTCG |
| | | CAAAGCGCTACAAGGCAGCGTTTACaGAATGCTGtCAGGC |
| | | CGCTGATAAAGCCGCTTGTCTGCTGCCTAAGCTTGATGAG |
| | | CTTCGCGACGAGGGTAAGGCTTCCAGCGCTAAACAACGC |
| | | CTGAAGTGTGCATCTTTGCAAAAATTTGGTGAACGAGCCT |
| | | TCAAGGCTTGGGCTGTTGCCAGGCTGAGTCAACGGTTCCC |
| | | GAAGGCGGAGTTTGCGGAGGTTTCCAAGTTGGTAACGGA |
| | | CCTGACAAAGGTACATACCGAATGCTGCCATGGCGACCT |
| | | GTTGGAGTGCGCCGATGATCGAGCGGATCTTGCCAAGTA |
| | | TATTTGCGAAAACCAAGACTCCATTTCCAGTAAACTTAAG |
| | | GAGTGTTGTGAGAAACCTCTCCTTGAAAAGAGCCATTGT |
| | | ATAGCGGAGGTCGAAAACGATGAAATGCCCGCTGATCTG |
| | | CCGTCACTCGCTGCTGACTTCGTTGAATCCAAGGATGTAT |
| | | GTAAAAACTATGCCGAAGCTAAGGACGTCTTTCTTGGAA |
| | | TGTTCTTGTATGAATACGCCCGACGGCATCCTGACTATTC |
| | | AGTTGTGCTCCTTCTTCGATTGGCTAAAACTTATGAGACG |
| | | ACACTTGAAAAGTGTTGTGCCGCCGCCGACCCACATGAG |
| | | TGCTACGCTAAGGTTTTCGACGAATTTAAGCCCTTGGTTG |
| | | AAGAACCGCAGAATCTCATTAAGCAAAATTGTGAGCTCT |
| | | TTGAACAACTCGGTGAATACAAATTTCAGAACGCGCTTCT |
| | | GGTTAGATATACGAAGAAAGTGCCACAGGTGAGCACCCC |
| | | TACATTGGTTGAGGTCAGTAGGAACCTCGGCAAGGTGGG |
| | | ATCTAAGTGCTGCAAACACCCGGAGGCAAAGAGAATGCC |
| | | TTGCGCTGAGGATTATCTCTCAGTGGTTCTGAATCAGCTT |
| | | TGCGTTCTTCACGAAAAGACTCCCGTCAGCGACCGAGTC |
| | | ACGAAGTGCTGTACCGAGAGCCTTGTTAATAGACGACCT |
| | | TGCTTCTCCGCGCTTGAGGTGGATGAGACCTACGTCCCAA |
| | | AGGAATTCAACGCCGAGACGTTCACTTTCCATGCCGATAT |
| | | TTGCACCTTGTCAGAAAAGGAGCGACAGATAAAAAAGCA |
| | | AACTGCTTTGGTGGAGCTGGTTAAGCATAAGCCTAAAGC |
| | | GACGAAGGAACAGCTCAAAGCCGTAATGGATGATTTCGC |
| | | TGCCTTCGTGGAGAAATGTTGTAAGGCCGATGACAAGGA |
| | | AACCTGCTTCGCTGAAGAGGGTAAAAAGCTCGTCGCCGC |
| | | TAGTCAGGCGGCACTTGGTTTGGGGGGCGGAAGCAGCGG |
| | | CGGCAGCGGCAGCCAAGGCCAGAGCGGCAGCAGGCACC |
| | | CTTGTAGACACGATCCTCACATCATCTGTTACAAGTTTGG |
| | | AGGAGGCAGTAGCGGTGGCAGTATTAGCAGCGGACTGCT |
| | | GAGTGGCAGAAGCGACAACATCGGCGGCAGCCAGGAGA |
| | | GACGGAGCCACGAGGTGAACCCCGCTGCCCACCTGACCG |
| | | GCGCCAACAGCAGTCTGACCGGCAGCGGCGGCCCTCTGC |
| | | TGTGGGAGACACAGCTGGGCCTGGCCTTTCTGAGAGGCC |
| | | TGTCCTACCACGACGGCGCCCTGGTCGTGACCAAGGCCG |
| | | GCTACTACTACATCTACAGCAAGGTGCAGCTGGGAGGCG |
| | | TGGGCTGCCCTCTGGGCCTGGCATCTACCATCACGCACGG |
| | | CCTGTACAAGCGGACCCCTAGATATCCTGAGGAACTGGA |
| | | ACTCCTGGTGTCCCAGCAGAGCCCTTGCGGCAGAGCTAC |
| | | AAGCAGCTCAAGAGTGTGGTGGGACAGCAGCTTCCTGGG |
| | | CGGAGTGGTCCACCTGGAAGCTGGAGAAGAAGTGGTGGT |
| | | CCGGGTGCTGGACGAGAGACTCGTGAGGCTGAGAGACGG |
| | | AACAAGAAGCTACTTCGGCGCTTTTATGGTGtga |
| 126 | ProC1498/ | METDTLLLWVLLLWVPGSTGGSGEQKLISEEDLGDAHKSEV |
| | ProC_cMyc-HSA- | AHRFKDLGEENFKALVLIAFAQYLQQCPFEDHVKLVNEVTE |
| | 21GS-1204DNI- | FAKTCVADESAENCDKSLHTLFGDKLCTVATLRETYGEMA |
| | LIGHT-mLm16 | DCCAKQEPERNECFLQHKDDNPNLPRLVRPEVDVMCTAFH |
| | Protein with signal | DNEETFLKKYLYEIARRHPYFYAPELLFFAKRYKAAFTECC |
| | sequence | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLSSAGGGSSGGSLSGRSDNIGSQERRSHEVNP |
| | | AAHLTGANSSLTGSGGPLLWETQLGLAFLRGLSYHDGALV |
| | | VTKAGYYYIYSKVQLGGVGCPLGLASTITHGLYKRTPRYPE |
| | | ELELLVSQQSPCGRATSSSRVWWDSSFLGGVVHLEAGEEVV |
| | | VRVLDERLVRLRDGTRSYFGAFMVGGGSISSGLLSGRSDNI |
| | | GGGSSGGSRHPCRHDPHIICYKF |
| 127 | ProC1498/ | atggaaaccgacacactgctgctgtgggtgctgcttttgtgggtgccaggatccacaggcGG |
| | ProC_cMyc-HSA- | CTCTGGCGAACAAAAACTCATTTCCGAGGAAGATCTTGG |
| | 21GS-1204DNI- | CGATGCACATAAAAGTGAGGTGGCCCACCGCTTTAAAGA |
| | LIGHT-mLm16 | CCTCGGCGAGGAGAACTTCAAGGCACTCGTACTTATTGCC |
| | DNA with coding | TTTGCACAATACCTGCAGCAATGTCCGTTTGAAGATCATG |
| | sequence for signal | TCAAGCTCGTAAATGAAGTGACAGAGTTCGCTAAGACCT |
| | peptide | GTGTAGCTGACGAAAGTGCCGAAAACTGCGACAAATCAT |
| | | TGCATACATTGTTCGGCGACAAGCTTTGTACAGTTGCCAC |
| | | GCTCCGCGAGACATACGGCGAAATGGCCGATTGTTGTGC |
| | | GAAACAGGAACCCGAAAGAAATGAGTGCTTCTTGCAACA |
| | | CAAAGACGATAATCCCAATCTTCCGCGACTGGTGAGACC |
| | | CGAAGTAGATGTAATGTGCACAGCTTTTCATGACAATGA |
| | | GGAAACCTTTCTTAAAAAATATCTGTACGAAATCGCCCGC |
| | | CGACACCCGTATTTCTATGCCCCAGAGCTGTTGTTTTTCG |
| | | CAAAGCGCTACAAGGCAGCGTTTACaGAATGCTGtCAGGC |
| | | CGCTGATAAAGCCGCTTGTCTtCTGCCTAAGCTTGATGAG |
| | | CTTCGCGACGAGGGTAAGGCTTCCAGCGCTAAACAACGC |
| | | CTGAAGTGTGCATCTTTGCAAAAATTTGGTGAACGAGCCT |
| | | TCAAGGCTTGGGCTGTTGCCAGGCTGAGTCAACGGTTCCC |
| | | GAAGGCGGAGTTTGCGGAGGTTTCCAAGTTGGTAACGGA |
| | | CCTGACAAAGGTACATACCGAATGCTGCCATGGCGACCT |
| | | GTTGGAGTGCGCCGATGATCGAGCGGATCTTGCCAAGTA |
| | | TATTTGCGAAAACCAAGACTCCATTTCCAGTAAACTTAAG |
| | | GAGTGTTGTGAGAAACCTCTCCTTGAAAAGAGCCATTGT |
| | | ATAGCGGAGGTCGAAAACGATGAAATGCCCGCTGATCTG |
| | | CCGTCACTCGCTGCTGACTTCGTTGAATCCAAGGATGTAT |
| | | GTAAAAACTATGCCGAAGCTAAGGACGTCTTTCTTGGAA |
| | | TGTTCTTGTATGAATACGCCCGACGGCATCCTGACTATTC |
| | | AGTTGTGCTCCTTCTTCGATTGGCTAAAACTTATGAGACG |
| | | ACACTTGAAAAGTGTTGTGCCGCtGCtGACCCACATGAGT |
| | | GCTACGCTAAGGTTTTCGACGAATTTAAGCCaTTGGTTGA |
| | | AGAACCGCAGAATCTCATTAAGCAAAATTGTGAGCTCTTT |
| | | GAACAACTCGGTGAATACAAATTTCAGAACGCGCTTCTG |
| | | GTTAGATATACGAAGAAAGTGCCACAGGTGAGCACCCCT |
| | | ACATTGGTTGAGGTCAGTAGGAACCTCGGCAAGGTGGGA |
| | | TCTAAGTGCTGCAAACACCCGGAGGCAAAGAGAATGCCT |
| | | TGCGCTGAGGATTATCTCTCAGTGGTTCTGAATCAGCTTT |
| | | GCGTTCTTCACGAAAAGACTCCCGTCAGCGACCGAGTCA |
| | | CGAAGTGCTGTACCGAGAGCCTTGTTAATAGACGACCTT |
| | | GCTTCTCCGCGCTTGAGGTGGATGAGACCTACGTCCCAAA |
| | | GGAATTCAACGCCGAGACGTTCACTTTCCATGCCGATATT |
| | | TGCACCTTGTCAGAAAAGGAGCGACAGATAAAAAAGCAA |
| | | ACTGCTTTGGTGGAGCTGGTTAAGCATAAGCCTAAAGCG |
| | | ACGAAGGAACAGCTCAAAGCCGTAATGGATGATTTCGCT |
| | | GCCTTCGTGGAGAAATGTTGTAAGGCCGATGACAAGGAA |
| | | ACCTGCTTCGCTGAAGAGGGTAAAAAGCTCGTCGCCGCT |
| | | AGTCAGGCGGCACTTGGTTTGTCCAGCGCCGGGGGCGGA |
| | | AGCAGCGGCGGCAGCTTAAGCGGCAGAAGCGACAACAT |
| | | CGGCAGCCAGGAGAGACGGAGCCACGAGGTGAACCCCG |
| | | CTGCCCACCTGACCGGCGCCAACAGCAGTCTGACCGGCA |
| | | GCGGCGGCCCTCTGCTGTGGGAGACACAGCTGGGCCTGG |
| | | CCTTTCTGAGAGGCCTGTCCTACCACGACGGCGCCCTGGT |
| | | CGTGACCAAGGCCGGCTACTACTACATCTACAGCAAGGT |
| | | GCAGCTGGGAGGCGTGGGCTGCCCTCTGGGCCTGGCATC |
| | | TACCATCACGCACGGCCTGTACAAGCGGACCCCTAGATA |
| | | TCCTGAGGAACTGGAACTCCTGGTGTCCCAGCAGAGCCC |
| | | TTGCGGCAGAGCTACAAGCAGCTCAAGAGTGTGGTGGGA |
| | | CAGCAGCTTCCTGGGCGGAGTGGTCCACCTGGAAGCTGG |
| | | AGAAGAAGTGGTGGTCCGGGTGCTGGACGAGAGACTCGT |
| | | GAGGCTGAGAGACGGAACAAGAAGCTACTTCGGCGCTTT |
| | | TATGGTGGGCGGAGGCTCCATTTCTAGCGGCCTGCTGAGC |
| | | GGCAGAAGCGATAACATCGGCGGaGGAAGCAGCGGAGGC |
| | | AGCAGACACCCCTGCAGACACGATCCTCACATCATCTGC |
| | | TACAAGTTCtga |
| 128 | ProC1162/ | METDTLLLWVLLLWVPGSTGQGQSGSRHPCRHDPHIICYKF |
| | ProC_mLm- | GGGSSGGSISSGLLSGRSDNIGGSQERRSHEVNPAAHLTGAN |
| | 16_1490_LIGHT_ | SSLTGSGGPLLWETQLGLAFLRGLSYHDGALVVTKAGYYYI |
| | 11linker_HSA-cMyc | YSKVQLGGVGCPLGLASTITHGLYKRTPRYPEELELLVSQQS |
| | Protein with signal | PCGRATSSSRVWWDSSFLGGVVHLEAGEEVVVRVLDERLV |
| | sequence | RLRDGTRSYFGAFMVSSAGGGSSGGSDAHKSEVAHRFKDL |
| | | GEENFKALVLIAFAQYLQQCPFEDHVKLVNEVTEFAKTCVA |
| | | DESAENCDKSLHTLFGDKLCTVATLRETYGEMADCCAKQE |
| | | PERNECFLQHKDDNPNLPRLVRPEVDVMCTAFHDNEETFLK |
| | | KYLYEIARRHPYFYAPELLFFAKRYKAAFTECCQAADKAAC |
| | | LLPKLDELRDEGKASSAKQRLKCASLQKFGERAFKAWAVA |
| | | RLSQRFPKAEFAEVSKLVTDLTKVHTECCHGDLLECADDRA |
| | | DLAKYICENQDSISSKLKECCEKPLLEKSHCIAEVENDEMPA |
| | | DLPSLAADFVESKDVCKNYAEAKDVFLGMFLYEYARRHPD |
| | | YSVVLLLRLAKTYETTLEKCCAAADPHECYAKVFDEFKPLV |
| | | EEPQNLIKQNCELFEQLGEYKFQNALLVRYTKKVPQVSTPT |
| | | LVEVSRNLGKVGSKCCKHPEAKRMPCAEDYLSVVLNQLCV |
| | | LHEKTPVSDRVTKCCTESLVNRRPCFSALEVDETYVPKEFN |
| | | AETFTFHADICTLSEKERQIKKQTALVELVKHKPKATKEQLK |
| | | AVMDDFAAFVEKCCKADDKETCFAEEGKKLVAASQAALG |
| | | LSGGEQKLISEEDL |
| 129 | ProC1162/ | atggaaaccgacacactgctgctgtgggtgctgcttttgtgggtgccaggatccacaggcCA |
| | ProC_mLm- | AGGGCAATCCGGATCTCGGCATCCGTGTCGGCATGACCC |
| | 16_1490_LIGHT_ | TCACATCATATGCTATAAGTTTGGGGGGGGATCCTCAGGT |
| | 11linker_HSA-cMyc | GGTTCCATTTCTTCTGGTTTGTTGTCTGGAAGATCAGACA |
| | DNA with coding | ACATCGGCGGATCTCAAGAAAGACGCTCTCATGAGGTGA |
| | sequence for signal | ATCCTGCCGCGCACCTTACTGGGGCAAACTCCAGTCTGAC |
| | peptide | CGGATCAGGTGGGCCATTGCTTTGGGAGACACAGTTGGG |
| | | ACTGGCATTCCTTCGAGGCCTCAGCTACCATGATGGCGCC |
| | | CTGGTCGTAACGAAAGCTGGGTATTACTATATATATAGCA |
| | | AAGTCCAACTGGGGGGGGTTGGGTGTCCCCTGGGGCTCG |
| | | CGTCAACCATCACCCACGGACTCTATAAGAGAACACCAA |
| | | GATACCCAGAAGAGTTGGAACTTCTCGTGTCTCAACAAA |
| | | GTCCATGTGGGAGGGCAACATCTTCTTCACGCGTATGGTG |
| | | GGATTCATCCTTTCTCGGTGGCGTTGTCCATCTTGAAGCA |
| | | GGGGAGGAAGTTGTTGTCAGAGTCTTGGATGAAAGACTC |
| | | GTGAGATTGCGGGATGGTACTAGATCCTACTTCGGCGCGT |
| | | TCATGGTCTCCTCAGCGGGAGGTGGGAGTAGTGGCGGCT |
| | | CAGACGCCCATAAATCCGAAGTGGCACATCGATTTAAGG |
| | | ACCTCGGGGAGGAGAACTTCAAAGCTCTGGTACTCATCG |
| | | CCTTTGCTCAGTACTTGCAGCAATGCCCCTTCGAGGATCA |
| | | TGTCAAACTCGTGAACGAGGTTACGGAGTTCGCTAAAAC |
| | | ATGTGTTGCTGACGAGTCTGCAGAGAACTGTGACAAATC |
| | | CCTCCACACGCTGTTCGGTGATAAACTGTGTACGGTGGCT |
| | | ACCCTCAGGGAAACCTACGGAGAGATGGCCGATTGTTGC |
| | | GCCAAACAGGAGCCTGAGAGGAACGAATGCTTTCTTCAA |
| | | CACAAGGATGACAATCCTAACTTGCCTAGGCTGGTTCGG |
| | | CCCGAGGTCGATGTGATGTGCACAGCGTTTCACGACAAC |
| | | GAAGAAACATTCCTGAAGAAATACTTGTACGAGATTGCT |
| | | AGGCGACACCCATATTTCTACGCGCCGGAGCTTCTCTTCT |
| | | TTGCGAAGCGCTACAAGGCTGCATTTACAGAGTGCTGCC |
| | | AAGCCGCTGATAAGGCGGCCTGTCTTCTCCCCAAGCTCG |
| | | ATGAACTCCGAGATGAAGGGAAAGCTTCATCAGCGAAAC |
| | | AAAGATTGAAATGTGCTTCCCTCCAAAAGTTTGGAGAAC |
| | | GAGCCTTTAAGGCTTGGGCAGTGGCACGGCTCAGTCAGC |
| | | GCTTTCCTAAGGCTGAATTTGCCGAAGTGTCCAAGCTTGT |
| | | AACGGATCTCACTAAAGTTCATACTGAATGCTGCCACGG |
| | | AGACCTTCTCGAATGCGCGGACGATCGCGCGGACTTGGC |
| | | GAAATATATATGCGAGAATCAAGATAGTATCAGCAGTAA |
| | | ACTCAAAGAGTGCTGCGAGAAGCCTCTCCTCGAAAAGAG |
| | | CCACTGTATCGCCGAGGTGGAAAATGATGAGATGCCTGC |
| | | GGACTTGCCATCCCTTGCCGCAGACTTTGTCGAATCAAAA |
| | | GACGTTTGCAAGAATTACGCGGAGGCAAAAGATGTATTC |
| | | CTTGGCATGTTCTTGTACGAATACGCACGGCGCCACCCTG |
| | | ACTATTCAGTAGTGTTGCTCTTGAGACTCGCTAAAACATA |
| | | CGAAACGACGCTTGAGAAATGTTGCGCAGCAGCCGATCC |
| | | CCACGAGTGTTACGCAAAGGTGTTCGACGAGTTTAAACC |
| | | CCTCGTTGAAGAACCTCAAAACCTGATAAAACAAAATTG |
| | | TGAGTTGTTCGAGCAGTTGGGAGAGTACAAGTTTCAGAA |
| | | TGCTCTCCTGGTTCGGTACACCAAGAAGGTCCCACAAGTG |
| | | TCCACGCCCACCCTCGTAGAGGTATCACGGAACCTTGGC |
| | | AAGGTCGGTAGCAAGTGCTGCAAACACCCAGAAGCTAAG |
| | | CGCATGCCATGCGCTGAAGACTATCTGTCTGTGGTGCTTA |
| | | ATCAATTGTGTGTACTGCATGAGAAAACTCCTGTGTCCGA |
| | | CCGGGTTACCAAGTGCTGTACCGAGTCACTCGTCAACCG |
| | | GCGACCTTGTTTTTCTGCGCTGGAGGTTGATGAGACGTAT |
| | | GTTCCGAAAGAATTCAACGCCGAGACTTTCACCTTCCATG |
| | | CTGATATATGCACACTCAGTGAAAAAGAACGACAAATAA |
| | | AGAAGCAAACCGCATTGGTCGAGCTGGTCAAGCATAAAC |
| | | CCAAAGCTACAAAAGAACAATTGAAGGCTGTTATGGATG |
| | | ACTTTGCCGCGTTCGTAGAGAAATGCTGCAAGGCAGATG |
| | | ATAAAGAGACATGTTTCGCCGAAGAGGGCAAGAAACTGG |
| | | TGGCCGCCTCTCAAGCTGCACTTGGGCTCTCTGGAGGCGA |
| | | ACAAAAGCTTATAAGCGAAGAGGACTTGTGA |
| 130 | ProC1163/ | METDTLLLWVLLLWVPGSTGQGQSGSRHPCRHDPHIICYKF |
| | ProC_mLm- | GGGSSGGSISSGLLSGRSDNIGGSQERRSHEVNPAAHLTGAN |
| | 16_1490_LIGHT_ | SSLTGSGGPLLWETQLGLAFLRGLSYHDGALVVTKAGYYYI |
| | i21lnker_HSA-cMyc | YSKVQLGGVGCPLGLASTITHGLYKRTPRYPEELELLVSQQS |
| | Protein with signal | PCGRATSSSRVWWDSSFLGGVVHLEAGEEVVVRVLDERLV |
| | sequence | RLRDGTRSYFGAFMVSSAGGGSSGGSSAGGGSSGGSDAHK |
| | | SEVAHRFKDLGEENFKALVLIAFAQYLQQCPFEDHVKLVNE |
| | | VTEFAKTCVADESAENCDKSLHTLFGDKLCTVATLRETYGE |
| | | MADCCAKQEPERNECFLQHKDDNPNLPRLVRPEVDVMCTA |
| | | FHDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 131 | ProC1163/ | atggaaaccgacacactgctgctgtgggtgctgcttttgtgggtgccaggatccacaggcCA |
| | ProC_mLm- | GGGACAGTCCGGATCTAGGCATCCATGTCGGCATGACCC |
| | 16_1490_LIGHT_ | ACATATCATATGCTACAAATTCGGTGGAGGATCTTCTGGA |
| | 21linker_HSA-cMyc | GGCTCAATTTCATCAGGACTCCTGTCCGGGCGCTCCGACA |
| | DNA with coding | ATATTGGTGGCTCCCAGGAGAGGAGATCACATGAAGTCA |
| | sequence for signal | ACCCGGCCGCCCATCTTACTGGTGCCAATAGTAGTCTTAC |
| | peptide | AGGTTCTGGCGGACCCTTGCTTTGGGAGACCCAGCTGGG |
| | | ACTGGCTTTCCTTAGAGGCCTGAGTTATCACGACGGGGCG |
| | | TTGGTCGTTACTAAAGCCGGGTATTACTATATATACTCTA |
| | | AGGTTCAGCTCGGCGGTGTAGGATGCCCACTGGGTCTTGC |
| | | GTCTACAATTACACATGGTCTGTATAAGAGGACTCCCAG |
| | | ATATCCAGAAGAACTGGAATTGCTTGTTTCCCAGCAATCT |
| | | CCGTGCGGACGCGCAACGAGCTCCAGTAGGGTGTGGTGG |
| | | GACTCCAGCTTCCTGGGTGGCGTTGTCCATTTGGAGGCGG |
| | | GAGAGGAAGTAGTGGTGCGGGTCCTTGATGAACGCCTTG |
| | | TACGACTTCGGGACGGTACAAGAAGCTACTTTGGGGCTTT |
| | | CATGGTATCTTCTGCAGGGGGGGGGTCATCCGGCGGAAG |
| | | TTCAGCGGGGGGGGGATCCTCCGGTGGCAGTGATGCACA |
| | | TAAAAGTGAGGTGGCCCACCGCTTTAAAGACCTCGGCGA |
| | | GGAGAACTTCAAGGCACTCGTACTTATTGCCTTTGCACAA |
| | | TACCTGCAGCAATGTCCGTTTGAAGATCATGTCAAGCTCG |
| | | TAAATGAAGTGACAGAGTTCGCTAAGACCTGTGTAGCTG |
| | | ACGAAAGTGCCGAAAACTGCGACAAATCATTGCATACAT |
| | | TGTTCGGCGACAAGCTTTGTACAGTTGCCACGCTCCGCGA |
| | | GACATACGGCGAAATGGCCGATTGTTGTGCGAAACAGGA |
| | | ACCCGAAAGAAATGAGTGCTTCTTGCAACACAAAGACGA |
| | | TAATCCCAATCTTCCGCGACTGGTGAGACCCGAAGTAGA |
| | | TGTAATGTGCACAGCTTTTCATGACAATGAGGAAACCTTT |
| | | CTTAAAAAATATCTGTACGAAATCGCCCGCCGACACCCG |
| | | TATTTCTATGCCCCAGAGCTGTTGTTTTTCGCAAAGCGCT |
| | | ACAAGGCAGCGTTTACCGAATGCTGCCAGGCCGCTGATA |
| | | AAGCCGCTTGTCTGCTGCCTAAGCTTGATGAGCTTCGCGA |
| | | CGAGGGTAAGGCTTCCAGCGCTAAACAACGCCTGAAGTG |
| | | TGCATCTTTGCAAAAATTTGGTGAACGAGCCTTCAAGGCT |
| | | TGGGCTGTTGCCAGGCTGAGTCAACGGTTCCCGAAGGCG |
| | | GAGTTTGCGGAGGTTTCCAAGTTGGTAACGGACCTGACA |
| | | AAGGTACATACCGAATGCTGCCATGGCGACCTGTTGGAG |
| | | TGCGCCGATGATCGAGCGGATCTTGCCAAGTATATTTGCG |
| | | AAAACCAAGACTCCATTTCCAGTAAACTTAAGGAGTGTT |
| | | GTGAGAAACCTCTCCTTGAAAAGAGCCATTGTATAGCGG |
| | | AGGTCGAAAACGATGAAATGCCCGCTGATCTGCCGTCAC |
| | | TCGCTGCTGACTTCGTTGAATCCAAGGATGTATGTAAAAA |
| | | CTATGCCGAAGCTAAGGACGTCTTTCTTGGAATGTTCTTG |
| | | TATGAATACGCCCGACGGCATCCTGACTATTCAGTTGTGC |
| | | TCCTTCTTCGATTGGCTAAAACTTATGAGACGACACTTGA |
| | | AAAGTGTTGTGCCGCCGCCGACCCACATGAGTGCTACGC |
| | | TAAGGTTTTCGACGAATTTAAGCCCTTGGTTGAAGAACCG |
| | | CAGAATCTCATTAAGCAAAATTGTGAGCTCTTTGAACAAC |
| | | TCGGTGAATACAAATTTCAGAACGCGCTTCTGGTTAGATA |
| | | TACGAAGAAAGTGCCACAGGTGAGCACCCCTACATTGGT |
| | | TGAGGTCAGTAGGAACCTCGGCAAGGTGGGATCTAAGTG |
| | | CTGCAAACACCCGGAGGCAAAGAGAATGCCTTGCGCTGA |
| | | GGATTATCTCTCAGTGGTTCTGAATCAGCTTTGCGTTCTTC |
| | | ACGAAAAGACTCCCGTCAGCGACCGAGTCACGAAGTGCT |
| | | GTACCGAGAGCCTTGTTAATAGACGACCTTGCTTCTCCGC |
| | | GCTTGAGGTGGATGAGACCTACGTCCCAAAGGAATTCAA |
| | | CGCCGAGACGTTCACTTTCCATGCCGATATTTGCACCTTG |
| | | TCAGAAAAGGAGCGACAGATAAAAAAGCAAACTGCTTTG |
| | | GTGGAGCTGGTTAAGCATAAGCCTAAAGCGACGAAGGAA |
| | | CAGCTCAAAGCCGTAATGGATGATTTCGCTGCCTTCGTGG |
| | | AGAAATGTTGTAAGGCCGATGACAAGGAAACCTGCTTCG |
| | | CTGAAGAGGGTAAAAAGCTCGTCGCCGCTAGTCAGGCGG |
| | | CACTTGGTTTGTCAGGTGGGGAGCAGAAACTTATCTCCGA |
| | | AGAGGATTTGTAA |
| 132 | ProC1164/ | METDTLLLWVLLLWVPGSTGQGQSGSRHPCRHDPHIICYKF |
| | ProC_mLm- | GGGSSGGSISSGLLSGRSDNIGGSQERRSHEVNPAAHLTGAN |
| | 16_1490_LIGHT_ | SSLTGSGGPLLWETQLGLAFLRGLSYHDGALVVTKAGYYYI |
| | IgG4 WT hinge_HSA- | YSKVQLGGVGCPLGLASTITHGLYKRTPRYPEELELLVSQQS |
| | cMyc | PCGRATSSSRVWWDSSFLGGVVHLEAGEEVVVRVLDERLV |
| | Protein with signal | RLRDGTRSYFGAFMVESKYGPPCPSCPAPEFLGGPSDAHKS |
| | sequence | EVAHRFKDLGEENFKALVLIAFAQYLQQCPFEDHVKLVNEV |
| | | TEFAKTCVADESAENCDKSLHTLFGDKLCTVATLRETYGEM |
| | | ADCCAKQEPERNECFLQHKDDNPNLPRLVRPEVDVMCTAF |
| | | HDNEETFLKKYLYEIARRHPYFYAPELLFFAKRYKAAFTEC |
| | | CQAADKAACLLPKLDELRDEGKASSAKQRLKCASLQKFGE |
| | | RAFKAWAVARLSQRFPKAEFAEVSKLVTDLTKVHTECCHG |
| | | DLLECADDRADLAKYICENQDSISSKLKECCEKPLLEKSHCI |
| | | AEVENDEMPADLPSLAADFVESKDVCKNYAEAKDVFLGMF |
| | | LYEYARRHPDYSVVLLLRLAKTYETTLEKCCAAADPHECY |
| | | AKVFDEFKPLVEEPQNLIKQNCELFEQLGEYKFQNALLVRY |
| | | TKKVPQVSTPTLVEVSRNLGKVGSKCCKHPEAKRMPCAED |
| | | YLSVVLNQLCVLHEKTPVSDRVTKCCTESLVNRRPCFSALE |
| | | VDETYVPKEFNAETFTFHADICTLSEKERQIKKQTALVELVK |
| | | HKPKATKEQLKAVMDDFAAFVEKCCKADDKETCFAEEGK |
| | | KLVAASQAALGLSGGEQKLISEEDL |
| 133 | ProC1164/ | atggaaaccgacacactgctgctgtgggtgctgcttttgtgggtgccaggatccacaggcCA |
| | ProC_mLm- | AGGCCAGAGTGGGAGTAGGCATCCATGTCGCCATGATCC |
| | 16_1490_LIGHT_ | TCACATTATATGCTATAAATTTGGTGGGGGAAGCAGTGGT |
| | IgG4 WT hinge_HSA- | GGGTCCATCAGTAGTGGGCTGTTGTCCGGTCGGAGTGAT |
| | cMyc | AATATTGGAGGCTCCCAGGAACGCAGGAGTCATGAAGTT |
| | DNA with coding | AACCCGGCGGCACATCTTACGGGTGCGAACTCTAGTCTTA |
| | sequence for signal | CCGGGAGTGGGGGGCCCCTGCTTTGGGAGACACAGCTTG |
| | peptide | GGCTCGCTTTCCTCAGGGGACTCTCTTACCATGATGGCGC |
| | | ACTGGTAGTAACCAAAGCTGGATACTATTATATTTACTCT |
| | | AAAGTTCAATTGGGGGGCGTTGGCTGCCCACTCGGCCTC |
| | | GCATCTACTATCACCCATGGGTTGTATAAGCGGACCCCTA |
| | | GATACCCTGAGGAACTTGAGCTTTTGGTTTCTCAACAGTC |
| | | ACCTTGCGGTAGGGCTACCTCATCAAGCCGCGTTTGGTGG |
| | | GACAGTTCATTTCTCGGGGGCGTAGTCCATCTGGAGGCA |
| | | GGTGAGGAAGTAGTGGTTCGAGTGCTCGACGAGCGCCTC |
| | | GTACGACTCCGGGATGGGACGAGAAGTTATTTCGGCGCC |
| | | TTTATGGTAGAGTCAAAATACGGGCCTCCCTGTCCCTCTT |
| | | GTCCGGCACCCGAATTTCTTGGCGGGCCCTCTGATGCCCA |
| | | CAAGTCTGAAGTTGCGCATAGATTTAAAGACCTCGGAGA |
| | | GGAGAATTTTAAAGCACTCGTGCTTATCGCATTTGCGCAG |
| | | TACCTGCAGCAGTGCCCTTTCGAGGACCACGTCAAGCTTG |
| | | TCAACGAGGTGACAGAATTCGCCAAAACATGTGTCGCCG |
| | | ATGAGTCTGCCGAGAACTGTGACAAAAGTTTGCATACCC |
| | | TGTTCGGCGACAAACTCTGCACTGTAGCAACTCTTAGGGA |
| | | AACATACGGGGAAATGGCAGACTGTTGCGCGAAACAGGA |
| | | GCCAGAACGAAACGAATGTTTCTTGCAGCACAAGGATGA |
| | | TAACCCTAATCTCCCGAGGTTGGTCAGGCCCGAAGTCGA |
| | | CGTAATGTGCACGGCTTTCCATGACAATGAGGAAACCTTC |
| | | CTCAAGAAGTACCTCTACGAAATAGCACGAAGACATCCG |
| | | TATTTCTATGCTCCTGAGCTGCTCTTTTTCGCTAAACGCTA |
| | | TAAGGCCGCATTCACGGAGTGTTGTCAAGCCGCCGATAA |
| | | GGCTGCTTGCCTTCTGCCCAAGTTGGACGAACTGCGCGAT |
| | | GAGGGGAAAGCATCATCAGCAAAGCAACGCCTGAAATGC |
| | | GCTAGTTTGCAAAAATTCGGGGAACGCGCCTTCAAAGCT |
| | | TGGGCTGTCGCGCGGCTCTCACAGAGGTTCCCCAAGGCT |
| | | GAGTTCGCCGAAGTTTCCAAGCTGGTTACTGATTTGACCA |
| | | AAGTGCACACAGAATGTTGCCACGGCGACCTGCTTGAGT |
| | | GTGCGGATGACCGCGCAGATCTCGCGAAGTATATATGCG |
| | | AAAACCAGGATTCAATTAGCTCTAAATTGAAAGAATGTT |
| | | GTGAGAAACCTCTGCTTGAAAAGTCACACTGCATTGCGG |
| | | AGGTGGAAAATGATGAAATGCCCGCAGATCTCCCCTCTTT |
| | | GGCAGCGGACTTCGTGGAGAGTAAAGACGTCTGTAAGAA |
| | | TTACGCCGAGGCGAAGGATGTATTCCTGGGGATGTTTCTC |
| | | TATGAATACGCTCGCAGACATCCTGACTACTCTGTGGTGC |
| | | TGCTGTTGCGCCTCGCTAAGACCTACGAAACAACCCTGG |
| | | AAAAATGTTGTGCCGCAGCTGATCCACACGAATGCTATG |
| | | CTAAAGTCTTTGATGAATTTAAGCCTTTGGTTGAGGAGCC |
| | | CCAGAACCTGATAAAACAGAACTGTGAATTGTTCGAGCA |
| | | ACTTGGAGAGTATAAATTCCAAAACGCGCTTCTCGTGCG |
| | | GTACACCAAGAAGGTGCCTCAAGTCAGTACTCCAACCCT |
| | | TGTGGAGGTAAGTCGCAATCTCGGTAAAGTTGGCAGTAA |
| | | ATGTTGTAAGCATCCTGAAGCGAAGCGCATGCCTTGTGC |
| | | AGAGGACTATCTGTCAGTAGTTCTTAACCAGCTGTGTGTG |
| | | CTTCATGAGAAAACACCCGTGTCCGACAGGGTGACAAAG |
| | | TGTTGCACTGAGAGTCTCGTGAACCGGAGACCTTGTTTCT |
| | | CCGCCCTGGAAGTGGACGAAACCTATGTGCCGAAGGAAT |
| | | TCAATGCTGAAACGTTTACGTTCCATGCCGACATCTGCAC |
| | | GCTTAGCGAGAAAGAGAGACAGATAAAGAAGCAAACCG |
| | | CCTTGGTGGAACTCGTTAAGCACAAGCCAAAAGCAACCA |
| | | AAGAACAACTCAAAGCGGTCATGGACGATTTCGCTGCAT |
| | | TTGTAGAGAAATGCTGCAAAGCGGATGACAAGGAGACGT |
| | | GTTTCGCTGAAGAGGGAAAAAAGCTCGTGGCAGCATCCC |
| | | AGGCTGCACTTGGTCTGTCTGGTGGTGAGCAAAAACTGA |
| | | TTTCAGAGGAGGATTTGTGA |
| 134 | ProC2076/ | METDTLLLWVLLLWVPGSTGGSGEQKLISEEDLGDAHKSEV |
| | ProC_cMyc-HSA- | AHRFKDLGEENFKALVLIAFAQYLQQCPFEDHVKLVNEVTE |
| | mLm16-1490- | FAKTCVADESAENCDKSLHTLFGDKLCTVATLRETYGEMA |
| | mhLIGHT | DCCAKQEPERNECFLQHKDDNPNLPRLVRPEVDVMCTAFH |
| | Protein with signal | DNEETFLKKYLYEIARRHPYFYAPELLFFAKRYKAAFTECC |
| | sequence | QAADKAACLLPKLDELRDEGKASSAKQRLKCASLQKFGER |
| | | AFKAWAVARLSQRFPKAEFAEVSKLVTDLTKVHTECCHGD |
| | | LLECADDRADLAKYICENQDSISSKLKECCEKPLLEKSHCIA |
| | | EVENDEMPADLPSLAADFVESKDVCKNYAEAKDVFLGMFL |
| | | YEYARRHPDYSVVLLLRLAKTYETTLEKCCAAADPHECYA |
| | | KVFDEFKPLVEEPQNLIKQNCELFEQLGEYKFQNALLVRYT |
| | | KKVPQVSTPTLVEVSRNLGKVGSKCCKHPEAKRMPCAEDY |
| | | LSVVLNQLCVLHEKTPVSDRVTKCCTESLVNRRPCFSALEV |
| | | DETYVPKEFNAETFTFHADICTLSEKERQIKKQTALVELVKH |
| | | KPKATKEQLKAVMDDFAAFVEKCCKADDKETCFAEEGKKL |
| | | VAASQAALGLGGGSSGGSGSQGQSGSRHPCRHDPHIICYKF |
| | | GGGSSGGSISSGLLSGRSDNIGGSQERRSHEVNPAAHLTGAN |
| | | SSLTGSGGPLLWETQLGLAFLRGLSYHDGALVVTKTGYYYI |
| | | YSKVQLGGVGCPLGLAGTITHGLYKRTPRYPEELELLVSQQ |
| | | SPCGRATSSSRVWWDSSFLGGVVHLEAGEEVVVRVLGKRL |
| | | VRLRDGTRSYFGAFMV |
| 135 | ProC2076/ | atggaaaccgacacactgctgctgtgggtgctgcttttgtgggtgccaggatccacaggcGG |
| | ProC_cMyc-HSA- | CTCTGGCGAACAAAAACTCATTTCCGAGGAAGATCTTGG |
| | mLm16-1490- | CGATGCACATAAAAGTGAGGTGGCCCACCGCTTTAAAGA |
| | mbLIGHT | CCTCGGCGAGGAGAACTTCAAGGCACTCGTACTTATTGCC |
| | DNA with coding | TTTGCACAATACCTGCAGCAATGTCCGTTTGAAGATCATG |
| | sequence for signal | TCAAGCTCGTAAATGAAGTGACAGAGTTCGCTAAGACCT |
| | peptide | GTGTAGCTGACGAAAGTGCCGAAAACTGCGACAAATCAT |
| | | TGCATACATTGTTCGGCGACAAGCTTTGTACAGTTGCCAC |
| | | GCTCCGCGAGACATACGGCGAAATGGCCGATTGTTGTGC |
| | | GAAACAGGAACCCGAAAGAAATGAGTGCTTCTTGCAACA |
| | | CAAAGACGATAATCCCAATCTTCCGCGACTGGTGAGACC |
| | | CGAAGTAGATGTAATGTGCACAGCTTTTCATGACAATGA |
| | | GGAAACCTTTCTTAAAAAATATCTGTACGAAATCGCCCGC |
| | | CGACACCCGTATTTCTATGCCCCAGAGCTGTTGTTTTTCG |
| | | CAAAGCGCTACAAGGCAGCGTTTACaGAATGCTGtCAGGC |
| | | CGCTGATAAAGCCGCTTGTCTGCTGCCTAAGCTTGATGAG |
| | | CTTCGCGACGAGGGTAAGGCTTCCAGCGCTAAACAACGC |
| | | CTGAAGTGTGCATCTTTGCAAAAATTTGGTGAACGAGCCT |
| | | TCAAGGCTTGGGCTGTTGCCAGGCTGAGTCAACGGTTCCC |
| | | GAAGGCGGAGTTTGCGGAGGTTTCCAAGTTGGTAACGGA |
| | | CCTGACAAAGGTACATACCGAATGCTGCCATGGCGACCT |
| | | GTTGGAGTGCGCCGATGATCGAGCGGATCTTGCCAAGTA |
| | | TATTTGCGAAAACCAAGACTCCATTTCCAGTAAACTTAAG |
| | | GAGTGTTGTGAGAAACCTCTCCTTGAAAAGAGCCATTGT |
| | | ATAGCGGAGGTCGAAAACGATGAAATGCCCGCTGATCTG |
| | | CCGTCACTCGCTGCTGACTTCGTTGAATCCAAGGATGTAT |
| | | GTAAAAACTATGCCGAAGCTAAGGACGTCTTTCTTGGAA |
| | | TGTTCTTGTATGAATACGCCCGACGGCATCCTGACTATTC |
| | | AGTTGTGCTCCTTCTTCGATTGGCTAAAACTTATGAGACG |
| | | ACACTTGAAAAGTGTTGTGCCGCCGCCGACCCACATGAG |
| | | TGCTACGCTAAGGTTTTCGACGAATTTAAGCCCTTGGTTG |
| | | AAGAACCGCAGAATCTCATTAAGCAAAATTGTGAGCTCT |
| | | TTGAACAACTCGGTGAATACAAATTTCAGAACGCGCTTCT |
| | | GGTTAGATATACGAAGAAAGTGCCACAGGTGAGCACCCC |
| | | TACATTGGTTGAGGTCAGTAGGAACCTCGGCAAGGTGGG |
| | | ATCTAAGTGCTGCAAACACCCGGAGGCAAAGAGAATGCC |
| | | TTGCGCTGAGGATTATCTCTCAGTGGTTCTGAATCAGCTT |
| | | TGCGTTCTTCACGAAAAGACTCCCGTCAGCGACCGAGTC |
| | | ACGAAGTGCTGTACCGAGAGCCTTGTTAATAGACGACCT |
| | | TGCTTCTCCGCGCTTGAGGTGGATGAGACCTACGTCCCAA |
| | | AGGAATTCAACGCCGAGACGTTCACTTTCCATGCCGATAT |
| | | TTGCACCTTGTCAGAAAAGGAGCGACAGATAAAAAAGCA |
| | | AACTGCTTTGGTGGAGCTGGTTAAGCATAAGCCTAAAGC |
| | | GACGAAGGAACAGCTCAAAGCCGTAATGGATGATTTCGC |
| | | TGCCTTCGTGGAGAAATGTTGTAAGGCCGATGACAAGGA |
| | | AACCTGCTTCGCTGAAGAGGGTAAAAAGCTCGTCGCCGC |
| | | TAGTCAGGCGGCACTTGGTTTGGGGGGCGGAAGCAGCGG |
| | | CGGCAGCGGCAGCCAAGGCCAGAGCGGCAGCAGGCACC |
| | | CTTGTAGACACGATCCTCACATCATCTGTTACAAGTTTGG |
| | | AGGAGGCAGTAGCGGTGGCAGTATTAGCAGCGGACTGCT |
| | | GAGTGGCAGAAGCGACAACATCGGCGGCAGCCAAGAAC |
| | | GCCGATCACACGAAGTGAACCCTGCTGCTCACTTGACTG |
| | | GAGCTAACTCCAGCCTTACGGGGAGCGGGGGTCCTTTGC |
| | | TCTGGGAAACGCAGCTGGGTCTCGCGTTTCTTAGAGGGCT |
| | | GAGTTATCACGATGGCGCCTTGGTCGTCACGAAGACTGG |
| | | GTACTACTATATTTATAGTAAGGTACAGTTGGGGGGAGT |
| | | GGGGTGCCCTCTCGGCCTGGCTGGGACGATCACGCATGG |
| | | GCTCTACAAGCGCACGCCTAGGTATCCGGAGGAGTTGGA |
| | | ACTGTTGGTCTCACAGCAGTCCCCGTGCGGGCGAGCGAC |
| | | TTCCTCCAGTAGAGTGTGGTGGGACTCTAGCTTTCTCGGG |
| | | GGAGTGGTTCATCTTGAGGCAGGAGAGGAAGTGGTGGTA |
| | | CGGGTTTTGGGCAAGAGATTGGTGAGACTGCGGGATGGT |
| | | ACAAGATCTTATTTTGGTGCTTTCATGGTCtgaaccggttagtaatg |
| | | agtttgatatctcgacaatcaacctctggattacaaaatttgtgaaagattgactggta |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An activatable cytokine construct (ACC) comprising:
a first monomer construct comprising a first cytokine protein (CP1), a first cleavable moiety (CM1), and a first steric masking moiety (SMM1), wherein the CM1 is positioned between the CP1 and the SMM1;
a second monomer construct comprising a second cytokine protein (CP2), a second cleavable moiety (CM2), and a second steric masking moiety (SMM2), wherein the CM2 is positioned between the CP2 and the SMM2; and
a third monomer construct comprising a third cytokine protein (CP3), a third cleavable moiety (CM3), and a third steric masking moiety (SMM3), wherein the CM3 is positioned between the CP3 and the SMM3,
wherein:
the CP1, the CP2, and the CP3 bind to one another thereby forming a trimer of the first, the second, and the third monomer constructs; and
the SMM1, the SMM2, and the SMM3 are globular molecules.

2. The ACC of claim 1, wherein the CP1, the CP2, and the CP3 are the same cytokine and/or wherein the SMM1, the SMM2, and the SMM3 are the same globular molecule.

3. The ACC of claim 2, wherein the cytokine is a member of tumor necrosis factor or tumor necrosis factor super family.

4. The ACC of claim 2, wherein the CP1, the CP2 and the CP3 are tumor necrosis factor superfamily member 14 (TNFSF14).

5. The ACC of claim 2, wherein each of the CP1, the CP2, and the CP3 comprises a sequence that is at least 80%, 90%, 95%, or 99% identical to SEQ ID NO: 54.

6. (canceled)

7. The ACC of claim 1, wherein the globular molecule is an albumin.

8. The ACC of claim 7, wherein the albumin is a human serum albumin.

9. (canceled)

10. The ACC of claim 1, wherein the first monomer construct comprises at least one linker, the second monomer construct comprises at least one linker, and/or the third monomer construct comprises at least one linker.

11. The ACC of claim 10, wherein the at least one linker comprises a linker L1 disposed between the CP1 and the CM1, and/or a linker L2 between the CM1 and the SMM1; or wherein the at least one linker comprises a linker L3 disposed between the CP2 and the CM2, and/or a linker L4 between the CM2 and the SMM2; or wherein the at least one linker comprises a linker L5 disposed between the CP3 and the CM3, and/or a linker L6 between the CM3 and the SMM3.

12. (canceled)

13. (canceled)

14. (canceled)

15. (canceled)

16. The ACC of claim 1, wherein:
the first monomer construct further comprises a first affinity masking moiety (AMM1) and optionally a fourth cleavable moiety (CM4) positioned between the AMM1 and the CP1,
the second monomer construct further comprises a second affinity masking moiety (AMM2) and optionally a fifth cleavable moiety (CM5) positioned between the AMM2 and the CP2, and
the third monomer construct further comprises a third affinity masking moiety (AMM3) and optionally a sixth cleavable moiety (CM6) positioned between the AMM3 and the CP3.

17. The ACC of claim 16, wherein the AMM1, the AMM2, and the AMM3 are the same, optionally wherein each of AMM1, the AMM2, and the AMM3 comprises a sequence of SEQ ID NO: 61.

18. (canceled)

19. The ACC of claim 16, wherein the each of AMM1, the AMM2, and the AMM3 comprises a sequence that at least 80%, 90%, 95%, or 99% identical to SEQ ID NO: 61.

20. The ACC of claim 1, wherein the CM1, the CM2, and the CM3 comprise a substrate of the same protease.

21. The ACC of claim 1, wherein the CM1, the CM2, and the CM3 comprise substrates of different proteases.

22. The ACC of claim 1, wherein each of the CM1, the CM2, and the CM3 comprises a sequence that is at least 95% identical to SEQ ID NO: 62 or 63.

23. The ACC of claim 16, wherein the CM4, the CM5, and the CM6 comprise a substrate of the same protease, optionally wherein each of the CM4, the CM5, and the CM6 comprises a sequence that is at least 95% identical to SEQ ID NO: 62 or 63.

24. The ACC of claim 16, wherein the CM4, the CM5, and the CM6 comprise substrates of different proteases.

25. (canceled)

26. (canceled)

27. The ACC of claim 20, wherein the protease(s) is/are selected from the group consisting of: ADAMS, ADAM9, ADAM10, ADAM12, ADAM15, ADAM17/TAC, ADAMDEC1, ADAMTS1, ADAMTS4, ADAMTS5, BACE, Renin, Cathepsin D, Cathepsin E, Caspase 1, Caspase 2, Caspase 3, Caspase 4, Caspase 5, Caspase 6, Caspase 7, Caspase 8, Caspase 9, Caspase 10, Caspase 14, Cathepsin B, Cathepsin C, Cathepsin K, Cathespin L, Cathepsin S, Cathepsin V/L2, Cathepsin X/Z/P, Cruzipain, Legumain, Otubain-2, KLK4, KLK5, KLK6, KLK7, KLK8, KLK10, KLK11, KLK13, KLK14, Meprin, Neprilysin, PSMA, BMP-1, MMP-1, MMP-2, MMP-3, MMP-7, MMP-9, MMP-10, MMP-11, MMP-12, MMP-13, MMP-14, MMP-15, MMP-16, MMP-17, MMP-19, MMP-20, MMP-23, MMP-24, MMP-26, MMP-27, activated protein C, cathepsin A, cathepsin G, Chymase, FVIIa, FIXa, FXa, FXIa, FXIIa, Elastase, Granzyme B, Guanidinobenzoatase, HtrA1, human neutrophil lyase, lactoferrin, marapsin, NS3/4A, PACE4, Plasmin, PSA, tPA, thrombin, tryptase, uPA, DESC1, DPP-4, FAP, Hepsin, Matriptase-2, MT-SP1/Matripase, TMPRSS2, TMPRSS3, and TMPRSS4.

28. The ACC of claim 16, wherein the first monomer construct further comprises a linker L7 between the AMM1 and the CM4 and/or a linker L8 between the CM4 and the CP1, the second monomer construct further comprises a linker L9 between the AMM2 and the CM5 and/or a linker L10 between the CM5 and the CP2, and/or the third monomer construct further comprises a linker L11 between the AMM3 and the CM6 and/or a linker L12 between the CM6 and the CP3.

29. (canceled)

30. (canceled)

31. The ACC of claim 11, wherein each of the linkers L1-L12 has a total length of 2 to 30 amino acids.

32. The ACC of claim 11, wherein each of the linkers L1-L12 independently comprises a sequence of any one of SEQ ID NO: 64-69, 75-77, GGS, SGG, GSG, GS, or G.

33. The ACC of claim 1, wherein in a N- to C-terminal direction:
the first monomer construct comprises the CP1, the CM1, and the SMM1,
the second monomer construct comprises the CP2, the CM2, and the SMM2, and
the third monomer construct comprises the CP3, the CM3, and the SMM3.

34. The ACC of claim 1, wherein in a N- to C-terminal direction:
the first monomer construct comprises the SMM1, the CM1, and the CP1,
the second monomer construct comprises the SMM2, the CM2, and the CP2, and
the third monomer construct comprises the SMM3, the CM3, and the CP3.

35. The ACC of claim 16, wherein in a N- to C-terminal direction:
the first monomer construct comprises the AMM41, the CM4, the CP1, the CM1, and the SMM1,
the second monomer construct comprises the AMM2, the CM5, the CP2, the CM2, and the SMM2, and
the third monomer construct comprises the AMM3, the CM6, the CP3, the CM3, and the SMM3.

36. The ACC of claim 16, wherein in a N- to C-terminal direction:
the first monomer construct comprises the SMM1, the AMM1, the CM1, and the CP1;
the second monomer construct comprises the SMM2, the AMM2, the CM2, and the CP2, and
the third monomer construct comprises the SMM3, the AMM3, the CM3, and the CP3.

37. The ACC of claim 16, wherein in a N- to C-terminal direction:
the first monomer construct comprises, the AMM1, the SMM1, the CM1, and the CP1;
the second monomer construct comprises the AMM2, the SMM2, the CM2, and the CP2, and
the third monomer construct comprises the AMM3, the SMM3, the CM3, and the CP3.

38. The ACC of claim 16, wherein in a N- to C-terminal direction:
the first monomer construct comprises the SMM1, the CM1, the CP1, the CM4, and AMM1,
the second monomer construct comprises the SMM2, the CM2, the CP2, the CM5, and AMM2, and
the third monomer construct comprises the SMM3, the CM3, the CP3, the CM6, and the AMM3.

39. The ACC of claim 16, wherein in a N- to C-terminal direction:
the first monomer construct comprises the CP1, the CM1, the AMM1, and the SMM1;
the second monomer construct comprises the CP2, the CM2, the AMM2, and the SMM2; and
the third monomer construct comprises the CP3, the CM3, the AMM3, and the SMM3.

40. The ACC of claim 16, wherein in a N- to C-terminal direction:
the first monomer construct comprises the CP1, the CM1, the SMM1, and the AMM1;
the first monomer construct comprises the CP2, the CM2, the SMM2, and the AMM2; and
the first monomer construct comprises the CP3, the CM3, the SMM3, and the AMM3.

41. The ACC of claim 1, wherein, in an inactive state, the ACC is characterized by having a reduced level of an activity of at least one of the CP1, the CP2, the CP3, or the trimer thereof as compared to a control level of the activity of at least one of the CP1, the CP2, the CP3, or the trimer thereof, or
wherein the ACC is characterized by at least a 2-fold, 5-fold, 10-fold, 500-fold, 10³-fold, 10⁴-fold, 10⁵-fold or 10⁶-fold reduction in the activity of the trimer of CP1, CP2 and CP3 as compared to the activity of a control trimer of CP1, CP2, and CP3 that does not comprise a steric masking moiety or an affinity masking moiety, and
optionally wherein the activity is activation of herpes virus entry mediator (HVEM) or wherein the activity is activation of lymphotoxin beta receptor or wherein the activity is activation of herpes virus ent mediator (HVEM) and activation of lymphotoxin beta receptor.

42. (canceled)

43. (canceled)

44. (canceled)

45. (canceled)

46. The ACC of claim 41, wherein the control trimer of CP1, CP2, and CP3 results from activation of the ACC.

47. An activatable cytokine construct (ACC) comprising:
a first monomer construct comprising a first cytokine protein (CP1), a first cleavable moiety (CM1), and a first affinity masking moiety (AMM1), wherein the CM1 is positioned between the CP1 and the AMM1;
a second monomer construct comprising a second cytokine protein (CP2), a second cleavable moiety (CM2), and a second affinity masking moiety (AMM2), wherein the CM2 is positioned between the CP2 and the AMM2; and
a third monomer construct comprising a third cytokine protein (CP3), a third cleavable moiety (CM3), and a third affinity steric masking moiety (AMM3), wherein the CM3 is positioned between the CP3 and the AMM3,
wherein the CP1, the CP2, and the CP3 bind to one another thereby forming a trimer of the first, the second, and the third monomer constructs.

48. The ACC of claim 47, wherein the first monomer construct, the second monomer construct, and the third monomer construct are identical, or wherein the ACC does not comprise any domain that facilitates formation of a trimer other than CP1, CP2 and CP3 or wherein the ACC does not comprise any domain other than the CP1, the CP2, and the CP3 that covalently links the first, the second, and the third monomer constructs, or wherein the ACC does not comprise a coil-coiled domain, an Fc domain, or a domain other than the CP1, the CP2, or the CP3 that is capable forming a disulfide bond.

49. (canceled)

50. (canceled)

51. (canceled)

52. The ACC of claim 47, wherein the CP1, CP2, and CP3 are identical and each comprises the amino acid sequence of SEQ ID NO: 54.

53. The ACC of claim 47, wherein the first monomer construct, the second monomer construct, and the third monomer construct are identical and each monomer comprises:
a. the amino acid sequence of SEQ ID NO: 54; and
b. an AMM comprising an amino acid sequence that is at least 95% identical to that of SEQ ID NO: 61; and
c. an SMM comprising an albumin.

54. A composition comprising the ACC of claim 1, optionally wherein the composition is a pharmaceutical composition.

55. (canceled)

56. A container, vial, syringe, injector pen, or kit comprising at least one dose of the composition of claim 54.

57. A nucleic acid encoding a polypeptide that comprises at least one of the first monomer construct, the second monomer construct, or the third monomer construct of the ACC of claim 1.

58. The nucleic acid of claim 57, comprising a sequence of any one of SEQ D NOs: 9, 11, 13, 25, 31, 41, 43, 45, or 47.

59. A set of nucleic acids that together encode polypeptides that comprise the first monomer construct, the second monomer construct, and the third monomer construct in the ACC of claim 1.

60. A vector comprising the nucleic acid or a set of nucleic acids of claim 57.

61. A cell comprising the nucleic acid of claim 57.

62. A method of treating a subject in need thereof comprising administering to the subject a therapeutically effective amount of the ACC of claim 1, optionally further comprising administering an immune checkpoint inhibitor, optionally wherein the immune checkpoint inhibitor is an anti-PD-1 antibody or anti-PD-L1 antibody.

63. The method of claim 62, wherein the subject has been identified or diagnosed as having a cancer.

64. (canceled)

65. (canceled)

66. A method of producing an ACC comprising:
culturing the cell of claim 61 in a liquid culture medium under conditions sufficient to produce the ACC; and
recovering the ACC from the cell or the liquid culture medium.

67. The method of claim 66, further comprising purifying the recovered ACC using affinity chromatography.

68. The method of claim 66, further comprising formulating the recovered ACC into a pharmaceutical composition.


SEQUENCE LISTING (678 sequences)

SEQ ID NO: 1 - protein, 5 aa, synthetic construct
GQSGS

SEQ ID NO: 2 - protein, 172 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGHHHHHH

SEQ ID NO: 3 - DNA, 519 bp, synthetic construct
  source 1..519: mol_type = other DNA
caggaacggcgctcccacgaggtcaatccggcggctcatttgactggagccaactctagtcttaccgggagcggcggtccattgctgtgggagacgcaacttggattggcttttcttcggggattgtcttaccacgatggagcactcgttgttacgaaagccggttactactacatttattctaaggtccagttgggtggagttggctgtcccctgggacttgcaagcactatcactcacggactgtacaagaggacaccccgataccccgaggaactcgagctgcttgtgagccaacagtctccatgtgggcgcgcaactagttcttcacgggtgtggtgggactccagtttcctgggaggtgttgttcatctcgaggcaggtgaagaagtagttgtcagggtacttgatgaacggcttgttagactccgggatggaacgaggtcctatttcggtgcgttcatggtaagcgggggcggaagtgggggcgggtcaggtcatcatcaccaccatcattga

SEQ ID NO: 4 - protein, 217 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGHHHHHH

SEQ ID NO: 5 - DNA, 654 bp, synthetic construct
  source 1..654: mol_type = other DNA
caaggacaaagtggttcccgacacccgtgcaggcatgacccacatataatctgttataagtttggtggaggctcctccggcgggtctatcagttccggtctcctcagcggtcgcagtgataatatcggcggttctcaggaacgcaggtcacacgaggtaaatccagccgcccatttgactggcgctaatagttctcttacgggtagcggtggccctcttctgtgggaaacgcagttgggcttggctttccttagaggtctcagctatcacgacggcgccctcgtcgttacaaaggccgggtactattatatttattctaaggtacagcttggcggggtggggtgcccgctgggtcttgcatctacgatcactcatggtctctacaaaagaacaccaaggtatccagaagaacttgagctcctcgttagccaacagtccccctgcggacgggctacttcctctagccgggtatggtgggatagcagtttccttgggggggtcgtgcatctcgaggctggagaggaggtagtagtcagggtcttggatgagagattggtaaggcttcgagacggtacaagatcatactttggggccttcatggtgagcgggggtggcagcggcggtggcagcgggcatcatcaccatcaccattga

SEQ ID NO: 6 - protein, 176 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGSAWSHPQFEK

SEQ ID NO: 7 - DNA, 531 bp, synthetic construct
  source 1..531: mol_type = other DNA
caggaacgcaggtcccatgaagtaaacccggcggcccatcttacgggtgcaaattcaagcttgacaggcagcggagggccgctcctgtgggagacgcaacttgggcttgcgtttctccggggcctgtcatatcacgacggtgctcttgtggtaacgaaagcagggtactattatatatatagcaaggttcagttgggtggggttggatgcccgcttggactcgcgtccactattactcacgggttgtataagaggacaccgcgatacccagaggagctggaactccttgtttcacagcagtctccttgcgggagagccacatcttcatccagggtctggtgggactcttctttcttgggcggcgttgttcatctggaagcgggggaagaggttgtagtgagggtactcgacgagaggctggtgaggctgcgagacgggacccgctcatatttcggagccttcatggtatcaggagggggctccggtggggggtcaggaagcgcatggtcacaccctcagttcgagaaatga

SEQ ID NO: 8 - protein, 221 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGSAWSHPQFEK

SEQ ID NO: 9 - DNA, 666 bp, synthetic construct
  source 1..666: mol_type = other DNA
caaggacagagtggaagtcgacacccttgtaggcacgacccacatataatctgctataaatttggtgggggttccagcggtggaagtatcagctccggccttcttagtggcaggagtgacaatattggcggatctcaagagcgacgcagtcacgaggtaaatcctgcagcacacttgacaggtgctaacagctcacttaccggaagtggggggccgctgttgtgggagacgcagctcgggttggcctttcttcgagggctctcttatcacgacggtgcattggtggtgaccaaagcagggtattactacatatactctaaggtgcaactgggtggagtaggttgtccattggggctcgcctctacaataactcatggtctgtataagcgaaccccgcggtatcccgaagagcttgaactcctcgtcagccagcaatctccctgtggtcgggcaacatcatctagcagagtgtggtgggactcctcttttttgggtggtgtagtacatctggaagcgggcgaagaagtggtcgtccgagtgcttgacgaacgactcgttagactgcgagacggaactcggagctactttggagcttttatggttagcggtggtggttcaggaggtgggagtgggagtgcatggtcacacccgcaattcgagaaatga

SEQ ID NO: 10 - protein, 769 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 11 - DNA, 2310 bp, synthetic construct
  source 1..2310: mol_type = other DNA
caggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccatcaccaccaccactga

SEQ ID NO: 12 - protein, 814 aa, synthetic construct
QGQSGSRHPCRHDPHIICYKFGGGSSGGSISSGLLSGRCDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 13 - DNA, 2445 bp, synthetic construct
  source 1..2445: mol_type = other DNA
cagggccagtctggatccagacacccatgcagacacgacccccacatcatctgttacaaattcggcggaggcagctccggcggcagcatctccagcggactgctgagcggcagatgcgataacatcggcggaagccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgttgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgcggcggacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccatcaccaccaccactga

SEQ ID NO: 14 - protein, 814 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 15 - DNA, 2445 bp, synthetic construct
  source 1..2445: mol_type = other DNA
cagggccagtctggatccagacacccatgcagacacgacccccacatcatctgttacaaattcggcggaggcagctccggcggcagcatctccagcggactgctgagcggcagaagcgataacatcggcggaagccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgataacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgttgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgcggcggacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccatcaccaccaccactga

SEQ ID NO: 16 - protein, 762 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSSAGGGSSGGSLSGRSDNIGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 17 - DNA, 2289 bp, synthetic construct
  source 1..2289: mol_type = other DNA
gatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtccagcgccgggggcggaagcagcggcggcagcttaagcggcagaagcgacaacatcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtga

SEQ ID NO: 18 - protein, 775 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 19 - DNA, 2328 bp, synthetic construct
  source 1..2328: mol_type = other DNA
caggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagtggtggagaacaaaaactcatttccgaggaagatctttga

SEQ ID NO: 20 - protein, 820 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 21 - DNA, 2463 bp, synthetic construct
  source 1..2463: mol_type = other DNA
cagggacagtccggatctaggcatccatgtcggcatgacccacatatcatatgctacaaattcggtggaggatcttctggaggctcaatttcatcaggactcctgtccgggcgctccgacaatattggtggctcccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgtcatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagtggtggagaacaaaaactcatttccgaggaagatctttga

SEQ ID NO: 22 - protein, 775 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 23 - DNA, 2328 bp, synthetic construct
  source 1..2328: mol_type = other DNA
caggagaggcgctctcatgaagtaaaccccgccgcgcaccttaccggtgcgaattccagccttacgggctccgggggccctcttctgtgggaaacccaactcgggctggcgtttctcaggggtctcagttaccacgacggcgcgcttgtcgttactaaagcgggctactactatatctactccaaggtacagctcggtggtgtaggatgtccgcttgggcttgcctctaccatcacgcacggtctctataaaagaaccccaagataccccgaagagttggaactgcttgtttctcaacagtccccttgtggtcgggcaaccagttcatctcgggtgtggtgggatagtagctttctcggaggagtagtccacctggaagctggggaagaggtagtcgtccgggtactcgatgaacggctcgtgcgcctccgagatgggacccggtcttactttggggctttcatggttagctctgcggggggaggaagctccgggggtagttccgccggcggcggatctagtggcggttctgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 24 - protein, 775 aa, synthetic construct
QERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 25 - DNA, 2328 bp, synthetic construct
  source 1..2328: mol_type = other DNA
caagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtctccagcgccgggggcggaagcagcggcggcagcttaagcggcagaagcgacaacatcggcagcgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 26 - protein, 775 aa, synthetic construct
QERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 27 - DNA, 2328 bp, synthetic construct
  source 1..2328: mol_type = other DNA
caagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtcagctctgcggggggaggaagctccgggggtagttccgccggcggcggatctagtggcggttctgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 28 - protein, 820 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 29 - DNA, 2463 bp, synthetic construct
  source 1..2463: mol_type = other DNA
cagggacagtccggatctaggcatccatgtcggcatgacccacatatcatatgctacaaattcggtggaggatcttctggaggctcaatttcatcaggactcctgtccgggcgctccgacaatattggtggctcccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtcagctctgcggggggaggaagctccgggggtagttccgccggcggcggatctagtggcggttctgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 30 - protein, 820 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 31 - DNA, 2463 bp, synthetic construct
  source 1..2463: mol_type = other DNA
cagggacagtccggatctaggcatccatgtcggcatgacccacatatcatatgctacaaattcggtggaggatcttctggaggctcaatttcatcaggactcctgtccgggcgctccgacaatattggtggctcccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtctccagcgccgggggcggaagcagcggcggcagcttaagcggcagaagcgacaacatcggcagcgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 32 - protein, 810 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSGGGSGGSGEIAALKQEIAALKKEIAALKWEIAALKQGYYGGSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 33 - DNA, 2433 bp, synthetic construct
  source 1..2433: mol_type = other DNA
caagagcgaagatcccatgaagtaaaccctgcagctcaccttacaggagccaacagcagtctgacaggttctgggggccccttgttgtgggagacgcaactggggcttgcattcctccgagggctcagttaccacgacggcgcgcttgttgttacaaaagcaggatactactatatttactcaaaggtgcagctgggtggagtgggatgtccattgggcctggcctcaactataacccatggcctctataaaagaacgccccggtatcctgaggagctggagctgttggtctcacagcagtcaccgtgcggcagagccacatcatcctctcgcgtatggtgggactcttccttcctgggaggtgtagtccatctcgaggcaggtgaagaagtcgtagttcgcgtactcgatgaacgcctggttcggctgagggatgggactaggagctatttcggcgcatttatggtatcttctgcaggtggaggaagtggtggcggttccggtggttcaggggaaatcgcggcactcaaacaagagatagcggctttgaaaaaggagatcgcagccctgaaatgggaaatagcggccttgaagcagggctactatggcggcagcggcggcagcctaagcggacggtccgacaatatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccaccaccatcaccactga

SEQ ID NO: 34 - protein, 812 aa, synthetic construct
EIAALKQEIAALKKEIAALKWEIAALKQGYYGGSGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 35 - DNA, 2439 bp, synthetic construct
  source 1..2439: mol_type = other DNA
gaaatcgcggcactcaaacaagagatagcggctttgaaaaaggagatcgcagccctgaaatgggaaatagcggccttgaagcagggctactatgggggatccgggggttcacaggagcgacggtcccatgaggtgaatccggcagcgcatttgacgggggccaactcatccctgacaggttcaggtggccctctgttgtgggaaactcagctcggactggccttccttagaggtttgtcatatcatgacggagcacttgtagtcaccaaagctgggtattactacatatactctaaggtccagctgggtggggtgggctgtccacttggcttggcatctacgatcacgcatgggttgtacaaaagaactccacgatatccagaagaactcgaattgcttgtctcccaacaatctccttgtggcagggctacgtccagttcccgagtgtggtgggattcaagttttctcgggggcgtagtccatcttgaagcaggggaggaagtggtcgtccgagtgctggacgaacggttggttaggcttcgggatgggacaagaagttattttggggccttcatggtatccagcgccgggggcggaagcagcggcggcagcttaagcggcagaagcgacaacatcggcagcgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 36 - protein, 855 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSGGGSGGSGEIAALKQEIAALKKEIAALKWEIAALKQGYYGGSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 37 - DNA, 2568 bp, synthetic construct
  source 1..2568: mol_type = other DNA
caaggacagagcggctcacggcatccttgtagacacgacccacacatcatctgttacaagtttggaggcggcagcagcggcggctctatcagctctggccttctgagcggtcggagcgacaacatcggaggcagccaagagcgaagatcccatgaagtaaaccctgcagctcaccttacaggagccaacagcagtctgacaggttctgggggccccttgttgtgggagacgcaactggggcttgcattcctccgagggctcagttaccacgacggcgcgcttgttgttacaaaagcaggatactactatatttactcaaaggtgcagctgggtggagtgggatgtccattgggcctggcctcaactataacccatggcctctataaaagaacgccccggtatcctgaggagctggagctgttggtctcacagcagtcaccgtgcggcagagccacatcatcctctcgcgtatggtgggactcttccttcctgggaggtgtagtccatctcgaggcaggtgaagaagtcgtagttcgcgtactcgatgaacgcctggttcggctgagggatgggactaggagctatttcggcgcatttatggtatcttctgcaggtggaggaagtggtggcggttccggtggttcaggggaaatcgcggcactcaaacaagagatagcggctttgaaaaaggagatcgcagccctgaaatgggaaatagcggccttgaagcagggctactatggcggcagcggcggcagcctaagcggacggtccgacaatatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccaccaccatcaccactga

SEQ ID NO: 38 - protein, 858 aa, synthetic construct
GSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSSGGSGSQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSGGGSGGSGEIAALKQEIAALKKEIAALKWEIAALKQGYYGG

SEQ ID NO: 39 - DNA, 2577 bp, synthetic construct
  source 1..2577: mol_type = other DNA
ggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttggggggcggaagcagcggcggcagcggcagccaaggccagagcggcagcaggcacccttgtagacacgatcctcacatcatctgttacaagtttggaggaggcagtagcggtggcagtattagcagcggactgctgagtggcagaagcgacaacatcggcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtcttctgcaggtggaggaagtggtggcggttccggtggttcaggggaaatcgcggcactcaaacaagagatagcggctttgaaaaaggagatcgcagccctgaaatgggaaatagcggccttgaagcagggctactatggcggctga

SEQ ID NO: 40 - protein, 776 aa, synthetic construct
GSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSSAGGGSSGGSLSGRSDNIGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 41 - DNA, 2331 bp, synthetic construct
  source 1..2331: mol_type = other DNA
ggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtccagcgccgggggcggaagcagcggcggcagcttaagcggcagaagcgacaacatcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtga

SEQ ID NO: 42 - protein, 802 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGGGSISSGLLSGRSDNIGGGSSGGSRHPCRHDPHIICYKFSGGGSGGGSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSEQKLISEEDL

SEQ ID NO: 43 - DNA, 2409 bp, synthetic construct
  source 1..2409: mol_type = other DNA
caagagcgaagatcccatgaagtaaaccctgcagctcaccttacaggagccaacagcagtctgacaggttctgggggccccttgttgtgggagacgcaactggggcttgcattcctccgagggctcagttaccacgacggcgcgcttgttgttacaaaagcaggatactactatatttactcaaaggtgcagctgggtggagtgggatgtccattgggcctggcctcaactataacccatggcctctataaaagaacgccccggtatcctgaggagctggagctgttggtctcacagcagtcaccgtgcggcagagccacatcatcctctcgcgtatggtgggactcttccttcctgggaggtgtagtccatctcgaggcaggtgaagaagtcgtagttcgcgtactcgatgaacgcctggttcggctgagggatgggactaggagctatttcggcgcatttatggtaggcggcggctctatctcctccggcctgctgagcggcagaagcgacaacatcggcggaggcagctccggcggctctagacacccttgtagacacgatcctcacatcatctgttacaagttttctggtggaggaagtggtggcggttccggtgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagcgaacaaaaactcatttccgaggaagatctttga

SEQ ID NO: 44 - protein, 810 aa, synthetic construct
GSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSSGGSGSQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 45 - DNA, 2433 bp, synthetic construct
  source 1..2433: mol_type = other DNA
ggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttggggggcggaagcagcggcggcagcggcagccaaggccagagcggcagcaggcacccttgtagacacgatcctcacatcatctgttacaagtttggaggaggcagtagcggtggcagtattagcagcggactgctgagtggcagaagcgacaacatcggcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtga

SEQ ID NO: 46 - protein, 816 aa, synthetic construct
GSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSSAGGGSSGGSLSGRSDNIGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGGGSISSGLLSGRSDNIGGGSSGGSRHPCRHDPHIICYKF

SEQ ID NO: 47 - DNA, 2451 bp, synthetic construct
  source 1..2451: mol_type = other DNA
ggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtcttctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgctgctgacccacatgagtgctacgctaaggttttcgacgaatttaagccattggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtccagcgccgggggcggaagcagcggcggcagcttaagcggcagaagcgacaacatcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgggcggaggctccatttctagcggcctgctgagcggcagaagcgataacatcggcggaggaagcagcggaggcagcagacacccctgcagacacgatcctcacatcatctgctacaagttctga

SEQ ID NO: 48 - protein, 810 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 49 - DNA, 2433 bp, synthetic construct
  source 1..2433: mol_type = other DNA
caagggcaatccggatctcggcatccgtgtcggcatgaccctcacatcatatgctataagtttggggggggatcctcaggtggttccatttcttctggtttgttgtctggaagatcagacaacatcggcggatctcaagaaagacgctctcatgaggtgaatcctgccgcgcaccttactggggcaaactccagtctgaccggatcaggtgggccattgctttgggagacacagttgggactggcattccttcgaggcctcagctaccatgatggcgccctggtcgtaacgaaagctgggtattactatatatatagcaaagtccaactggggggggttgggtgtcccctggggctcgcgtcaaccatcacccacggactctataagagaacaccaagatacccagaagagttggaacttctcgtgtctcaacaaagtccatgtgggagggcaacatcttcttcacgcgtatggtgggattcatcctttctcggtggcgttgtccatcttgaagcaggggaggaagttgttgtcagagtcttggatgaaagactcgtgagattgcgggatggtactagatcctacttcggcgcgttcatggtctcctcagcgggaggtgggagtagtggcggctcagacgcccataaatccgaagtggcacatcgatttaaggacctcggggaggagaacttcaaagctctggtactcatcgcctttgctcagtacttgcagcaatgccccttcgaggatcatgtcaaactcgtgaacgaggttacggagttcgctaaaacatgtgttgctgacgagtctgcagagaactgtgacaaatccctccacacgctgttcggtgataaactgtgtacggtggctaccctcagggaaacctacggagagatggccgattgttgcgccaaacaggagcctgagaggaacgaatgctttcttcaacacaaggatgacaatcctaacttgcctaggctggttcggcccgaggtcgatgtgatgtgcacagcgtttcacgacaacgaagaaacattcctgaagaaatacttgtacgagattgctaggcgacacccatatttctacgcgccggagcttctcttctttgcgaagcgctacaaggctgcatttacagagtgctgccaagccgctgataaggcggcctgtcttctccccaagctcgatgaactccgagatgaagggaaagcttcatcagcgaaacaaagattgaaatgtgcttccctccaaaagtttggagaacgagcctttaaggcttgggcagtggcacggctcagtcagcgctttcctaaggctgaatttgccgaagtgtccaagcttgtaacggatctcactaaagttcatactgaatgctgccacggagaccttctcgaatgcgcggacgatcgcgcggacttggcgaaatatatatgcgagaatcaagatagtatcagcagtaaactcaaagagtgctgcgagaagcctctcctcgaaaagagccactgtatcgccgaggtggaaaatgatgagatgcctgcggacttgccatcccttgccgcagactttgtcgaatcaaaagacgtttgcaagaattacgcggaggcaaaagatgtattccttggcatgttcttgtacgaatacgcacggcgccaccctgactattcagtagtgttgctcttgagactcgctaaaacatacgaaacgacgcttgagaaatgttgcgcagcagccgatccccacgagtgttacgcaaaggtgttcgacgagtttaaacccctcgttgaagaacctcaaaacctgataaaacaaaattgtgagttgttcgagcagttgggagagtacaagtttcagaatgctctcctggttcggtacaccaagaaggtcccacaagtgtccacgcccaccctcgtagaggtatcacggaaccttggcaaggtcggtagcaagtgctgcaaacacccagaagctaagcgcatgccatgcgctgaagactatctgtctgtggtgcttaatcaattgtgtgtactgcatgagaaaactcctgtgtccgaccgggttaccaagtgctgtaccgagtcactcgtcaaccggcgaccttgtttttctgcgctggaggttgatgagacgtatgttccgaaagaattcaacgccgagactttcaccttccatgctgatatatgcacactcagtgaaaaagaacgacaaataaagaagcaaaccgcattggtcgagctggtcaagcataaacccaaagctacaaaagaacaattgaaggctgttatggatgactttgccgcgttcgtagagaaatgctgcaaggcagatgataaagagacatgtttcgccgaagagggcaagaaactggtggccgcctctcaagctgcacttgggctctctggaggcgaacaaaagcttataagcgaagaggacttgtga

SEQ ID NO: 50 - protein, 820 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 51 - DNA, 2463 bp, synthetic construct
  source 1..2463: mol_type = other DNA
cagggacagtccggatctaggcatccatgtcggcatgacccacatatcatatgctacaaattcggtggaggatcttctggaggctcaatttcatcaggactcctgtccgggcgctccgacaatattggtggctcccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatcttctgcagggggggggtcatccggcggaagttcagcgggggggggatcctccggtggcagtgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtcaggtggggagcagaaacttatctccgaagaggatttgtaa

SEQ ID NO: 52 - protein, 820 aa, synthetic construct
QGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVESKYGPPCPSCPAPEFLGGPSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 53 - DNA, 2463 bp, synthetic construct
  source 1..2463: mol_type = other DNA
caaggccagagtgggagtaggcatccatgtcgccatgatcctcacattatatgctataaatttggtgggggaagcagtggtgggtccatcagtagtgggctgttgtccggtcggagtgataatattggaggctcccaggaacgcaggagtcatgaagttaacccggcggcacatcttacgggtgcgaactctagtcttaccgggagtggggggcccctgctttgggagacacagcttgggctcgctttcctcaggggactctcttaccatgatggcgcactggtagtaaccaaagctggatactattatatttactctaaagttcaattggggggcgttggctgcccactcggcctcgcatctactatcacccatgggttgtataagcggacccctagataccctgaggaacttgagcttttggtttctcaacagtcaccttgcggtagggctacctcatcaagccgcgtttggtgggacagttcatttctcgggggcgtagtccatctggaggcaggtgaggaagtagtggttcgagtgctcgacgagcgcctcgtacgactccgggatgggacgagaagttatttcggcgcctttatggtagagtcaaaatacgggcctccctgtccctcttgtccggcacccgaatttcttggcgggccctctgatgcccacaagtctgaagttgcgcatagatttaaagacctcggagaggagaattttaaagcactcgtgcttatcgcatttgcgcagtacctgcagcagtgccctttcgaggaccacgtcaagcttgtcaacgaggtgacagaattcgccaaaacatgtgtcgccgatgagtctgccgagaactgtgacaaaagtttgcataccctgttcggcgacaaactctgcactgtagcaactcttagggaaacatacggggaaatggcagactgttgcgcgaaacaggagccagaacgaaacgaatgtttcttgcagcacaaggatgataaccctaatctcccgaggttggtcaggcccgaagtcgacgtaatgtgcacggctttccatgacaatgaggaaaccttcctcaagaagtacctctacgaaatagcacgaagacatccgtatttctatgctcctgagctgctctttttcgctaaacgctataaggccgcattcacggagtgttgtcaagccgccgataaggctgcttgccttctgcccaagttggacgaactgcgcgatgaggggaaagcatcatcagcaaagcaacgcctgaaatgcgctagtttgcaaaaattcggggaacgcgccttcaaagcttgggctgtcgcgcggctctcacagaggttccccaaggctgagttcgccgaagtttccaagctggttactgatttgaccaaagtgcacacagaatgttgccacggcgacctgcttgagtgtgcggatgaccgcgcagatctcgcgaagtatatatgcgaaaaccaggattcaattagctctaaattgaaagaatgttgtgagaaacctctgcttgaaaagtcacactgcattgcggaggtggaaaatgatgaaatgcccgcagatctcccctctttggcagcggacttcgtggagagtaaagacgtctgtaagaattacgccgaggcgaaggatgtattcctggggatgtttctctatgaatacgctcgcagacatcctgactactctgtggtgctgctgttgcgcctcgctaagacctacgaaacaaccctggaaaaatgttgtgccgcagctgatccacacgaatgctatgctaaagtctttgatgaatttaagcctttggttgaggagccccagaacctgataaaacagaactgtgaattgttcgagcaacttggagagtataaattccaaaacgcgcttctcgtgcggtacaccaagaaggtgcctcaagtcagtactccaacccttgtggaggtaagtcgcaatctcggtaaagttggcagtaaatgttgtaagcatcctgaagcgaagcgcatgccttgtgcagaggactatctgtcagtagttcttaaccagctgtgtgtgcttcatgagaaaacacccgtgtccgacagggtgacaaagtgttgcactgagagtctcgtgaaccggagaccttgtttctccgccctggaagtggacgaaacctatgtgccgaaggaattcaatgctgaaacgtttacgttccatgccgacatctgcacgcttagcgagaaagagagacagataaagaagcaaaccgccttggtggaactcgttaagcacaagccaaaagcaaccaaagaacaactcaaagcggtcatggacgatttcgctgcatttgtagagaaatgctgcaaagcggatgacaaggagacgtgtttcgctgaagagggaaaaaagctcgtggcagcatcccaggctgcacttggtctgtctggtggtgagcaaaaactgatttcagaggaggatttgtga

SEQ ID NO: 54 - protein, 156 aa, synthetic construct
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 55 - protein, 156 aa, synthetic construct
QERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMV

SEQ ID NO: 56 - protein, 585 aa, synthetic construct [sequence omitted]

SEQ ID NO: 57 - protein, 31 aa, synthetic construct
EIAALKQEIAALKKEIAALKWEIAALKQGYY

SEQ ID NO: 58 - protein, 6 aa, synthetic construct
HHHHHH

SEQ ID NO: 59 - protein, 10 aa, synthetic construct
EQKLISEEDL

SEQ ID NO: 60 - protein, 10 aa, synthetic construct
SAWSHPQFEK

SEQ ID NO: 61 - protein, 15 aa, synthetic construct
RHPCRHDPHIICYKF

SEQ ID NO: 62 - protein, 13 aa, synthetic construct
ISSGLLSGRSDNI

SEQ ID NO: 63 - protein, 8 aa, synthetic construct
LSGRSDNI

SEQ ID NO: 64 - protein, 11 aa, synthetic construct
SSAGGGSSGGS

SEQ ID NO: 65 - protein, 21 aa, synthetic construct
SSAGGGSSGGSSAGGGSSGGS

SEQ ID NO: 66 - protein, 10 aa, synthetic construct
SGGGSGGGSG

SEQ ID NO: 67 - protein, 10 aa, synthetic construct
GGGSSGGSGS

SEQ ID NO: 68 - protein, 8 aa, synthetic construct
GGGSSGGS

SEQ ID NO: 69 - protein, 11 aa, synthetic construct
SGGGSGGGSGS

SEQ ID NO: 70 - protein, 4 aa, synthetic construct
QSGS

SEQ ID NO: 71 - protein, 7 aa, synthetic construct
QGQSGQG

SEQ ID NO: 72 - protein, 6 aa, synthetic construct
GQSGQG

SEQ ID NO: 73 - protein, 5 aa, synthetic construct
QSGQG

SEQ ID NO: 74 - protein, 4 aa, synthetic construct
SGQG

SEQ ID NO: 75 - protein, 4 aa, synthetic construct
GGGS

SEQ ID NO: 76 - protein, 6 aa, synthetic construct
QGQSGS

SEQ ID NO: 77 - protein, 21 aa, synthetic construct
ESKYGPPCPSCPAPEFLGGPS

SEQ ID NO: 78 - protein, 20 aa, synthetic construct
METDTLLLWVLLLWVPGSTG

SEQ ID NO: 79 - protein, 240 aa, synthetic construct
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 80 - protein, 6 aa, synthetic construct
QGQSGQ

SEQ ID NO: 81 - protein, 13 aa, synthetic construct
ISSGLLSGRCDNI

SEQ ID NO: 82 - protein, 192 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGHHHHHH

SEQ ID NO: 83 - DNA, 579 bp, synthetic construct
  source 1..579: mol_type = other DNA
atggaaaccgacacactgctgctgtgggtgctgcttttgtgggtgccaggatccacaggccaggaacggcgctcccacgaggtcaatccggcggctcatttgactggagccaactctagtcttaccgggagcggcggtccattgctgtgggagacgcaacttggattggcttttcttcggggattgtcttaccacgatggagcactcgttgttacgaaagccggttactactacatttattctaaggtccagttgggtggagttggctgtcccctgggacttgcaagcactatcactcacggactgtacaagaggacaccccgataccccgaggaactcgagctgcttgtgagccaacagtctccatgtgggcgcgcaactagttcttcacgggtgtggtgggactccagtttcctgggaggtgttgttcatctcgaggcaggtgaagaagtagttgtcagggtacttgatgaacggcttgttagactccgggatggaacgaggtcctatttcggtgcgttcatggtaagcgggggcggaagtgggggcgggtcaggtcatcatcaccaccatcattga

SEQ ID NO: 84 - protein, 237 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGHHHHHH

SEQ ID NO: 85 - DNA, 714 bp, synthetic construct
  source 1..714: mol_type = other DNA
atggaaaccgacacactgctgctgtgggtgctgcttttgtgggtgccaggatccacaggccaaggacaaagtggttcccgacacccgtgcaggcatgacccacatataatctgttataagtttggtggaggctcctccggcgggtctatcagttccggtctcctcagcggtcgcagtgataatatcggcggttctcaggaacgcaggtcacacgaggtaaatccagccgcccatttgactggcgctaatagttctcttacgggtagcggtggccctcttctgtgggaaacgcagttgggcttggctttccttagaggtctcagctatcacgacggcgccctcgtcgttacaaaggccgggtactattatatttattctaaggtacagcttggcggggtggggtgcccgctgggtcttgcatctacgatcactcatggtctctacaaaagaacaccaaggtatccagaagaacttgagctcctcgttagccaacagtccccctgcggacgggctacttcctctagccgggtatggtgggatagcagtttccttgggggggtcgtgcatctcgaggctggagaggaggtagtagtcagggtcttggatgagagattggtaaggcttcgagacggtacaagatcatactttggggccttcatggtgagcgggggtggcagcggcggtggcagcgggcatcatcaccatcaccattga

SEQ ID NO: 86 - protein, 196 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGSAWSHPQFEK

SEQ ID NO: 87 - DNA, 591 bp, synthetic construct
  source 1..591: mol_type = other DNA
atggaaaccgacacactgctgctgtgggtgctgcttttgtgggtgccaggatccacaggccaggaacgcaggtcccatgaagtaaacccggcggcccatcttacgggtgcaaattcaagcttgacaggcagcggagggccgctcctgtgggagacgcaacttgggcttgcgtttctccggggcctgtcatatcacgacggtgctcttgtggtaacgaaagcagggtactattatatatatagcaaggttcagttgggtggggttggatgcccgcttggactcgcgtccactattactcacgggttgtataagaggacaccgcgatacccagaggagctggaactccttgtttcacagcagtctccttgcgggagagccacatcttcatccagggtctggtgggactcttctttcttgggcggcgttgttcatctggaagcgggggaagaggttgtagtgagggtactcgacgagaggctggtgaggctgcgagacgggacccgctcatatttcggagccttcatggtatcaggagggggctccggtggggggtcaggaagcgcatggtcacaccctcagttcgagaaatga

SEQ ID NO: 88 - protein, 241 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSGGGSGGGSGSAWSHPQFEK

SEQ ID NO: 89 - DNA, 726 bp, synthetic construct
  source 1..726: mol_type = other DNA
atggaaaccgacacactgctgctgtgggtgctgcttttgtgggtgccaggatccacaggccaaggacagagtggaagtcgacacccttgtaggcacgacccacatataatctgctataaatttggtgggggttccagcggtggaagtatcagctccggccttcttagtggcaggagtgacaatattggcggatctcaagagcgacgcagtcacgaggtaaatcctgcagcacacttgacaggtgctaacagctcacttaccggaagtggggggccgctgttgtgggagacgcagctcgggttggcctttcttcgagggctctcttatcacgacggtgcattggtggtgaccaaagcagggtattactacatatactctaaggtgcaactgggtggagtaggttgtccattggggctcgcctctacaataactcatggtctgtataagcgaaccccgcggtatcccgaagagcttgaactcctcgtcagccagcaatctccctgtggtcgggcaacatcatctagcagagtgtggtgggactcctcttttttgggtggtgtagtacatctggaagcgggcgaagaagtggtcgtccgagtgcttgacgaacgactcgttagactgcgagacggaactcggagctactttggagcttttatggttagcggtggtggttcaggaggtgggagtgggagtgcatggtcacacccgcaattcgagaaatga

SEQ ID NO: 90 - protein, 789 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 91 - DNA, 2370 bp, synthetic construct
  source 1..2370: mol_type = other DNA
atggaaaccgacacactgctgctgtgggtgctgcttttgtgggtgccaggatccacaggccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccatcaccaccaccactga

SEQ ID NO: 92 - protein, 834 aa, synthetic construct
METDTLLLWVLLLWVPGSTGQGQSGSRHPCRHDPHIICYKFGGGSSGGSISSGLLSGRCDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 93 - DNA, 2505 bp, synthetic construct
  source 1..2505: mol_type = other DNA
atggaaaccgacacactgctgctgtgggtgctgcttttgtgggtgccaggatccacaggccagggccagtctggatccagacacccatgcagacacgacccccacatcatctgttacaaattcggcggaggcagctccggcggcagcatctccagcggactgctgagcggcagatgcgataacatcggcggaagccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgttgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgcggcggacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccatcaccaccaccactga

SEQ ID NO: 94 - protein, 834 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 95 - DNA, 2505 bp, synthetic construct
  source 1..2505: mol_type = other DNA
atggaaaccgacacactgctgctgtgggtgctgcttttgtgggtgccaggatccacaggccagggccagtctggatccagacacccatgcagacacgacccccacatcatctgttacaaattcggcggaggcagctccggcggcagcatctccagcggactgctgagcggcagaagcgataacatcggcggaagccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgataacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgttgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgcggcggacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccatcaccaccaccactga

SEQ ID NO: 96 - protein, 782 aa, synthetic construct
METDTLLLWVLLLWVPGSTGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSSAGGGSSGGSLSGRSDNIGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 97 - DNA, 2349 bp, synthetic construct
  source 1..2349: mol_type = other DNA
atggaaaccgacacactgctgctgtgggtgctgcttttgtgggtgccaggatccacaggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtccagcgccgggggcggaagcagcggcggcagcttaagcggcagaagcgacaacatcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtga

SEQ ID NO: 98 - protein, 795 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 99 - DNA, 2388 bp, synthetic construct
  source 1..2388: mol_type = other DNA
atggaaaccgacacactgctgctgtgggtgctgcttttgtgggtgccaggatccacaggccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagtggtggagaacaaaaactcatttccgaggaagatctttga

SEQ ID NO: 100 - protein, 840 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 101 - DNA, 2523 bp, synthetic construct
  source 1..2523: mol_type = other DNA
atggaaaccgacacactgctgctgtgggtgctgcttttgtgggtgccaggatccacaggccagggacagtccggatctaggcatccatgtcggcatgacccacatatcatatgctacaaattcggtggaggatcttctggaggctcaatttcatcaggactcctgtccgggcgctccgacaatattggtggctcccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatccagcgccgggggcggaagcagcggcggcagcttaagcggcagaagcgacaacatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgtcatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagtggtggagaacaaaaactcatttccgaggaagatctttga

SEQ ID NO: 102 - protein, 795 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 103 - DNA, 2388 bp, synthetic construct
  source 1..2388: mol_type = other DNA
atggaaaccgacacactgctgctgtgggtgctgcttttgtgggtgccaggatccacaggccaggagaggcgctctcatgaagtaaaccccgccgcgcaccttaccggtgcgaattccagccttacgggctccgggggccctcttctgtgggaaacccaactcgggctggcgtttctcaggggtctcagttaccacgacggcgcgcttgtcgttactaaagcgggctactactatatctactccaaggtacagctcggtggtgtaggatgtccgcttgggcttgcctctaccatcacgcacggtctctataaaagaaccccaagataccccgaagagttggaactgcttgtttctcaacagtccccttgtggtcgggcaaccagttcatctcgggtgtggtgggatagtagctttctcggaggagtagtccacctggaagctggggaagaggtagtcgtccgggtactcgatgaacggctcgtgcgcctccgagatgggacccggtcttactttggggctttcatggttagctctgcggggggaggaagctccgggggtagttccgccggcggcggatctagtggcggttctgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 104 - protein, 795 aa, synthetic construct
METDTLLLWVLLLWVPGSTGQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 105 - DNA, 2388 bp, synthetic construct
  source 1..2388: mol_type = other DNA
atggaaaccgacacactgctgctgtgggtgctgcttttgtgggtgccaggatccacaggccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtctccagcgccgggggcggaagcagcggcggcagcttaagcggcagaagcgacaacatcggcagcgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 106 - protein, 795 aa, synthetic construct
METDTLLLWVLLLWVPGSTGQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 107 - DNA, 2388 bp, synthetic construct
  source 1..2388: mol_type = other DNA
atggaaaccgacacactgctgctgtgggtgctgcttttgtgggtgccaggatccacaggccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtcagctctgcggggggaggaagctccgggggtagttccgccggcggcggatctagtggcggttctgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 108 - protein, 840 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 109 - DNA, 2523 bp, synthetic construct
  source 1..2523: mol_type = other DNA
atggaaaccgacacactgctgctgtgggtgctgcttttgtgggtgccaggatccacaggccagggacagtccggatctaggcatccatgtcggcatgacccacatatcatatgctacaaattcggtggaggatcttctggaggctcaatttcatcaggactcctgtccgggcgctccgacaatattggtggctcccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtcagctctgcggggggaggaagctccgggggtagttccgccggcggcggatctagtggcggttctgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 110 - protein, 840 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 111 - DNA, 2523 bp, synthetic construct
  source 1..2523: mol_type = other DNA
atggaaaccgacacactgctgctgtgggtgctgcttttgtgggtgccaggatccacaggccagggacagtccggatctaggcatccatgtcggcatgacccacatatcatatgctacaaattcggtggaggatcttctggaggctcaatttcatcaggactcctgtccgggcgctccgacaatattggtggctcccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtctccagcgccgggggcggaagcagcggcggcagcttaagcggcagaagcgacaacatcggcagcgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 112 - protein, 830 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSGGGSGGSGEIAALKQEIAALKKEIAALKWEIAALKQGYYGGSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 113 - DNA, 2493 bp, synthetic construct
  source 1..2493: mol_type = other DNA
atggaaaccgacacactgctgctgtgggtgctgcttttgtgggtgccaggatccacaggccaagagcgaagatcccatgaagtaaaccctgcagctcaccttacaggagccaacagcagtctgacaggttctgggggccccttgttgtgggagacgcaactggggcttgcattcctccgagggctcagttaccacgacggcgcgcttgttgttacaaaagcaggatactactatatttactcaaaggtgcagctgggtggagtgggatgtccattgggcctggcctcaactataacccatggcctctataaaagaacgccccggtatcctgaggagctggagctgttggtctcacagcagtcaccgtgcggcagagccacatcatcctctcgcgtatggtgggactcttccttcctgggaggtgtagtccatctcgaggcaggtgaagaagtcgtagttcgcgtactcgatgaacgcctggttcggctgagggatgggactaggagctatttcggcgcatttatggtatcttctgcaggtggaggaagtggtggcggttccggtggttcaggggaaatcgcggcactcaaacaagagatagcggctttgaaaaaggagatcgcagccctgaaatgggaaatagcggccttgaagcagggctactatggcggcagcggcggcagcctaagcggacggtccgacaatatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccaccaccatcaccactga

SEQ ID NO: 114 - protein, 832 aa, synthetic construct
METDTLLLWVLLLWVPGSTGEIAALKQEIAALKKEIAALKWEIAALKQGYYGGSGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 115 - DNA, 2499 bp, synthetic construct
  source 1..2499: mol_type = other DNA
atggaaaccgacacactgctgctgtgggtgctgcttttgtgggtgccaggatccacaggcgaaatcgcggcactcaaacaagagatagcggctttgaaaaaggagatcgcagccctgaaatgggaaatagcggccttgaagcagggctactatgggggatccgggggttcacaggagcgacggtcccatgaggtgaatccggcagcgcatttgacgggggccaactcatccctgacaggttcaggtggccctctgttgtgggaaactcagctcggactggccttccttagaggtttgtcatatcatgacggagcacttgtagtcaccaaagctgggtattactacatatactctaaggtccagctgggtggggtgggctgtccacttggcttggcatctacgatcacgcatgggttgtacaaaagaactccacgatatccagaagaactcgaattgcttgtctcccaacaatctccttgtggcagggctacgtccagttcccgagtgtggtgggattcaagttttctcgggggcgtagtccatcttgaagcaggggaggaagtggtcgtccgagtgctggacgaacggttggttaggcttcgggatgggacaagaagttattttggggccttcatggtatccagcgccgggggcggaagcagcggcggcagcttaagcggcagaagcgacaacatcggcagcgatgctcataaaagtgaggtcgcgcacagattcaaagacctcggcgaggaaaactttaaagccctcgtattgatagctttcgcccagtacctccagcagtgcccattcgaggatcacgtgaaactcgtgaatgaagtaacggaatttgccaaaacgtgcgtggccgacgagagtgccgaaaattgcgacaagtcacttcacaccctttttggtgacaaattgtgtactgtggcgacgcttagggaaacatacggagaaatggccgattgctgtgccaaacaagagcctgaaagaaacgaatgctttcttcagcacaaagacgataaccctaacttgccgagactggtgagacctgaagtagacgtcatgtgtaccgcgttccatgacaacgaagagactttcttgaaaaaatacctttatgaaatagcacggaggcacccttatttttacgctccagagctcctcttctttgctaaacggtacaaagcggcgtttaccgaatgttgccaagctgcggataaagctgcatgtcttctcccgaagttggatgagctgagagatgagggtaaagcgtcttccgctaaacaacgacttaaatgcgcttctctgcagaaatttggcgaacgagcgttcaaagcctgggccgttgcgcgcctttcacagcggtttccgaaagccgagttcgccgaggtttcaaagctggttacggacttgacaaaggtgcatacagaatgctgccacggagacctgctggagtgcgccgatgatcgcgctgatttggctaaatatatttgcgaaaatcaggacagcatcagctccaagttgaaggagtgttgtgaaaaaccgctgctggagaagtcacactgcatagccgaggtggaaaacgacgagatgccggcagacctccccagccttgccgcggatttcgtcgaatcaaaggatgtatgcaaaaattacgcggaagcaaaagatgtatttctgggaatgttcctgtacgagtacgctaggcgacatcccgactacagcgttgttctgctcttgagacttgcgaagacgtacgagactacgctcgagaagtgctgtgcagctgcggacccccatgagtgttatgctaaagtcttcgacgaatttaagcctttggtggaagagccacagaacctgatcaaacaaaattgtgaactgttcgaacaattgggggagtataaatttcagaacgctctccttgttcggtatacaaaaaaagtaccgcaggtaagtacgccgacactcgtggaagtaagcaggaacctcggaaaggttggatcaaaatgctgcaagcatccggaggctaagcgcatgccatgcgctgaggattatctttcagtggtcctgaaccaattgtgcgtgttgcacgagaaaactccagtaagcgatagagttaccaaatgttgtacagagagcctggttaatcgacgaccttgcttcagcgccttggaagtcgatgagacgtatgtgccgaaggaatttaacgccgaaactttcacttttcatgcagatatctgtacattgagcgaaaaggaaaggcaaattaaaaaacaaactgcgcttgtggaattggtcaagcacaagcctaaagccacaaaggaacagctcaaagcggtaatggacgattttgcggcgttcgtagagaagtgttgcaaggcggacgataaagaaacgtgctttgctgaggagggaaaaaagcttgttgctgccagtcaagcggcgcttggcttgagtggtggagaacaaaaactcatttccgaggaagatctttga

SEQ ID NO: 116 - protein, 875 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSGGGSGGSGEIAALKQEIAALKKEIAALKWEIAALKQGYYGGSGGSLSGRSDNIGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSHHHHHH

SEQ ID NO: 117 - DNA, 2628 bp, synthetic construct
  source 1..2628: mol_type = other DNA
atggaaaccgacacactgctgctgtgggtgctgcttttgtgggtgccaggatccacaggccaaggacagagcggctcacggcatccttgtagacacgacccacacatcatctgttacaagtttggaggcggcagcagcggcggctctatcagctctggccttctgagcggtcggagcgacaacatcggaggcagccaagagcgaagatcccatgaagtaaaccctgcagctcaccttacaggagccaacagcagtctgacaggttctgggggccccttgttgtgggagacgcaactggggcttgcattcctccgagggctcagttaccacgacggcgcgcttgttgttacaaaagcaggatactactatatttactcaaaggtgcagctgggtggagtgggatgtccattgggcctggcctcaactataacccatggcctctataaaagaacgccccggtatcctgaggagctggagctgttggtctcacagcagtcaccgtgcggcagagccacatcatcctctcgcgtatggtgggactcttccttcctgggaggtgtagtccatctcgaggcaggtgaagaagtcgtagttcgcgtactcgatgaacgcctggttcggctgagggatgggactaggagctatttcggcgcatttatggtatcttctgcaggtggaggaagtggtggcggttccggtggttcaggggaaatcgcggcactcaaacaagagatagcggctttgaaaaaggagatcgcagccctgaaatgggaaatagcggccttgaagcagggctactatggcggcagcggcggcagcctaagcggacggtccgacaatatcggcagcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagccaccaccaccatcaccactga

SEQ ID NO: 118 - protein, 878 aa, synthetic construct
METDTLLLWVLLLWVPGSTGGSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSSGGSGSQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSGGGSGGSGEIAALKQEIAALKKEIAALKWEIAALKQGYYGG

SEQ ID NO: 119 - DNA, 2637 bp, synthetic construct
  source 1..2637: mol_type = other DNA
atggaaaccgacacactgctgctgtgggtgctgcttttgtgggtgccaggatccacaggcggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttggggggcggaagcagcggcggcagcggcagccaaggccagagcggcagcaggcacccttgtagacacgatcctcacatcatctgttacaagtttggaggaggcagtagcggtggcagtattagcagcggactgctgagtggcagaagcgacaacatcggcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtcttctgcaggtggaggaagtggtggcggttccggtggttcaggggaaatcgcggcactcaaacaagagatagcggctttgaaaaaggagatcgcagccctgaaatgggaaatagcggccttgaagcagggctactatggcggctga

SEQ ID NO: 120 - protein, 796 aa, synthetic construct
METDTLLLWVLLLWVPGSTGGSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSSAGGGSSGGSLSGRSDNIGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 121 - DNA, 2391 bp, synthetic construct
  source 1..2391: mol_type = other DNA
atggaaaccgacacactgctgctgtgggtgctgcttttgtgggtgccaggatccacaggcggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtccagcgccgggggcggaagcagcggcggcagcttaagcggcagaagcgacaacatcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtga

SEQ ID NO: 122 - protein, 822 aa, synthetic construct
METDTLLLWVLLLWVPGSTGQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGGGSISSGLLSGRSDNIGGGSSGGSRHPCRHDPHIICYKFSGGGSGGGSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSEQKLISEEDL

SEQ ID NO: 123 - DNA, 2469 bp, synthetic construct
  source 1..2469: mol_type = other DNA
atggaaaccgacacactgctgctgtgggtgctgcttttgtgggtgccaggatccacaggccaagagcgaagatcccatgaagtaaaccctgcagctcaccttacaggagccaacagcagtctgacaggttctgggggccccttgttgtgggagacgcaactggggcttgcattcctccgagggctcagttaccacgacggcgcgcttgttgttacaaaagcaggatactactatatttactcaaaggtgcagctgggtggagtgggatgtccattgggcctggcctcaactataacccatggcctctataaaagaacgccccggtatcctgaggagctggagctgttggtctcacagcagtcaccgtgcggcagagccacatcatcctctcgcgtatggtgggactcttccttcctgggaggtgtagtccatctcgaggcaggtgaagaagtcgtagttcgcgtactcgatgaacgcctggttcggctgagggatgggactaggagctatttcggcgcatttatggtaggcggcggctctatctcctccggcctgctgagcggcagaagcgacaacatcggcggaggcagctccggcggctctagacacccttgtagacacgatcctcacatcatctgttacaagttttctggtggaggaagtggtggcggttccggtgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgagcgaacaaaaactcatttccgaggaagatctttga

SEQ ID NO: 124 - protein, 830 aa, synthetic construct
METDTLLLWVLLLWVPGSTGGSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSSGGSGSQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 125 - DNA, 2493 bp, synthetic construct
  source 1..2493: mol_type = other DNA
atggaaaccgacacactgctgctgtgggtgctgcttttgtgggtgccaggatccacaggcggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttggggggcggaagcagcggcggcagcggcagccaaggccagagcggcagcaggcacccttgtagacacgatcctcacatcatctgttacaagtttggaggaggcagtagcggtggcagtattagcagcggactgctgagtggcagaagcgacaacatcggcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgtga

SEQ ID NO: 126 - protein, 836 aa, synthetic construct
METDTLLLWVLLLWVPGSTGGSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSSAGGGSSGGSLSGRSDNIGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGGGSISSGLLSGRSDNIGGGSSGGSRHPCRHDPHIICYKF

SEQ ID NO: 127 - DNA, 2511 bp, synthetic construct
  source 1..2511: mol_type = other DNA
atggaaaccgacacactgctgctgtgggtgctgcttttgtgggtgccaggatccacaggcggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtcttctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgctgctgacccacatgagtgctacgctaaggttttcgacgaatttaagccattggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtccagcgccgggggcggaagcagcggcggcagcttaagcggcagaagcgacaacatcggcagccaggagagacggagccacgaggtgaaccccgctgcccacctgaccggcgccaacagcagtctgaccggcagcggcggccctctgctgtgggagacacagctgggcctggcctttctgagaggcctgtcctaccacgacggcgccctggtcgtgaccaaggccggctactactacatctacagcaaggtgcagctgggaggcgtgggctgccctctgggcctggcatctaccatcacgcacggcctgtacaagcggacccctagatatcctgaggaactggaactcctggtgtcccagcagagcccttgcggcagagctacaagcagctcaagagtgtggtgggacagcagcttcctgggcggagtggtccacctggaagctggagaagaagtggtggtccgggtgctggacgagagactcgtgaggctgagagacggaacaagaagctacttcggcgcttttatggtgggcggaggctccatttctagcggcctgctgagcggcagaagcgataacatcggcggaggaagcagcggaggcagcagacacccctgcagacacgatcctcacatcatctgctacaagttctga

SEQ ID NO: 128 - protein, 830 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 129 - DNA, 2493 bp, synthetic construct
  source 1..2493: mol_type = other DNA
atggaaaccgacacactgctgctgtgggtgctgcttttgtgggtgccaggatccacaggccaagggcaatccggatctcggcatccgtgtcggcatgaccctcacatcatatgctataagtttggggggggatcctcaggtggttccatttcttctggtttgttgtctggaagatcagacaacatcggcggatctcaagaaagacgctctcatgaggtgaatcctgccgcgcaccttactggggcaaactccagtctgaccggatcaggtgggccattgctttgggagacacagttgggactggcattccttcgaggcctcagctaccatgatggcgccctggtcgtaacgaaagctgggtattactatatatatagcaaagtccaactggggggggttgggtgtcccctggggctcgcgtcaaccatcacccacggactctataagagaacaccaagatacccagaagagttggaacttctcgtgtctcaacaaagtccatgtgggagggcaacatcttcttcacgcgtatggtgggattcatcctttctcggtggcgttgtccatcttgaagcaggggaggaagttgttgtcagagtcttggatgaaagactcgtgagattgcgggatggtactagatcctacttcggcgcgttcatggtctcctcagcgggaggtgggagtagtggcggctcagacgcccataaatccgaagtggcacatcgatttaaggacctcggggaggagaacttcaaagctctggtactcatcgcctttgctcagtacttgcagcaatgccccttcgaggatcatgtcaaactcgtgaacgaggttacggagttcgctaaaacatgtgttgctgacgagtctgcagagaactgtgacaaatccctccacacgctgttcggtgataaactgtgtacggtggctaccctcagggaaacctacggagagatggccgattgttgcgccaaacaggagcctgagaggaacgaatgctttcttcaacacaaggatgacaatcctaacttgcctaggctggttcggcccgaggtcgatgtgatgtgcacagcgtttcacgacaacgaagaaacattcctgaagaaatacttgtacgagattgctaggcgacacccatatttctacgcgccggagcttctcttctttgcgaagcgctacaaggctgcatttacagagtgctgccaagccgctgataaggcggcctgtcttctccccaagctcgatgaactccgagatgaagggaaagcttcatcagcgaaacaaagattgaaatgtgcttccctccaaaagtttggagaacgagcctttaaggcttgggcagtggcacggctcagtcagcgctttcctaaggctgaatttgccgaagtgtccaagcttgtaacggatctcactaaagttcatactgaatgctgccacggagaccttctcgaatgcgcggacgatcgcgcggacttggcgaaatatatatgcgagaatcaagatagtatcagcagtaaactcaaagagtgctgcgagaagcctctcctcgaaaagagccactgtatcgccgaggtggaaaatgatgagatgcctgcggacttgccatcccttgccgcagactttgtcgaatcaaaagacgtttgcaagaattacgcggaggcaaaagatgtattccttggcatgttcttgtacgaatacgcacggcgccaccctgactattcagtagtgttgctcttgagactcgctaaaacatacgaaacgacgcttgagaaatgttgcgcagcagccgatccccacgagtgttacgcaaaggtgttcgacgagtttaaacccctcgttgaagaacctcaaaacctgataaaacaaaattgtgagttgttcgagcagttgggagagtacaagtttcagaatgctctcctggttcggtacaccaagaaggtcccacaagtgtccacgcccaccctcgtagaggtatcacggaaccttggcaaggtcggtagcaagtgctgcaaacacccagaagctaagcgcatgccatgcgctgaagactatctgtctgtggtgcttaatcaattgtgtgtactgcatgagaaaactcctgtgtccgaccgggttaccaagtgctgtaccgagtcactcgtcaaccggcgaccttgtttttctgcgctggaggttgatgagacgtatgttccgaaagaattcaacgccgagactttcaccttccatgctgatatatgcacactcagtgaaaaagaacgacaaataaagaagcaaaccgcattggtcgagctggtcaagcataaacccaaagctacaaaagaacaattgaaggctgttatggatgactttgccgcgttcgtagagaaatgctgcaaggcagatgataaagagacatgtttcgccgaagagggcaagaaactggtggccgcctctcaagctgcacttgggctctctggaggcgaacaaaagcttataagcgaagaggacttgtga

SEQ ID NO: 130 - protein, 840 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVSSAGGGSSGGSSAGGGSSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 131 - DNA, 2523 bp, synthetic construct
  source 1..2523: mol_type = other DNA
atggaaaccgacacactgctgctgtgggtgctgcttttgtgggtgccaggatccacaggccagggacagtccggatctaggcatccatgtcggcatgacccacatatcatatgctacaaattcggtggaggatcttctggaggctcaatttcatcaggactcctgtccgggcgctccgacaatattggtggctcccaggagaggagatcacatgaagtcaacccggccgcccatcttactggtgccaatagtagtcttacaggttctggcggacccttgctttgggagacccagctgggactggctttccttagaggcctgagttatcacgacggggcgttggtcgttactaaagccgggtattactatatatactctaaggttcagctcggcggtgtaggatgcccactgggtcttgcgtctacaattacacatggtctgtataagaggactcccagatatccagaagaactggaattgcttgtttcccagcaatctccgtgcggacgcgcaacgagctccagtagggtgtggtgggactccagcttcctgggtggcgttgtccatttggaggcgggagaggaagtagtggtgcgggtccttgatgaacgccttgtacgacttcgggacggtacaagaagctactttggggctttcatggtatcttctgcagggggggggtcatccggcggaagttcagcgggggggggatcctccggtggcagtgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttaccgaatgctgccaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttgtcaggtggggagcagaaacttatctccgaagaggatttgtaa

SEQ ID NO: 132 - protein, 840 aa, synthetic construct
METDTLLLWVLLLWVPGSTGQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVESKYGPPCPSCPAPEFLGGPSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGEQKLISEEDL

SEQ ID NO: 133 - DNA, 2523 bp, synthetic construct
  source 1..2523: mol_type = other DNA
atggaaaccgacacactgctgctgtgggtgctgcttttgtgggtgccaggatccacaggccaaggccagagtgggagtaggcatccatgtcgccatgatcctcacattatatgctataaatttggtgggggaagcagtggtgggtccatcagtagtgggctgttgtccggtcggagtgataatattggaggctcccaggaacgcaggagtcatgaagttaacccggcggcacatcttacgggtgcgaactctagtcttaccgggagtggggggcccctgctttgggagacacagcttgggctcgctttcctcaggggactctcttaccatgatggcgcactggtagtaaccaaagctggatactattatatttactctaaagttcaattggggggcgttggctgcccactcggcctcgcatctactatcacccatgggttgtataagcggacccctagataccctgaggaacttgagcttttggtttctcaacagtcaccttgcggtagggctacctcatcaagccgcgtttggtgggacagttcatttctcgggggcgtagtccatctggaggcaggtgaggaagtagtggttcgagtgctcgacgagcgcctcgtacgactccgggatgggacgagaagttatttcggcgcctttatggtagagtcaaaatacgggcctccctgtccctcttgtccggcacccgaatttcttggcgggccctctgatgcccacaagtctgaagttgcgcatagatttaaagacctcggagaggagaattttaaagcactcgtgcttatcgcatttgcgcagtacctgcagcagtgccctttcgaggaccacgtcaagcttgtcaacgaggtgacagaattcgccaaaacatgtgtcgccgatgagtctgccgagaactgtgacaaaagtttgcataccctgttcggcgacaaactctgcactgtagcaactcttagggaaacatacggggaaatggcagactgttgcgcgaaacaggagccagaacgaaacgaatgtttcttgcagcacaaggatgataaccctaatctcccgaggttggtcaggcccgaagtcgacgtaatgtgcacggctttccatgacaatgaggaaaccttcctcaagaagtacctctacgaaatagcacgaagacatccgtatttctatgctcctgagctgctctttttcgctaaacgctataaggccgcattcacggagtgttgtcaagccgccgataaggctgcttgccttctgcccaagttggacgaactgcgcgatgaggggaaagcatcatcagcaaagcaacgcctgaaatgcgctagtttgcaaaaattcggggaacgcgccttcaaagcttgggctgtcgcgcggctctcacagaggttccccaaggctgagttcgccgaagtttccaagctggttactgatttgaccaaagtgcacacagaatgttgccacggcgacctgcttgagtgtgcggatgaccgcgcagatctcgcgaagtatatatgcgaaaaccaggattcaattagctctaaattgaaagaatgttgtgagaaacctctgcttgaaaagtcacactgcattgcggaggtggaaaatgatgaaatgcccgcagatctcccctctttggcagcggacttcgtggagagtaaagacgtctgtaagaattacgccgaggcgaaggatgtattcctggggatgtttctctatgaatacgctcgcagacatcctgactactctgtggtgctgctgttgcgcctcgctaagacctacgaaacaaccctggaaaaatgttgtgccgcagctgatccacacgaatgctatgctaaagtctttgatgaatttaagcctttggttgaggagccccagaacctgataaaacagaactgtgaattgttcgagcaacttggagagtataaattccaaaacgcgcttctcgtgcggtacaccaagaaggtgcctcaagtcagtactccaacccttgtggaggtaagtcgcaatctcggtaaagttggcagtaaatgttgtaagcatcctgaagcgaagcgcatgccttgtgcagaggactatctgtcagtagttcttaaccagctgtgtgtgcttcatgagaaaacacccgtgtccgacagggtgacaaagtgttgcactgagagtctcgtgaaccggagaccttgtttctccgccctggaagtggacgaaacctatgtgccgaaggaattcaatgctgaaacgtttacgttccatgccgacatctgcacgcttagcgagaaagagagacagataaagaagcaaaccgccttggtggaactcgttaagcacaagccaaaagcaaccaaagaacaactcaaagcggtcatggacgatttcgctgcatttgtagagaaatgctgcaaagcggatgacaaggagacgtgtttcgctgaagagggaaaaaagctcgtggcagcatcccaggctgcacttggtctgtctggtggtgagcaaaaactgatttcagaggaggatttgtga

SEQ ID NO: 134 - protein, 830 aa, synthetic construct
METDTLLLWVLLLWVPGSTGGSGEQKLISEEDLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSSGGSGSQGQSGSRHPCRHDPHIICYKFGGGSSGGSISSGLLSGRSDNIGGSQERRSHEVNPAAHLTGANSSLTGSGGPLLWETQLGLAFLRGLSYHDGALVVTKTGYYYIYSKVQLGGVGCPLGLAGTITHGLYKRTPRYPEELELLVSQQSPCGRATSSSRVWWDSSFLGGVVHLEAGEEVVVRVLGKRLVRLRDGTRSYFGAFMV

SEQ ID NO: 135 - DNA, 2566 bp, synthetic construct
  source 1..2566: mol_type = other DNA
atggaaaccgacacactgctgctgtgggtgctgcttttgtgggtgccaggatccacaggcggctctggcgaacaaaaactcatttccgaggaagatcttggcgatgcacataaaagtgaggtggcccaccgctttaaagacctcggcgaggagaacttcaaggcactcgtacttattgcctttgcacaatacctgcagcaatgtccgtttgaagatcatgtcaagctcgtaaatgaagtgacagagttcgctaagacctgtgtagctgacgaaagtgccgaaaactgcgacaaatcattgcatacattgttcggcgacaagctttgtacagttgccacgctccgcgagacatacggcgaaatggccgattgttgtgcgaaacaggaacccgaaagaaatgagtgcttcttgcaacacaaagacgataatcccaatcttccgcgactggtgagacccgaagtagatgtaatgtgcacagcttttcatgacaatgaggaaacctttcttaaaaaatatctgtacgaaatcgcccgccgacacccgtatttctatgccccagagctgttgtttttcgcaaagcgctacaaggcagcgtttacagaatgctgtcaggccgctgataaagccgcttgtctgctgcctaagcttgatgagcttcgcgacgagggtaaggcttccagcgctaaacaacgcctgaagtgtgcatctttgcaaaaatttggtgaacgagccttcaaggcttgggctgttgccaggctgagtcaacggttcccgaaggcggagtttgcggaggtttccaagttggtaacggacctgacaaaggtacataccgaatgctgccatggcgacctgttggagtgcgccgatgatcgagcggatcttgccaagtatatttgcgaaaaccaagactccatttccagtaaacttaaggagtgttgtgagaaacctctccttgaaaagagccattgtatagcggaggtcgaaaacgatgaaatgcccgctgatctgccgtcactcgctgctgacttcgttgaatccaaggatgtatgtaaaaactatgccgaagctaaggacgtctttcttggaatgttcttgtatgaatacgcccgacggcatcctgactattcagttgtgctccttcttcgattggctaaaacttatgagacgacacttgaaaagtgttgtgccgccgccgacccacatgagtgctacgctaaggttttcgacgaatttaagcccttggttgaagaaccgcagaatctcattaagcaaaattgtgagctctttgaacaactcggtgaatacaaatttcagaacgcgcttctggttagatatacgaagaaagtgccacaggtgagcacccctacattggttgaggtcagtaggaacctcggcaaggtgggatctaagtgctgcaaacacccggaggcaaagagaatgccttgcgctgaggattatctctcagtggttctgaatcagctttgcgttcttcacgaaaagactcccgtcagcgaccgagtcacgaagtgctgtaccgagagccttgttaatagacgaccttgcttctccgcgcttgaggtggatgagacctacgtcccaaaggaattcaacgccgagacgttcactttccatgccgatatttgcaccttgtcagaaaaggagcgacagataaaaaagcaaactgctttggtggagctggttaagcataagcctaaagcgacgaaggaacagctcaaagccgtaatggatgatttcgctgccttcgtggagaaatgttgtaaggccgatgacaaggaaacctgcttcgctgaagagggtaaaaagctcgtcgccgctagtcaggcggcacttggtttggggggcggaagcagcggcggcagcggcagccaaggccagagcggcagcaggcacccttgtagacacgatcctcacatcatctgttacaagtttggaggaggcagtagcggtggcagtattagcagcggactgctgagtggcagaagcgacaacatcggcggcagccaagaacgccgatcacacgaagtgaaccctgctgctcacttgactggagctaactccagccttacggggagcgggggtcctttgctctgggaaacgcagctgggtctcgcgtttcttagagggctgagttatcacgatggcgccttggtcgtcacgaagactgggtactactatatttatagtaaggtacagttggggggagtggggtgccctctcggcctggctgggacgatcacgcatgggctctacaagcgcacgcctaggtatccggaggagttggaactgttggtctcacagcagtccccgtgcgggcgagcgacttcctccagtagagtgtggtgggactctagctttctcgggggagtggttcatcttgaggcaggagaggaagtggtggtacgggttttgggcaagagattggtgagactgcgggatggtacaagatcttattttggtgctttcatggtctgaaccggttagtaatgagtttgatatctcgacaatcaacctctggattacaaaatttgtgaaagattgactggta

SEQ ID NO: 136 - protein, 5 aa, synthetic construct
GQSGQ

SEQ ID NO: 137 - protein, 4 aa, synthetic construct
QSGQ

SEQ ID NO: 138 - protein, 5 aa, synthetic construct
QGQSG

SEQ ID NO: 139 - protein, 4 aa, synthetic construct
QGQS

SEQ ID NO: 140 - protein, 10 aa, synthetic construct
AANALAHGLF

SEQ ID NO: 141 - protein, 4 aa, synthetic construct
AANL

SEQ ID NO: 142 - protein, 10 aa, synthetic construct
AANLGSGGSS

SEQ ID NO: 143 - protein, 5 aa, synthetic construct
AAPRS

SEQ ID NO: 144 - protein, 6 aa, synthetic construct
AAPRSF

SEQ ID NO: 145 - protein, 8 aa, synthetic construct
AARGPAIH

SEQ ID NO: 146 - protein, 8 aa, synthetic construct
AAYHLVSQ

SEQ ID NO: 147 - protein, 10 aa, synthetic construct
AFPDMRSVRS

SEQ ID NO: 148 - protein, 7 aa, synthetic construct
AFQALRM

SEQ ID NO: 149 - protein, 6 aa, synthetic construct
AFRHLR

SEQ ID NO: 150 - protein, 8 aa, synthetic construct
AGLGISST

SEQ ID NO: 151 - protein, 8 aa, synthetic construct
AGLGVVER

SEQ ID NO: 152 - protein, 4 aa, synthetic construct
AGPR

SEQ ID NO: 153 - protein, 4 aa, synthetic construct
AHGL

SEQ ID NO: 154 - protein, 5 aa, synthetic construct
AHGLF

SEQ ID NO: 155 - protein, 7 aa, synthetic construct
AHQALRM

SEQ ID NO: 156 - protein, 10 aa, synthetic construct
AIPRVRLFDV

SEQ ID NO: 157 - protein, 5 aa, synthetic construct
ALAHG

SEQ ID NO: 158 - protein, 6 aa, synthetic construct
ALAHGL

SEQ ID NO: 159 - protein, 7 aa, synthetic construct
ALAHGLF

SEQ ID NO: 160 - protein, 12 aa, synthetic construct
ALAHGLFAPRSF

SEQ ID NO: 161 - protein, 13 aa, synthetic construct
ALAHGLFSGRSAN

SEQ ID NO: 162 - protein, 12 aa, synthetic construct
ALAHGLPTFVHL

SEQ ID NO: 163 - protein, 8 aa, synthetic construct
ALGLLRLP

SEQ ID NO: 164 - protein, 10 aa, synthetic construct
ALPSVKMVSE

SEQ ID NO: 165 - protein, 5 aa, synthetic construct
ALRAP

SEQ ID NO: 166 - protein, 7 aa, synthetic construct
ANQALRM

SEQ ID NO: 167 - protein, 8 aa, synthetic construct
ANQALRMA

SEQ ID NO: 168 - protein, 10 aa, synthetic construct
APPLVKSMVV

SEQ ID NO: 169 - protein, 10 aa, synthetic construct
APPSFKLVNA

SEQ ID NO: 170 - protein, 4 aa, synthetic construct
APRS

SEQ ID NO: 171 - protein, 11 aa, synthetic construct
APRSALAHGLF

SEQ ID NO: 172 - protein, 5 aa, synthetic construct
APRSF

SEQ ID NO: 173 - protein, 8 aa, synthetic construct
AQFVLTEG

SEQ ID NO: 174 - protein, 8 aa, synthetic construct
AQNLLGMV

SEQ ID NO: 175 - protein, 4 aa, synthetic construct
ARGP

SEQ ID NO: 176 - protein, 5 aa, synthetic construct
ARGPS

SEQ ID NO: 177 - protein, 6 aa, synthetic construct
ARGPSF

SEQ ID NO: 178 - protein, 7 aa, synthetic construct
ARGPSFK

SEQ ID NO: 179 - protein, 8 aa, synthetic construct
ASGLLRFP

SEQ ID NO: 180 - protein, 10 aa, synthetic construct
ASPTMKTVGL

SEQ ID NO: 181 - protein, 8 aa, synthetic construct
AVGLLAPP

SEQ ID NO: 182 - protein, 18 aa, synthetic construct
AVGLLAPPGGLSGRSANI

SEQ ID NO: 183 - protein, 18 aa, synthetic construct
AVGLLAPPGGLSGRSANP

SEQ ID NO: 184 - protein, 18 aa, synthetic construct
AVGLLAPPGGLSGRSDDH

SEQ ID NO: 185 - protein, 18 aa, synthetic construct
AVGLLAPPGGLSGRSDIH

SEQ ID NO: 186 - protein, 18 aa, synthetic construct
AVGLLAPPGGLSGRSDNH

SEQ ID NO: 187 - protein, 18 aa, synthetic construct
AVGLLAPPGGLSGRSDNI

SEQ ID NO: 188 - protein, 18 aa, synthetic construct
AVGLLAPPGGLSGRSDNP

SEQ ID NO: 189 - protein, 18 aa, synthetic construct
AVGLLAPPGGLSGRSDQH

SEQ ID NO: 190 - protein, 18 aa, synthetic construct
AVGLLAPPGGLSGRSDTH

SEQ ID NO: 191 - protein, 18 aa, synthetic construct
AVGLLAPPGGLSGRSDYH

SEQ ID NO: 192 - protein, 17 aa, synthetic construct
AVGLLAPPGGLSGRSNI

SEQ ID NO: 193 - protein, 18 aa, synthetic construct
AVGLLAPPGGLSGRSNIG

SEQ ID NO: 194 - protein, 19 aa, synthetic construct
AVGLLAPPGGLSGRSNIGS

SEQ ID NO: 195 - protein, 22 aa, synthetic construct
AVGLLAPPGGTSTSGRSANPRG

SEQ ID NO: 196 - protein, 17 aa, synthetic construct
AVGLLAPPSGRSANPRG

SEQ ID NO: 197 - protein, 18 aa, synthetic construct
AVGLLAPPTSGRSANPRG

SEQ ID NO: 198 - protein, 10 aa, synthetic construct
AVPKVRVVPE

SEQ ID NO: 199 - protein, 7 aa, synthetic construct
CGPPLGR

SEQ ID NO: 200 - protein, 7 aa, synthetic construct
CSPPLGR

SEQ ID NO: 201 - protein, 10 aa, synthetic construct
DEVDGSGGSS

SEQ ID NO: 202 - skipped

SEQ ID NO: 203 - protein, 8 aa, synthetic construct
DISHWRRS

SEQ ID NO: 204 - protein, 7 aa, synthetic construct
DLAHPLL

SEQ ID NO: 205 - protein, 10 aa, synthetic construct
DLPLVKSLPS

SEQ ID NO: 206 - skipped

SEQ ID NO: 207 - protein, 12 aa, synthetic construct
DRLSGRSANHKK

SEQ ID NO: 208 - protein, 12 aa, synthetic construct
DRLSGRSDNHKK

SEQ ID NO: 209 - protein, 10 aa, synthetic construct
DRPEMKSLSG

SEQ ID NO: 210 - protein, 10 aa, synthetic construct
DRPKVKTMDF

SEQ ID NO: 211 - protein, 8 aa, synthetic construct
DVAQFVLT

SEQ ID NO: 212 - protein, 10 aa, synthetic construct
DVPPMKTLRP

SEQ ID NO: 213 - protein, 8 aa, synthetic construct
DWLYWMGI

SEQ ID NO: 214 - protein, 8 aa, synthetic construct
DWLYWMSI

SEQ ID NO: 215 - protein, 8 aa, synthetic construct
DWLYWPGI

SEQ ID NO: 216 - protein, 8 aa, synthetic construct
DWLYWPSI

SEQ ID NO: 217 - protein, 10 aa, synthetic construct
EAPKVKALPK

SEQ ID NO: 218 - protein, 10 aa, synthetic construct
EHPRVKVVSE

SEQ ID NO: 219 - protein, 10 aa, synthetic construct
EKPRMKLFQG

SEQ ID NO: 220 - protein, 8 aa, synthetic construct
EPQALAMS

SEQ ID NO: 221 - protein, 10 aa, synthetic construct
EQPEVKMVKG

SEQ ID NO: 222 - protein, 10 aa, synthetic construct
ERPGVKSLVL

SEQ ID NO: 223 - protein, 8 aa, synthetic construct
ESLPVVAV

SEQ ID NO: 224 - protein, 10 aa, synthetic construct
ESPVMKSMAL

SEQ ID NO: 225 - protein, 5 aa, synthetic construct
ESRRW

SEQ ID NO: 226 - protein, 6 aa, synthetic construct
ESRRWM

SEQ ID NO: 227 - protein, 7 aa, synthetic construct
ESRRWMP

SEQ ID NO: 228 - protein, 10 aa, synthetic construct
ETPSVKTMGR

SEQ ID NO: 229 - protein, 12 aa, synthetic construct
ETPSVKTMGRSS

SEQ ID NO: 230 - protein, 10 aa, synthetic construct
FPRPLGITGL

SEQ ID NO: 231 - protein, 8 aa, synthetic construct
FRLLDWQW

SEQ ID NO: 232 - protein, 8 aa, synthetic construct
GCGPPLGR

SEQ ID NO: 233 - protein, 8 aa, synthetic construct
GCSPPLGR

SEQ ID NO: 234 - protein, 10 aa, synthetic construct
GFPHMKTFQH

SEQ ID NO: 235 - protein, 12 aa, synthetic construct
GFPHMKTFQHSS

SEQ ID NO: 236 - protein, 8 aa, synthetic construct
GGGPPLGR

SEQ ID NO: 237 - protein, 7 aa, synthetic construct
GGPPLGR

SEQ ID NO: 238 - protein, 10 aa, synthetic construct
GGQPSGMWGW

SEQ ID NO: 239 - protein, 7 aa, synthetic construct
GGSIDGR

SEQ ID NO: 240 - protein, 8 aa, synthetic construct
GGSPPLGR

SEQ ID NO: 241 - protein, 8 aa, synthetic construct
GGWHTGRN

SEQ ID NO: 242 - protein, 5 aa, synthetic construct
GIAGQ

SEQ ID NO: 243 - protein, 10 aa, synthetic construct
GLGTPRGLFA

SEQ ID NO: 244 - protein, 6 aa, synthetic construct
GLPTFV

SEQ ID NO: 245 - protein, 7 aa, synthetic construct
GLPTFVH

SEQ ID NO: 246 - protein, 8 aa, synthetic construct
GLPTFVHL

SEQ ID NO: 247 - protein, 12 aa, synthetic construct
GLPTFVHLPRQV

SEQ ID NO: 248 - protein, 12 aa, synthetic construct
GLSGRSDNHGSS

SEQ ID NO: 249 - protein, 8 aa, synthetic construct
GPEGLRVG

SEQ ID NO: 250 - protein, 8 aa, synthetic construct
GPLGIAGI

SEQ ID NO: 251 - protein, 12 aa, synthetic construct
GPLNGRSDNHKA

SEQ ID NO: 252 - protein, 12 aa, synthetic construct
GPLNGRSDNHKK

SEQ ID NO: 253 - protein, 12 aa, synthetic construct
GPLNGRSDNHKR

SEQ ID NO: 254 - protein, 12 aa, synthetic construct
GPLNGRSDNHQA

SEQ ID NO: 255 - protein, 12 aa, synthetic construct
GPLNGRSDNHQK

SEQ ID NO: 256 - protein, 12 aa, synthetic construct
GPLNGRSDNHQR

SEQ ID NO: 257 - protein, 12 aa, synthetic construct
GPLNGRSDNHRA

SEQ ID NO: 258 - protein, 12 aa, synthetic construct
GPLNGRSDNHRK

SEQ ID NO: 259 - protein, 12 aa, synthetic construct
GPLNGRSDNHRR

SEQ ID NO: 260 - protein, 12 aa, synthetic construct
GPLSGRSDNHKA

SEQ ID NO: 261 - protein, 12 aa, synthetic construct
GPLSGRSDNHKK

SEQ ID NO: 262 - protein, 12 aa, synthetic construct
GPLSGRSDNHKR

SEQ ID NO: 263 - protein, 12 aa, synthetic construct
GPLSGRSDNHQA

SEQ ID NO: 264 - protein, 12 aa, synthetic construct
GPLSGRSDNHQK

SEQ ID NO: 265 - protein, 12 aa, synthetic construct
GPLSGRSDNHQR

SEQ ID NO: 266 - protein, 12 aa, synthetic construct
GPLSGRSDNHRA

SEQ ID NO: 267 - protein, 12 aa, synthetic construct
GPLSGRSDNHRK

SEQ ID NO: 268 - protein, 12 aa, synthetic construct
GPLSGRSDNHRR

SEQ ID NO: 269 - protein, 6 aa, synthetic construct
GPPLGR

SEQ ID NO: 270 - protein, 8 aa, synthetic construct
GPQGIAGQ

SEQ ID NO: 271 - protein, 8 aa, synthetic construct
GPQGLLGA

SEQ ID NO: 272 - protein, 6 aa, synthetic construct
GPRSFG

SEQ ID NO: 273 - protein, 7 aa, synthetic construct
GPRSFGL

SEQ ID NO: 274 - protein, 8 aa, synthetic construct
GPSHLVLT

SEQ ID NO: 275 - protein, 4 aa, synthetic construct
GPTN

SEQ ID NO: 276 - protein, 11 aa, synthetic construct
GPTNALAHGLF

SEQ ID NO: 277 - protein, 5 aa, synthetic construct
GRSML

SEQ ID NO: 278 - protein, 6 aa, synthetic construct
GRSMLL

SEQ ID NO: 279 - protein, 7 aa, synthetic construct
GRSMLLG

SEQ ID NO: 280 - protein, 8 aa, synthetic construct
GRSMLLGG

SEQ ID NO: 281 - protein, 8 aa, synthetic construct
GRSMLLGP

SEQ ID NO: 282 - protein, 8 aa, synthetic construct
GRSMLLGS

SEQ ID NO: 283 - protein, 7 aa, synthetic construct
GRSMLLP

SEQ ID NO: 284 - protein, 8 aa, synthetic construct
GRSMLLPG

SEQ ID NO: 285 - protein, 8 aa, synthetic construct
GRSMLLPP

SEQ ID NO: 286 - protein, 8 aa, synthetic construct
GRSMLLPS

SEQ ID NO: 287 - protein, 7 aa, synthetic construct
GRSMLLS

SEQ ID NO: 288 - protein, 8 aa, synthetic construct
GRSMLLSG

SEQ ID NO: 289 - protein, 8 aa, synthetic construct
GRSMLLSP

SEQ ID NO: 290 - protein, 8 aa, synthetic construct
GRSMLLSS

SEQ ID NO: 291 - protein, 6 aa, synthetic construct
GRSMLM

SEQ ID NO: 292 - protein, 7 aa, synthetic construct
GRSMLMG

SEQ ID NO: 293 - protein, 8 aa, synthetic construct
GRSMLMGG

SEQ ID NO: 294 - protein, 8 aa, synthetic construct
GRSMLMGP

SEQ ID NO: 295 - protein, 8 aa, synthetic construct
GRSMLMGS

SEQ ID NO: 296 - protein, 7 aa, synthetic construct
GRSMLMP

SEQ ID NO: 297 - protein, 8 aa, synthetic construct
GRSMLMPG

SEQ ID NO: 298 - protein, 8 aa, synthetic construct
GRSMLMPP

SEQ ID NO: 299 - protein, 8 aa, synthetic construct
GRSMLMPS

SEQ ID NO: 300 - protein, 7 aa, synthetic construct
GRSMLMS

SEQ ID NO: 301 - protein, 8 aa, synthetic construct
GRSMLMSG

SEQ ID NO: 302 - protein, 8 aa, synthetic construct
GRSMLMSP

SEQ ID NO: 303 - protein, 8 aa, synthetic construct
GRSMLMSS

SEQ ID NO: 304 - protein, 8 aa, synthetic construct
GSGPPLGR

SEQ ID NO: 305 - protein, 7 aa, synthetic construct
GSPPLGR

SEQ ID NO: 306 - protein, 8 aa, synthetic construct
GSSPPLGR

SEQ ID NO: 307 - protein, 12 aa, synthetic construct
GTGRGPSWVGSS

SEQ ID NO: 308 - protein, 8 aa, synthetic construct
HMMQYARH

SEQ ID NO: 309 - protein, 8 aa, synthetic construct
HTGRSGAL

SEQ ID NO: 310 - protein, 5 aa, synthetic construct
HVPRQ

SEQ ID NO: 311 - protein, 6 aa, synthetic construct
HVPRQV

SEQ ID NO: 312 - protein, 11 aa, synthetic construct
HVPRQVAPRSF

SEQ ID NO: 313 - protein, 11 aa, synthetic construct
HVPRQVLSGRS

SEQ ID NO: 314 - protein, 13 aa, synthetic construct
HVPRQVLSGRSAN

SEQ ID NO: 315 - protein, 8 aa, synthetic construct
HWHLGPPT

SEQ ID NO: 316 - protein, 8 aa, synthetic construct
IANLLSMV

SEQ ID NO: 317 - protein, 4 aa, synthetic construct
IDGR

SEQ ID NO: 318 - protein, 4 aa, synthetic construct
IEGR

SEQ ID NO: 319 - protein, 8 aa, synthetic construct
ILNLLSMV

SEQ ID NO: 320 - protein, 8 aa, synthetic construct
ILPRSPAF

SEQ ID NO: 321 - protein, 8 aa, synthetic construct
IPFSWSRF

SEQ ID NO: 322 - protein, 8 aa, synthetic construct
IQNLLSMV

SEQ ID NO: 323 - protein, 5 aa, synthetic construct
ISSGL

SEQ ID NO: 324 - protein, 6 aa, synthetic construct
ISSGLL

SEQ ID NO: 325 - protein, 7 aa, synthetic construct
ISSGLLS

SEQ ID NO: 326 - protein, 13 aa, synthetic construct
ISSGLLSGRSANI

SEQ ID NO: 327 - protein, 13 aa, synthetic construct
ISSGLLSGRSANP

SEQ ID NO: 328 - protein, 15 aa, synthetic construct
ISSGLLSGRSANPRG

SEQ ID NO: 329 - protein, 13 aa, synthetic construct
ISSGLLSGRSDDH

SEQ ID NO: 330 - protein, 13 aa, synthetic construct
ISSGLLSGRSDIH

SEQ ID NO: 331 - protein, 13 aa, synthetic construct
ISSGLLSGRSDNH

SEQ ID NO: 332 - protein, 13 aa, synthetic construct
ISSGLLSGRSDNI

SEQ ID NO: 333 - protein, 13 aa, synthetic construct
ISSGLLSGRSDNP

SEQ ID NO: 334 - protein, 13 aa, synthetic construct
ISSGLLSGRSDQH

SEQ ID NO: 335 - protein, 13 aa, synthetic construct
ISSGLLSGRSDTH

SEQ ID NO: 336 - protein, 13 aa, synthetic construct
ISSGLLSGRSDYH

SEQ ID NO: 337 - protein, 13 aa, synthetic construct
ISSGLLSGRSGNH

SEQ ID NO: 338 - protein, 12 aa, synthetic construct
ISSGLLSGRSNI

SEQ ID NO: 339 - protein, 13 aa, synthetic construct
ISSGLLSGRSNIG

SEQ ID NO: 340 - protein, 14 aa, synthetic construct
ISSGLLSGRSNIGS

SEQ ID NO: 341 - protein, 8 aa, synthetic construct
ISSGLLSS

SEQ ID NO: 342 - protein, 22 aa, synthetic construct
ISSGLLSSGGSGGSLSGRSDNH

SEQ ID NO: 343 - protein, 22 aa, synthetic construct
ISSGLLSSGGSGGSLSGRSGNH

SEQ ID NO: 344 - protein, 7 aa, synthetic construct
ISSGLSS

SEQ ID NO: 345 - protein, 6 aa, synthetic construct
IVSRSA

SEQ ID NO: 346 - protein, 12 aa, synthetic construct
KGLTGRSDRHQA

SEQ ID NO: 347 - protein, 10 aa, synthetic construct
KGPKVKVVTL

SEQ ID NO: 348 - protein, 12 aa, synthetic construct
KNLYGRSENNGN

SEQ ID NO: 349 - protein, 8 aa, synthetic construct
KRMPVQFL

SEQ ID NO: 350 - protein, 8 aa, synthetic construct
LAAPLGLL

SEQ ID NO: 351 - protein, 4 aa, synthetic construct
LAHG

SEQ ID NO: 352 - protein, 5 aa, synthetic construct
LAHGL

SEQ ID NO: 353 - protein, 6 aa, synthetic construct
LAHGLF

SEQ ID NO: 354 - protein, 9 aa, synthetic construct
LAPLGLQRR

SEQ ID NO: 355 - protein, 5 aa, synthetic construct
LARAG

SEQ ID NO: 356 - protein, 6 aa, synthetic construct
LARAGI

SEQ ID NO: 357 - protein, 6 aa, synthetic construct
LARAGL

SEQ ID NO: 358 - protein, 8 aa, synthetic construct
LKAAPRWA

SEQ ID NO: 359 - protein, 8 aa, synthetic construct
LKAAPRWF

SEQ ID NO: 360 - protein, 8 aa, synthetic construct
LKAAPVWA

SEQ ID NO: 361 - protein, 8 aa, synthetic construct
LKAAPVWF

SEQ ID NO: 362 - protein, 8 aa, synthetic construct
LKGRSYYY

SEQ ID NO: 363 - protein, 8 aa, synthetic construct
LLAPSHRA

SEQ ID NO: 364 - protein, 8 aa, synthetic construct
LLEALRAL

SEQ ID NO: 365 - protein, 8 aa, synthetic construct
LLESLRAL

SEQ ID NO: 366 - protein, 8 aa, synthetic construct
LLLPAHGG

SEQ ID NO: 367 - protein, 8 aa, synthetic construct
LLLPLLGS

SEQ ID NO: 368 - protein, 8 aa, synthetic construct
LLNALRAL

SEQ ID NO: 369 - protein, 8 aa, synthetic construct
LLNSLRAL

SEQ ID NO: 370 - protein, 8 aa, synthetic construct
LLQALRAL

SEQ ID NO: 371 - protein, 8 aa, synthetic construct
LLQSLRAL

SEQ ID NO: 372 - protein, 8 aa, synthetic construct
LLSALRAL

SEQ ID NO: 373 - protein, 8 aa, synthetic construct
LLSSLRAL

SEQ ID NO: 374 - protein, 8 aa, synthetic construct
LNGRSDNH

SEQ ID NO: 375 - protein, 7 aa, synthetic construct
LPAGLLL

SEQ ID NO: 376 - protein, 8 aa, synthetic construct
LPAGLLLR

SEQ ID NO: 377 - protein, 8 aa, synthetic construct
LPAHLVLL

SEQ ID NO: 378 - protein, 8 aa, synthetic construct
LPAHLVLV

SEQ ID NO: 379 - protein, 8 aa, synthetic construct
LPGGLSPW

SEQ ID NO: 380 - protein, 8 aa, synthetic construct
LPSHLVLL

SEQ ID NO: 381 - protein, 8 aa, synthetic construct
LPSHLVLV

SEQ ID NO: 382 - protein, 5 aa, synthetic construct
LPTFV

SEQ ID NO: 383 - protein, 6 aa, synthetic construct
LPTFVH

SEQ ID NO: 384 - protein, 7 aa, synthetic construct
LPTFVHL

SEQ ID NO: 385 - protein, 5 aa, synthetic construct
LRSGW

SEQ ID NO: 386 - protein, 4 aa, synthetic construct
LSGR

SEQ ID NO: 387 - protein, 5 aa, synthetic construct
LSGRS

SEQ ID NO: 388 - protein, 6 aa, synthetic construct
LSGRSA

SEQ ID NO: 389 - protein, 12 aa, synthetic construct
LSGRSALAHGLF

SEQ ID NO: 390 - protein, 7 aa, synthetic construct
LSGRSAN

SEQ ID NO: 391 - protein, 8 aa, synthetic construct
LSGRSANI

SEQ ID NO: 392 - protein, 8 aa, synthetic construct
LSGRSANP

SEQ ID NO: 393 - protein, 6 aa, synthetic construct
LSGRSD

SEQ ID NO: 394 - protein, 7 aa, synthetic construct
LSGRSDD

SEQ ID NO: 395 - protein, 8 aa, synthetic construct
LSGRSDDH

SEQ ID NO: 396 - protein, 7 aa, synthetic construct
LSGRSDI

SEQ ID NO: 397 - protein, 8 aa, synthetic construct
LSGRSDIH

SEQ ID NO: 398 - protein, 7 aa, synthetic construct
LSGRSDN

SEQ ID NO: 399 - protein, 8 aa, synthetic construct
LSGRSDNH

SEQ ID NO: 400 - protein, 8 aa, synthetic construct
LSGRSDNH

SEQ ID NO: 401 - protein, 18 aa, synthetic construct
LSGRSDNHGGAVGLLAPP

SEQ ID NO: 402 - protein, 22 aa, synthetic construct
LSGRSDNHGGSGGSISSGLLSS

SEQ ID NO: 403 - protein, 22 aa, synthetic construct
LSGRSDNHGGSGGSQNQALRMA

SEQ ID NO: 404 - protein, 20 aa, synthetic construct
LSGRSDNHGGVHMPLGFLGP

SEQ ID NO: 405 - protein, 8 aa, synthetic construct
LSGRSDNI

SEQ ID NO: 406 - protein, 8 aa, synthetic construct
LSGRSDNP

SEQ ID NO: 407 - protein, 7 aa, synthetic construct
LSGRSDQ

SEQ ID NO: 408 - protein, 8 aa, synthetic construct
LSGRSDQH

SEQ ID NO: 409 - protein, 7 aa, synthetic construct
LSGRSDT

SEQ ID NO: 410 - protein, 8 aa, synthetic construct
LSGRSDTH

SEQ ID NO: 411 - protein, 7 aa, synthetic construct
LSGRSDY

SEQ ID NO: 412 - protein, 8 aa, synthetic construct
LSGRSDYH

SEQ ID NO: 413 - protein, 8 aa, synthetic construct
LSGRSENH

SEQ ID NO: 414 - protein, 6 aa, synthetic construct
LSGRSG

SEQ ID NO: 415 - protein, 7 aa, synthetic construct
LSGRSGN

SEQ ID NO: 416 - protein, 8 aa, synthetic construct
LSGRSGNH

SEQ ID NO: 417 - protein, 22 aa, synthetic construct
LSGRSGNHGGSGGSISSGLLSS

SEQ ID NO: 418 - protein, 22 aa, synthetic construct
LSGRSGNHGGSGGSQNQALRMA

SEQ ID NO: 419 - protein, 8 aa, synthetic construct
LSGRSGNP

SEQ ID NO: 420 - protein, 8 aa, synthetic construct
LSGRSVTQ

SEQ ID NO: 421 - protein, 8 aa, synthetic construct
LSQARWRK

SEQ ID NO: 422 - protein, 8 aa, synthetic construct
LTFPTYIF

SEQ ID NO: 423 - protein, 8 aa, synthetic construct
LTFPTYWF

SEQ ID NO: 424 - protein, 8 aa, synthetic construct
LTGRSDRH

SEQ ID NO: 425 - protein, 7 aa, synthetic construct
LTGRSGA

SEQ ID NO: 426 - protein, 7 aa, synthetic construct
LTGRSGA

SEQ ID NO: 427 - protein, 8 aa, synthetic construct
LYAAPRWA

SEQ ID NO: 428 - protein, 8 aa, synthetic construct
LYAAPRWF

SEQ ID NO: 429 - protein, 8 aa, synthetic construct
LYAAPVWA

SEQ ID NO: 430 - protein, 8 aa, synthetic construct
LYAAPVWF

SEQ ID NO: 431 - protein, 8 aa, synthetic construct
LYGRSENN

SEQ ID NO: 432 - protein, 8 aa, synthetic construct
MDAFLESS

SEQ ID NO: 433 - protein, 8 aa, synthetic construct
MGLFSEAG

SEQ ID NO: 434 - protein, 6 aa, synthetic construct
MGPWFM

SEQ ID NO: 435 - protein, 8 aa, synthetic construct
MIAPVAYR

SEQ ID NO: 436 - protein, 6 aa, synthetic construct
MLRSGW

SEQ ID NO: 437 - protein, 7 aa, synthetic construct
MLRSGWR

SEQ ID NO: 438 - protein, 8 aa, synthetic construct
MLRSGWRG

SEQ ID NO: 439 - protein, 8 aa, synthetic construct
MLRSGWRL

SEQ ID NO: 440 - protein, 8 aa, synthetic construct
MLRSGWRS

SEQ ID NO: 441 - protein, 8 aa, synthetic construct
MTFPTYIF

SEQ ID NO: 442 - protein, 8 aa, synthetic construct
MTFPTYWF

SEQ ID NO: 443 - protein, 12 aa, synthetic construct
NHRIGRSDNHRR

SEQ ID NO: 444 - protein, 10 aa, synthetic construct
NMPSFKLVTG

SEQ ID NO: 445 - protein, 12 aa, synthetic construct
NTLSGRSENHSG

SEQ ID NO: 446 - protein, 12 aa, synthetic construct
NTLSGRSGNHGS

SEQ ID NO: 447 - protein, 10 aa, synthetic construct
NZPRVRLVLP

SEQ ID NO: 448 - protein, 8 aa, synthetic construct
PAGLWLDP

SEQ ID NO: 449 - protein, 5 aa, synthetic construct
PAGRR

SEQ ID NO: 450 - protein, 6 aa, synthetic construct
PAGRRS

SEQ ID NO: 451 - protein, 7 aa, synthetic construct
PAGRRSL

SEQ ID NO: 452 - protein, 8 aa, synthetic construct
PASLWYTQ

SEQ ID NO: 453 - protein, 8 aa, synthetic construct
PESRRWMP

SEQ ID NO: 454 - protein, 6 aa, synthetic construct
PFHLSR

SEQ ID NO: 455 - protein, 6 aa, synthetic construct
PHGFFQ

SEQ ID NO: 456 - protein, 7 aa, synthetic construct
PLARAGI

SEQ ID NO: 457 - protein, 7 aa, synthetic construct
PLARAGL

SEQ ID NO: 458 - protein, 4 aa, synthetic construct
PLGL

SEQ ID NO: 459 - protein, 6 aa, synthetic construct
PLGLAG

SEQ ID NO: 460 - protein, 6 aa, synthetic construct
PLGLWA

SEQ ID NO: 461 - protein, 7 aa, synthetic construct
PLGVRGK

SEQ ID NO: 462 - protein, 8 aa, synthetic construct
PLTGRSGG

SEQ ID NO: 463 - protein, 12 aa, synthetic construct
PLTGRSGGGGSS

SEQ ID NO: 464 - protein, 5 aa, synthetic construct
PPLGR

SEQ ID NO: 465 - protein, 10 aa, synthetic construct
PPPDMKLFPG

SEQ ID NO: 466 - protein, 10 aa, synthetic construct
PPPEVRSFSV

SEQ ID NO: 467 - protein, 10 aa, synthetic construct
PPPVLKLLEW

SEQ ID NO: 468 - protein, 12 aa, synthetic construct
PPSIARSDNLAN

SEQ ID NO: 469 - protein, 8 aa, synthetic construct
PQHRIVSF

SEQ ID NO: 470 - protein, 8 aa, synthetic construct
PRFKIIGG

SEQ ID NO: 471 - protein, 8 aa, synthetic construct
PRFRIIGG

SEQ ID NO: 472 - protein, 10 aa, synthetic construct
PRPFVKSVDQ

SEQ ID NO: 473 - protein, 4 aa, synthetic construct
PRQV

SEQ ID NO: 474 - protein, 4 aa, synthetic construct
PRSF

SEQ ID NO: 475 - protein, 10 aa, synthetic construct
PSPPVKMMPE

SEQ ID NO: 476 - protein, 10 aa, synthetic construct
PTNGGSGGSS

SEQ ID NO: 477 - protein, 4 aa, synthetic construct
PTNL

SEQ ID NO: 478 - protein, 10 aa, synthetic construct
PTNLGSGGSS

SEQ ID NO: 479 - protein, 8 aa, synthetic construct
PVGYTSSL

SEQ ID NO: 480 - protein, 10 aa, synthetic construct
PVPRLKLIKD

SEQ ID NO: 481 - protein, 8 aa, synthetic construct
PVQPIGPQ

SEQ ID NO: 482 - protein, 8 aa, synthetic construct
QALAMSAI

SEQ ID NO: 483 - protein, 7 aa, synthetic construct
QFQALRM

SEQ ID NO: 484 - protein, 10 aa, synthetic construct
QGPMFKSLWD

SEQ ID NO: 485 - protein, 8 aa, synthetic construct
QGRAITFI

SEQ ID NO: 486 - protein, 7 aa, synthetic construct
QHQALRM

SEQ ID NO: 487 - protein, 8 aa, synthetic construct
QNQALRIA

SEQ ID NO: 488 - protein, 7 aa, synthetic construct
QNQALRM

SEQ ID NO: 489 - protein, 8 aa, synthetic construct
QNQALRMA

SEQ ID NO: 490 - protein, 22 aa, synthetic construct
QNQALRMAGGSGGSLSGRSDNH

SEQ ID NO: 491 - protein, 22 aa, synthetic construct
QNQALRMAGGSGGSLSGRSGNH

SEQ ID NO: 492 - protein, 6 aa, synthetic construct
QSRRVP

SEQ ID NO: 493 - protein, 7 aa, synthetic construct
QSRRVPL

SEQ ID NO: 494 - protein, 7 aa, synthetic construct
QSRRVPV

SEQ ID NO: 495 - protein, 6 aa, synthetic construct
QTRRVP

SEQ ID NO: 496 - protein, 7 aa, synthetic construct
QTRRVPL

SEQ ID NO: 497 - protein, 7 aa, synthetic construct
QTRRVPV

SEQ ID NO: 498 - protein, 8 aa, synthetic construct
QYIVSRSA

SEQ ID NO: 499 - protein, 6 aa, synthetic construct
RALRAP

SEQ ID NO: 500 - protein, 10 aa, synthetic construct
REPFMKSLPW

SEQ ID NO: 501 - protein, 6 aa, synthetic construct
RFPLKV

SEQ ID NO: 502 - protein, 10 aa, synthetic construct
RFPSLKSFPL

SEQ ID NO: 503 - protein, 7 aa, synthetic construct
RFPYGVW

SEQ ID NO: 504 - protein, 8 aa, synthetic construct
RFYRNQFF

SEQ ID NO: 505 - protein, 4 aa, synthetic construct
RGPA

SEQ ID NO: 506 - protein, 8 aa, synthetic construct
RGPAFNPM

SEQ ID NO: 507 - protein, 8 aa, synthetic construct
RGPATPIM

SEQ ID NO: 508 - protein, 7 aa, synthetic construct
RGPKLYW

SEQ ID NO: 509 - protein, 6 aa, synthetic construct
RHLAKL

SEQ ID NO: 510 - protein, 8 aa, synthetic construct
RIGRSDNH

SEQ ID NO: 511 - protein, 8 aa, synthetic construct
RKMPNITV

SEQ ID NO: 512 - protein, 14 aa, synthetic construct
RKSSIIIRMRDVVL

SEQ ID NO: 513 - protein, 8 aa, synthetic construct
RKTVQHWW

SEQ ID NO: 514 - protein, 8 aa, synthetic construct
RLGRSDNN

SEQ ID NO: 515 - protein, 8 aa, synthetic construct
RMHLRSLG

SEQ ID NO: 516 - protein, 8 aa, synthetic construct
RPLARAGI

SEQ ID NO: 517 - protein, 8 aa, synthetic construct
RPLARAGL

SEQ ID NO: 518 - protein, 12 aa, synthetic construct
RPLNGRSDNHKA

SEQ ID NO: 519 - protein, 12 aa, synthetic construct
RPLNGRSDNHKK

SEQ ID NO: 520 - protein, 12 aa, synthetic construct
RPLNGRSDNHKR

SEQ ID NO: 521 - protein, 12 aa, synthetic construct
RPLNGRSDNHQA

SEQ ID NO: 522 - protein, 12 aa, synthetic construct
RPLNGRSDNHQK

SEQ ID NO: 523 - protein, 12 aa, synthetic construct
RPLNGRSDNHQR

SEQ ID NO: 524 - protein, 12 aa, synthetic construct
RPLNGRSDNHRA

SEQ ID NO: 525 - protein, 12 aa, synthetic construct
RPLNGRSDNHRK

SEQ ID NO: 526 - protein, 12 aa, synthetic construct
RPLNGRSDNHRR

SEQ ID NO: 527 - protein, 12 aa, synthetic construct
RPLSGRSDNHKA

SEQ ID NO: 528 - protein, 12 aa, synthetic construct
RPLSGRSDNHKK

SEQ ID NO: 529 - protein, 12 aa, synthetic construct
RPLSGRSDNHKR

SEQ ID NO: 530 - protein, 12 aa, synthetic construct
RPLSGRSDNHQA

SEQ ID NO: 531 - protein, 12 aa, synthetic construct
RPLSGRSDNHQK

SEQ ID NO: 532 - protein, 12 aa, synthetic construct
RPLSGRSDNHQR

SEQ ID NO: 533 - protein, 12 aa, synthetic construct
RPLSGRSDNHRA

SEQ ID NO: 534 - protein, 12 aa, synthetic construct
RPLSGRSDNHRK

SEQ ID NO: 535 - protein, 12 aa, synthetic construct
RPLSGRSDNHRR

SEQ ID NO: 536 - protein, 8 aa, synthetic construct
RPSPMWAY

SEQ ID NO: 537 - protein, 8 aa, synthetic construct
RRHDGLRA

SEQ ID NO: 538 - protein, 8 aa, synthetic construct
RRHDGLRS

SEQ ID NO: 539 - protein, 8 aa, synthetic construct
RSLVFAPI

SEQ ID NO: 540 - protein, 8 aa, synthetic construct
RSPSRLKC

SEQ ID NO: 541 - protein, 10 aa, synthetic construct
RVPKVKVMLD

SEQ ID NO: 542 - protein, 8 aa, synthetic construct
SAGFSLPA

SEQ ID NO: 543 - protein, 8 aa, synthetic construct
SAPAVESE

SEQ ID NO: 544 - protein, 10 aa, synthetic construct
SAPYFRMMDM

SEQ ID NO: 545 - protein, 8 aa, synthetic construct
SARGPSRW

SEQ ID NO: 546 - protein, 8 aa, synthetic construct
SCGPPLGR

SEQ ID NO: 547 - protein, 8 aa, synthetic construct
SCSPPLGR

SEQ ID NO: 548 - protein, 8 aa, synthetic construct
SGGPLGVR

SEQ ID NO: 549 - protein, 8 aa, synthetic construct
SGGPPLGR

SEQ ID NO: 550 - protein, 7 aa, synthetic construct
SGPPLGR

SEQ ID NO: 551 - protein, 4 aa, synthetic construct
SGRS

SEQ ID NO: 552 - protein, 5 aa, synthetic construct
SGRSA

SEQ ID NO: 553 - protein, 6 aa, synthetic construct
SGRSAN

SEQ ID NO: 554 - protein, 7 aa, synthetic construct
SGRSANI

SEQ ID NO: 555 - protein, 7 aa, synthetic construct
SGRSANP

SEQ ID NO: 556 - protein, 9 aa, synthetic construct
SGRSANPRG

SEQ ID NO: 557 - protein, 5 aa, synthetic construct
SGRSD

SEQ ID NO: 558 - protein, 6 aa, synthetic construct
SGRSDD

SEQ ID NO: 559 - protein, 7 aa, synthetic construct
SGRSDDH

SEQ ID NO: 560 - protein, 6 aa, synthetic construct
SGRSDI

SEQ ID NO: 561 - protein, 7 aa, synthetic construct
SGRSDIH

SEQ ID NO: 562 - protein, 6 aa, synthetic construct
SGRSDN

SEQ ID NO: 563 - protein, 7 aa, synthetic construct
SGRSDNI

SEQ ID NO: 564 - protein, 7 aa, synthetic construct
SGRSDNP

SEQ ID NO: 565 - protein, 6 aa, synthetic construct
SGRSDQ

SEQ ID NO: 566 - protein, 7 aa, synthetic construct
SGRSDQH

SEQ ID NO: 567 - protein, 6 aa, synthetic construct
SGRSDT

SEQ ID NO: 568 - protein, 7 aa, synthetic construct
SGRSDTH

SEQ ID NO: 569 - protein, 6 aa, synthetic construct
SGRSDY

SEQ ID NO: 570 - protein, 7 aa, synthetic construct
SGRSDYH

SEQ ID NO: 571 - protein, 5 aa, synthetic construct
SGRSG

SEQ ID NO: 572 - protein, 6 aa, synthetic construct
SGRSGN

SEQ ID NO: 573 - protein, 7 aa, synthetic construct
SGRSGNH

SEQ ID NO: 574 - protein, 8 aa, synthetic construct
SGSPPLGR

SEQ ID NO: 575 - protein, 8 aa, synthetic construct
SIARSDNL

SEQ ID NO: 576 - protein, 14 aa, synthetic construct
SISSGLLSGRSDNI

SEQ ID NO: 577 - protein, 6 aa, synthetic construct
SNPFKY

SEQ ID NO: 578 - protein, 8 aa, synthetic construct
SPLPLRVP

SEQ ID NO: 579 - protein, 8 aa, synthetic construct
SPLPLRVP

SEQ ID NO: 580 - protein, 8 aa, synthetic construct
SPLTGRSG

SEQ ID NO: 581 - protein, 6 aa, synthetic construct
SPPLGR

SEQ ID NO: 582 - protein, 5 aa, synthetic construct
SRRVP

SEQ ID NO: 583 - protein, 6 aa, synthetic construct
SRRVPL

SEQ ID NO: 584 - protein, 6 aa, synthetic construct
SRRVPV

SEQ ID NO: 585 - protein, 8 aa, synthetic construct
SSGPPLGR

SEQ ID NO: 586 - protein, 7 aa, synthetic construct
SSPPLGR

SEQ ID NO: 587 - protein, 8 aa, synthetic construct
SSRGPAYL

SEQ ID NO: 588 - protein, 9 aa, synthetic construct
SSRHRRALD

SEQ ID NO: 589 - protein, 15 aa, synthetic construct
SSSFDKGKYKKGDDA

SEQ ID NO: 590 - protein, 15 aa, synthetic construct
SSSFDKGKYKRGDDA

SEQ ID NO: 591 - protein, 8 aa, synthetic construct
SSSPPLGR

SEQ ID NO: 592 - protein, 8 aa, synthetic construct
STFPFGMF

SEQ ID NO: 593 - protein, 6 aa, synthetic construct
STVFHM

SEQ ID NO: 594 - protein, 6 aa, synthetic construct
SVHHLI

SEQ ID NO: 595 - protein, 8 aa, synthetic construct
SVSGLLSH

SEQ ID NO: 596 - protein, 8 aa, synthetic construct
SVSGLLSS

SEQ ID NO: 597 - protein, 8 aa, synthetic construct
SVSGLRSH

SEQ ID NO: 598 - protein, 8 aa, synthetic construct
SVSGLRSS

SEQ ID NO: 599 - protein, 4 aa, synthetic construct
TARG

SEQ ID NO: 600 - protein, 5 aa, synthetic construct
TARGP

SEQ ID NO: 601 - protein, 12 aa, synthetic construct
TARGPALAHGLF

SEQ ID NO: 602 - protein, 6 aa, synthetic construct
TARGPS

SEQ ID NO: 603 - protein, 7 aa, synthetic construct
TARGPSF

SEQ ID NO: 604 - protein, 8 aa, synthetic construct
TARGPSFK

SEQ ID NO: 605 - protein, 7 aa, synthetic construct
TARGPSW

SEQ ID NO: 606 - protein, 7 aa, synthetic construct
TARGPSW

SEQ ID NO: 607 - protein, 10 aa, synthetic construct
TARGPVPRQV

SEQ ID NO: 608 - protein, 4 aa, synthetic construct
TFVH

SEQ ID NO: 609 - protein, 12 aa, synthetic construct
TGLSGRSVTQTS

SEQ ID NO: 610 - protein, 8 aa, synthetic construct
TGRGPSWV

SEQ ID NO: 611 - protein, 12 aa, synthetic construct
TLRLGRSDNNKN

SEQ ID NO: 612 - protein, 8 aa, synthetic construct
TLSGLRSP

SEQ ID NO: 613 - protein, 8 aa, synthetic construct
TSGRSANP

SEQ ID NO: 614 - protein, 8 aa, synthetic construct
TSGRSGNP

SEQ ID NO: 615 - protein, 12 aa, synthetic construct
TSLSGRSANPRG

SEQ ID NO: 616 - protein, 12 aa, synthetic construct
TSLSGRSGNPRG

SEQ ID NO: 617 - protein, 8 aa, synthetic construct
TSSGLRSP

SEQ ID NO: 618 - protein, 12 aa, synthetic construct
TSTSGRSANPRG

SEQ ID NO: 619 - protein, 22 aa, synthetic construct
TSTSGRSANPRGGGAVGLLAPP

SEQ ID NO: 620 - protein, 24 aa, synthetic construct
TSTSGRSANPRGGGVHMPLGFLGP

SEQ ID NO: 621 - protein, 12 aa, synthetic construct
TSTSGRSGNPRG

SEQ ID NO: 622 - protein, 8 aa, synthetic construct
TVSGLRSP

SEQ ID NO: 623 - protein, 8 aa, synthetic construct
VAGRSMRP

SEQ ID NO: 624 - protein, 10 aa, synthetic construct
VAPQLKSLVP

SEQ ID NO: 625 - protein, 8 aa, synthetic construct
VAQFVLTE

SEQ ID NO: 626 - protein, 10 aa, synthetic construct
VHMPLGFLGP

SEQ ID NO: 627 - protein, 20 aa, synthetic construct
VHMPLGFLGPGGLSGRSDNH

SEQ ID NO: 628 - protein, 24 aa, synthetic construct
VHMPLGFLGPGGTSTSGRSANPRG

SEQ ID NO: 629 - protein, 10 aa, synthetic construct
VLPELRSVFS

SEQ ID NO: 630 - protein, 8 aa, synthetic construct
VLSKQMSF

SEQ ID NO: 631 - protein, 7 aa, synthetic construct
VPAGRRS

SEQ ID NO: 632 - protein, 8 aa, synthetic construct
VPAGRRSL

SEQ ID NO: 633 - protein, 4 aa, synthetic construct
VPRQ

SEQ ID NO: 634 - protein, 5 aa, synthetic construct
VPRQV

SEQ ID NO: 635 - protein, 5 aa, synthetic construct
VSRSA

SEQ ID NO: 636 - protein, 8 aa, synthetic construct
VVPEGRRS

SEQ ID NO: 637 - protein, 8 aa, synthetic construct
WATPRPMR

SEQ ID NO: 638 - protein, 8 aa, synthetic construct
WDHPISLL

SEQ ID NO: 639 - skipped

SEQ ID NO: 640 - protein, 10 aa, synthetic construct
YDPZVKVVLA

SEQ ID NO: 641 - protein, 8 aa, synthetic construct
YGAGLGVV

SEQ ID NO: 642 - protein, 7 aa, synthetic construct
YIVSRSA

SEQ ID NO: 643 - protein, 8 aa, synthetic construct
YKKFVGSL

SEQ ID NO: 644 - protein, 10 aa, synthetic construct
YVPRVKALEM

SEQ ID NO: 645 - protein, 5 aa, synthetic construct
  REGION >5: note = The entire sequence of amino acids 1-5 can be repeated one or more times
GSGGS

SEQ ID NO: 646 - protein, 5 aa, synthetic construct
  REGION >5: note = The entire sequence of amino acids 1-5 can be repeated one or more times
GGGGS

SEQ ID NO: 647 - protein, 4 aa, synthetic construct
  REGION >4: note = The entire sequence of amino acids 1-4 can be repeated one or more times
GGGS

SEQ ID NO: 648 - protein, 4 aa, synthetic construct
GGSG

SEQ ID NO: 649 - protein, 5 aa, synthetic construct
GGSGG

SEQ ID NO: 650 - protein, 5 aa, synthetic construct
GSGSG

SEQ ID NO: 651 - protein, 5 aa, synthetic construct
GSGGG

SEQ ID NO: 652 - protein, 5 aa, synthetic construct
GGGSG

SEQ ID NO: 653 - protein, 5 aa, synthetic construct
GSSSG

SEQ ID NO: 654 - protein, 11 aa, synthetic construct
GSSGGSGGSGG

SEQ ID NO: 655 - protein, 4 aa, synthetic construct
GGGS

SEQ ID NO: 656 - protein, 8 aa, synthetic construct
GGGSGGGS

SEQ ID NO: 657 - protein, 12 aa, synthetic construct
GGGSGGGSGGGS

SEQ ID NO: 658 - protein, 15 aa, synthetic construct
GGGGSGGGGSGGGGS

SEQ ID NO: 659 - protein, 20 aa, synthetic construct
GGGGSGGGGSGGGGSGGGGS

SEQ ID NO: 660 - protein, 25 aa, synthetic construct
GGGGSGGGGSGGGGSGGGGSGGGGS

SEQ ID NO: 661 - protein, 10 aa, synthetic construct
GGGGSGGGGS

SEQ ID NO: 662 - protein, 5 aa, synthetic construct
GGGGS

SEQ ID NO: 663 - protein, 7 aa, synthetic construct
GGGGSGS

SEQ ID NO: 664 - protein, 17 aa, synthetic construct
GGGGSGGGGSGGGGSGS

SEQ ID NO: 665 - protein, 12 aa, synthetic construct
GGSLDPKGGGGS

SEQ ID NO: 666 - protein, 20 aa, synthetic construct
PKSCDKTHTCPPCPAPELLG

SEQ ID NO: 667 - protein, 17 aa, synthetic construct
SKYGPPCPPCPAPEFLG

SEQ ID NO: 668 - protein, 12 aa, synthetic construct
GKSSGSGSESKS

SEQ ID NO: 669 - protein, 14 aa, synthetic construct
GSTSGSGKSSEGKG

SEQ ID NO: 670 - protein, 18 aa, synthetic construct
GSTSGSGKSSEGSGSTKG

SEQ ID NO: 671 - protein, 18 aa, synthetic construct
GSTSGSGKPGSGEGSTKG

SEQ ID NO: 672 - protein, 16 aa, synthetic construct
GSTSGSGKPGSSEGST

SEQ ID NO: 673 - protein, 6 aa, synthetic construct
GSSGGS

SEQ ID NO: 674 - protein, 4 aa, synthetic construct
ESKY

SEQ ID NO: 675 - protein, 4 aa, synthetic construct
SGGG

SEQ ID NO: 676 - protein, 8 aa, synthetic construct
GPQGTAGQ

SEQ ID NO: 677 - protein, 7 aa, synthetic construct
YGAGLGW

SEQ ID NO: 678 - protein, 5 aa, synthetic construct
SGGGG